(19) United States
(12) Patent Application Publication
(10) Pub. No.: US 2024/0300937 A1
(43) Pub. Date: Sep. 12, 2024

(54) Substituted 6-(Pyrimidin-4-yl)Quinoline Compounds as Cyclin Dependent Kinase Inhibitors

(71) Applicant: BeiGene, Ltd., Camana Bay (KY)

(72) Inventors: Jing LI, Beijing (CN); Wenqing Xu, Beijing (CN); Zhiwei Wang, Beijing (CN)

(73) Assignee: BeiGene, Ltd., Camana Bay (KY)

(21) Appl. No.: 18/623,847

(22) Filed: Apr. 1, 2024

Related U.S. Application Data
(63) Continuation of application No. PCT/CN2023/091483, filed on Apr. 28, 2023.

(30) Foreign Application Priority Data
Apr. 29, 2022 (WO) PCT/CN2022/090344
Jul. 15, 2022 (WO) PCT/CN2022/105964
Mar. 30, 2023 (WO) PCT/CN2023/085071

(51) Int. Cl. C07D 413/14; A61K 31/506; A61K 31/5377; A61K 31/5386; A61K 31/553; C07D 405/14; C07D 451/14; C07D 471/10; C07D 498/18

(52) U.S. Cl. CPC C07D 413/14; A61K 31/506; A61K 31/5377; A61K 31/5386; A61K 31/553; C07D 405/14; C07D 451/14; C07D 471/10; C07D 498/18

(57) ABSTRACT

This disclosure provides compounds containing 6-(pyrimidin-4-yl)quinoline structure, the use thereof for selectively inhibiting the activity of CDK4, and pharmaceutical compositions comprising the compounds as treatment of various diseases including cancer.

### FIELD OF THE INVENTION

This disclosure provides compounds containing 6-(pyrimidin-4-yl)quinoline structure, the use thereof for selectively inhibiting the activity of cyclin-dependent kinase 4 (CDK4), and pharmaceutical compositions comprising the compounds as the treatment of various diseases including cancer.

### BACKGROUND OF THE INVENTION

Human kinase is a large group of enzymes that add phosphate groups (PO₄³⁻) to other molecules in human body [1. FASEB J. 1995 May; 9(8):576-96. 2. Enzyme Res. 2011; 2011: 794089.]. There are more than 500 kinase-encoding genes that exist in the human genome and their substrates including proteins, lipids, and nucleic acids 13. Cell Signal, 20(4 September; 16(9):983-9. 4. Cell. 2017 Aug. 10:170(4):605-635.]. Kinase mis-regulation is identified in many diseases including cancer, autoimmunity, neurological disorders, diabetes and cardiovascular disease. For example, the mutated kinases can become constitutively active and thus cause diverse cellular anomalies, leading to cancer initiation or growth. Using small molecular inhibitors to inhibit kinase activity is proved to be a successful method to treat cancer and other disease [5. Expert Rev Anticancer Ther. 2018 December; 18(12):1249-1270.]. Up to now, there are more than 70 kinase inhibitors have been approved by FDA, EMA or CDE as drugs [6. Nat Rev Drug Discov. 2018 May; 17(5):353-377.].

Protein kinase family takes a majority fraction of the kinase superfamily. For protein targets, protein kinases can phosphorylate the amino acids including serine, threonine, tyrosine and histidine. [7. Science. 2002 Dec. 6:298(5600):1912-34.] Protein kinases play a major role in cellular activation processes, through reversible phosphorylation and dephosphorylation of proteins, by the antagonistic action of kinases and phosphatases, is an important component of cell signaling because the phosphorylated and unphosphorylated states of the target protein can have different levels of activity. [8. Biochimie. 2014 December; 107 Pt B:167-87. 9. Clin Transl Oncol. 2006 March; 8(3):153-60.] Different protein kinases including EGFR, BTK, ALK, JAK, PI3K and CDK are proved to be good targets for cancer drug development.

Excessively activated cell cycle is a common feature of human cancer [10. Nat Rev Cancer. 2009 March; 9(3):153-66.]. While cyclins are among the most important core cell cycle regulators. There are four basic cyclin types found in humans including G1 cyclins, G1/S cyclins. S cyclins and M cyclins. To drive the cell cycle forward, a cyclin must activate or inactivate many target proteins inside of the cell. And these cyclins drive the events of the cell cycle majorly by partnering with a family of enzymes called the cyclin-dependent kinases (CDKs). CDK kinase itself is inactive, but binding with a cyclin can activate it, making the CDK/cyclin complex a functional holoenzyme and allowing it to modify target proteins [11. Orphanet J Rare Dis. 2020 Aug. 6:15(1):203. 12. J Mol Biol. 1999 Apr. 16; 287(5):821-8.]. There are 26 serine/threonine protein kinases that form a CDK and CDK-like branch of the CMGC subfamily of the human kinome; of these, 21 are classified as CDKs. Among all the currently identified CDKs, CDK1, CDK2, CDK4 and CDK6 are considered as the direct modulator of cell cycle majorly by phosphorylating and inactivating retinoblastoma protein and releasing E2F transcription factors, and E2F downstream pathway is critical in regulating the initiation of DNA replication. And CDK4/6 is essential for G1 early initiation and G1/S transition. [13. Cell Death Differ. 1998 February; 5(2):13240. 14. Oncogene. 2016 Sep. 15; 35(37):4829-35.]

CDK4/6 related pathway is commonly disregulated in many different cancer types such as breast cancer, lung cancer and pancreatic cancer. And there are 4 approved CDK4/6 inhibitors including palbociclib, ribociclib, abemaciclib and trilaciclib which have been approved by FDA or CDE to be used as either single agent or combo with endocrine therapy to treat HR+, Her2− breast cancer. This approach shows good efficacy in the clinic while hematopoietic toxicity like neutropenia and leukopenia which may limit the clinical application of CDK4/6 dual inhibitors. And emerging data indicating that inhibition of CDK6/Cyclin D3 may cause the clinical observed hematologic toxicity [15. Cell. 2004 Aug. 20:118(4):493-504. 16. Haematologica. 2021 Oct. 1; 106(10):2624-2632.] while CDK4/Cyclin D1 is the oncogenic driver in different cancers [17. Nat Commun. 2019 Dec. 20:10(1):5817. 18. 18. Cancer Cell. 2006 January; 9(1):23-32.]. Development of a selective CDK4 selective inhibitor might show improved efficacy, mitigated hematologic toxicity and expanded clinical usage in many cancers including but not limited to breast cancer, lung cancer, pancreatic cancer, prostate cancer, bone cancer, liver cancer and endometrial cancer.

Thus there remains great need to develop a selective CDK4 inhibitor. Here, the inventors of the instant invention found the selective CDK4 inhibitor compounds with high CDK4 selectivity all other kinases including CDK6, which potentially lead to better efficacy, improved toxicity profile and potential to overcome resistance mechanisms, and the like.

### SUMMARY OF THE INVENTION

One objective of the present invention is to provide compounds and derivatives which function to act as CDK4 inhibitors, and methods of preparation and uses thereof.
Aspect 1. A compound of formula (1):

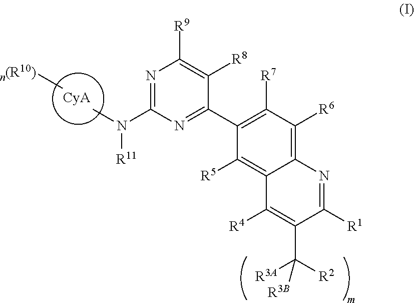

or a N-oxide thereof, or a pharmaceutically acceptable salt thereof, or a stereoisomer thereof, or a tautomer thereof, or a deuterated analog thereof, or a prodrug thereof.
wherein:
ring CyA is a 3- to 8-membered ring, said ring comprising 0, 1 or 2 heteroatom(s) independently selected from nitrogen, oxygen, or optionally oxidized sulfur as ring member(s); said ring is optionally substituted with at least one substituent R^{1d};
n is 0, 1, 2, 3, 4 or 5;
m is 0 or 1; provided that when m=0, the

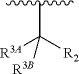

moiety as a whole is replaced with H;
R¹ is H, halogen, —C₁₋₈alkyl, —C₂₋₈alkenyl, —C₂₋₈alkynyl, cycloalkyl, haloalkyl, heterocyclyl, aryl, heteroaryl, —CN, —OR^{1a}, —COR^{1a}, —CO₂R^{1a}, —CONR^{1a}R^{1b}, —NR^{1a}R^{1b}, —NR^{1a}—COR^{1b}, —NR^{1a}CO₂R^{1b} or —NR^{1a}CONR^{1b}R^{1c}; wherein each of said —C₁₋₈alkyl, —C₂₋₈ alkenyl, —C₂₋₈alkynyl, cycloalkyl, heterocyclyl, aryl or heteroaryl is optionally substituted with at least one substituent R^{1d};
R^{1a}, R^{1b} and R^{1c} are each independently selected from hydrogen, —C₁₋₈ alkyl, cycloalkyl, heterocyclyl, aryl or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1f};
R^{1d} and R^{1f} are each independently selected from hydrogen, halogen, hydroxy, —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈ alkyl, —C₁₋₈alkoxy, -haloC₁₋₈alkoxy, —C₂₋₈alkenyl, —C₂₋₈ alkynyl, cycloalkyl, halocycloalkyl, heterocyclyl, haloheterocyclyl, aryl, haloaryl, heteroaryl or haloheteroaryl;
R² is hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{2d};
R^{2a}, R^{2b} and R^{2c} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}R^{2h}SO₂R^{2h}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, oxo, —C₁₋₈alkyl, -haloC₁₋₈ alkyl, —C₂₋₈alkoxy, -haloC₂₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl; or
(two R^{2d}) or (two R^{2f}) together with the atom(s) to which they are attached, form a 3- to 12-membered ring, said ring comprising 0, 1 or 2 heteroatom(s) independently selected from nitrogen, oxygen, or optionally oxidized sulfur as ring member(s), said ring is optionally substituted with at least one substituent selected from the group consisting of halogen, hydroxy, oxo, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{2g}, R^{2h} and R²ⁱ are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈ alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3A} and R^{3B} are each independently hydrogen, halogen, —C₁₋₈alkyl, —C₂₋₈alkenyl, —C₂₋₈alkynyl, cycloalkyl, heterocyclyl, aryl, heteroaryl or —CN; wherein each of said —C₁₋₈alkyl, —C₂₋₈alkenyl, —C₂₋₈ alkynyl, cycloalkyl, heterocyclyl, aryl or heteroaryl is optionally substituted with at least one substituent R^{3c}; or
R^{3A} and R^{3B} together with the atom to which they are attached, form acyl (—C(═O)—) or a 3- to 12-membered ring, said ring comprising 0, 1 or 2 heteroatom(s) independently selected from nitrogen, oxygen, or optionally oxidized sulfur as ring member(s), said ring is optionally substituted with at least one substituent R^{3c};
R^{3c} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{3d}, —SO₂R^{3d}, —SO₂NR^{3d}R^{3c}, —COR^{3d}, —CO₂R^{3d}, —CONR^{3d}R^{3c}, —NO₂, —NR^{3d}R^{3c}, —NR^{3d}COR^{3c}, —NR^{3d}CO₂R^{3c}, —NR^{3d}CONR^{3e}R^{3f}, or —NR^{3d}SO₂R^{3c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈alkyl, -haloC₁₋₈ alkyl, —C₂₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{3d}, R^{3e} and R^{3f} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈ alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁴ is hydrogen, halogen, —C₁₋₈alkyl, —C₂₋₈alkenyl, —C₂₋₈alkynyl, cycloalkyl or heterocyclyl; wherein each of said —C₁₋₈alkyl, —C₂₋₈alkenyl, —C₂₋₈alkynyl, cycloalkyl or heterocyclyl is optionally substituted with at least one substituent R⁴;
R^{4a} is each independently selected from hydrogen, halogen, —C₁₋₈alkyl, —C₂₋₈alkenyl, —C₂₋₈alkynyl, cycloalkyl, heterocyclyl, aryl, heteroaryl, oxo, —CN, —OR^{4b}, —SO₂R^{4b}, —SONR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent selected from the group consisting of halogen, hydroxy, —C₁₋₈ alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent selected from the group consisting of halogen, hydroxy, —C₁₋₈ alkyl, -haloC₁₋₈alkyl, —C₁₋₈alkoxy, -haloC₁₋₈ alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
R⁵, R⁶, R⁷, R⁸ and R⁹ are each independently selected from H, halogen, —C₁₋₈alkyl, —C₂₋₈alkenyl, —C₂₋₈ alkynyl, cycloalkyl, heterocyclyl, aryl, heteroaryl, —CN, —OR^{5a}, —COR^{5a}, —CO₂R^{5b}, —CONR^{5a}R^{5b}, —NR^{5a}R^{5b}, —NR^{5a}COR^{5b}, —NR^{5a}CO₂R^{5b} or —NR^{5a}CONR^{5b}R^{5c}; wherein each of said —C₁₋₈alkyl, —C₂₋₈alkenyl, —C₂₋₈ alkynyl, cycloalkyl, heterocyclyl, aryl or heteroaryl is optionally substituted with at least one substituent R^{5d};
R^{5a}, R^{5b} and R^{5c} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent R^{5f};
R^{5d} and R^{5f} are each independently selected from hydrogen, halogen, hydroxy, —C₁₋₈alkyl, -haloC₁₋₈ alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈ alkyl, -haloC₁₋₈alkyl, —C₁₋₈alkoxy, -haloC₁₋₈alkoxy, —C₂₋₈alkenyl, —C₂₋₈ alkynyl, cycloalkyl, halocycloalkyl, heterocyclyl, haloheterocyclyl, aryl, haloaryl, heteroaryl or haloheteroaryl;
R¹⁰ is selected from H, halogen, —C₁₋₈alkyl, —C₂₋₈alkenyl, —C₂₋₈alkynyl, cycloalkyl, heterocyclyl, aryl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}; wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{10d};
R^{10a}, R^{10b} and R^{10c} are each independently selected from hydrogen, —C₁₋₈alkyl, —C₂₋₈alkenyl, —C₂₋₈ alkynyl, cycloalkyl, heterocyclyl, aryl or heteroaryl, wherein each of said —C₁₋₈alkyl, —C₂₋₈alkenyl, —C₂₋₈ alkynyl, cycloalkyl, heterocyclyl, aryl or heteroaryl is optionally substituted with at least one substituent R^{10f};
R^{10d} and R^{10f} are each independently selected from hydrogen, halogen, hydroxy, —C₁₋₈ alkyl, -haloC₁₋₈ alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈ alkyl, -haloC₁₋₈alkyl, —C₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈ alkynyl, cycloalkyl, halocycloalkyl, heterocyclyl, haloheterocyclyl, aryl, haloaryl, heteroaryl or haloheteroaryl;
R¹¹ is selected from H, —C₁₋₈alkyl, —C₂₋₈alkenyl, —C₂₋₈alkynyl; wherein each of said —C₂₋₈alkyl, —C₂₋₈ alkenyl or —C₂₋₈alkynyl is optionally substituted with at least one substituent R^{11a};
R^{11a} is selected from hydrogen, halogen, hydroxy, —C₁₋₈alkyl, -haloC₁₋₈alkyl, —C₂₋₈alkoxy, —C₂₋₈ alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₁₋₈alkyl, -haloC₁₋₈alkyl, —C₁₋₈alkoxy, —C₂₋₈alkenyl, —C₂₋₈ alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl.

Aspect 2. The compound of Aspect 1, wherein the compound is selected from formula (IIa), (IIb), (IIc), (IId) or (IIe):

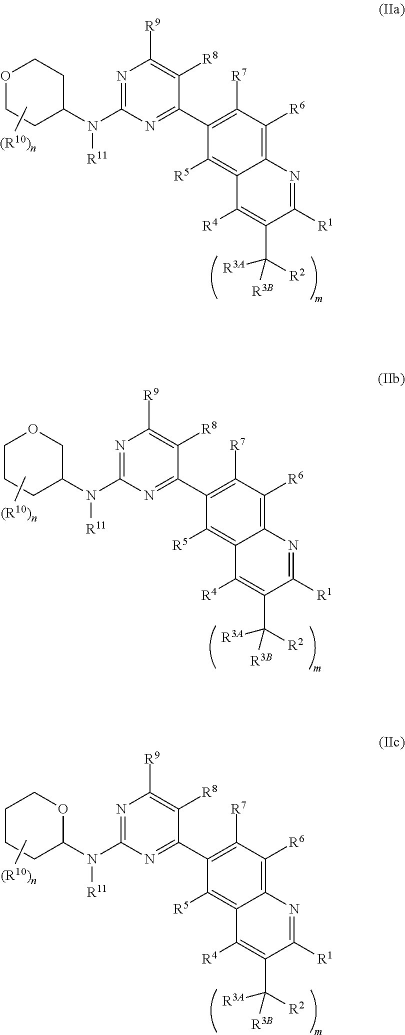

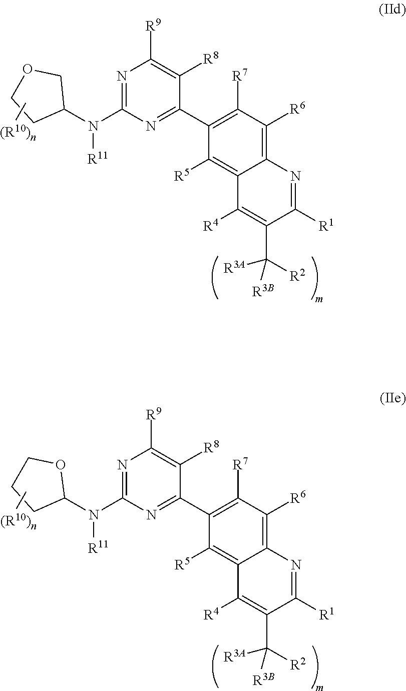

wherein, R¹, R², R^{3A}, R^{3B}, R⁴, R⁵, R⁶, R⁷, R⁸, R⁹, R¹⁰, R¹¹, m and n are each defined as Aspect 1;
preferably, the corn und is selected from formula (IIf), (IIg), (IIh) or (IIi):

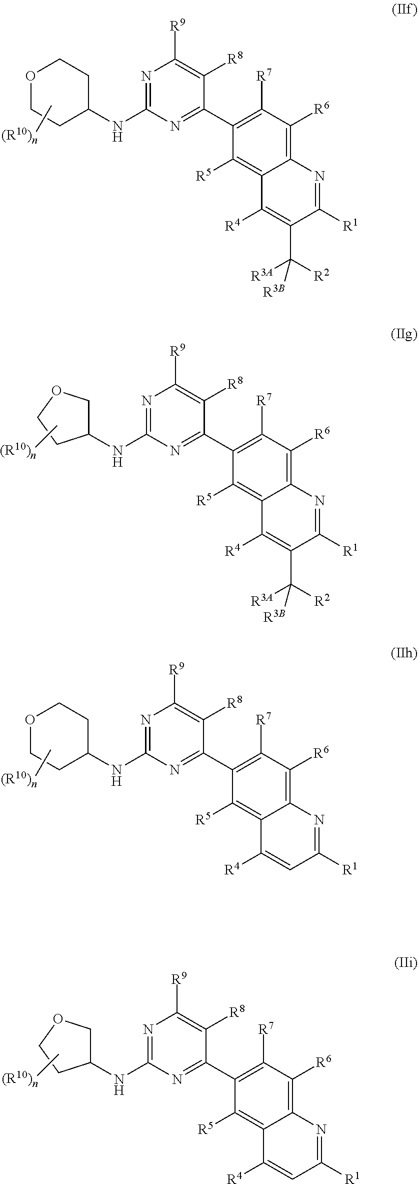

wherein, R¹, R², R^{3A}, R^{3B}, R⁴, R⁵, R⁶, R⁷, R⁸, R⁹, R¹⁰ and n are each defined as Aspect 1;
more preferably, the compound is selected from formula (IIj), (IIk), (IIl) or (IIm):

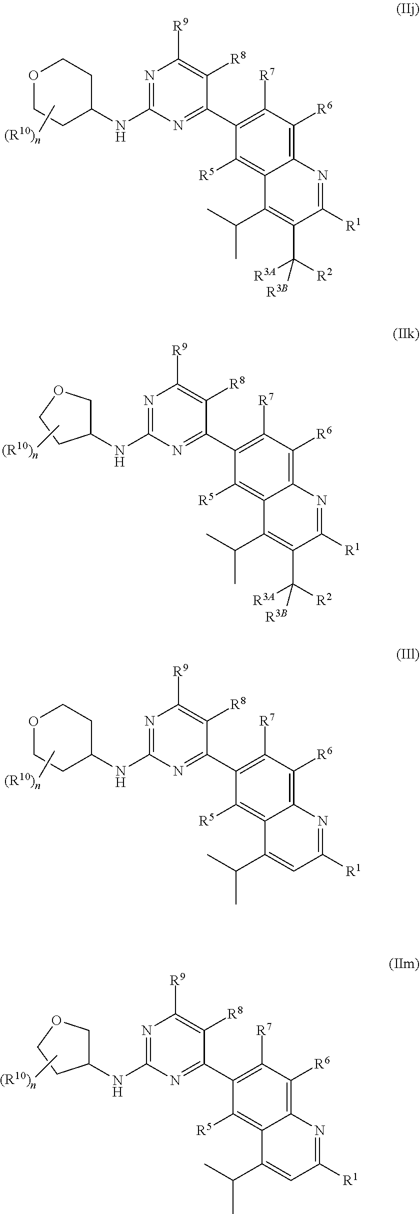

wherein, R¹, R², R³, R^{3A}, R^{3B}, R⁵, R⁶, R⁷, R⁸, R⁹, R¹⁰ and n are each defined as Aspect 1;
even more preferably, the compound is selected from formula (IIn), (IIo), (IIp) or (IIq):

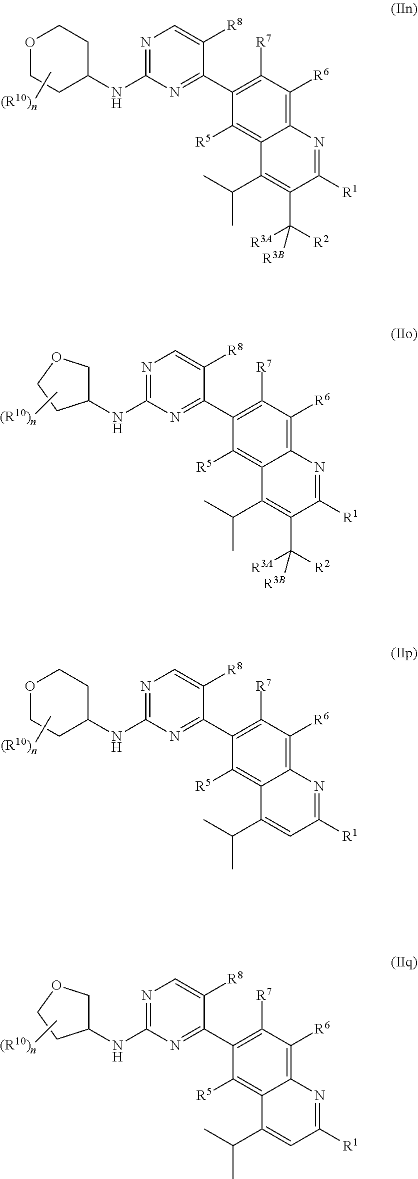

wherein, R¹, R², R^{3A}, R^{3B}, R⁵, R⁶, R⁷, R⁸, R¹⁰, m and n are each defined as Aspect 1.

Aspect 3. The compound of anyone of the preceding Aspects, wherein ring CyA is a 3-, 4-, 5-, 6-, 7- or 8-membered ring, said ring comprising 0, 1 or 2 heteroatom(s) independently selected from nitrogen, oxygen, or optionally oxidized sulfur as ring member(s); said ring is optionally substituted with 0, 1, 2, 3, 4 or 5 R¹⁰; said ring is a saturated or unsaturated ring;
preferably CyA is a 3-, 4-, 5-, 6-, 7- or 8-membered saturated ring, said ring comprising 0, 1 or 2 heteroatom(s) independently selected from nitrogen, oxygen, or optionally oxidized sulfur as ring member(s); said ring is optionally substituted with 0, 1, 2 or 3 R¹⁰;
more preferably CyA is a 5-, 6- or 7-membered saturated ring, said ring comprising 1 or 2 heteroatom(s) independently selected from nitrogen or oxygen as ring member(s); said ring is optionally substituted with 0, 1. 2 or 3 R¹⁰;
even more preferably, CyA is a ring selected from tetrahydrofuranyl or tetrahydropyranyl; said ring is optionally substituted with 0, 1, 2 or 3 R¹⁰.

Aspect 4. The compound of anyone of the preceding Aspects, wherein ring CyA is

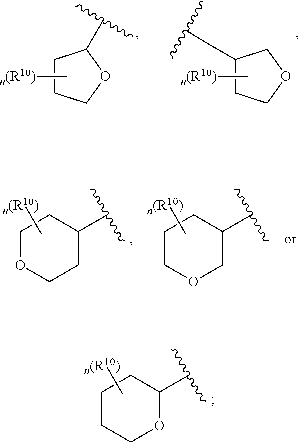

preferably, CyA is

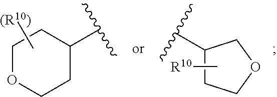

more preferably, CyA is

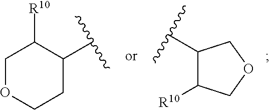

even more preferably, CyA

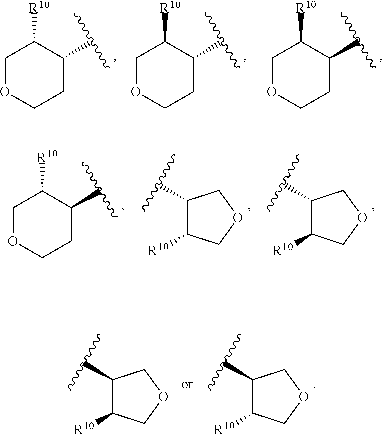

Aspect 5. The compound of anyone of the preceding Aspects, wherein R¹⁰ is selected from —H, —F, —Cl, —Br, —I, methyl, ethyl, propyl, butyl, pentyl, hexyl, heptyl, octyl, —C₂₋₈ alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{10a}, —COR^{10a}, —CO₂R^{10a}, —CONR^{10a}R^{10b}, —NR^{10a}R^{10b}, —NR^{10a}COR^{10b}, —NR^{10a}CO₂R^{10b} or —NR^{10a}CONR^{10b}R^{10c}; wherein each of said methyl, ethyl, propyl, butyl, pentyl, hexyl, heptyl, octyl, —C₂₋₈ alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10d};
R^{10a}, R^{10b} and R^{10c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{10f};
R^{10d} and R^{10f} are each independently selected from hydrogen, —F, —Cl, —Br, —I, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R¹⁰ is selected from —H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OH or —NH₂;
more preferably, R¹⁰ is —OH.

Aspect 6. The compound of anyone of the preceding Aspects, wherein the

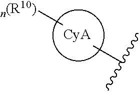

moiety is

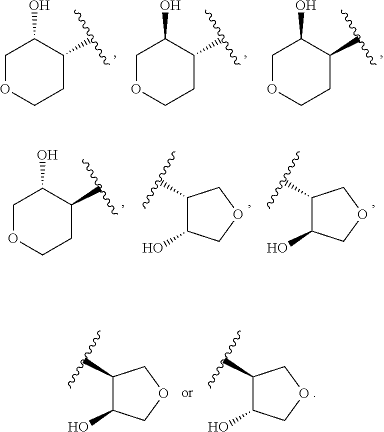

Aspect 7. The compound of anyone of the preceding Aspects, wherein R¹ is H, —F, —Cl, —Br, —I, methyl, ethyl propyl, butyl, pentyl, hexyl, heptyl, octyl —C₂₋₈alkenyl, —C₂₋₈alkynyl, cyclopropyl, cyclobutyl, cyclopentyl, cyclohexyl, cycloheptyl, cyclooctyl, haloalkyl, heterocyclyl, —CN, —OR^{1a}, —COR^{1a}, —CO₂R^{1a}, —CONR^{1a}R^{1b}, —NR^{1a}R^{1b}, —NR^{1a}COR^{1b}, —NR^{1a}CO₂R^{1b} or —NR^{1a}CONR^{1b}R^{1c} wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, haloalkyl or heterocyclyl is optionally substituted with at least one substituent R^{1d};
R^{1a}, R^{1b} and R^{1c} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, phenyl or heteroaryl is optionally substituted with at least one substituent R^{1f};
R^{1d} and R^{1f} are each independently selected from hydrogen, —F, —Cl, —Br, —I, hydroxy, methyl, ethyl, propyl, butyl, pentyl, hexyl, heptyl, octyl, -haloC₂₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₂₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl heteroaryl or haloheteroaryl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
preferably, R¹ is H, —F, —Cl, —Br, —I, methyl, ethyl, propyl, butyl, pentyl, hexyl, heptyl, octyl, —C₂₋₈ alkenyl, —C₂₋₈alkynyl, cyclopropyl, cyclobutyl, cyclopentyl, cyclohexyl, cycloheptyl, cyclooctyl, haloalkyl, heterocyclyl or —CN;
more preferably, R¹ is H, methyl, ethyl, n-propyl, iso-propyl, n-butyl, sec-butyl, iso-butyl or tert-butyl;
in another embodiment, R¹ is

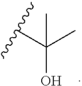 [SMILES: *C(C)(C)O]

Aspect 8. The compound of anyone of the preceding Aspects, wherein R² is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2a}, —SO₂R^{2a}, —SO₂NR^{2a}R^{2b}, —COR^{2a}, —CO₂R^{2a}, —CONR^{2a}R^{2b}, —NR^{2a}R^{2b}, —NR^{2a}COR^{2b}, —NR^{2a}CO₂R^{2b}, —NR^{2a}CONR^{2b}R^{2c}, or —NR^{2a}SO₂R^{2b}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocycyl, phenyl or heteroaryl is optionally substituted with at least one substituent R^{2d};
R^{2a}, R^{2b} and R^{2c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{2g}, —SO₂R^{2g}, —SO₂NR^{2g}R^{2h}, —COR^{2g}, —CO₂R^{2g}, —CONR^{2g}R^{2h}, —NO₂, —NR^{2g}R^{2h}, —NR^{2g}COR^{2h}, —NR^{2g}CO₂R^{2h}, —NR^{2g}CONR^{2h}R²ⁱ, or —NR^{2g}SO₂R^{2h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocycyl, phenyl or heteroaryl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R^{2d}) or (two R^{2f}) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{2g}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₂₋₈alkyl, methoxy, ethoxy, propoxy, butoxy, pentoxy, hexoxy, hepthoxy, octoxy, —C₂₋₈alkenyl, —C₂₋₈ alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 9. The compound of anyone of the preceding Aspects, wherein R² is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxarepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2a}, —COR^{2a}, —CO—R^{2b}, —CONR^{2a}R^{2b}, —NR^{2a}R^{2b}, —NR^{2a}COR^{2b}; wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d};
R^{2a} and R^{2b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OR^{2g}, —SO₂R^{2g}, —COR^{2g}, —CO₂R^{2g}, —CONR^{2g}R^{2h}, —NO₂, —NR^{2g}R^{2h} or —NR^{2g}COR^{2h}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl; or
when adjacent or geminal, (two R²) or (two R²¹) together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is optionally substituted with at least one substituent —F, —Cl, —Br, —I, hydroxy, oxo, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{2f}, R^{2h} and R²ⁱ are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₂₋₈alkyl, methoxy, ethoxy, propoxy, butoxy, pentoxy, hexoxy, hepthoxy, octoxy, —C₂₋₈alkenyl, —C₂₋₈ alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl.

Aspect 10. The compound of anyone of the preceding Aspects, wherein R² is hydrogen, methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, —OR^{2a}, —NR^{2a}R^{2b} or —NR^{2a}COR^{2b}; wherein each of said methyl, ethyl, propyl, butyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2d};
R^{2a} and R^{2b} are each independently selected from hydrogen, methyl, ethyl, propyl (n-propyl or iso-propyl), butyl (n-butyl, sec-butyl, iso-butyl or tert-butyl), pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl, wherein each of said methyl, ethyl, propyl, butyl, pentyl, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl or phenyl is optionally substituted with at least one substituent R^{2f}; or
(R^{2a} and R^{2b}), (R^{2b} and R^{2c}) or (R^{2a} and R^{2c}), together with the atom(s) to which they are attached, form a 3-, 4-, 5-, 6-, 7-, 8-, 9-, 10-, 11- or 12-membered ring, said ring comprising 0, 1 or 2 heteroatom(s) independently selected from nitrogen or oxygen as ring member(s), said ring is optionally substituted with at least one substituent R^{2f};
R^{2d} and R^{2f} are each independently selected from hydrogen, —F, —Cl, —Br, —I, methyl, ethyl, propyl, butyl, pentyl, —CF₃, —CF₂H, —CFH₂, —CH₂CF₃, —CF₂CH₃, —CH₂OH, —CH(CH₃)OH, —C(CH₃)₂OH, —CH₂CH₂OH, cyclopropyl, cyclobutyl, cyclopentyl, cyclohexyl, pyrrolidinyl, morpholinyl, thiomorpholinyl, tetrahydrofuranyl, tetrahydropyranyl, oxazepanyl, oxetanyl, azetidinyl, oxa-azaspiro[4.4]nonanyl, hexahydro-1H-furo[3,4-c]pyrrolyl, octahydropyrrolo[3,4-c]pyrrolyl, diazaspiro[4.5]decanyl, oxa-azaspiro[4.5]decanyl, azabicyclo[3.3.1]nonanyl, piperidinyl, piperazinyl, oxa-azaspiro[2.5]octanyl, oxa-azabicyclo[3.1.1]heptanyl, oxa-azabicyclo[2.2.1]heptanyl, diazaspiro[5.5]undecanyl, oxa-azabicyclo[3.3.1]nonanyl, azabicyclo[3.2.1]octanyl, azabicyclo[2.1.1]hexanyl, pyridinyl, pyrimidinyl, pyrazolyl, oxa-azabicyclo[3.2.1]octanyl, phenyl, oxo, —CN, —OH, methoxy, ethoxy, propoxy, butoxy, pentoxy, hexoxy, hepthoxy, octoxy, —SO₂Me, —SO₂Et, —SO₂C₃H₂, —COMe, —COEt, —COC₃H₇, —NH₂, —NHCH₃, —N(CH₃)₂, —NHC₂H₇, —NHC₃H₇, —NHC₄H₉, —CONH₂, —CONHCH₃, —CON(CH₃)₂, —CONHC₂H₅, —CONHC₃H₇, —CONHC₄H₉.

Aspect 11. The compound of anyone of the preceding Aspects, wherein R² is —H, -Me, —OMe, —OH, —NH₂, —NHCH₃, —N(CH₃)₂, —NHCH(CH₃)₂, —NHC(CH₃)₃, —NHCOCH₃,

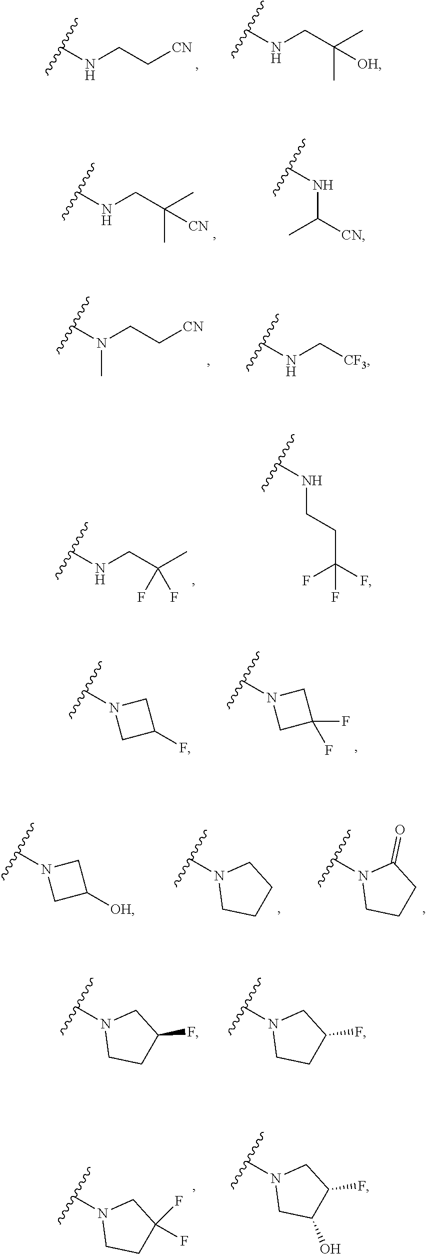

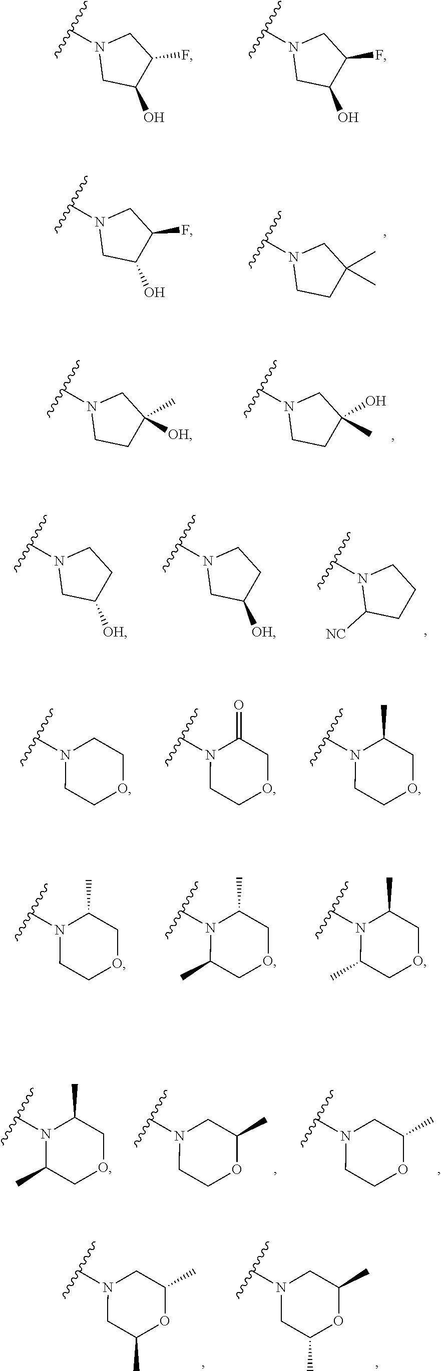

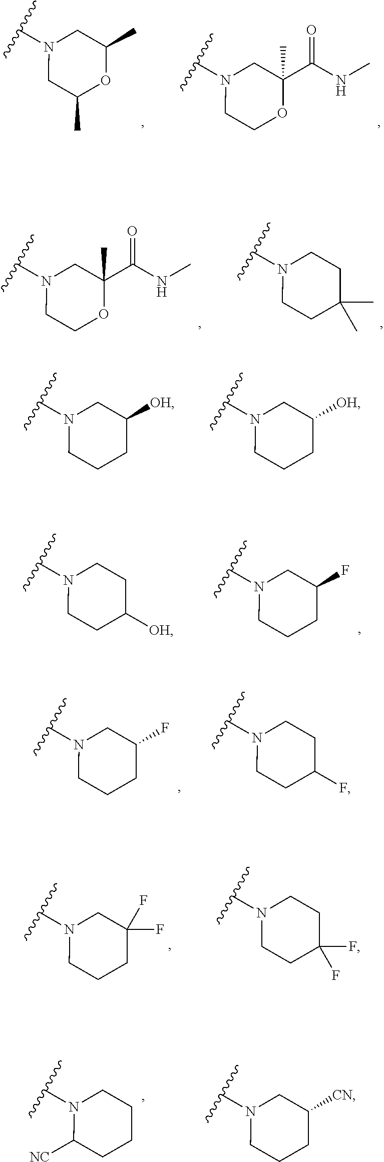

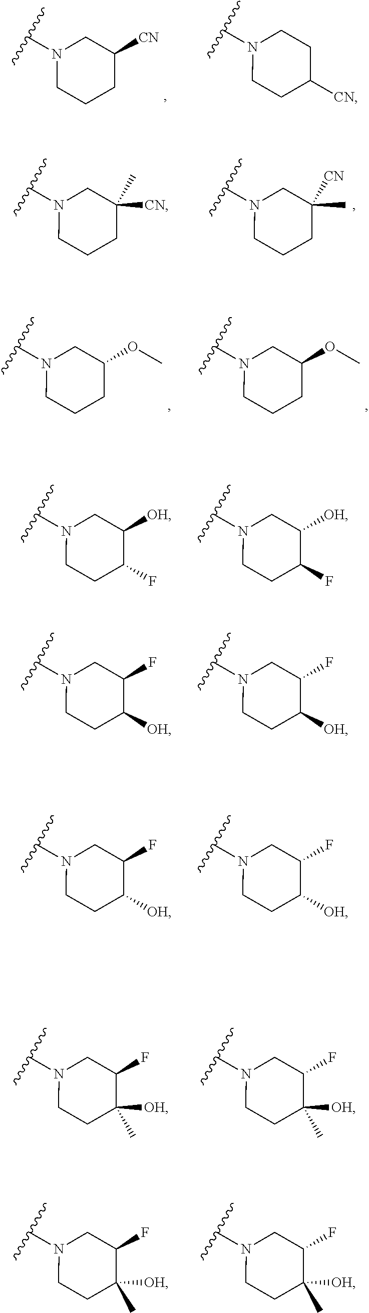

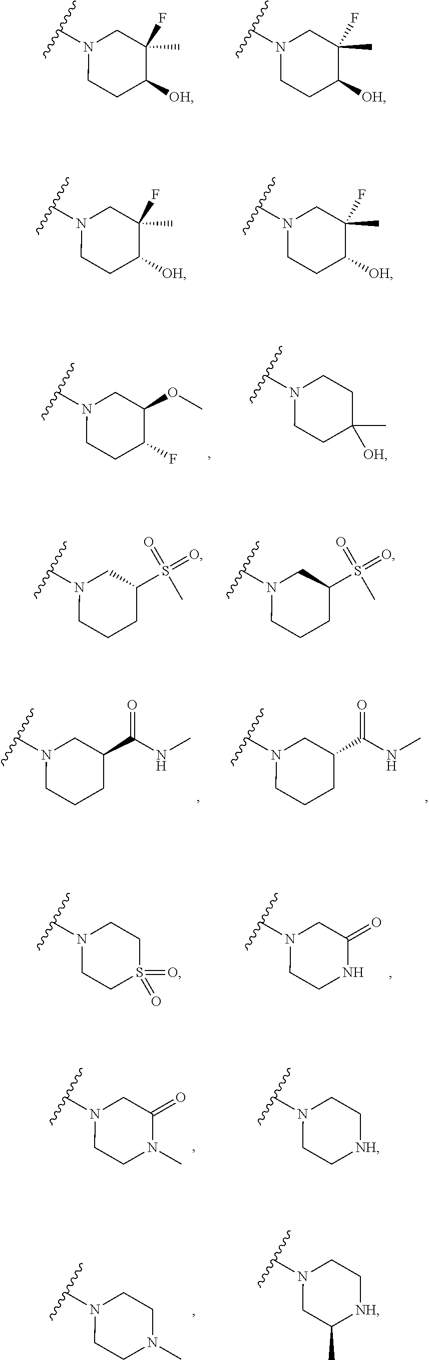

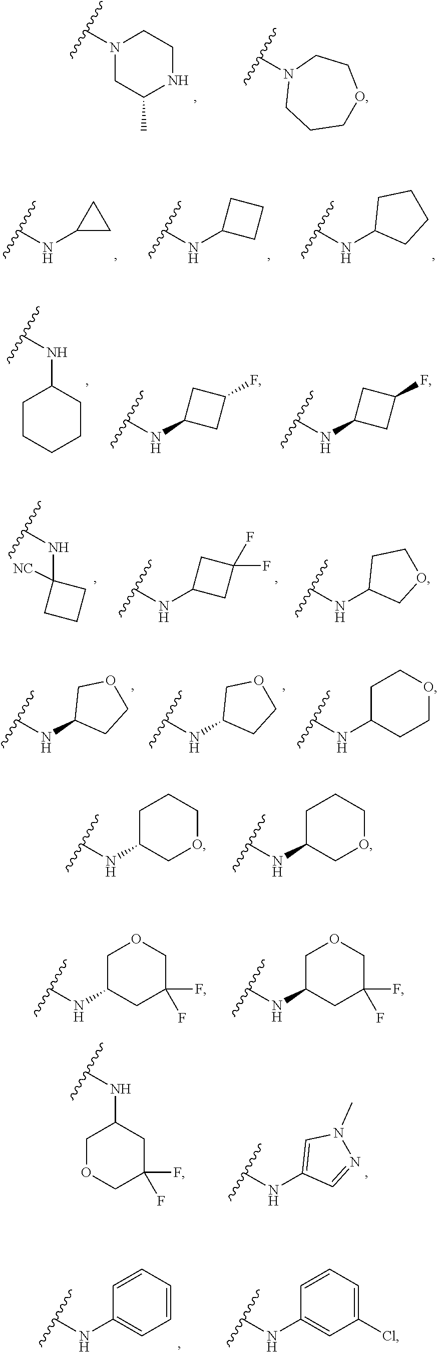

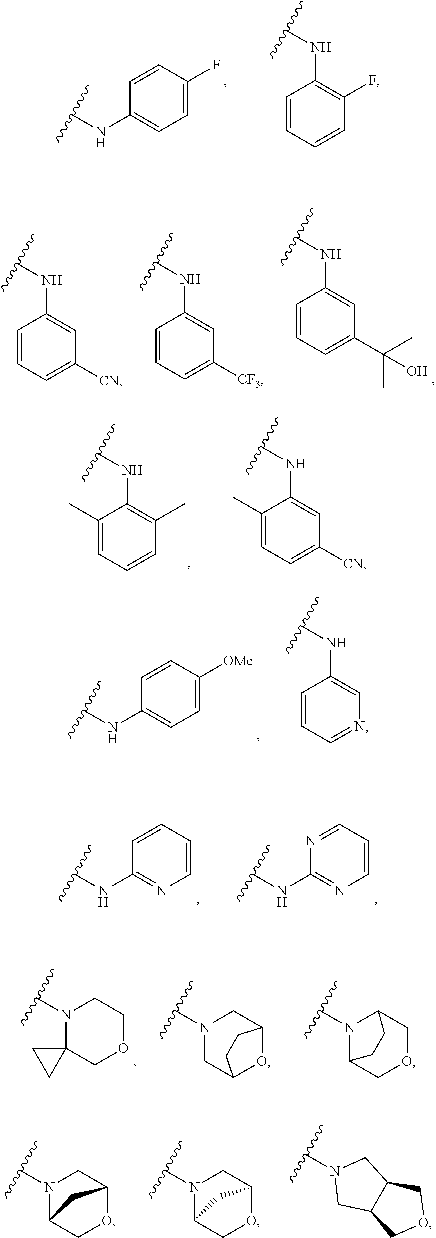

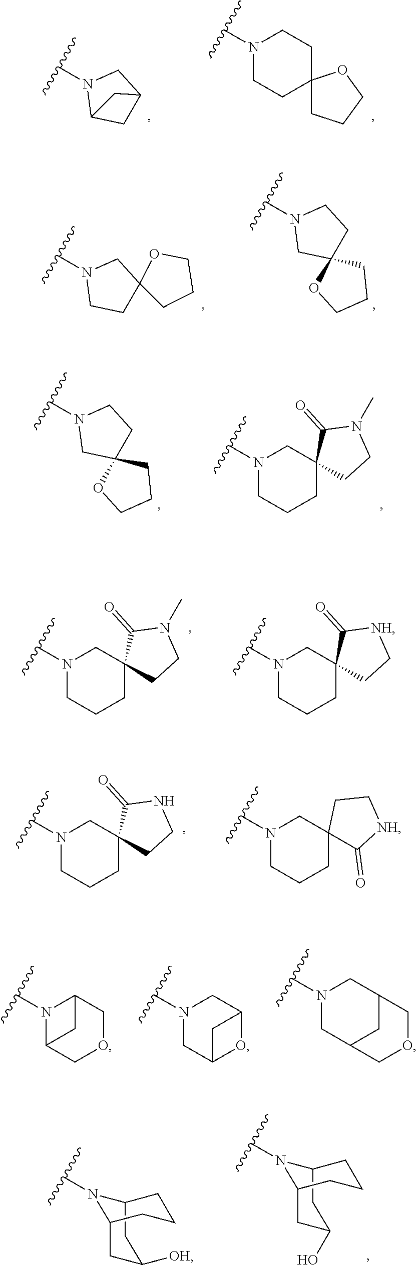

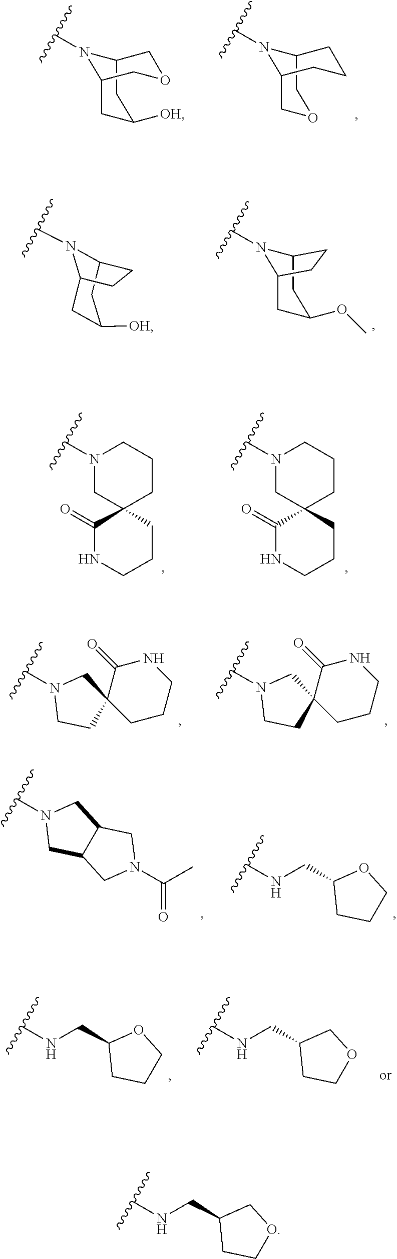

Aspect 12. The compound of anyone of the preceding Aspects-, wherein R^{3A} and R^{3B} are, each independently hydrogen, —F, —O, —Br, —I, methyl, ethyl, propyl, butyl, pentyl, hexyl, heptyl, octyl, —C₂₋₈ alkenyl, —C₂₋₈alky, cyclopropyl, cyclobutyl, cyclopentyl, cyclohexyl, cycloheptyl, cyclooctyl, heterocyclyl, phenyl, heteroaryl or —CN; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{3c}; or
R^{3A} and R^{3B} together with the atom to which they are attached, form acyl (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is mono-ring, spiro ring, fused ring or bridged ring, said ring is optionally substituted with at least one substituent R^{3c};
R^{3c} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{3d}, —SO₂R^{3d}, —SO₂NR^{3d}R^{3e}, —COR^{3d}, —CO₂R^{3d}, —CONR^{3d}R^{3e}, —NO₂, —NR^{3d}R^{3e}, —NR^{3d}COR^{3e}, —NR^{3d}CO₂R^{3e}, —NR^{3d}CONR^{3e}R^{3f}, or —NR^{3d}SO₂R^{3e}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₁₋₈alkyl, —C₂₋₈alkoxy, —C₂₋₈alkenyl, —C₂₋₈ alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{3d}, R^{3e} and R^{3f} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent —F, —Cl, —Br, —I, hydroxy, methyl, ethyl, propyl, butyl, pentyl, hexyl, heptyl, octyl, -haloC₂₋₈alkyl, methoxy, ethoxy, propoxy, butoxy, pentoxy, hexoxy, hepthoxy, octoxy, —C₂₋₈alkenyl, —C₂₋₈ alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
preferably, R^{3A} and R^{3B} are each independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl or —CN; or
R^{3A} and R^{3B} together with the atom to which they are attached, form acyl (—C(═O)—) or a 3-, 4-, 5-, 6-, 7-, 8-, 9-, 10-, 11- or 12-membered ring, said ring comprising 0, 1 or 2 heteroatom(s) independently selected from nitrogen, oxygen, or optionally oxidized sulfur as ring member(s), said ring is mono-ring, spiro ring, fused ring or bridged ring, said ring is optionally substituted with at least one substituent R^{3c};
R^{3c} is each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl, oxo, —CN, —OR, —COR^{3d}, —CO₂R^{3d}, —CONR^{3d}R^{3e}, —NO₂, —NR^{3d}R^{3e}, —NR^{3d}COR^{3e} or —SO₂R^{3d}; wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, heterocyclyl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl;
R^{3d} and R^{3e} are each independently selected from hydrogen, methyl, ethyl, propyl, butyl, pentyl, hexyl, heptyl, octyl, cyclopropyl, cyclobutyl, cyclopentyl, cyclohexyl, cycloheptyl or cyclooctyl, wherein each of said methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent —F, —Cl, —Br, —I, hydroxy, methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, aryl, haloaryl, heteroaryl or haloheteroaryl;
more preferably, R^{3A} and R^{3B} are each independently hydrogen, methyl, ethyl, propyl, butyl, pentyl; or
R^{3A} and R^{3B} together with the atom to which they are attached, form acyl (—C(═O)—) or a 3-, 4-, 5-, 6-, 7- or 8-membered ring, said ring comprising 0, 1 or 2 heteroatom(s) independently selected from nitrogen, oxygen or oxidized sulfur as ring member(s), said ring is mono-ring, spiro ring, fused ring or bridged ring, said ring is optionally substituted with at least one substituent selected from hydrogen, —F, methyl, ethyl, propyl, butyl, —CF₃, oxo or —CN.

Aspect 13. The compound of anyone of the preceding Aspects, wherein the

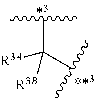

moiety is

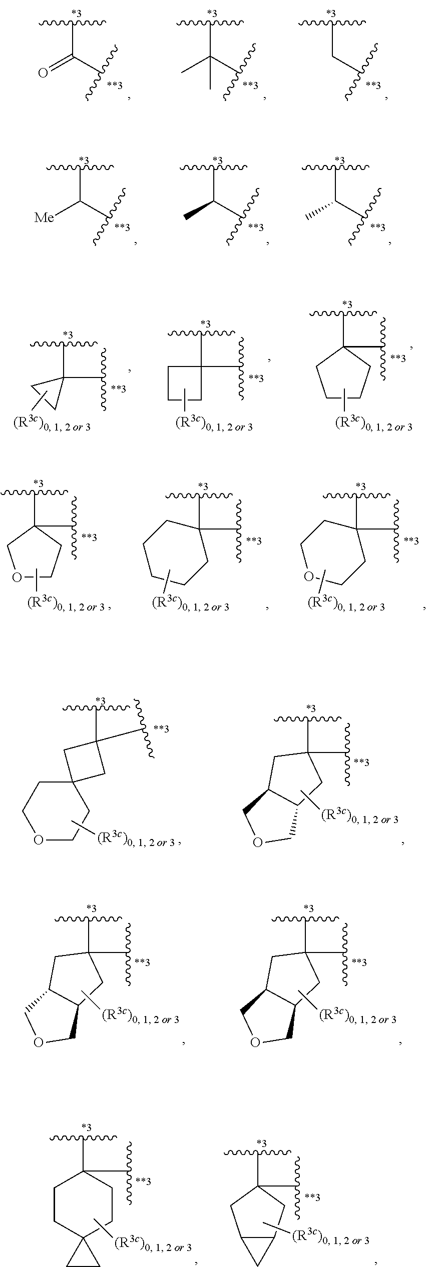

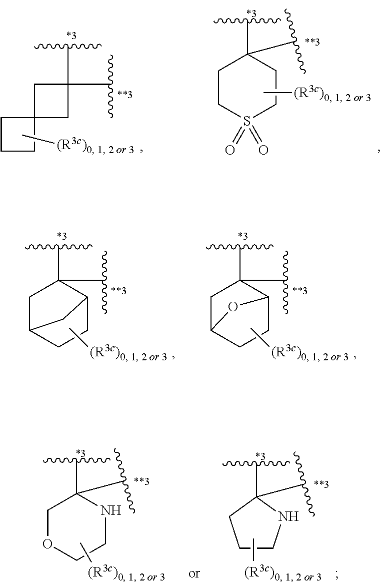

wherein *³ refers to the position attached to

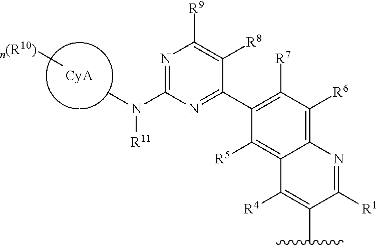

moiety, and
**³ refers to the position attached to the

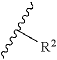

moiety;
preferably, the

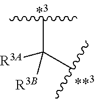

moiety is

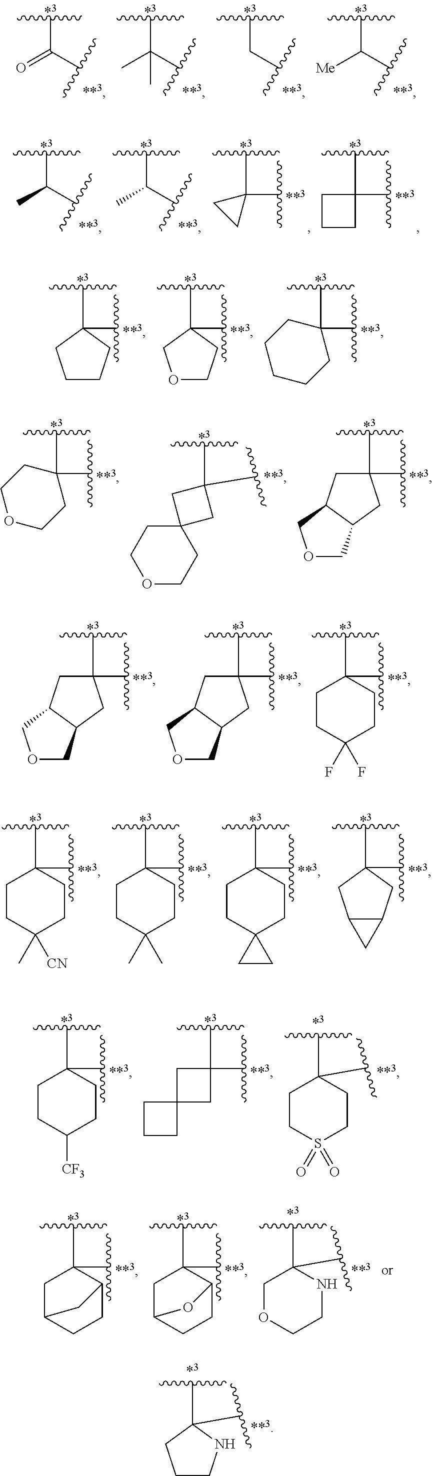

Aspect 14. The compound of anyone of the preceding Aspects, wherein the

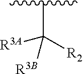

moiety is

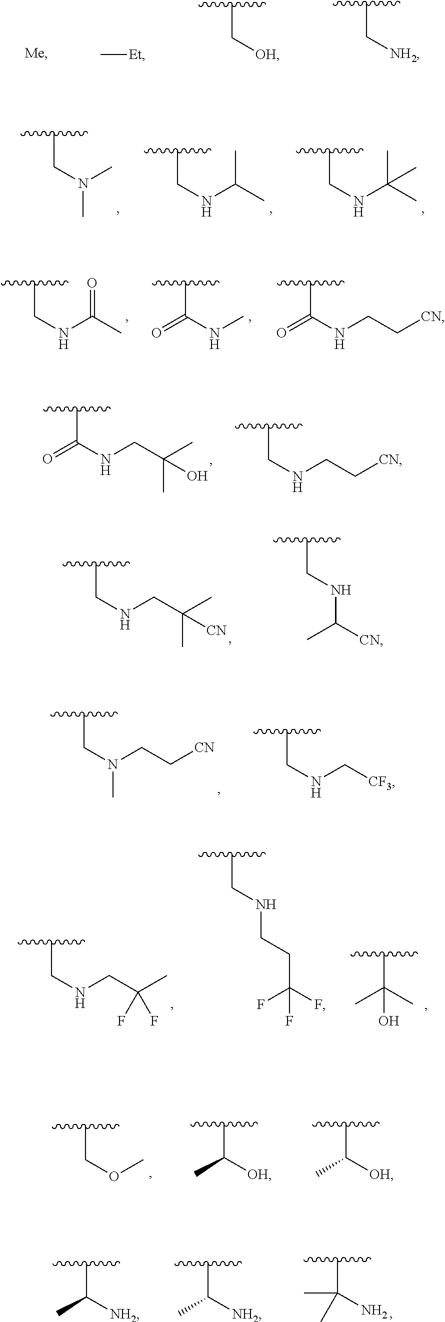

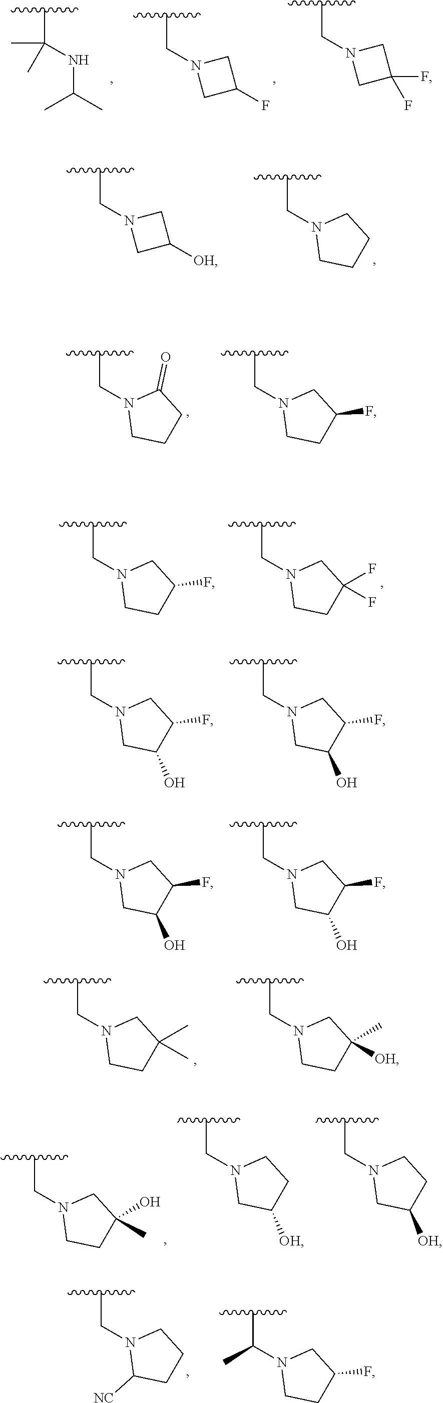

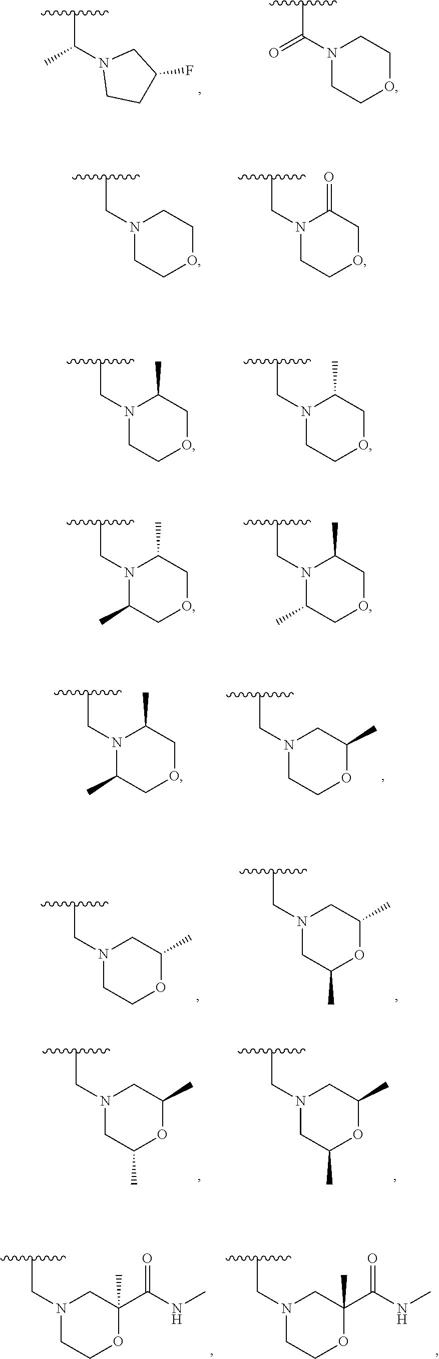

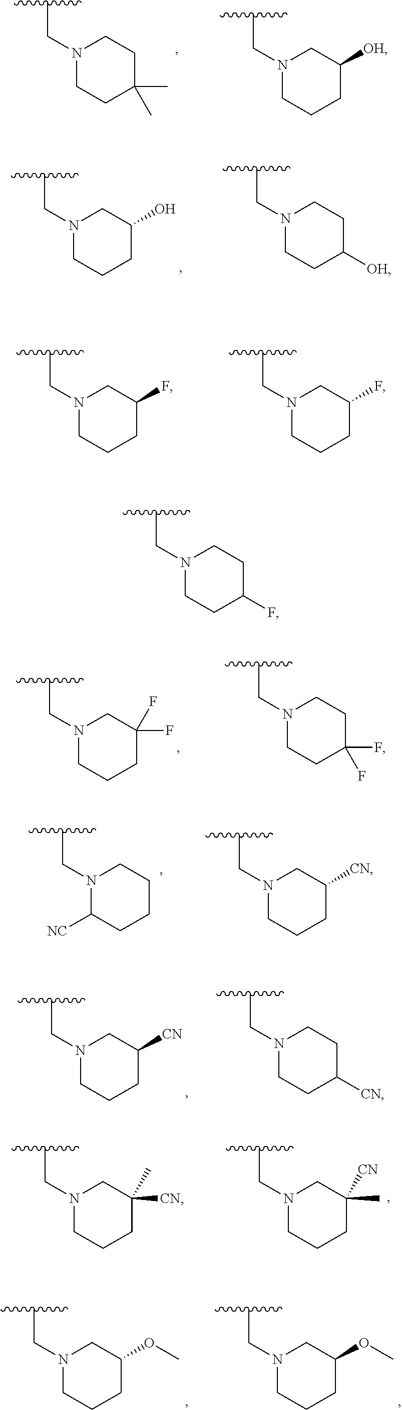

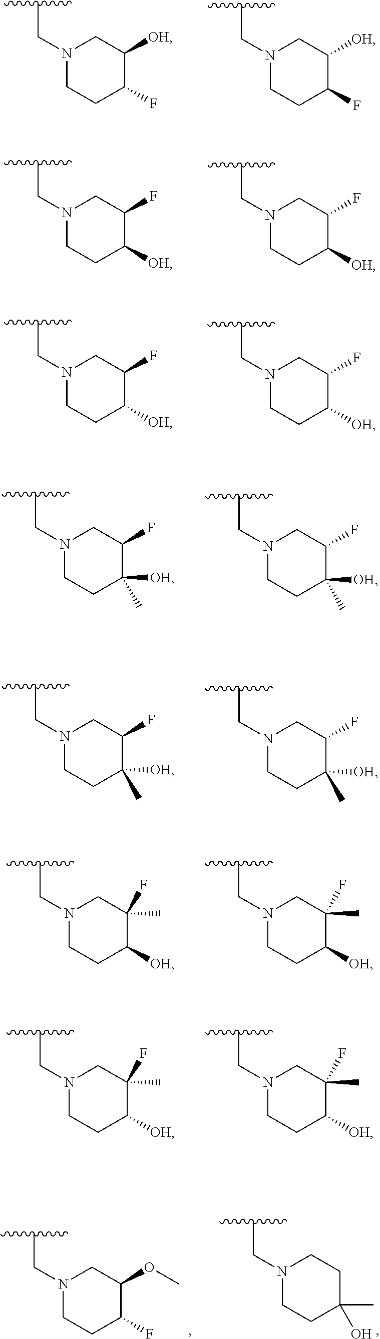

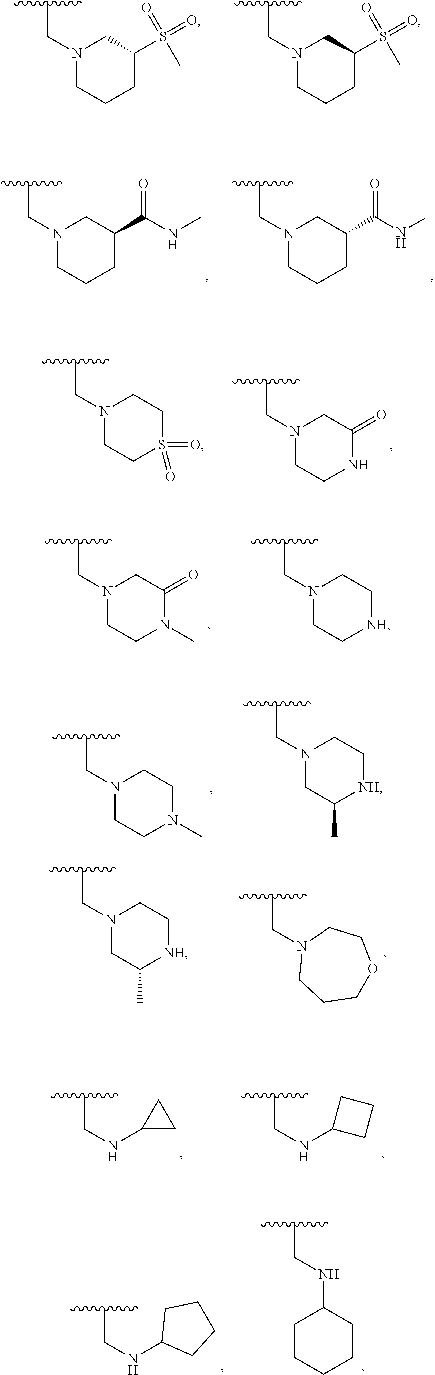

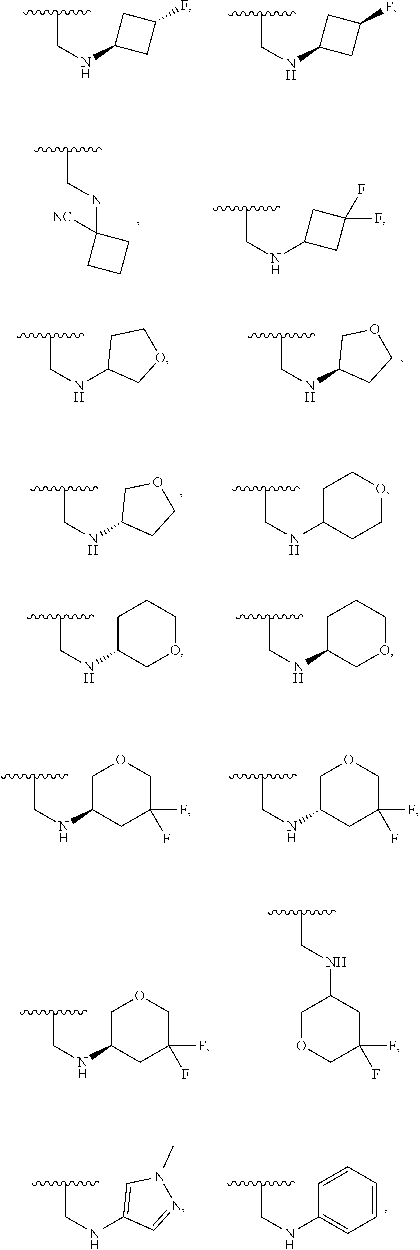

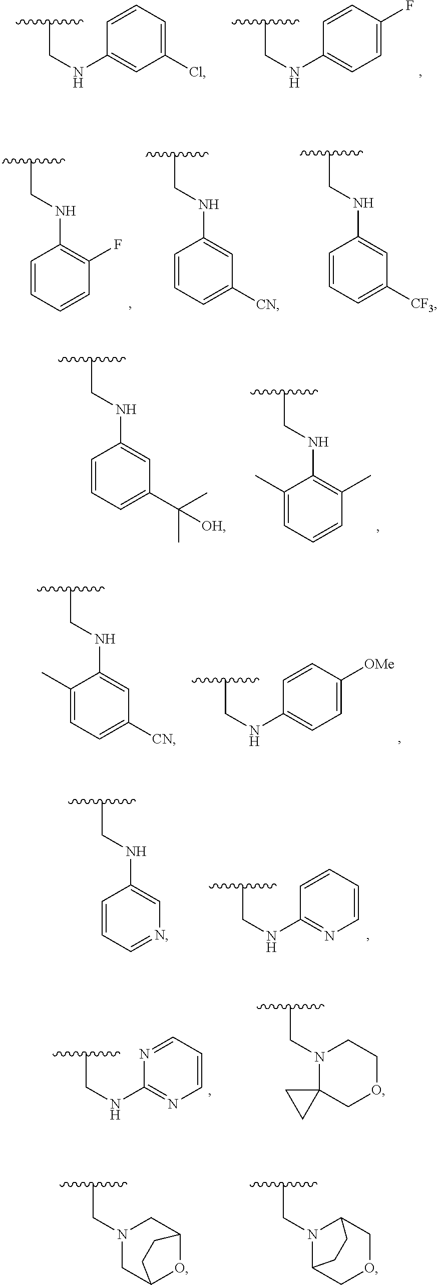

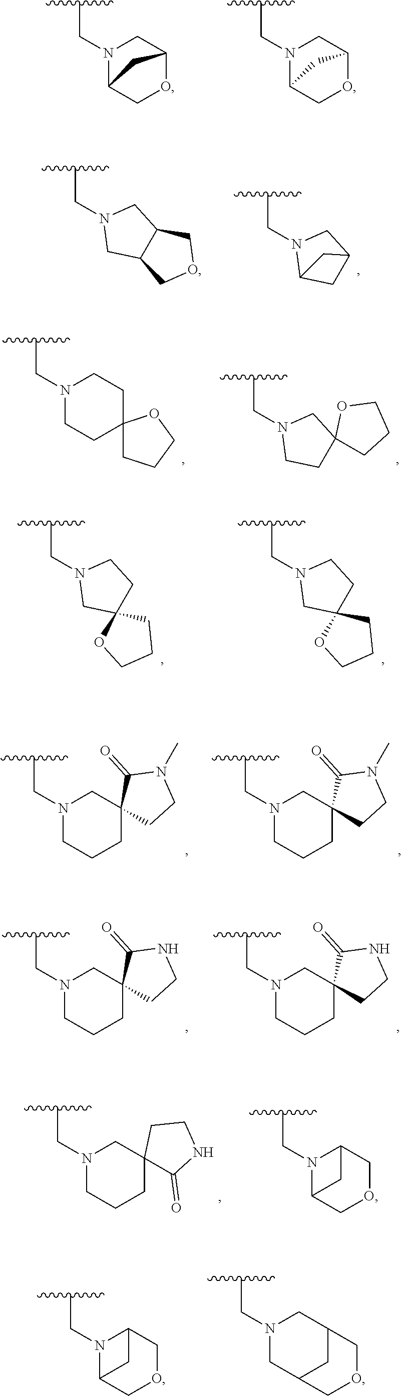

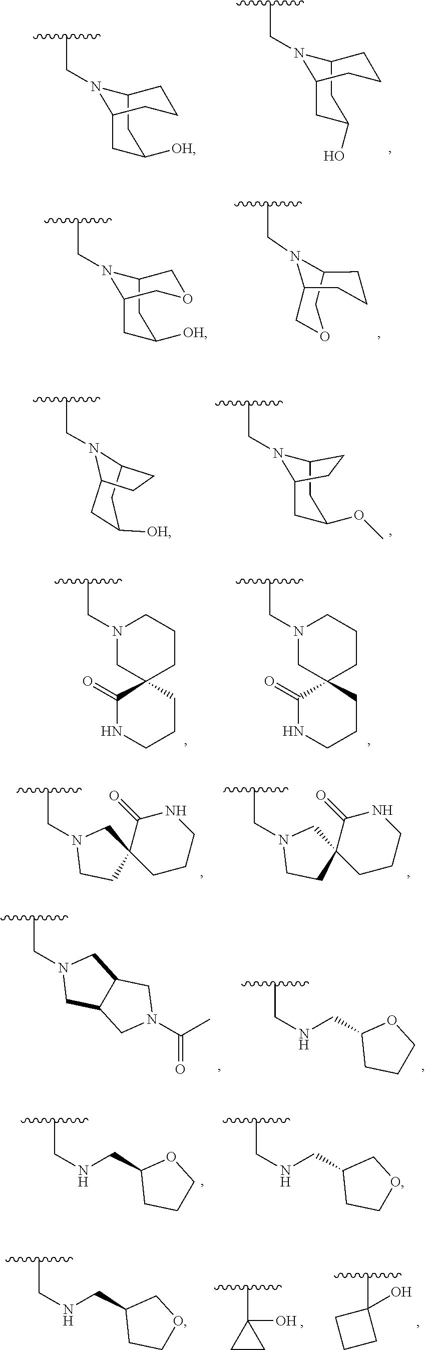

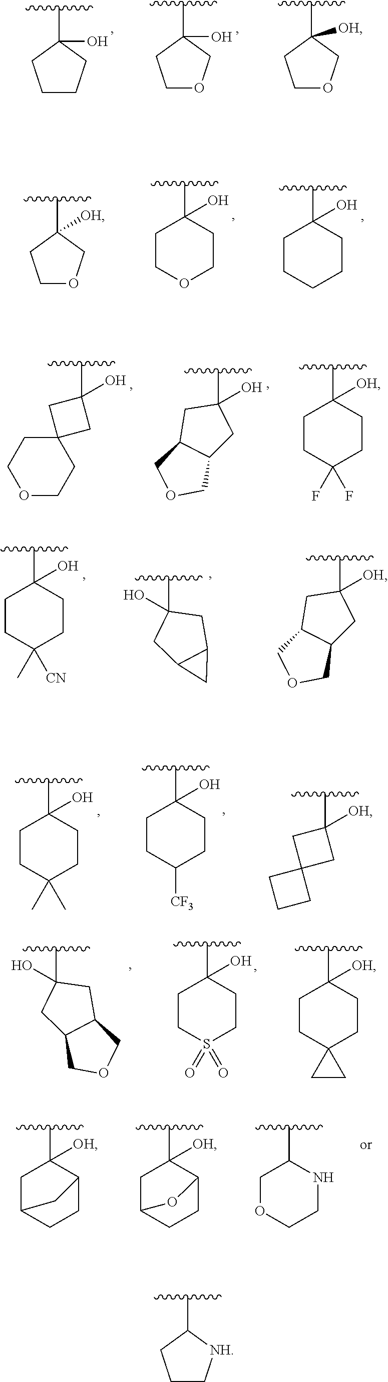

Aspect 15. The Compound of anyone of the preceding Aspects, wherein R⁴ is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl is optionally substituted with at least one substituent R^{4a};
R^{4a} is each independently selected from hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, oxo, —CN, —OR^{4b}, —SO₂R^{4b}, —SO₂NR^{4b}R^{4c}, —COR^{4b}, —CO₂R^{4b}, —CONR^{4b}R^{4c}, —NO₂, —NR^{4b}R^{4c}, —NR^{4b}COR^{4c}, —NR^{4b}CO₂R^{4c}, —NR^{4b}CONR^{4c}R^{4d} or —NR^{4b}SO₂R^{4c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈ alkynyl, cycloalkyl, halocycloalkyl, heterocyclyl, haloheterocyclyl, aryl, haloaryl, heteroaryl or haloheteroaryl;
R^{4b}, R^{4c} and R^{4d} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁴ is hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl or heterocyclyl;
more preferably, R⁴ is methyl, ethyl, n-propyl, iso-propyl, n-butyl, sec-butyl

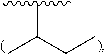 [SMILES: *C(C)CC]

iso-butyl

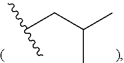 [SMILES: *CC(C)C]

tert-butyl

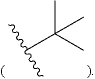 [SMILES: *C(C)(C)C]

Aspect 16. The compound of anyone of the preceding Aspects, wherein R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c}; wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5d};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl, wherein each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl is optionally substituted with at least one substituent R^{5f};
R^{5d} and R^{5f} are each independently selected from hydrogen, —F, —Cl, —Br, hydroxy, methyl, ethyl, propyl, butyl, pentyl, hexyl, heptyl, octyl, -haloC₁₋₈alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl, wherein each of said methyl, ethyl, propyl, butyl, pentyl, hexyl, heptyl, octyl, -haloC₁₋₈ alkyl, methoxy, ethoxy, propoxy, butoxy, pentoxy, hexoxy, hepthoxy, octoxy, —C₂₋₈alkenyl, —C₂₋₈alkynyl, cyclopropyl, cyclobutyl, cyclopentyl, cyclohexyl, cycloheptyl, cyclooctyl, halocycloalkyl, heterocyclyl, haloheterocyclyl, phenyl, haloaryl, heteroaryl or haloheteroaryl is optionally substituted with at least one substituent selected from the group consisting of halogen, hydroxy, —C₂₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl;
preferably, R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, —CN, —OR^{5a}, —COR^{5a}, —CO₂R^{5a}, —CONR^{5a}R^{5b}, —NR^{5a}R^{5b}, —NR^{5a}COR^{5b}, —NR^{5a}CO₂R^{5b} or —NR^{5a}CONR^{5b}R^{5c};
R^{5a}, R^{5b} and R^{5c} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl or heteroaryl;
more preferably, R⁵, R⁶, R⁷, R⁸ and R⁹ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, phenyl, heteroaryl, methoxy, ethoxy, propoxy, butoxy, pentoxy, hexoxy, hepthoxy, octoxy, —CN.

Aspect 17. The compound of anyone of the preceding Aspects, wherein R⁵, R⁶ and R⁷ are each independently selected from H, —F, —Cl, —Br, —I, methyl, ethyl, propyl, butyl, pentyl, hexyl, heptyl, octyl; and/or
R⁸ is selected from —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, methoxy, ethoxy, propoxy, butoxy, pentoxy, hexoxy, hepthoxy, octoxy, —CN; and/or
R⁹ is selected from H;
preferably, R⁵, R⁶ and R⁷ are each independently selected from H, —F, —Cl, methyl, ethyl, propyl, butyl; and/or
R⁸ is selected from —F, —Cl, methyl, ethyl, propyl, butyl, cyclopropyl, cyclobutyl, cyclopentyl, cyclohexyl, methoxy, ethoxy, propoxy, butoxy, —CN; and/or
R⁹ is selected from H.

Aspect 18. The compound of anyone of the preceding Aspects, wherein R¹¹ is selected from H, methyl, ethyl, propyl, butyl, pentyl, hexyl, heptyl, octyl, —C₂₋₈alkenyl or —C₂₋₈alkynyl; wherein each of said methyl, ethyl, propyl, butyl, pentyl, hexyl, heptyl, octyl, —C₂₋₈alkenyl or —C₂₋₈alkynyl is optionally substituted with at least one substituent R^{11a};
R^{11a} is selected from hydrogen, halogen, hydroxy, —C₁₋₈alkyl, -haloC₁₋₈alkyl, —C₁₋₈alkoxy, —C₂₋₈ alkenyl, —C₂₋₈alkynyl, cycloalkyl, halocycloalkyl, heterocyclyl, haloheterocyclyl, aryl, haloaryl, heteroaryl or haloheteroaryl, wherein each of said —C₂₋₈alkyl, -haloC₂₋₈alkyl, —C₁₋₈ alkoxy, —C₂₋₈alkenyl, —C₂₋₈ alkynyl, cycloalkyl, halocycloalkyl, heterocyclyl, haloheterocyclyl, aryl, haloaryl, heteroaryl or haloheteroaryl is optionally substituted with at least one substituent selected from the group consisting of halogen, hydroxy, —C₁₋₈alkyl, -haloC₁₋₈alkyl, —C₁₋₈alkoxy, -haloC₁₋₈alkoxy, —C₂₋₈alkenyl, —C₂₋₈alkynyl, cycloalkyl, halocycloalkyl, heterocyclyl, haloheterocyclyl, aryl, haloaryl, heteroaryl or haloheteroaryl,
preferably, R¹¹ is selected from H, methyl, ethyl, propyl, butyl, pentyl, hexyl, heptyl, octyl, —C₂₋₈alkenyl or —C₂₋₈alkynyl;
more preferably, R¹¹ is selected from H, methyl, ethyl, propyl, butyl;
even more preferably, R¹¹ is H.

Aspect 19. The compound of anyone of the preceding Aspects, wherein the compound is selected from

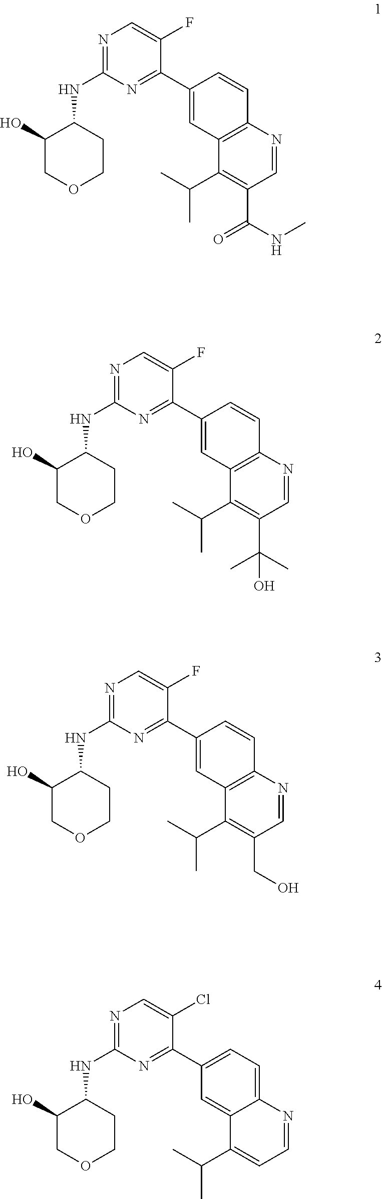 [SMILES: CNC(=O)c1cnc2ccc(-c3nc(N[C@@H]4CCOC[C@H]4O)ncc3F)cc2c1C(C)C ; CC(C)c1c(C(C)(C)O)cnc2ccc(-c3nc(N[C@@H]4CCOC[C@H]4O)ncc3F)cc12 ; CC(C)c1c(CO)cnc2ccc(-c3nc(N[C@@H]4CCOC[C@H]4O)ncc3F)cc12 ; CC(C)c1ccnc2ccc(-c3nc(N[C@@H]4CCOC[C@H]4O)ncc3Cl)cc12]

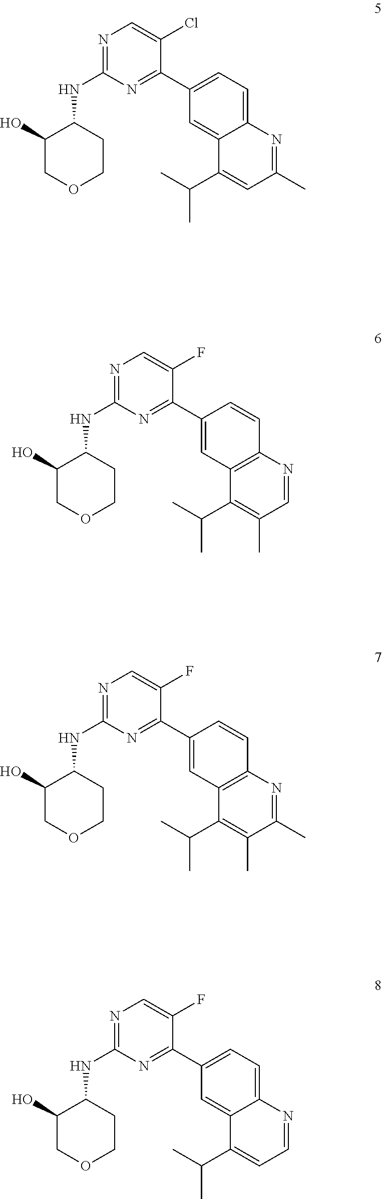 [SMILES: Cc1cc(C(C)C)c2cc(-c3nc(N[C@@H]4CCOC[C@H]4O)ncc3Cl)ccc2n1 ; Cc1cnc2ccc(-c3nc(N[C@@H]4CCOC[C@H]4O)ncc3F)cc2c1C(C)C ; Cc1nc2ccc(-c3nc(N[C@@H]4CCOC[C@H]4O)ncc3F)cc2c(C(C)C)c1C ; CC(C)c1ccnc2ccc(-c3nc(N[C@@H]4CCOC[C@H]4O)ncc3F)cc12]

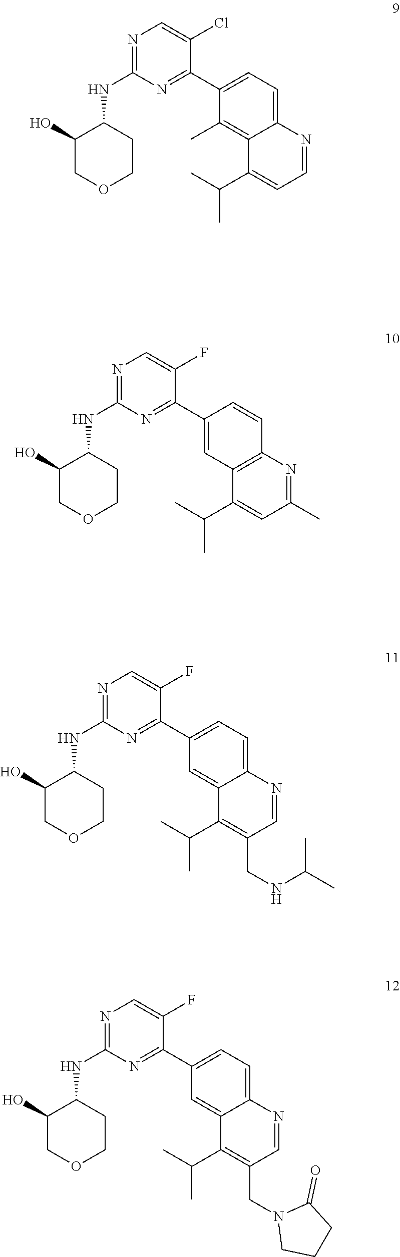 [SMILES: Cc1c(-c2nc(N[C@@H]3CCOC[C@H]3O)ncc2Cl)ccc2nccc(C(C)C)c12 ; Cc1cc(C(C)C)c2cc(-c3nc(N[C@@H]4CCOC[C@H]4O)ncc3F)ccc2n1 ; CC(C)NCc1cnc2ccc(-c3nc(N[C@@H]4CCOC[C@H]4O)ncc3F)cc2c1C(C)C ; CC(C)c1c(CN2CCCC2=O)cnc2ccc(-c3nc(N[C@@H]4CCOC[C@H]4O)ncc3F)cc12]

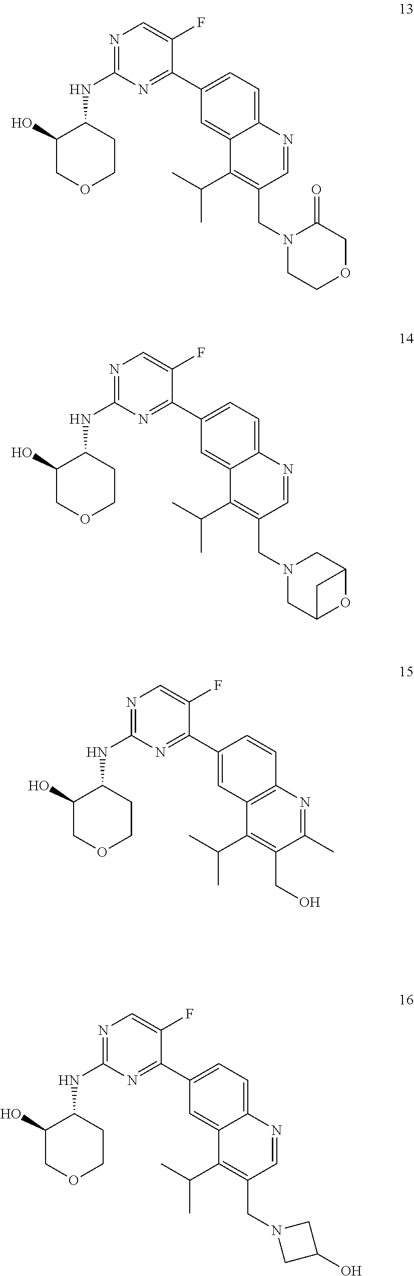 [SMILES: CC(C)c1c(CN2CCOCC2=O)cnc2ccc(-c3nc(N[C@@H]4CCOC[C@H]4O)ncc3F)cc12 ; CC(C)c1c(CN2CC3CC(C2)O3)cnc2ccc(-c3nc(N[C@@H]4CCOC[C@H]4O)ncc3F)cc12 ; Cc1nc2ccc(-c3nc(N[C@@H]4CCOC[C@H]4O)ncc3F)cc2c(C(C)C)c1CO ; CC(C)c1c(CN2CC(O)C2)cnc2ccc(-c3nc(N[C@@H]4CCOC[C@H]4O)ncc3F)cc12]

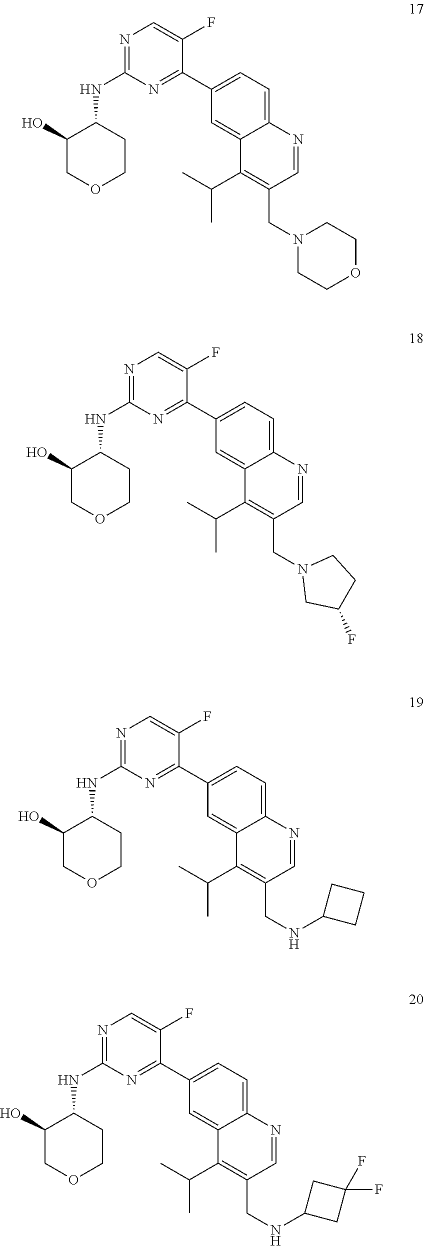 [SMILES: CC(C)c1c(CN2CCOCC2)cnc2ccc(-c3nc(N[C@@H]4CCOC[C@H]4O)ncc3F)cc12 ; CC(C)c1c(CN2CC[C@H](F)C2)cnc2ccc(-c3nc(N[C@@H]4CCOC[C@H]4O)ncc3F)cc12 ; CC(C)c1c(CNC2CCC2)cnc2ccc(-c3nc(N[C@@H]4CCOC[C@H]4O)ncc3F)cc12 ; CC(C)c1c(CNC2CC(F)(F)C2)cnc2ccc(-c3nc(N[C@@H]4CCOC[C@H]4O)ncc3F)cc12]

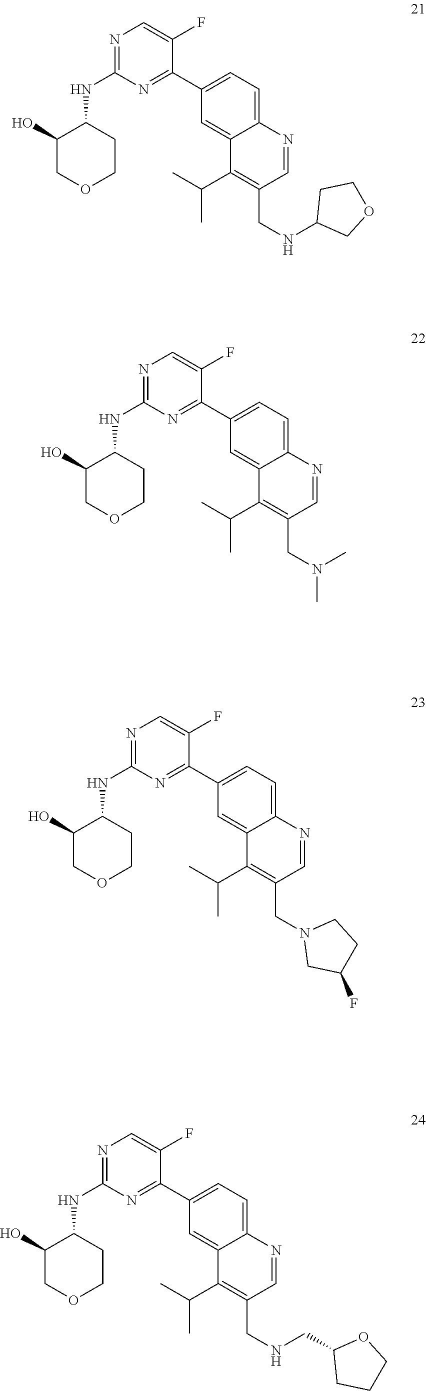 [SMILES: CC(C)c1c(CNC2CCOC2)cnc2ccc(-c3nc(N[C@@H]4CCOC[C@H]4O)ncc3F)cc12 ; CC(C)c1c(CN(C)C)cnc2ccc(-c3nc(N[C@@H]4CCOC[C@H]4O)ncc3F)cc12 ; CC(C)c1c(CN2CC[C@@H](F)C2)cnc2ccc(-c3nc(N[C@@H]4CCOC[C@H]4O)ncc3F)cc12 ; CC(C)c1c(CNC[C@H]2CCCO2)cnc2ccc(-c3nc(N[C@@H]4CCOC[C@H]4O)ncc3F)cc12]

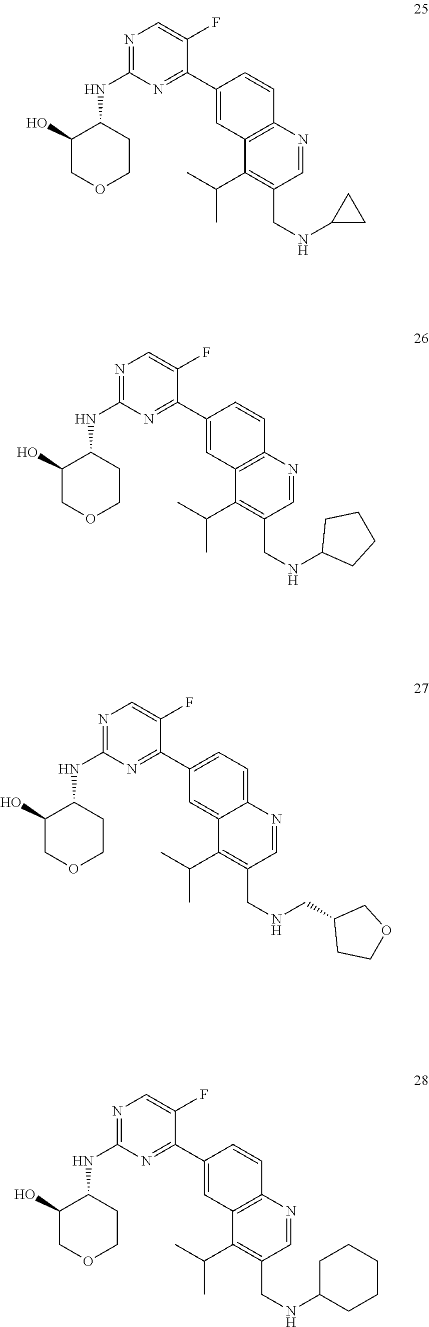 [SMILES: CC(C)c1c(CNC2CC2)cnc2ccc(-c3nc(N[C@@H]4CCOC[C@H]4O)ncc3F)cc12 ; CC(C)c1c(CNC2CCCC2)cnc2ccc(-c3nc(N[C@@H]4CCOC[C@H]4O)ncc3F)cc12 ; CC(C)c1c(CNC[C@H]2CCOC2)cnc2ccc(-c3nc(N[C@@H]4CCOC[C@H]4O)ncc3F)cc12 ; CC(C)c1c(CNC2CCCCC2)cnc2ccc(-c3nc(N[C@@H]4CCOC[C@H]4O)ncc3F)cc12]

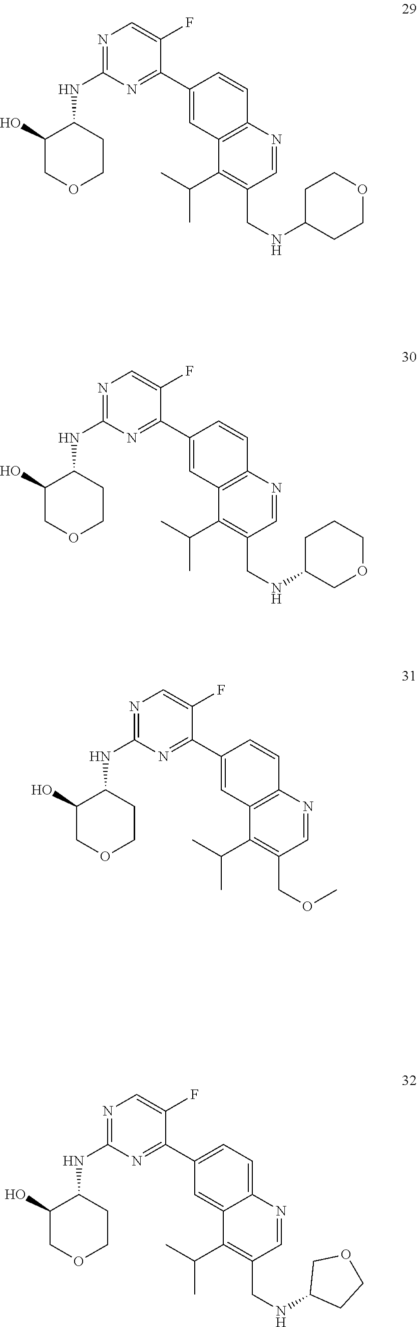 [SMILES: CC(C)c1c(CNC2CCOCC2)cnc2ccc(-c3nc(N[C@@H]4CCOC[C@H]4O)ncc3F)cc12 ; CC(C)c1c(CN[C@@H]2CCCOC2)cnc2ccc(-c3nc(N[C@@H]4CCOC[C@H]4O)ncc3F)cc12 ; COCc1cnc2ccc(-c3nc(N[C@@H]4CCOC[C@H]4O)ncc3F)cc2c1C(C)C ; CC(C)c1c(CN[C@H]2CCOC2)cnc2ccc(-c3nc(N[C@@H]4CCOC[C@H]4O)ncc3F)cc12]

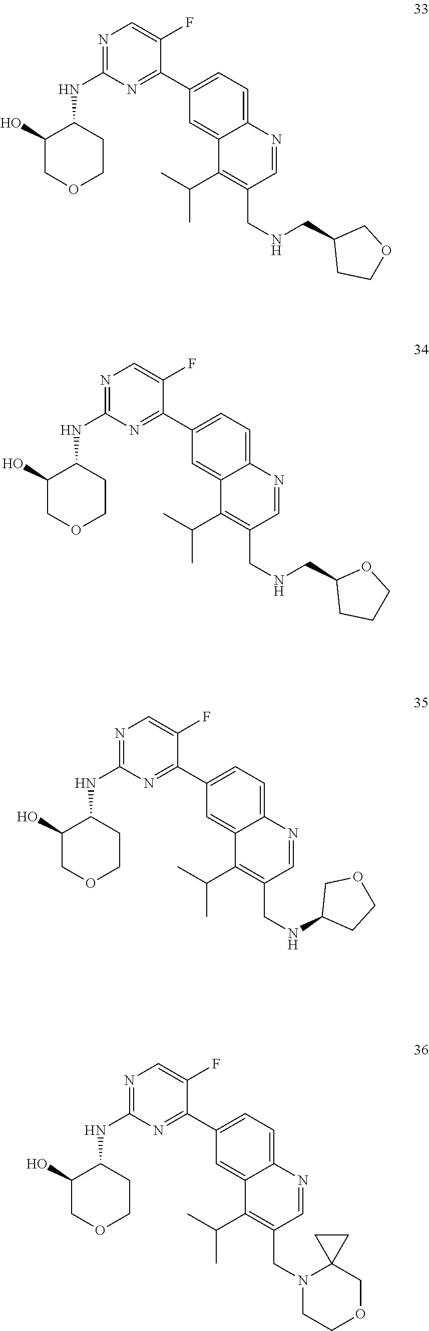 [SMILES: CC(C)c1c(CNC[C@@H]2CCOC2)cnc2ccc(-c3nc(N[C@@H]4CCOC[C@H]4O)ncc3F)cc12 ; CC(C)c1c(CNC[C@@H]2CCCO2)cnc2ccc(-c3nc(N[C@@H]4CCOC[C@H]4O)ncc3F)cc12 ; CC(C)c1c(CN[C@@H]2CCOC2)cnc2ccc(-c3nc(N[C@@H]4CCOC[C@H]4O)ncc3F)cc12 ; CC(C)c1c(CN2CCOCC23CC3)cnc2ccc(-c3nc(N[C@@H]4CCOC[C@H]4O)ncc3F)cc12]

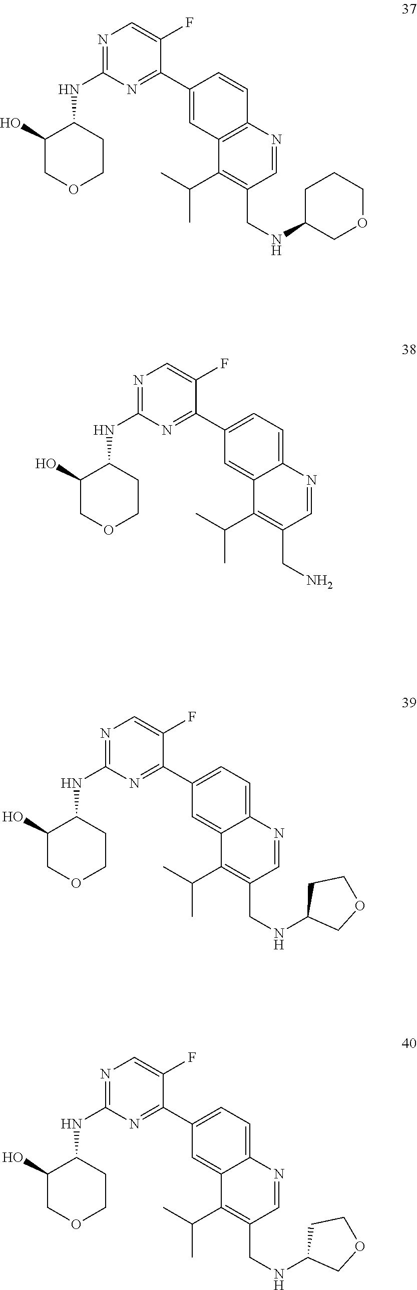 [SMILES: CC(C)c1c(CN[C@H]2CCCOC2)cnc2ccc(-c3nc(N[C@@H]4CCOC[C@H]4O)ncc3F)cc12 ; CC(C)c1c(CN)cnc2ccc(-c3nc(N[C@@H]4CCOC[C@H]4O)ncc3F)cc12 ; CC(C)c1c(CN[C@H]2CCOC2)cnc2ccc(-c3nc(N[C@@H]4CCOC[C@H]4O)ncc3F)cc12 ; CC(C)c1c(CN[C@@H]2CCOC2)cnc2ccc(-c3nc(N[C@@H]4CCOC[C@H]4O)ncc3F)cc12]

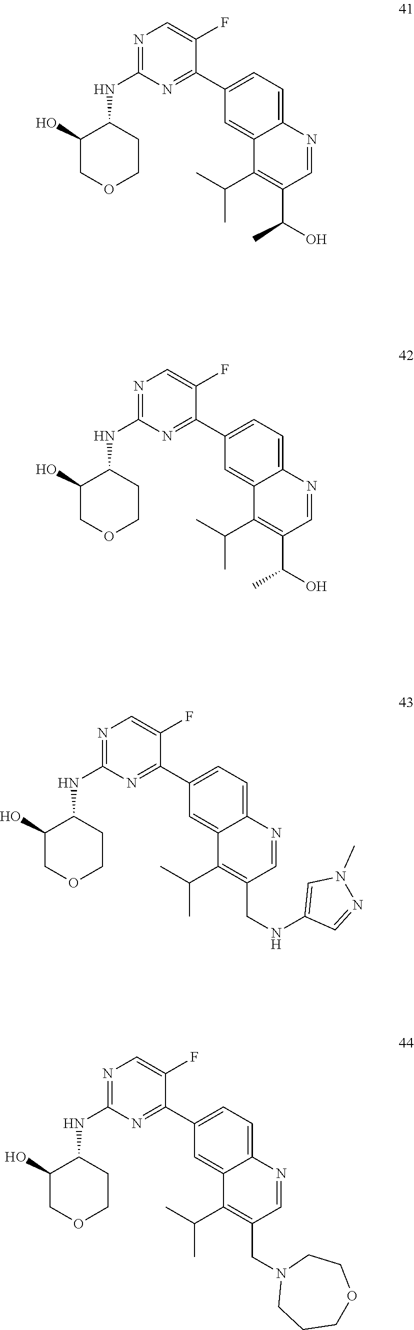 [SMILES: CC(C)c1c([C@H](C)O)cnc2ccc(-c3nc(N[C@@H]4CCOC[C@H]4O)ncc3F)cc12 ; CC(C)c1c([C@@H](C)O)cnc2ccc(-c3nc(N[C@@H]4CCOC[C@H]4O)ncc3F)cc12 ; CC(C)c1c(CNc2cnn(C)c2)cnc2ccc(-c3nc(N[C@@H]4CCOC[C@H]4O)ncc3F)cc12 ; CC(C)c1c(CN2CCCOCC2)cnc2ccc(-c3nc(N[C@@H]4CCOC[C@H]4O)ncc3F)cc12]

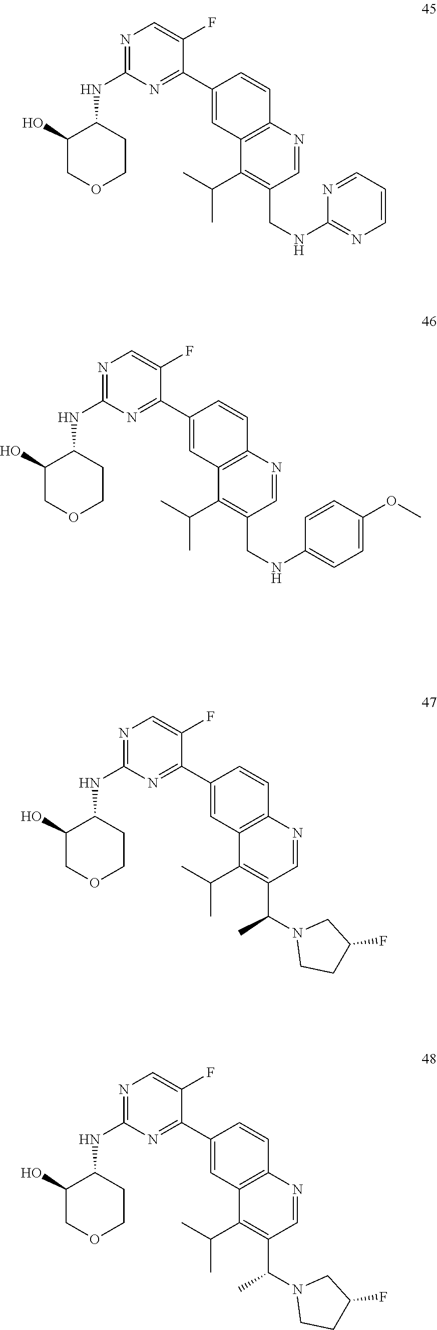 [SMILES: CC(C)c1c(CNc2ncccn2)cnc2ccc(-c3nc(N[C@@H]4CCOC[C@H]4O)ncc3F)cc12 ; COc1ccc(NCc2cnc3ccc(-c4nc(N[C@@H]5CCOC[C@H]5O)ncc4F)cc3c2C(C)C)cc1 ; CC(C)c1c([C@H](C)N2CC[C@@H](F)C2)cnc2ccc(-c3nc(N[C@@H]4CCOC[C@H]4O)ncc3F)cc12 ; CC(C)c1c([C@@H](C)N2CC[C@@H](F)C2)cnc2ccc(-c3nc(N[C@@H]4CCOC[C@H]4O)ncc3F)cc12]

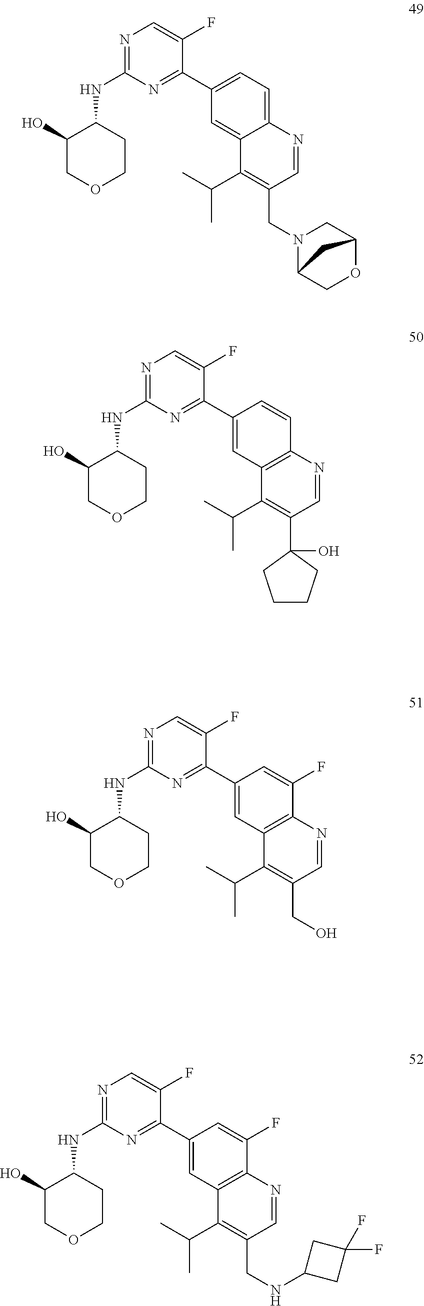 [SMILES: CC(C)c1c(CN2C[C@H]3C[C@@H]2CO3)cnc2ccc(-c3nc(N[C@@H]4CCOC[C@H]4O)ncc3F)cc12 ; CC(C)c1c(C2(O)CCCC2)cnc2ccc(-c3nc(N[C@@H]4CCOC[C@H]4O)ncc3F)cc12 ; CC(C)c1c(CO)cnc2c(F)cc(-c3nc(N[C@@H]4CCOC[C@H]4O)ncc3F)cc12 ; CC(C)c1c(CNC2CC(F)(F)C2)cnc2c(F)cc(-c3nc(N[C@@H]4CCOC[C@H]4O)ncc3F)cc12]

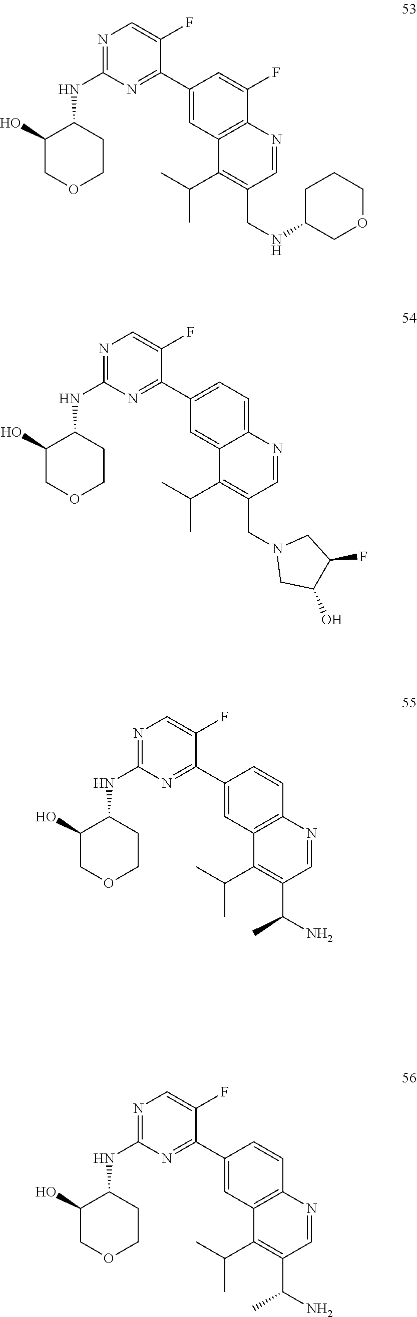 [SMILES: CC(C)c1c(CN[C@@H]2CCCOC2)cnc2c(F)cc(-c3nc(N[C@@H]4CCOC[C@H]4O)ncc3F)cc12 ; CC(C)c1c(CN2C[C@@H](O)[C@H](F)C2)cnc2ccc(-c3nc(N[C@@H]4CCOC[C@H]4O)ncc3F)cc12 ; CC(C)c1c([C@H](C)N)cnc2ccc(-c3nc(N[C@@H]4CCOC[C@H]4O)ncc3F)cc12 ; CC(C)c1c([C@@H](C)N)cnc2ccc(-c3nc(N[C@@H]4CCOC[C@H]4O)ncc3F)cc12]

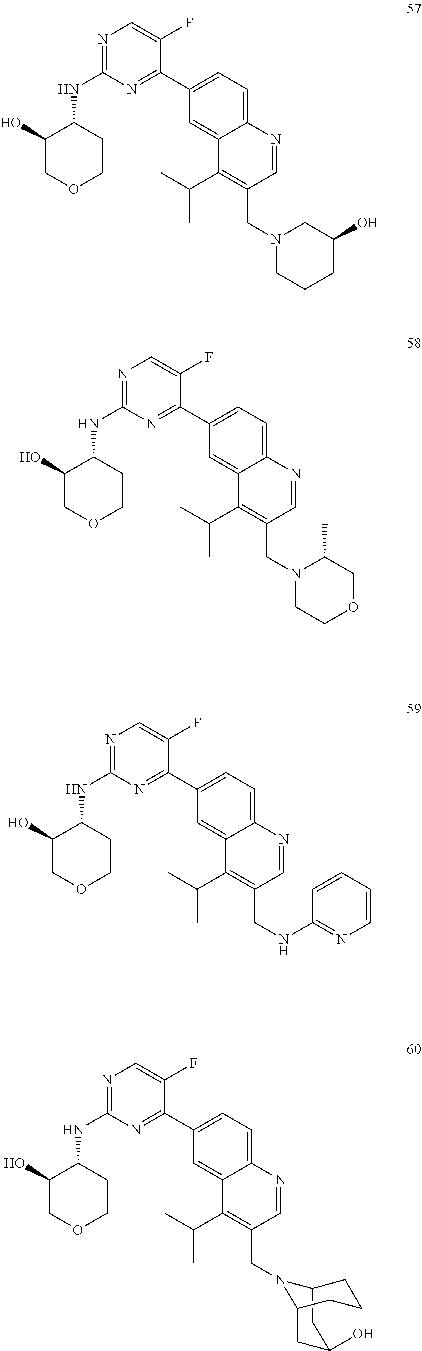 [SMILES: CC(C)c1c(CN2CCC[C@H](O)C2)cnc2ccc(-c3nc(N[C@@H]4CCOC[C@H]4O)ncc3F)cc12 ; CC(C)c1c(CN2CCOC[C@H]2C)cnc2ccc(-c3nc(N[C@@H]4CCOC[C@H]4O)ncc3F)cc12 ; CC(C)c1c(CNc2ccccn2)cnc2ccc(-c3nc(N[C@@H]4CCOC[C@H]4O)ncc3F)cc12 ; CC(C)c1c(CN2C3CCCC2CC(O)C3)cnc2ccc(-c3nc(N[C@@H]4CCOC[C@H]4O)ncc3F)cc12]

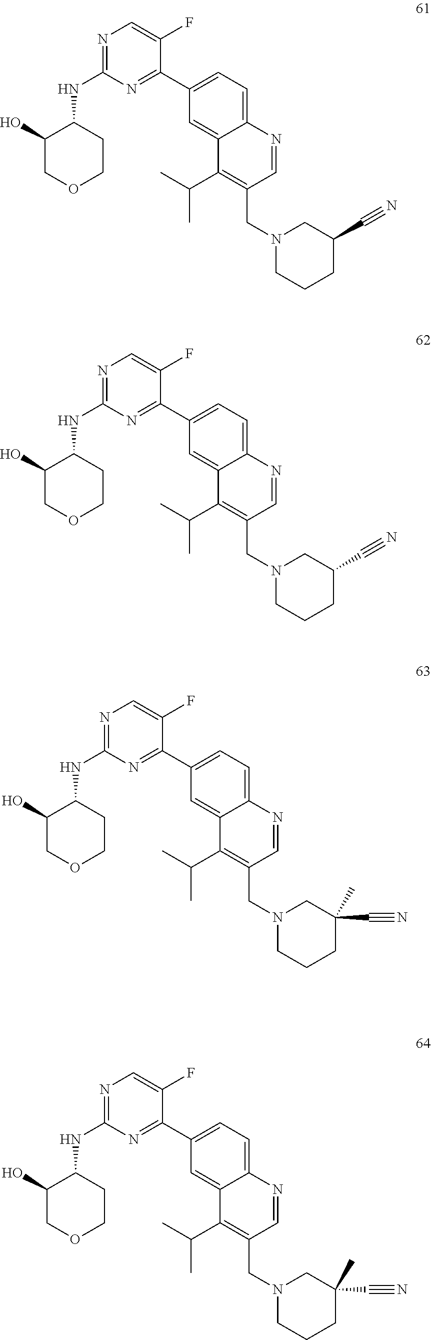 [SMILES: CC(C)c1c(CN2CCC[C@H](C#N)C2)cnc2ccc(-c3nc(N[C@@H]4CCOC[C@H]4O)ncc3F)cc12 ; CC(C)c1c(CN2CCC[C@@H](C#N)C2)cnc2ccc(-c3nc(N[C@@H]4CCOC[C@H]4O)ncc3F)cc12 ; CC(C)c1c(CN2CCC[C@](C)(C#N)C2)cnc2ccc(-c3nc(N[C@@H]4CCOC[C@H]4O)ncc3F)cc12 ; CC(C)c1c(CN2CCC[C@@](C)(C#N)C2)cnc2ccc(-c3nc(N[C@@H]4CCOC[C@H]4O)ncc3F)cc12]

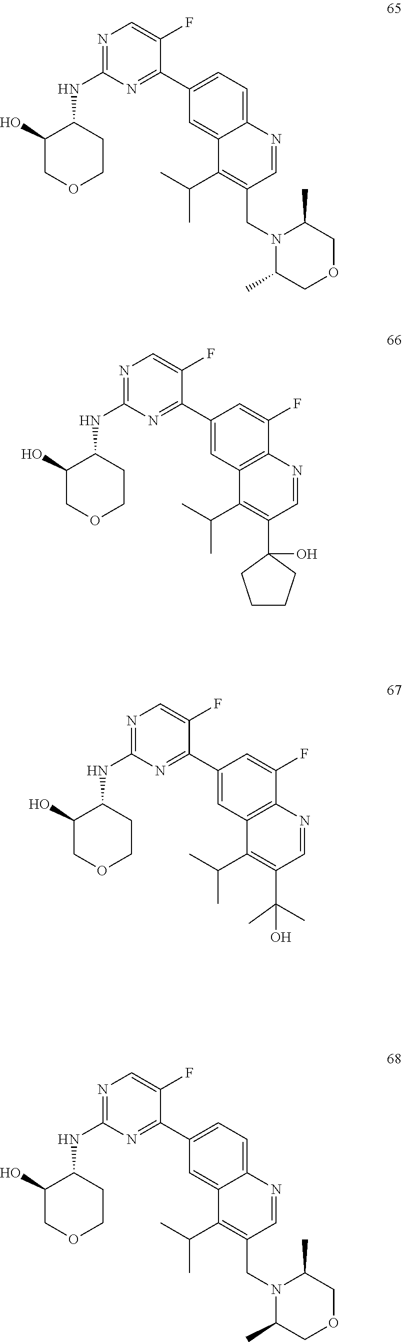 [SMILES: CC(C)c1c(CN2[C@@H](C)COC[C@@H]2C)cnc2ccc(-c3nc(N[C@@H]4CCOC[C@H]4O)ncc3F)cc12 ; CC(C)c1c(C2(O)CCCC2)cnc2c(F)cc(-c3nc(N[C@@H]4CCOC[C@H]4O)ncc3F)cc12 ; CC(C)c1c(C(C)(C)O)cnc2c(F)cc(-c3nc(N[C@@H]4CCOC[C@H]4O)ncc3F)cc12 ; CC(C)c1c(CN2[C@H](C)COC[C@@H]2C)cnc2ccc(-c3nc(N[C@@H]4CCOC[C@H]4O)ncc3F)cc12]

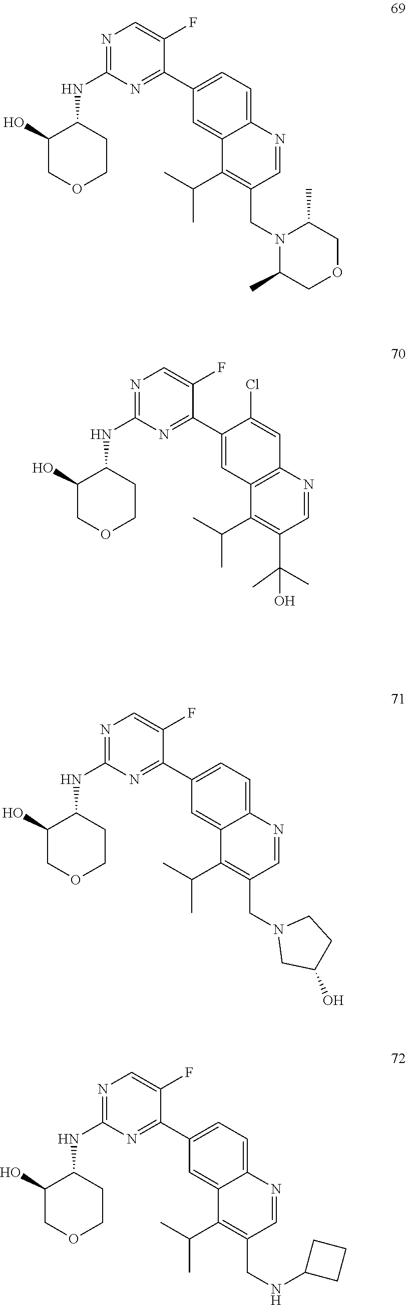 [SMILES: CC(C)c1c(CN2[C@H](C)COC[C@H]2C)cnc2ccc(-c3nc(N[C@@H]4CCOC[C@H]4O)ncc3F)cc12 ; CC(C)c1c(C(C)(C)O)cnc2cc(Cl)c(-c3nc(N[C@@H]4CCOC[C@H]4O)ncc3F)cc12 ; CC(C)c1c(CN2CC[C@H](O)C2)cnc2ccc(-c3nc(N[C@@H]4CCOC[C@H]4O)ncc3F)cc12 ; CC(C)c1c(CNC2CCC2)cnc2ccc(-c3nc(N[C@@H]4CCOC[C@H]4O)ncc3F)cc12]

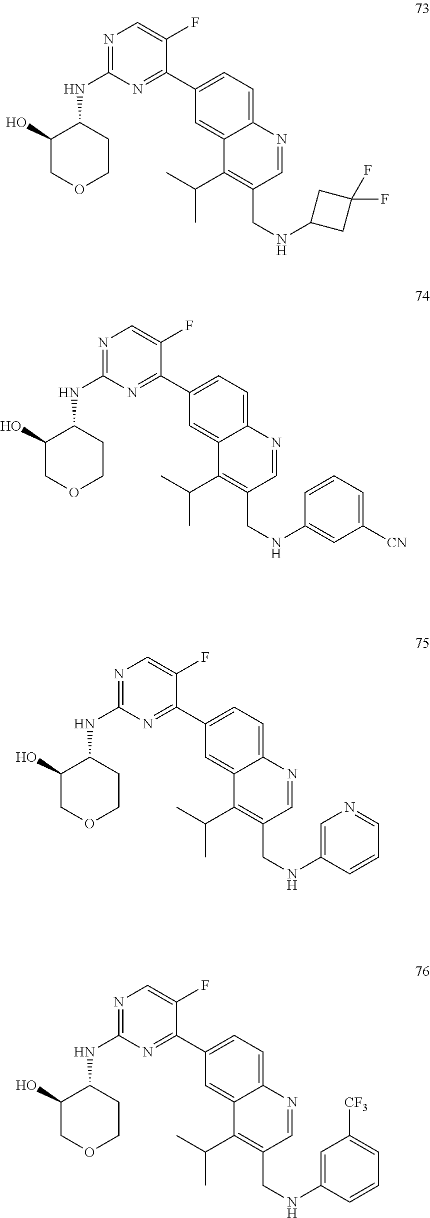 [SMILES: CC(C)c1c(CNC2CC(F)(F)C2)cnc2ccc(-c3nc(N[C@@H]4CCOC[C@H]4O)ncc3F)cc12 ; CC(C)c1c(CNc2cccc(C#N)c2)cnc2ccc(-c3nc(N[C@@H]4CCOC[C@H]4O)ncc3F)cc12 ; CC(C)c1c(CNc2cccnc2)cnc2ccc(-c3nc(N[C@@H]4CCOC[C@H]4O)ncc3F)cc12 ; CC(C)c1c(CNc2cccc(C(F)(F)F)c2)cnc2ccc(-c3nc(N[C@@H]4CCOC[C@H]4O)ncc3F)cc12]

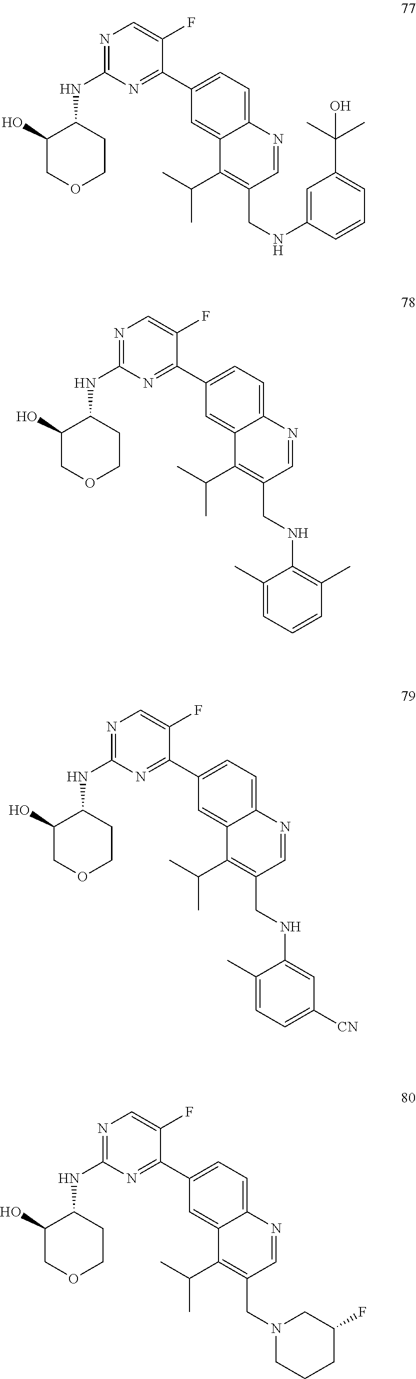 [SMILES: CC(C)c1c(CNc2cccc(C(C)(C)O)c2)cnc2ccc(-c3nc(N[C@@H]4CCOC[C@H]4O)ncc3F)cc12 ; Cc1cccc(C)c1NCc1cnc2ccc(-c3nc(N[C@@H]4CCOC[C@H]4O)ncc3F)cc2c1C(C)C ; Cc1ccc(C#N)cc1NCc1cnc2ccc(-c3nc(N[C@@H]4CCOC[C@H]4O)ncc3F)cc2c1C(C)C ; CC(C)c1c(CN2CCC[C@@H](F)C2)cnc2ccc(-c3nc(N[C@@H]4CCOC[C@H]4O)ncc3F)cc12]

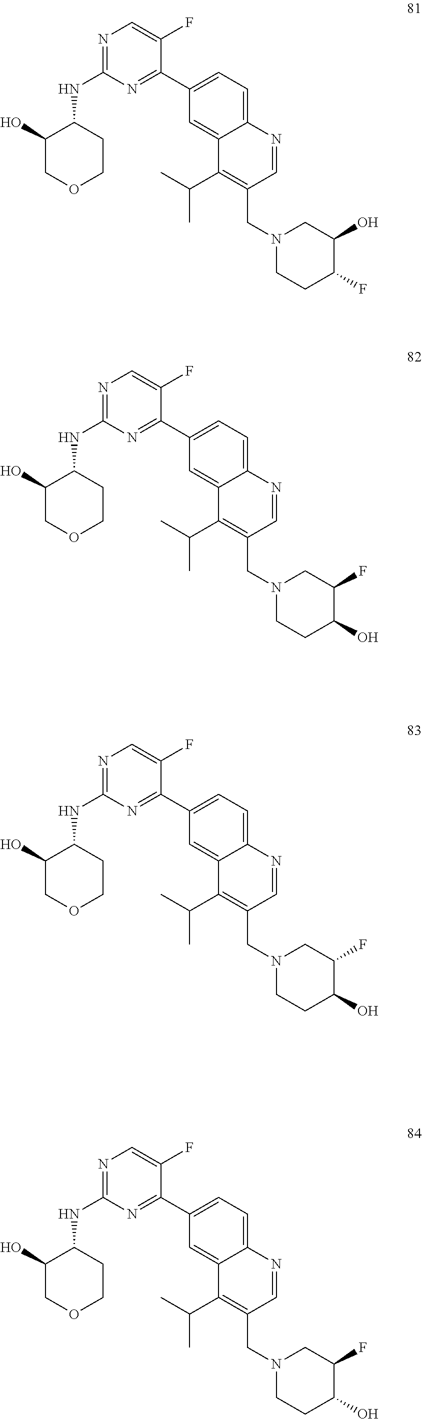 [SMILES: CC(C)c1c(CN2CC[C@@H](F)[C@H](O)C2)cnc2ccc(-c3nc(N[C@@H]4CCOC[C@H]4O)ncc3F)cc12 ; CC(C)c1c(CN2CC[C@H](O)[C@H](F)C2)cnc2ccc(-c3nc(N[C@@H]4CCOC[C@H]4O)ncc3F)cc12 ; CC(C)c1c(CN2CC[C@H](O)[C@@H](F)C2)cnc2ccc(-c3nc(N[C@@H]4CCOC[C@H]4O)ncc3F)cc12 ; CC(C)c1c(CN2CC[C@@H](O)[C@H](F)C2)cnc2ccc(-c3nc(N[C@@H]4CCOC[C@H]4O)ncc3F)cc12]

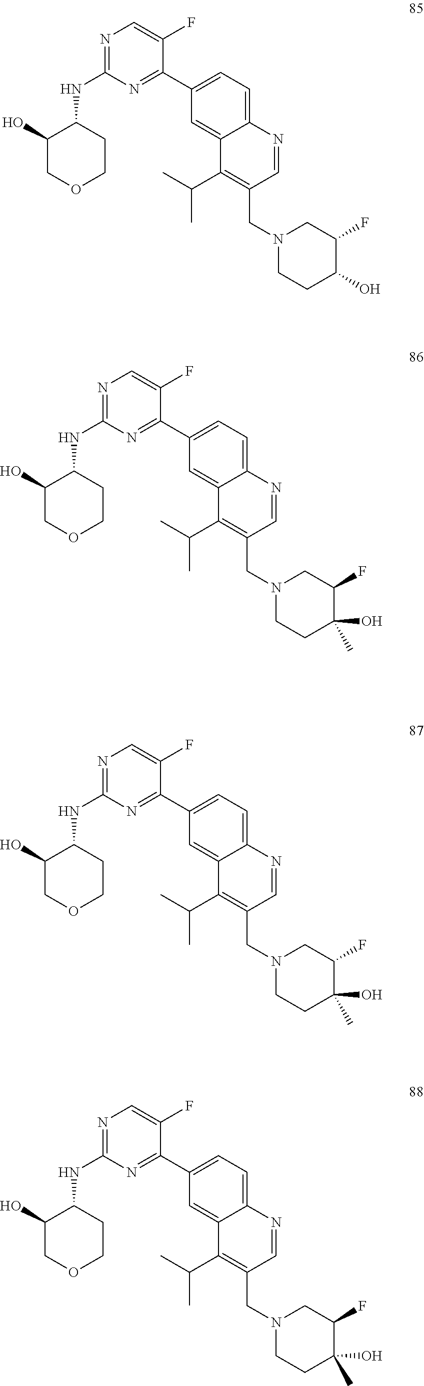 [SMILES: CC(C)c1c(CN2CC[C@@H](O)[C@@H](F)C2)cnc2ccc(-c3nc(N[C@@H]4CCOC[C@H]4O)ncc3F)cc12 ; CC(C)c1c(CN2CC[C@](C)(O)[C@H](F)C2)cnc2ccc(-c3nc(N[C@@H]4CCOC[C@H]4O)ncc3F)cc12 ; CC(C)c1c(CN2CC[C@](C)(O)[C@@H](F)C2)cnc2ccc(-c3nc(N[C@@H]4CCOC[C@H]4O)ncc3F)cc12 ; CC(C)c1c(CN2CC[C@@](C)(O)[C@H](F)C2)cnc2ccc(-c3nc(N[C@@H]4CCOC[C@H]4O)ncc3F)cc12]

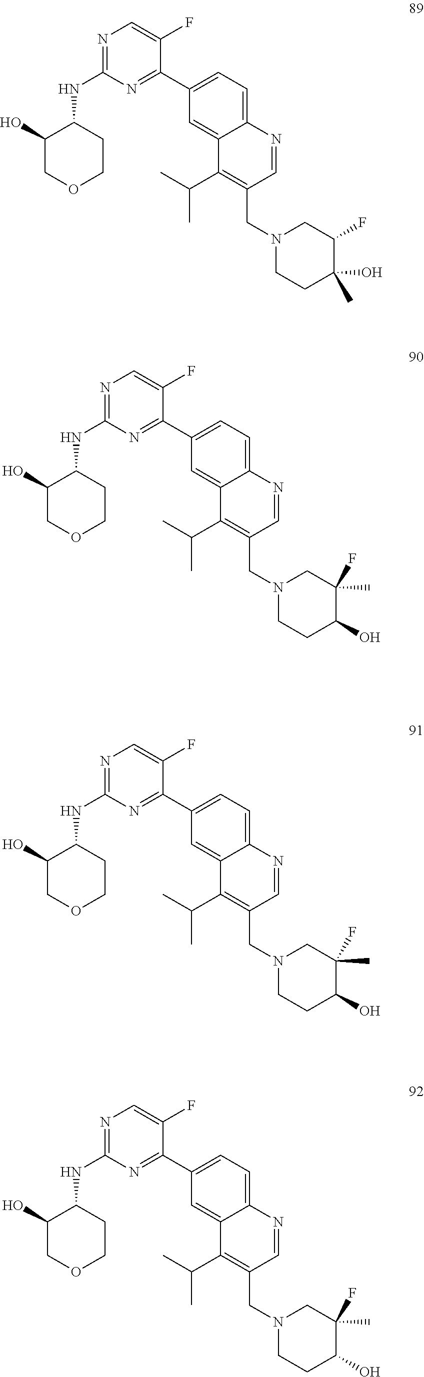 [SMILES: CC(C)c1c(CN2CC[C@@](C)(O)[C@@H](F)C2)cnc2ccc(-c3nc(N[C@@H]4CCOC[C@H]4O)ncc3F)cc12 ; CC(C)c1c(CN2CC[C@H](O)[C@](C)(F)C2)cnc2ccc(-c3nc(N[C@@H]4CCOC[C@H]4O)ncc3F)cc12 ; CC(C)c1c(CN2CC[C@H](O)[C@@](C)(F)C2)cnc2ccc(-c3nc(N[C@@H]4CCOC[C@H]4O)ncc3F)cc12 ; CC(C)c1c(CN2CC[C@@H](O)[C@](C)(F)C2)cnc2ccc(-c3nc(N[C@@H]4CCOC[C@H]4O)ncc3F)cc12]

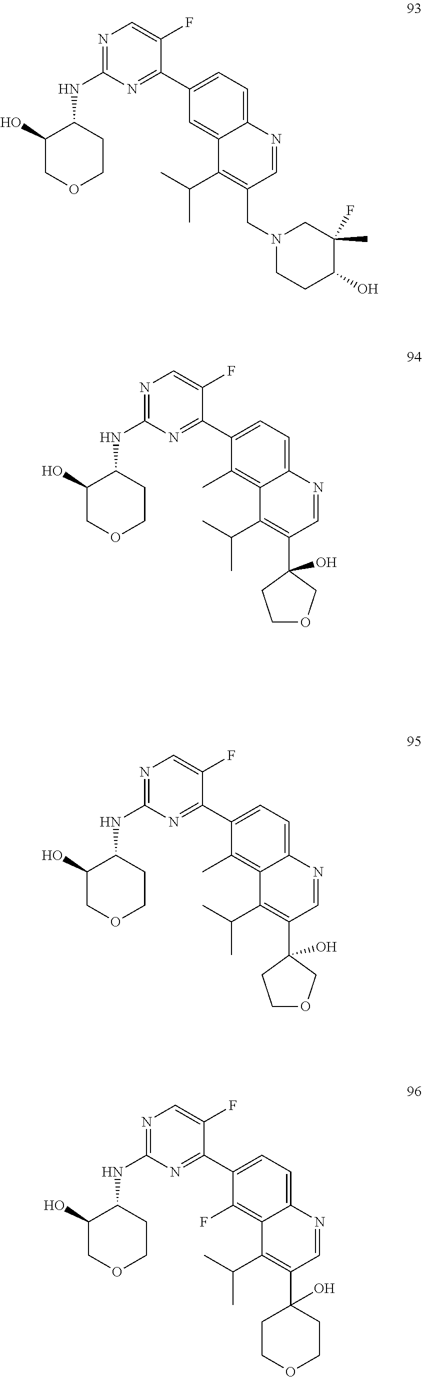 [SMILES: CC(C)c1c(CN2CC[C@@H](O)[C@@](C)(F)C2)cnc2ccc(-c3nc(N[C@@H]4CCOC[C@H]4O)ncc3F)cc12 ; Cc1c(-c2nc(N[C@@H]3CCOC[C@H]3O)ncc2F)ccc2ncc([C@@]3(O)CCOC3)c(C(C)C)c12 ; Cc1c(-c2nc(N[C@@H]3CCOC[C@H]3O)ncc2F)ccc2ncc([C@]3(O)CCOC3)c(C(C)C)c12 ; CC(C)c1c(C2(O)CCOCC2)cnc2ccc(-c3nc(N[C@@H]4CCOC[C@H]4O)ncc3F)c(F)c12]

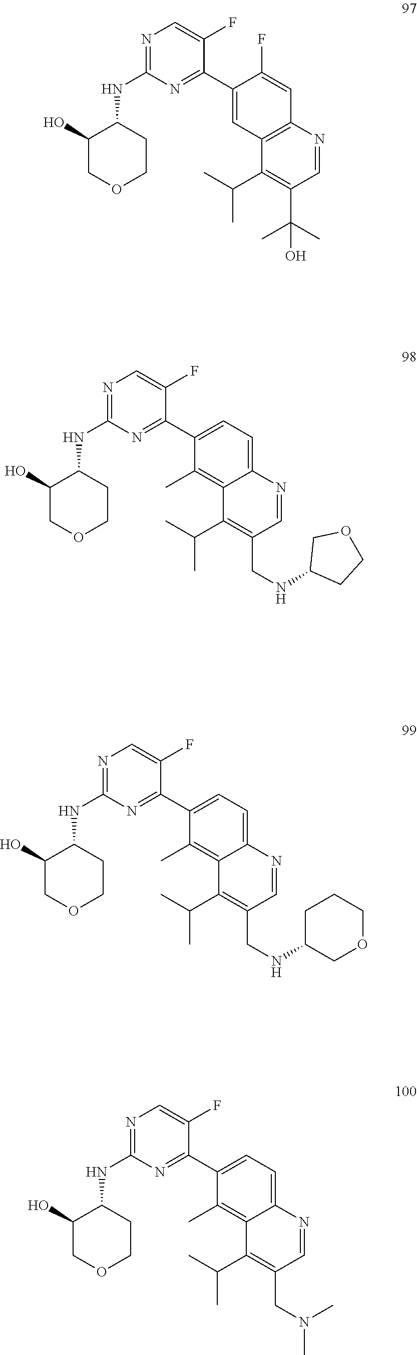 [SMILES: CC(C)c1c(C(C)(C)O)cnc2cc(F)c(-c3nc(N[C@@H]4CCOC[C@H]4O)ncc3F)cc12 ; Cc1c(-c2nc(N[C@@H]3CCOC[C@H]3O)ncc2F)ccc2ncc(CN[C@H]3CCOC3)c(C(C)C)c12 ; Cc1c(-c2nc(N[C@@H]3CCOC[C@H]3O)ncc2F)ccc2ncc(CN[C@@H]3CCCOC3)c(C(C)C)c12 ; Cc1c(-c2nc(N[C@@H]3CCOC[C@H]3O)ncc2F)ccc2ncc(CN(C)C)c(C(C)C)c12]

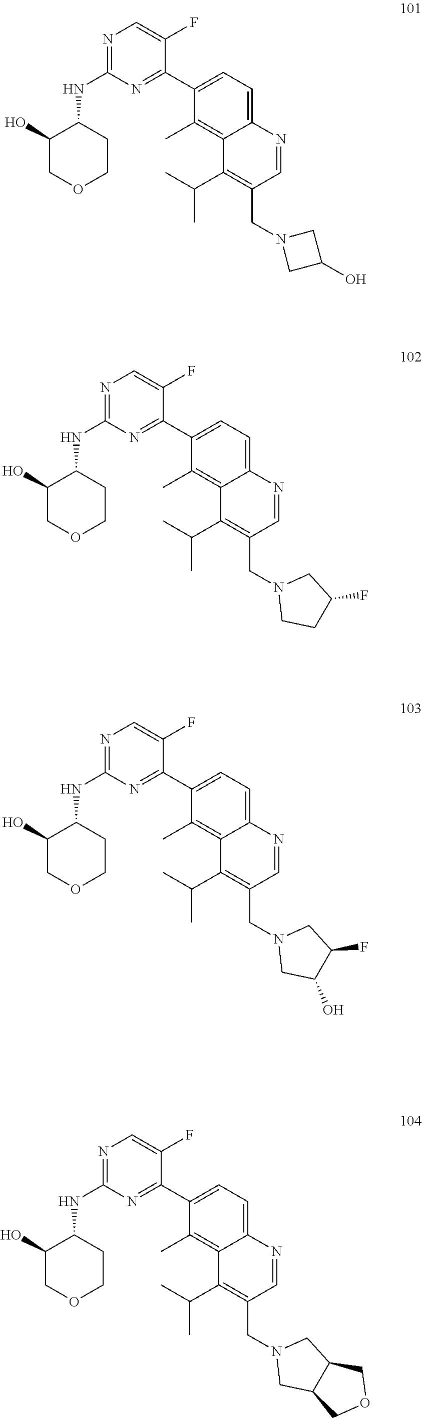 [SMILES: Cc1c(-c2nc(N[C@@H]3CCOC[C@H]3O)ncc2F)ccc2ncc(CN3CC(O)C3)c(C(C)C)c12 ; Cc1c(-c2nc(N[C@@H]3CCOC[C@H]3O)ncc2F)ccc2ncc(CN3CC[C@@H](F)C3)c(C(C)C)c12 ; Cc1c(-c2nc(N[C@@H]3CCOC[C@H]3O)ncc2F)ccc2ncc(CN3C[C@@H](O)[C@H](F)C3)c(C(C)C)c12 ; Cc1c(-c2nc(N[C@@H]3CCOC[C@H]3O)ncc2F)ccc2ncc(CN3C[C@H]4COC[C@H]4C3)c(C(C)C)c12]

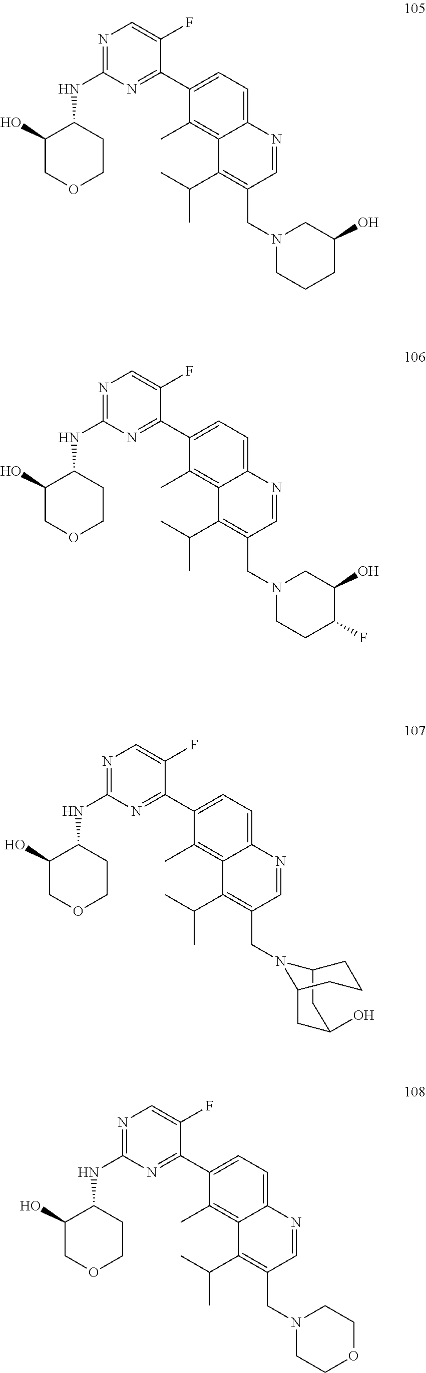 [SMILES: Cc1c(-c2nc(N[C@@H]3CCOC[C@H]3O)ncc2F)ccc2ncc(CN3CCC[C@H](O)C3)c(C(C)C)c12 ; Cc1c(-c2nc(N[C@@H]3CCOC[C@H]3O)ncc2F)ccc2ncc(CN3CC[C@@H](F)[C@H](O)C3)c(C(C)C)c12 ; Cc1c(-c2nc(N[C@@H]3CCOC[C@H]3O)ncc2F)ccc2ncc(CN3C4CCCC3CC(O)C4)c(C(C)C)c12 ; Cc1c(-c2nc(N[C@@H]3CCOC[C@H]3O)ncc2F)ccc2ncc(CN3CCOCC3)c(C(C)C)c12]

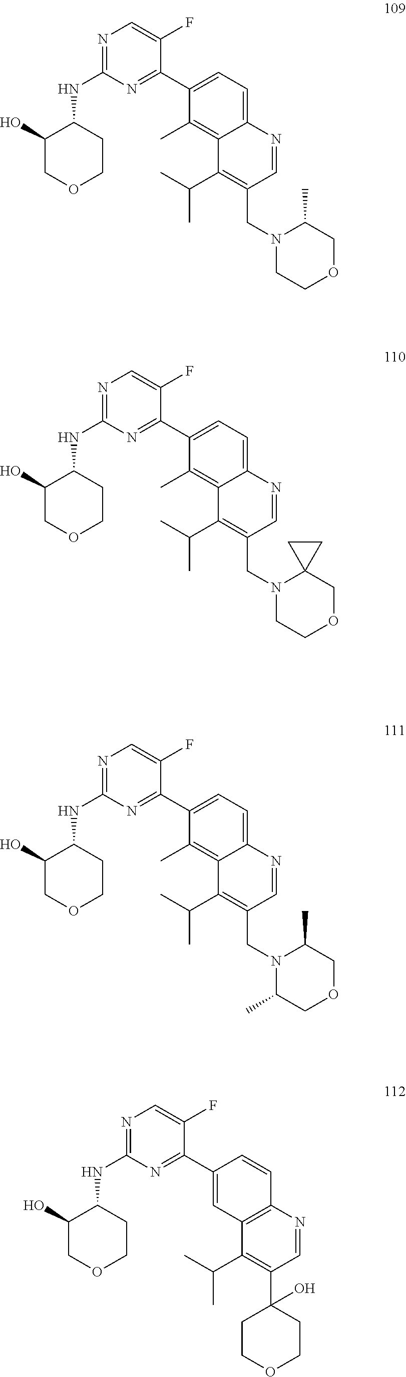 [SMILES: Cc1c(-c2nc(N[C@@H]3CCOC[C@H]3O)ncc2F)ccc2ncc(CN3CCOC[C@H]3C)c(C(C)C)c12 ; Cc1c(-c2nc(N[C@@H]3CCOC[C@H]3O)ncc2F)ccc2ncc(CN3CCOCC34CC4)c(C(C)C)c12 ; Cc1c(-c2nc(N[C@@H]3CCOC[C@H]3O)ncc2F)ccc2ncc(CN3[C@@H](C)COC[C@@H]3C)c(C(C)C)c12 ; CC(C)c1c(C2(O)CCOCC2)cnc2ccc(-c3nc(N[C@@H]4CCOC[C@H]4O)ncc3F)cc12]

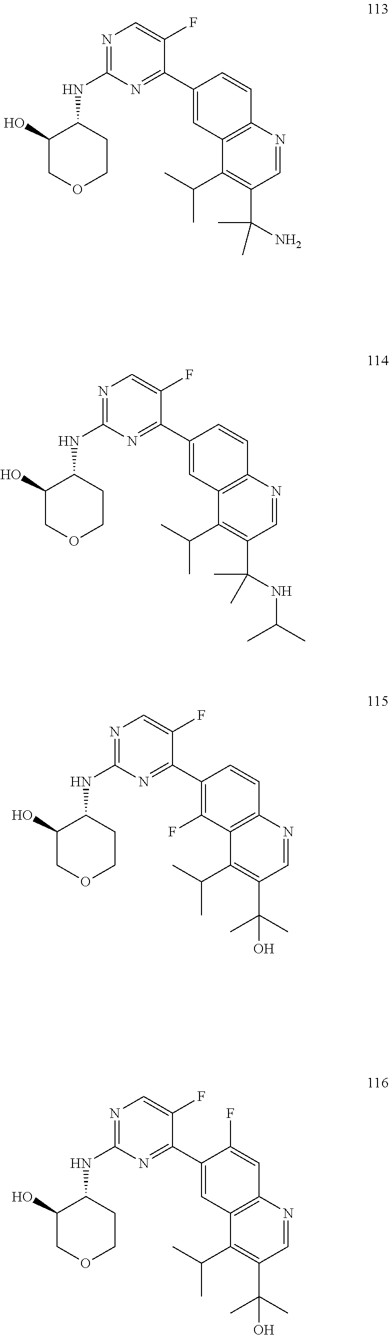 [SMILES: CC(C)c1c(C(C)(C)N)cnc2ccc(-c3nc(N[C@@H]4CCOC[C@H]4O)ncc3F)cc12 ; CC(C)NC(C)(C)c1cnc2ccc(-c3nc(N[C@@H]4CCOC[C@H]4O)ncc3F)cc2c1C(C)C ; CC(C)c1c(C(C)(C)O)cnc2ccc(-c3nc(N[C@@H]4CCOC[C@H]4O)ncc3F)c(F)c12 ; CC(C)c1c(C(C)(C)O)cnc2cc(F)c(-c3nc(N[C@@H]4CCOC[C@H]4O)ncc3F)cc12]

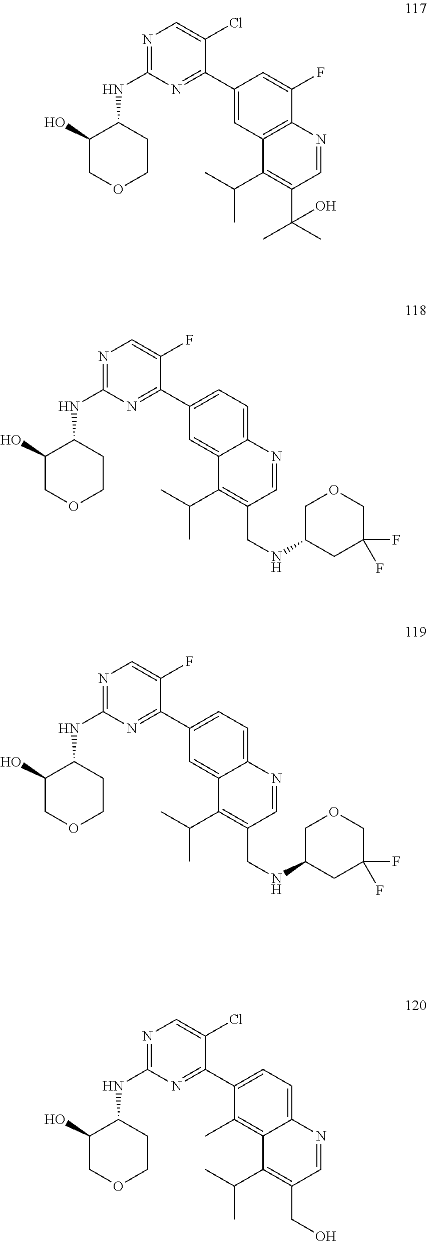 [SMILES: CC(C)c1c(C(C)(C)O)cnc2c(F)cc(-c3nc(N[C@@H]4CCOC[C@H]4O)ncc3Cl)cc12 ; CC(C)c1c(CN[C@@H]2COCC(F)(F)C2)cnc2ccc(-c3nc(N[C@@H]4CCOC[C@H]4O)ncc3F)cc12 ; CC(C)c1c(CN[C@H]2COCC(F)(F)C2)cnc2ccc(-c3nc(N[C@@H]4CCOC[C@H]4O)ncc3F)cc12 ; Cc1c(-c2nc(N[C@@H]3CCOC[C@H]3O)ncc2Cl)ccc2ncc(CO)c(C(C)C)c12]

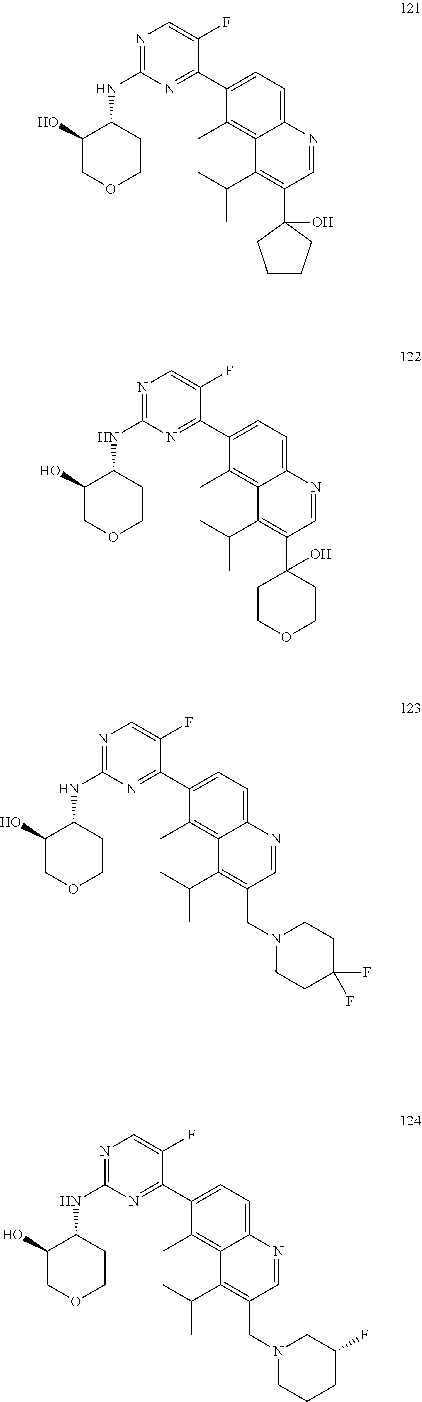 [SMILES: Cc1c(-c2nc(N[C@@H]3CCOC[C@H]3O)ncc2F)ccc2ncc(C3(O)CCCC3)c(C(C)C)c12 ; Cc1c(-c2nc(N[C@@H]3CCOC[C@H]3O)ncc2F)ccc2ncc(C3(O)CCOCC3)c(C(C)C)c12 ; Cc1c(-c2nc(N[C@@H]3CCOC[C@H]3O)ncc2F)ccc2ncc(CN3CCC(F)(F)CC3)c(C(C)C)c12 ; Cc1c(-c2nc(N[C@@H]3CCOC[C@H]3O)ncc2F)ccc2ncc(CN3CCC[C@@H](F)C3)c(C(C)C)c12]

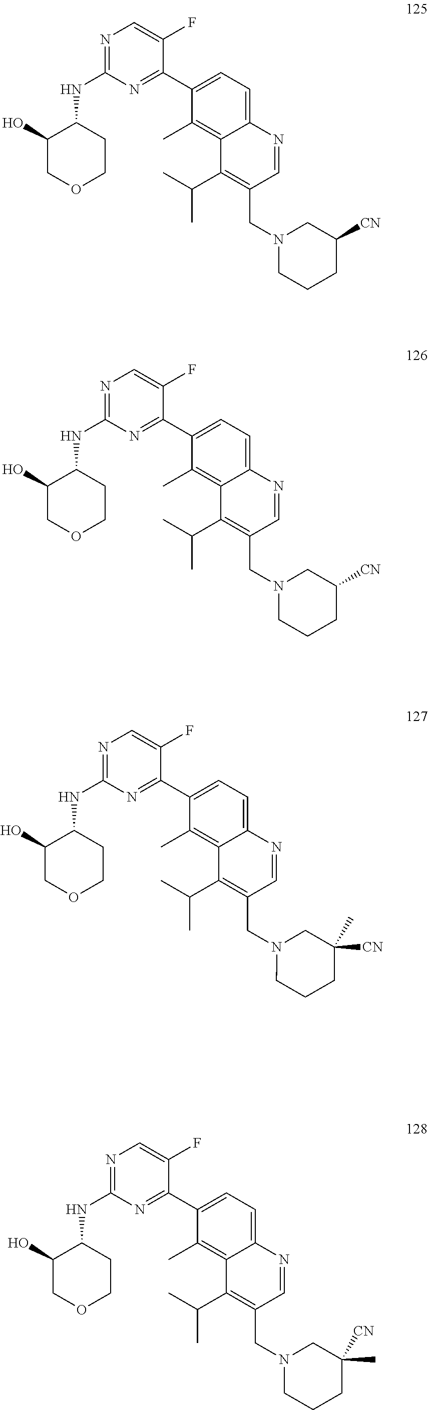 [SMILES: Cc1c(-c2nc(N[C@@H]3CCOC[C@H]3O)ncc2F)ccc2ncc(CN3CCC[C@H](C#N)C3)c(C(C)C)c12 ; Cc1c(-c2nc(N[C@@H]3CCOC[C@H]3O)ncc2F)ccc2ncc(CN3CCC[C@@H](C#N)C3)c(C(C)C)c12 ; Cc1c(-c2nc(N[C@@H]3CCOC[C@H]3O)ncc2F)ccc2ncc(CN3CCC[C@](C)(C#N)C3)c(C(C)C)c12 ; Cc1c(-c2nc(N[C@@H]3CCOC[C@H]3O)ncc2F)ccc2ncc(CN3CCC[C@@](C)(C#N)C3)c(C(C)C)c12]

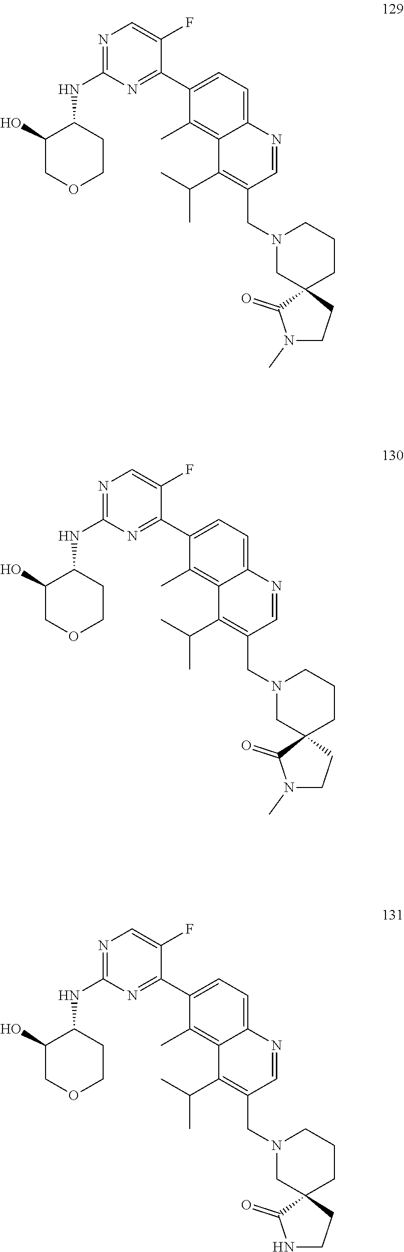 [SMILES: Cc1c(-c2nc(N[C@@H]3CCOC[C@H]3O)ncc2F)ccc2ncc(CN3CCC[C@@]4(CCN(C)C4=O)C3)c(C(C)C)c12 ; Cc1c(-c2nc(N[C@@H]3CCOC[C@H]3O)ncc2F)ccc2ncc(CN3CCC[C@]4(CCN(C)C4=O)C3)c(C(C)C)c12 ; Cc1c(-c2nc(N[C@@H]3CCOC[C@H]3O)ncc2F)ccc2ncc(CN3CCC[C@@]4(CCNC4=O)C3)c(C(C)C)c12]

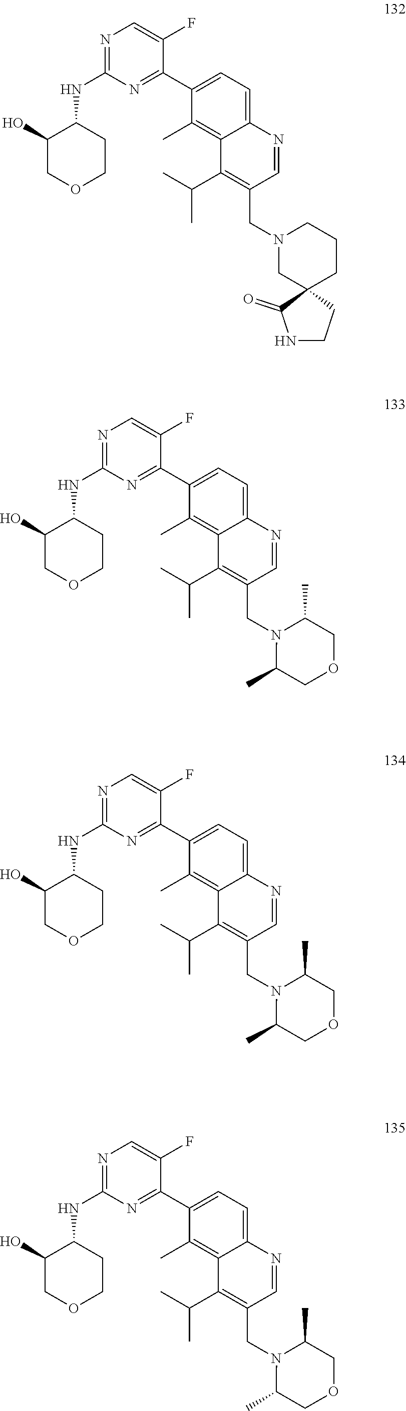 [SMILES: Cc1c(-c2nc(N[C@@H]3CCOC[C@H]3O)ncc2F)ccc2ncc(CN3CCC[C@]4(CCNC4=O)C3)c(C(C)C)c12 ; Cc1c(-c2nc(N[C@@H]3CCOC[C@H]3O)ncc2F)ccc2ncc(CN3[C@H](C)COC[C@H]3C)c(C(C)C)c12 ; Cc1c(-c2nc(N[C@@H]3CCOC[C@H]3O)ncc2F)ccc2ncc(CN3[C@H](C)COC[C@@H]3C)c(C(C)C)c12 ; Cc1c(-c2nc(N[C@@H]3CCOC[C@H]3O)ncc2F)ccc2ncc(CN3[C@@H](C)COC[C@@H]3C)c(C(C)C)c12]

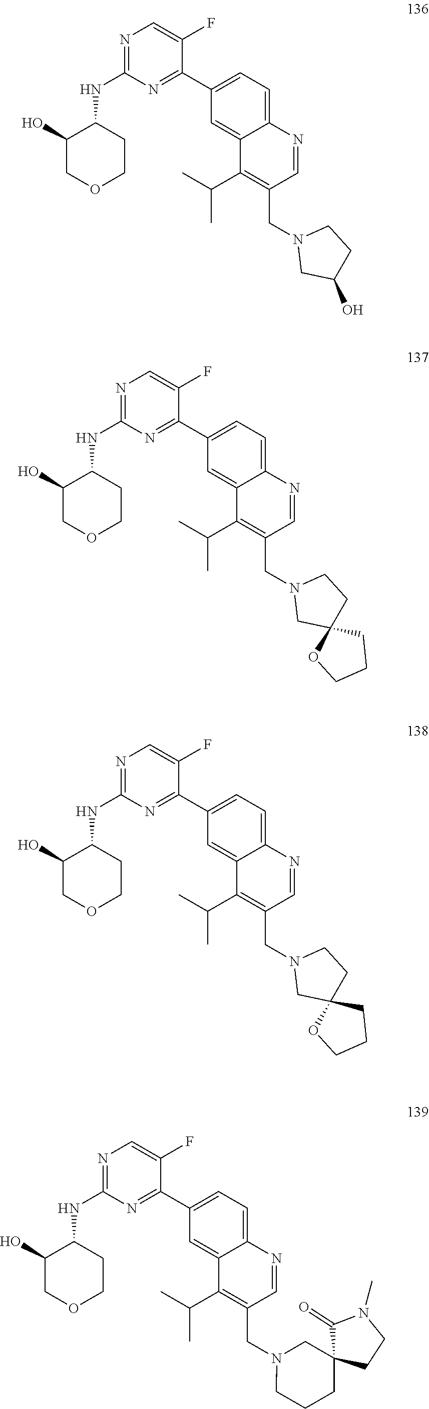 [SMILES: CC(C)c1c(CN2CC[C@@H](O)C2)cnc2ccc(-c3nc(N[C@@H]4CCOC[C@H]4O)ncc3F)cc12 ; CC(C)c1c(CN2CC[C@]3(CCCO3)C2)cnc2ccc(-c3nc(N[C@@H]4CCOC[C@H]4O)ncc3F)cc12 ; CC(C)c1c(CN2CC[C@@]3(CCCO3)C2)cnc2ccc(-c3nc(N[C@@H]4CCOC[C@H]4O)ncc3F)cc12 ; CC(C)c1c(CN2CCC[C@]3(CCN(C)C3=O)C2)cnc2ccc(-c3nc(N[C@@H]4CCOC[C@H]4O)ncc3F)cc12]

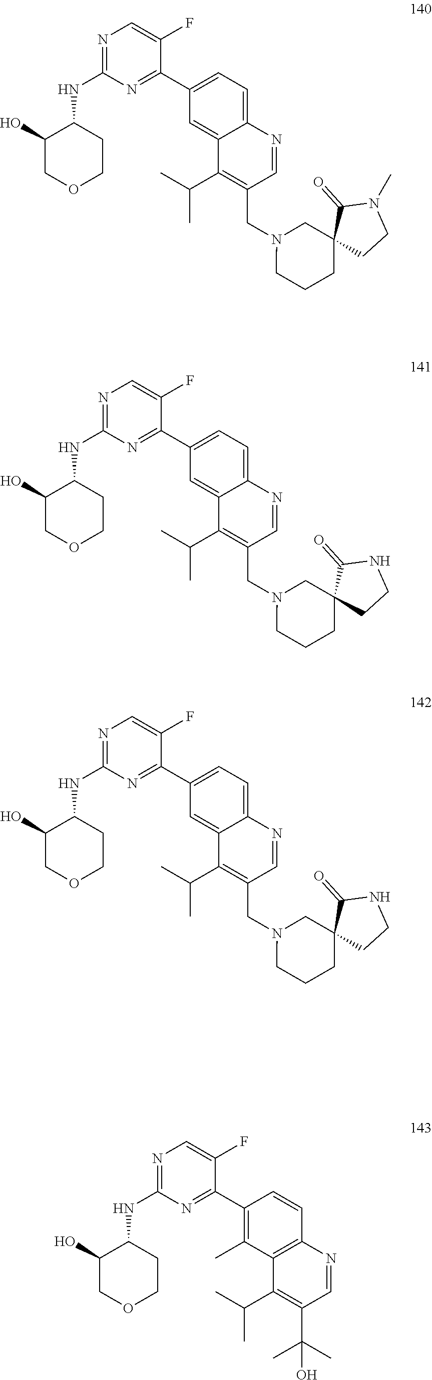 [SMILES: CC(C)c1c(CN2CCC[C@@]3(CCN(C)C3=O)C2)cnc2ccc(-c3nc(N[C@@H]4CCOC[C@H]4O)ncc3F)cc12 ; CC(C)c1c(CN2CCC[C@]3(CCNC3=O)C2)cnc2ccc(-c3nc(N[C@@H]4CCOC[C@H]4O)ncc3F)cc12 ; CC(C)c1c(CN2CCC[C@@]3(CCNC3=O)C2)cnc2ccc(-c3nc(N[C@@H]4CCOC[C@H]4O)ncc3F)cc12 ; Cc1c(-c2nc(N[C@@H]3CCOC[C@H]3O)ncc2F)ccc2ncc(C(C)(C)O)c(C(C)C)c12]

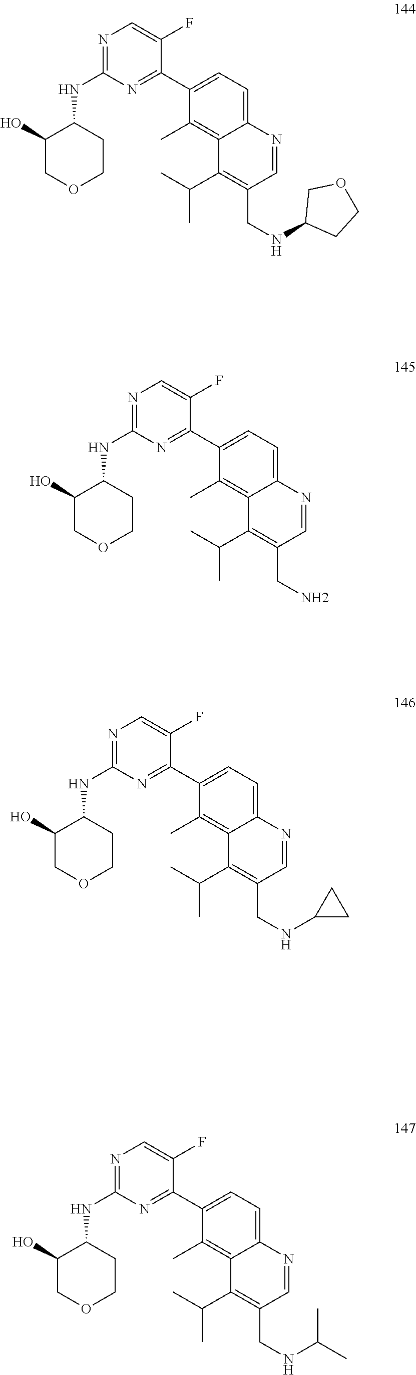 [SMILES: Cc1c(-c2nc(N[C@@H]3CCOC[C@H]3O)ncc2F)ccc2ncc(CN[C@@H]3CCOC3)c(C(C)C)c12 ; Cc1c(-c2nc(N[C@@H]3CCOC[C@H]3O)ncc2F)ccc2ncc(CN)c(C(C)C)c12 ; Cc1c(-c2nc(N[C@@H]3CCOC[C@H]3O)ncc2F)ccc2ncc(CNC3CC3)c(C(C)C)c12 ; Cc1c(-c2nc(N[C@@H]3CCOC[C@H]3O)ncc2F)ccc2ncc(CNC(C)C)c(C(C)C)c12]

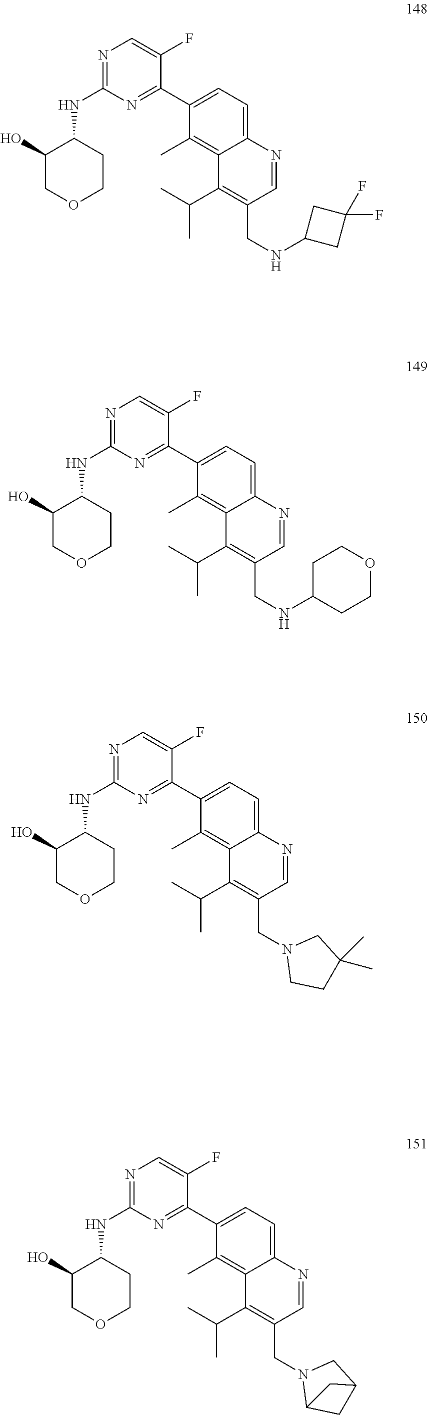 [SMILES: Cc1c(-c2nc(N[C@@H]3CCOC[C@H]3O)ncc2F)ccc2ncc(CNC3CC(F)(F)C3)c(C(C)C)c12 ; Cc1c(-c2nc(N[C@@H]3CCOC[C@H]3O)ncc2F)ccc2ncc(CNC3CCOCC3)c(C(C)C)c12 ; Cc1c(-c2nc(N[C@@H]3CCOC[C@H]3O)ncc2F)ccc2ncc(CN3CCC(C)(C)C3)c(C(C)C)c12 ; Cc1c(-c2nc(N[C@@H]3CCOC[C@H]3O)ncc2F)ccc2ncc(CN3CC4CC3C4)c(C(C)C)c12]

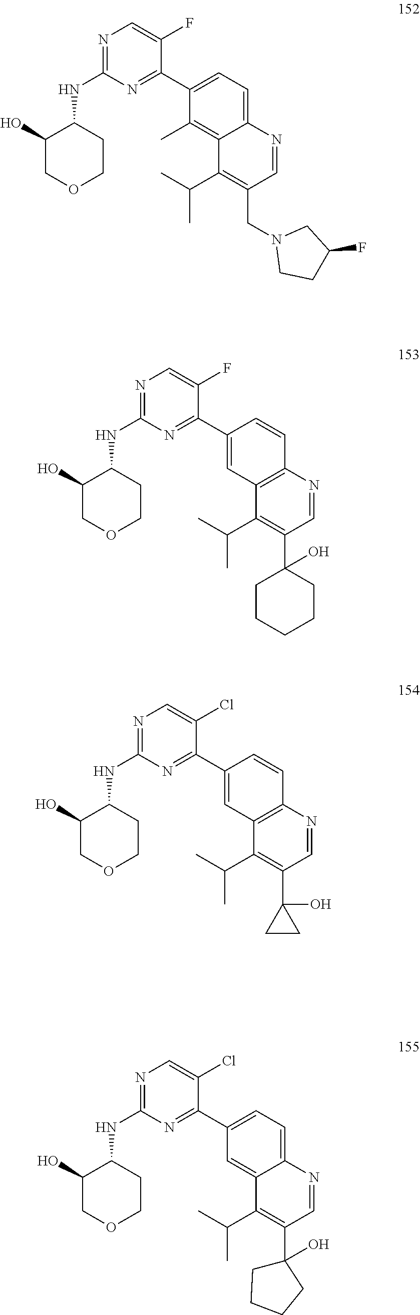 [SMILES: Cc1c(-c2nc(N[C@@H]3CCOC[C@H]3O)ncc2F)ccc2ncc(CN3CC[C@H](F)C3)c(C(C)C)c12 ; CC(C)c1c(C2(O)CCCCC2)cnc2ccc(-c3nc(N[C@@H]4CCOC[C@H]4O)ncc3F)cc12 ; CC(C)c1c(C2(O)CC2)cnc2ccc(-c3nc(N[C@@H]4CCOC[C@H]4O)ncc3Cl)cc12 ; CC(C)c1c(C2(O)CCCC2)cnc2ccc(-c3nc(N[C@@H]4CCOC[C@H]4O)ncc3Cl)cc12]

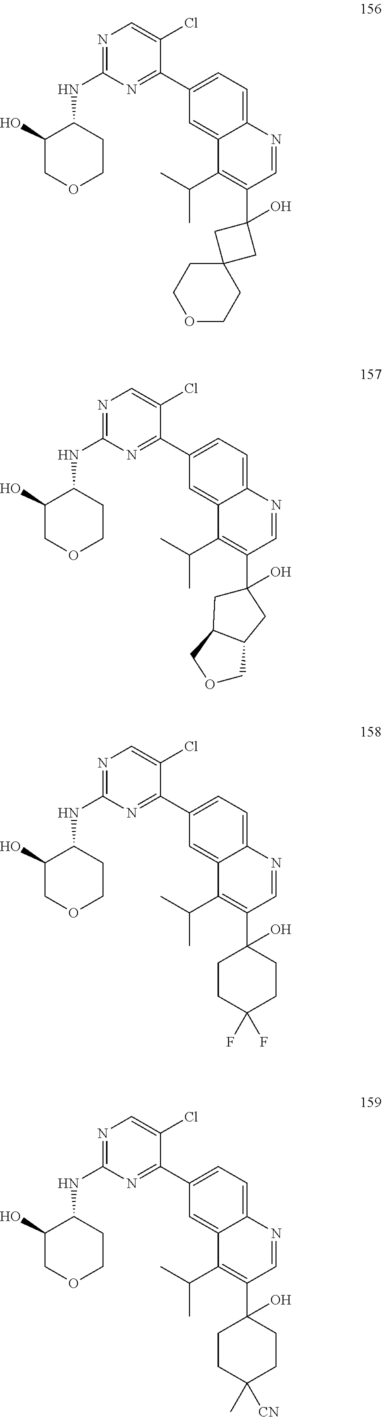 [SMILES: CC(C)c1c(C2(O)CC3(CCOCC3)C2)cnc2ccc(-c3nc(N[C@@H]4CCOC[C@H]4O)ncc3Cl)cc12 ; CC(C)c1c(C2(O)C[C@@H]3COC[C@H]3C2)cnc2ccc(-c3nc(N[C@@H]4CCOC[C@H]4O)ncc3Cl)cc12 ; CC(C)c1c(C2(O)CCC(F)(F)CC2)cnc2ccc(-c3nc(N[C@@H]4CCOC[C@H]4O)ncc3Cl)cc12 ; CC(C)c1c(C2(O)CCC(C)(C#N)CC2)cnc2ccc(-c3nc(N[C@@H]4CCOC[C@H]4O)ncc3Cl)cc12]

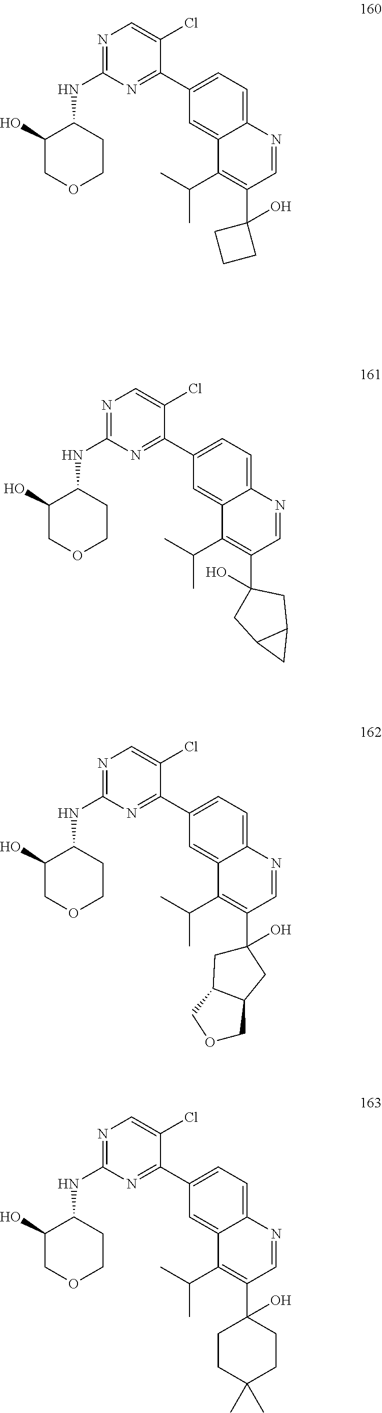 [SMILES: CC(C)c1c(C2(O)CCC2)cnc2ccc(-c3nc(N[C@@H]4CCOC[C@H]4O)ncc3Cl)cc12 ; CC(C)c1c(C2(O)CC3CC3C2)cnc2ccc(-c3nc(N[C@@H]4CCOC[C@H]4O)ncc3Cl)cc12 ; CC(C)c1c(C2(O)C[C@H]3COC[C@@H]3C2)cnc2ccc(-c3nc(N[C@@H]4CCOC[C@H]4O)ncc3Cl)cc12 ; CC(C)c1c(C2(O)CCC(C)(C)CC2)cnc2ccc(-c3nc(N[C@@H]4CCOC[C@H]4O)ncc3Cl)cc12]

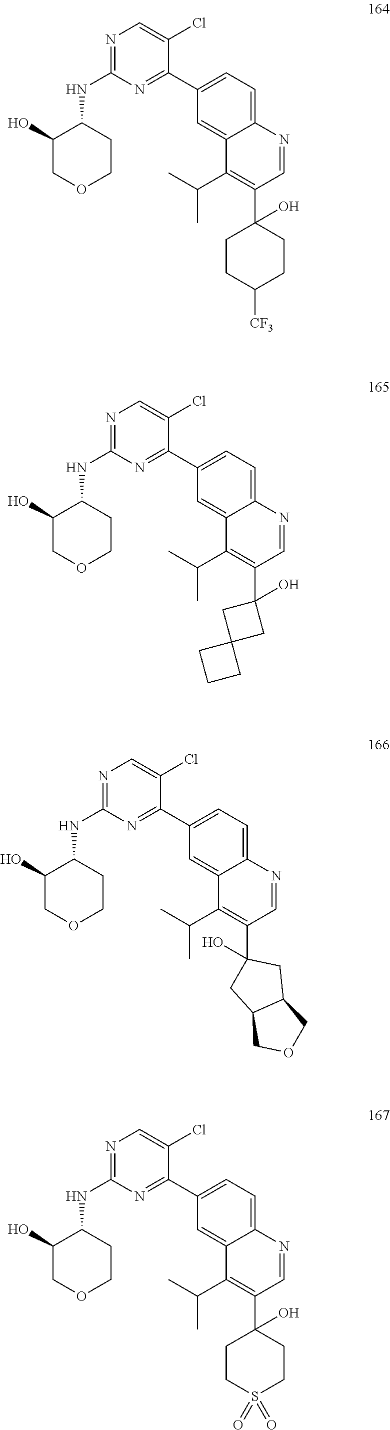 [SMILES: CC(C)c1c(C2(O)CCC(C(F)(F)F)CC2)cnc2ccc(-c3nc(N[C@@H]4CCOC[C@H]4O)ncc3Cl)cc12 ; CC(C)c1c(C2(O)CC3(CCC3)C2)cnc2ccc(-c3nc(N[C@@H]4CCOC[C@H]4O)ncc3Cl)cc12 ; CC(C)c1c(C2(O)C[C@H]3COC[C@H]3C2)cnc2ccc(-c3nc(N[C@@H]4CCOC[C@H]4O)ncc3Cl)cc12 ; CC(C)c1c(C2(O)CCS(=O)(=O)CC2)cnc2ccc(-c3nc(N[C@@H]4CCOC[C@H]4O)ncc3Cl)cc12]

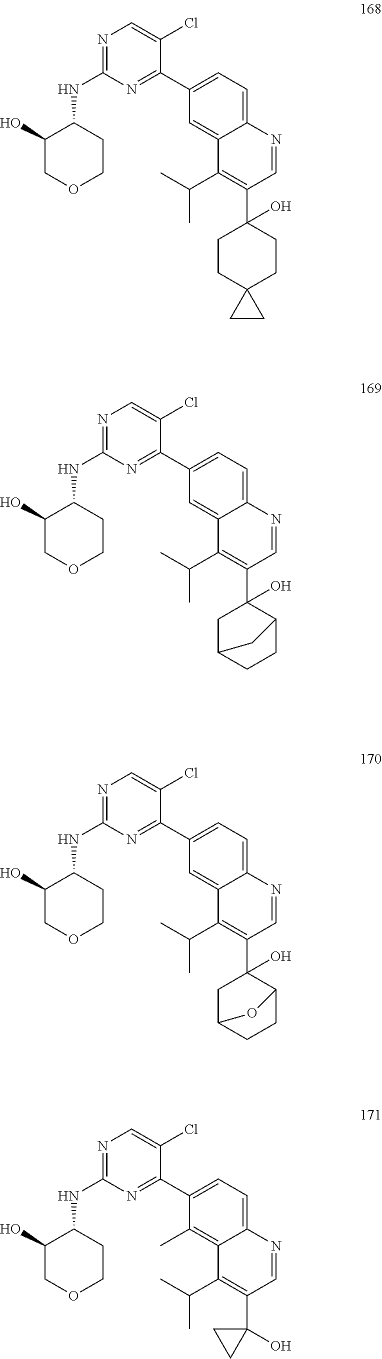 [SMILES: CC(C)c1c(C2(O)CCC3(CC3)CC2)cnc2ccc(-c3nc(N[C@@H]4CCOC[C@H]4O)ncc3Cl)cc12 ; CC(C)c1c(C2(O)CC3CCC2C3)cnc2ccc(-c3nc(N[C@@H]4CCOC[C@H]4O)ncc3Cl)cc12 ; CC(C)c1c(C2(O)CC3CCC2O3)cnc2ccc(-c3nc(N[C@@H]4CCOC[C@H]4O)ncc3Cl)cc12 ; Cc1c(-c2nc(N[C@@H]3CCOC[C@H]3O)ncc2Cl)ccc2ncc(C3(O)CC3)c(C(C)C)c12]

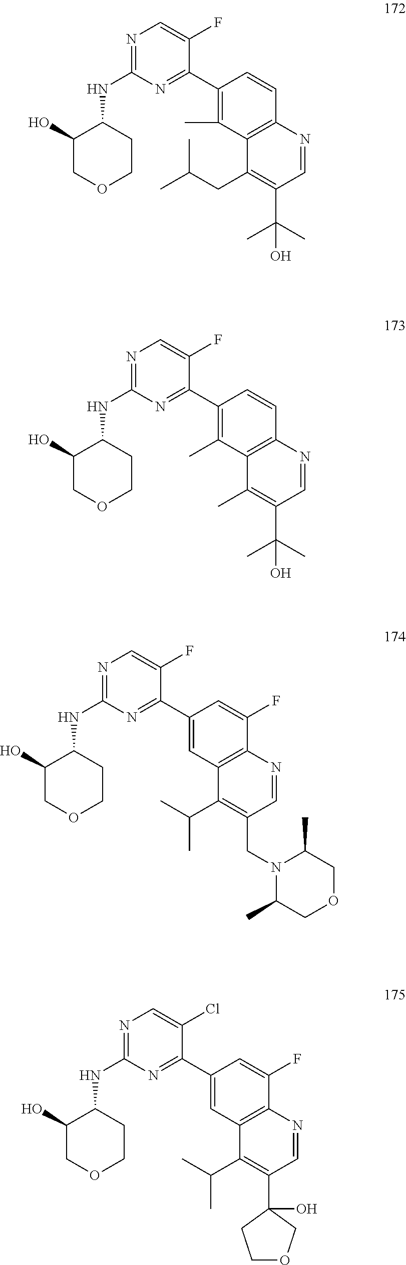 [SMILES: Cc1c(-c2nc(N[C@@H]3CCOC[C@H]3O)ncc2F)ccc2ncc(C(C)(C)O)c(CC(C)C)c12 ; Cc1c(-c2nc(N[C@@H]3CCOC[C@H]3O)ncc2F)ccc2ncc(C(C)(C)O)c(C)c12 ; CC(C)c1c(CN2[C@H](C)COC[C@@H]2C)cnc2c(F)cc(-c3nc(N[C@@H]4CCOC[C@H]4O)ncc3F)cc12 ; CC(C)c1c(C2(O)CCOC2)cnc2c(F)cc(-c3nc(N[C@@H]4CCOC[C@H]4O)ncc3Cl)cc12]

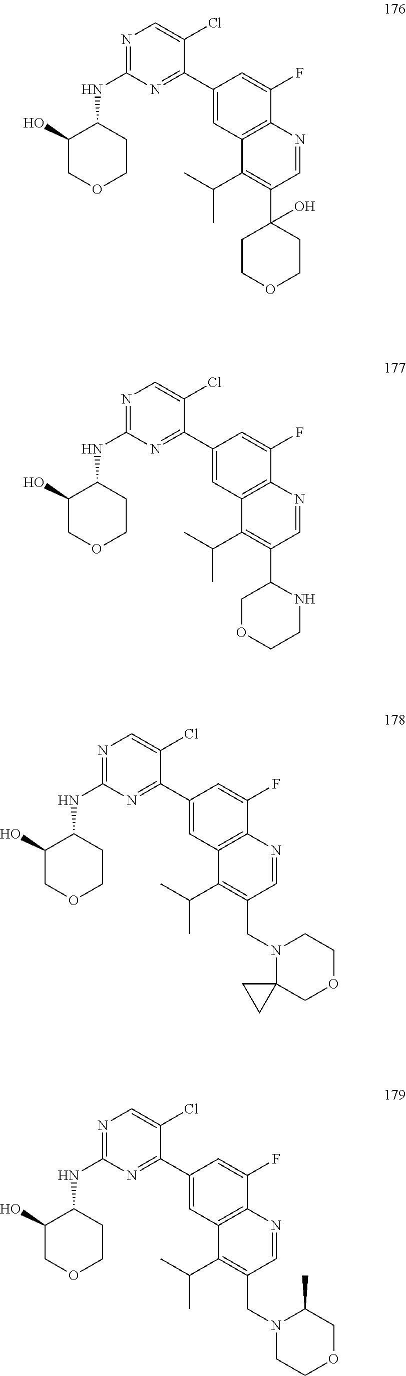 [SMILES: CC(C)c1c(C2(O)CCOCC2)cnc2c(F)cc(-c3nc(N[C@@H]4CCOC[C@H]4O)ncc3Cl)cc12 ; CC(C)c1c(C2COCCN2)cnc2c(F)cc(-c3nc(N[C@@H]4CCOC[C@H]4O)ncc3Cl)cc12 ; CC(C)c1c(CN2CCOCC23CC3)cnc2c(F)cc(-c3nc(N[C@@H]4CCOC[C@H]4O)ncc3Cl)cc12 ; CC(C)c1c(CN2CCOC[C@@H]2C)cnc2c(F)cc(-c3nc(N[C@@H]4CCOC[C@H]4O)ncc3Cl)cc12]

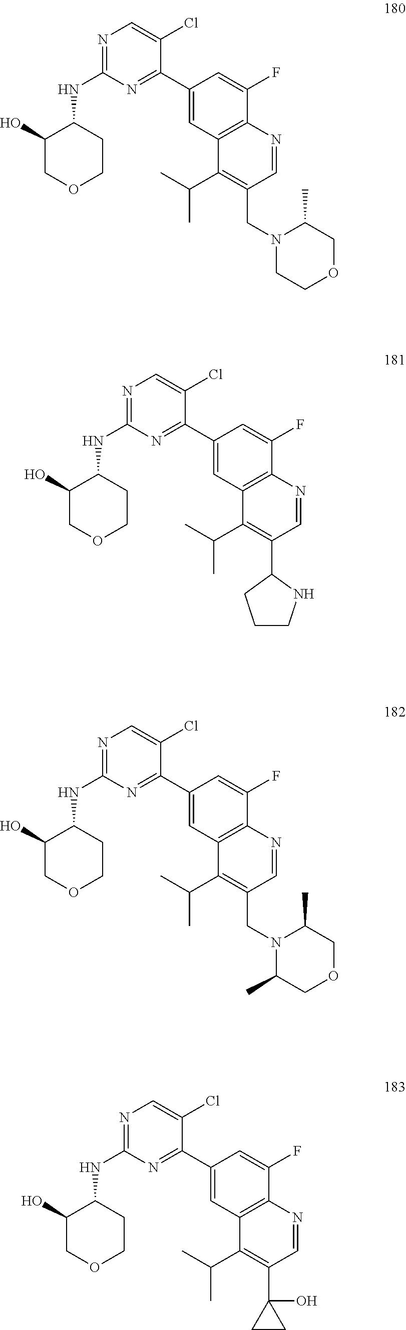 [SMILES: CC(C)c1c(CN2CCOC[C@H]2C)cnc2c(F)cc(-c3nc(N[C@@H]4CCOC[C@H]4O)ncc3Cl)cc12 ; CC(C)c1c(C2CCCN2)cnc2c(F)cc(-c3nc(N[C@@H]4CCOC[C@H]4O)ncc3Cl)cc12 ; CC(C)c1c(CN2[C@H](C)COC[C@@H]2C)cnc2c(F)cc(-c3nc(N[C@@H]4CCOC[C@H]4O)ncc3Cl)cc12 ; CC(C)c1c(C2(O)CC2)cnc2c(F)cc(-c3nc(N[C@@H]4CCOC[C@H]4O)ncc3Cl)cc12]

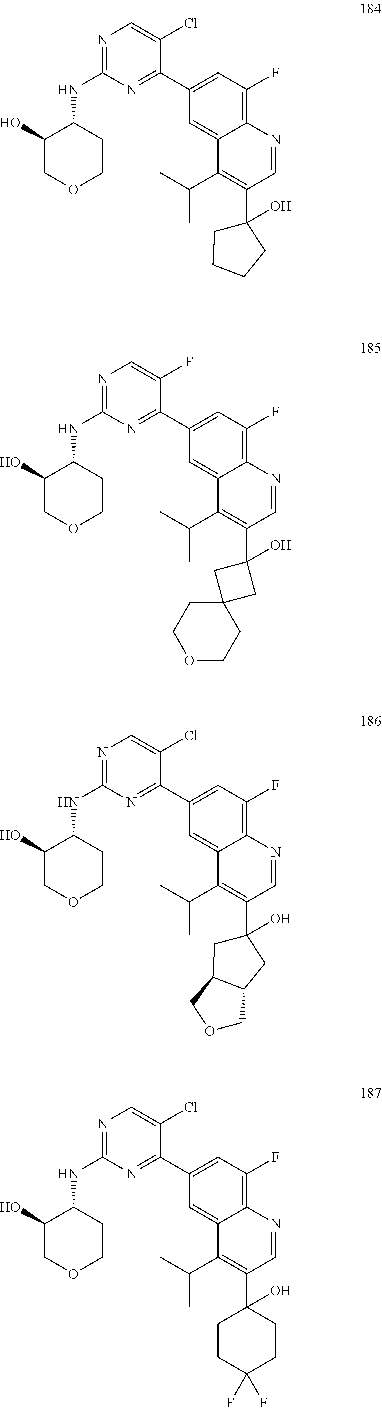 [SMILES: CC(C)c1c(C2(O)CCCC2)cnc2c(F)cc(-c3nc(N[C@@H]4CCOC[C@H]4O)ncc3Cl)cc12 ; CC(C)c1c(C2(O)CC3(CCOCC3)C2)cnc2c(F)cc(-c3nc(N[C@@H]4CCOC[C@H]4O)ncc3F)cc12 ; CC(C)c1c(C2(O)C[C@@H]3COC[C@H]3C2)cnc2c(F)cc(-c3nc(N[C@@H]4CCOC[C@H]4O)ncc3Cl)cc12 ; CC(C)c1c(C2(O)CCC(F)(F)CC2)cnc2c(F)cc(-c3nc(N[C@@H]4CCOC[C@H]4O)ncc3Cl)cc12]

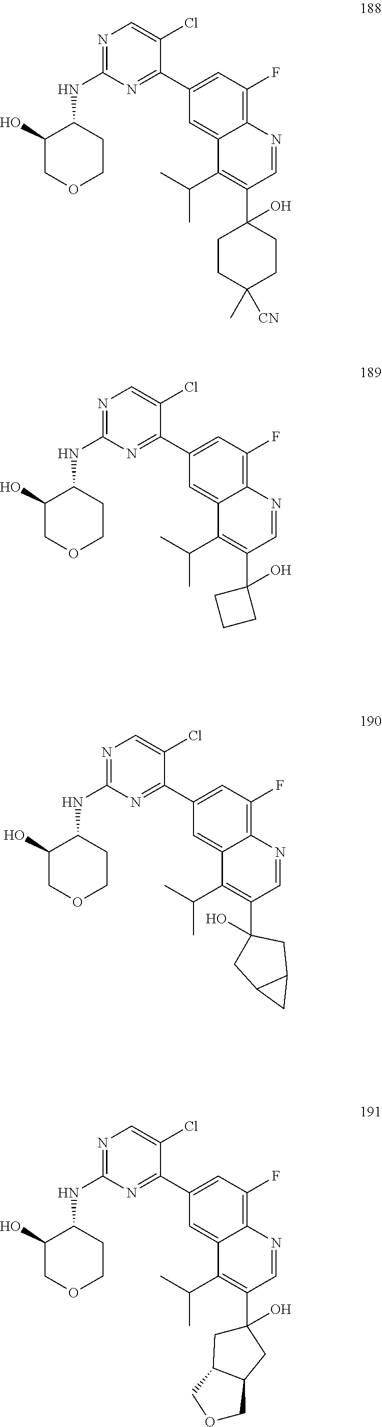 [SMILES: CC(C)c1c(C2(O)CCC(C)(C#N)CC2)cnc2c(F)cc(-c3nc(N[C@@H]4CCOC[C@H]4O)ncc3Cl)cc12 ; CC(C)c1c(C2(O)CCC2)cnc2c(F)cc(-c3nc(N[C@@H]4CCOC[C@H]4O)ncc3Cl)cc12 ; CC(C)c1c(C2(O)CC3CC3C2)cnc2c(F)cc(-c3nc(N[C@@H]4CCOC[C@H]4O)ncc3Cl)cc12 ; CC(C)c1c(C2(O)C[C@H]3COC[C@@H]3C2)cnc2c(F)cc(-c3nc(N[C@@H]4CCOC[C@H]4O)ncc3Cl)cc12]

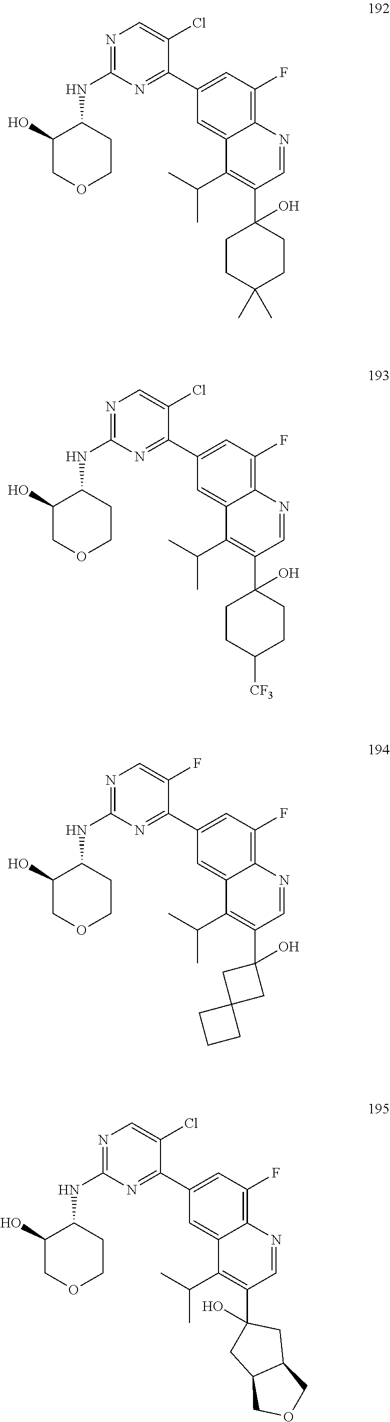 [SMILES: CC(C)c1c(C2(O)CCC(C)(C)CC2)cnc2c(F)cc(-c3nc(N[C@@H]4CCOC[C@H]4O)ncc3Cl)cc12 ; CC(C)c1c(C2(O)CCC(C(F)(F)F)CC2)cnc2c(F)cc(-c3nc(N[C@@H]4CCOC[C@H]4O)ncc3Cl)cc12 ; CC(C)c1c(C2(O)CC3(CCC3)C2)cnc2c(F)cc(-c3nc(N[C@@H]4CCOC[C@H]4O)ncc3F)cc12 ; CC(C)c1c(C2(O)C[C@H]3COC[C@H]3C2)cnc2c(F)cc(-c3nc(N[C@@H]4CCOC[C@H]4O)ncc3Cl)cc12]

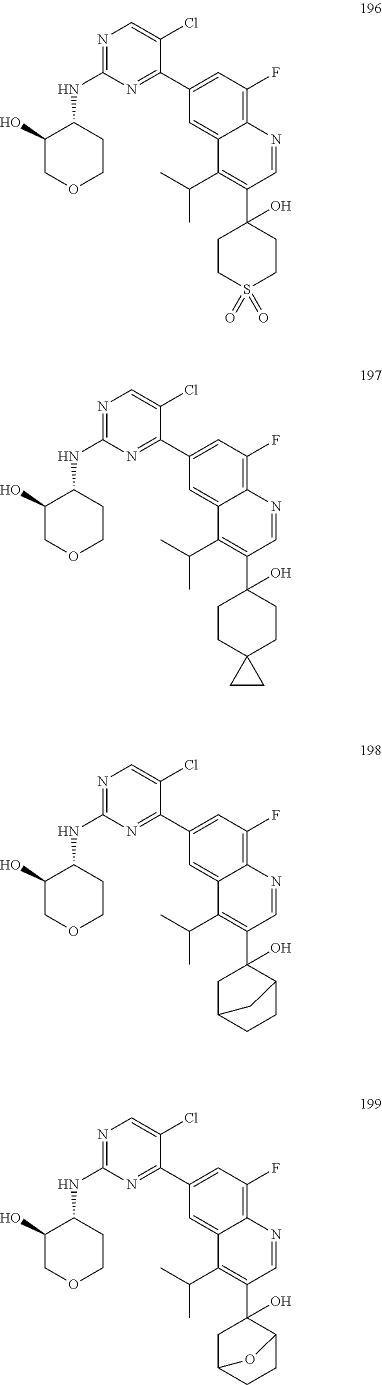 [SMILES: CC(C)c1c(C2(O)CCS(=O)(=O)CC2)cnc2c(F)cc(-c3nc(N[C@@H]4CCOC[C@H]4O)ncc3Cl)cc12 ; CC(C)c1c(C2(O)CCC3(CC3)CC2)cnc2c(F)cc(-c3nc(N[C@@H]4CCOC[C@H]4O)ncc3Cl)cc12 ; CC(C)c1c(C2(O)CC3CCC2C3)cnc2c(F)cc(-c3nc(N[C@@H]4CCOC[C@H]4O)ncc3Cl)cc12 ; CC(C)c1c(C2(O)CC3CCC2O3)cnc2c(F)cc(-c3nc(N[C@@H]4CCOC[C@H]4O)ncc3Cl)cc12]

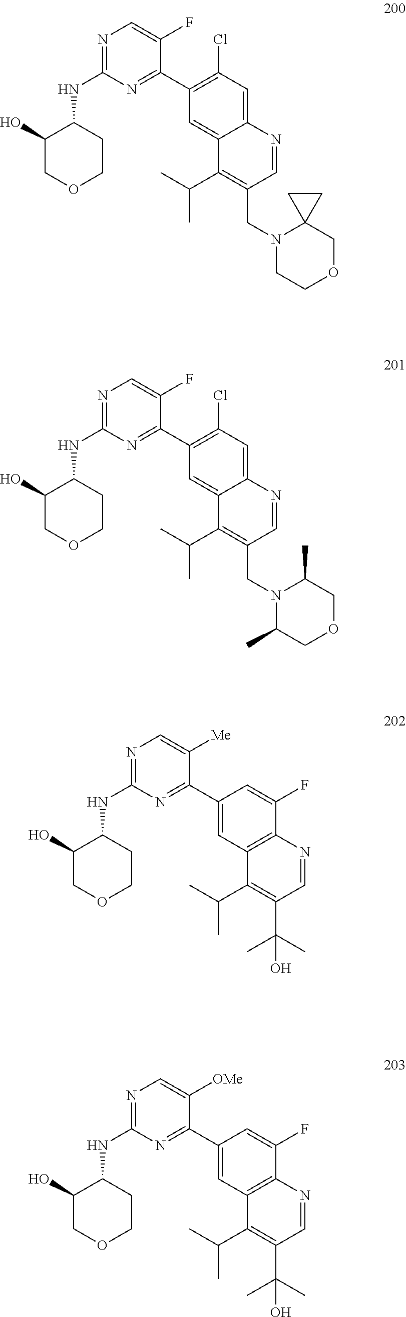 [SMILES: CC(C)c1c(CN2CCOCC23CC3)cnc2cc(Cl)c(-c3nc(N[C@@H]4CCOC[C@H]4O)ncc3F)cc12 ; CC(C)c1c(CN2[C@H](C)COC[C@@H]2C)cnc2cc(Cl)c(-c3nc(N[C@@H]4CCOC[C@H]4O)ncc3F)cc12 ; Cc1cnc(N[C@@H]2CCOC[C@H]2O)nc1-c1cc(F)c2ncc(C(C)(C)O)c(C(C)C)c2c1 ; COc1cnc(N[C@@H]2CCOC[C@H]2O)nc1-c1cc(F)c2ncc(C(C)(C)O)c(C(C)C)c2c1]

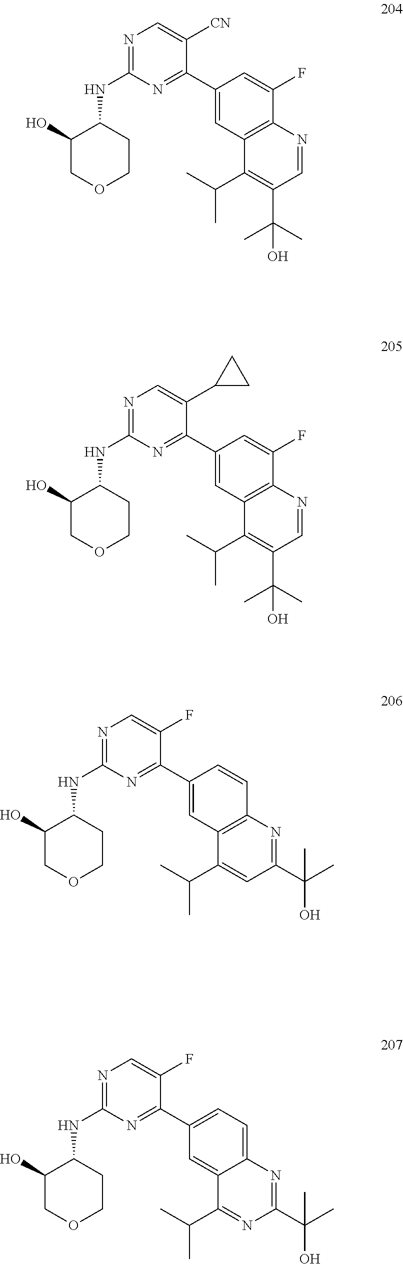 [SMILES: CC(C)c1c(C(C)(C)O)cnc2c(F)cc(-c3nc(N[C@@H]4CCOC[C@H]4O)ncc3C#N)cc12 ; CC(C)c1c(C(C)(C)O)cnc2c(F)cc(-c3nc(N[C@@H]4CCOC[C@H]4O)ncc3C3CC3)cc12 ; CC(C)c1cc(C(C)(C)O)nc2ccc(-c3nc(N[C@@H]4CCOC[C@H]4O)ncc3F)cc12 ; CC(C)c1nc(C(C)(C)O)nc2ccc(-c3nc(N[C@@H]4CCOC[C@H]4O)ncc3F)cc12]

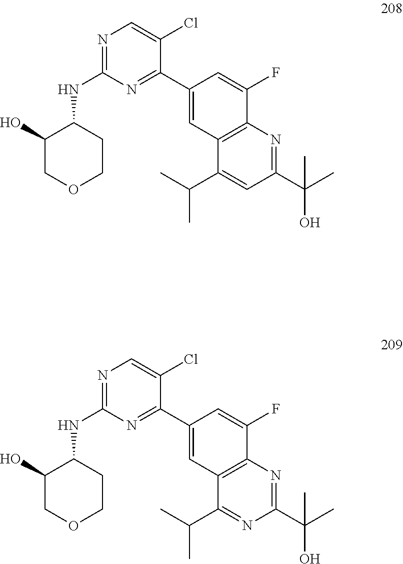 [SMILES: CC(C)c1cc(C(C)(C)O)nc2c(F)cc(-c3nc(N[C@@H]4CCOC[C@H]4O)ncc3Cl)cc12 ; CC(C)c1nc(C(C)(C)O)nc2c(F)cc(-c3nc(N[C@@H]4CCOC[C@H]4O)ncc3Cl)cc12]

Aspect 20. A pharmaceutical composition comprising a compound of any one of Aspects 1-19 or a pharmaceutically acceptable salt, stereoisomer, tautomer or prodrug thereof, together with a pharmaceutically acceptable excipient.

Aspect 21. A method of decreasing CDK4 activity by inhibition, which comprises administering to an individual the compound according to any one of Aspects 1-19, or a pharmaceutically acceptable salt thereof, including the compound of formula (1) or the specific compounds exemplified herein.

Aspect 22. The method of Aspect 21, wherein the disease is selected from cancer, preferred breast cancer, lung cancer, pancreatic cancer, prostate cancer, bone cancer, liver cancer and endometrial cancer.

Aspect 23. Use of a compound of any one of Aspects 1-19 or a pharmaceutically acceptable salt, stereoisomer, tautomer or prodrug thereof in the preparation of a medicament for treating a disease that can be affected by CDK4 modulation.

Aspect 24. The use of Aspect 23, wherein the disease is cancer, preferred breast cancer, lung cancer, pancreatic cancer, prostate cancer, bone cancer, liver cancer and endometrial cancer.

Aspect 25. A method of treating a disease or disorder in a patient comprising administering to the patient a therapeutically effective amount of the compound any one of Aspects 1-19, or a pharmaceutically acceptable salt thereof as a CDK4 kinase inhibitor, wherein the disease or disorder is associated with inhibition of CDK4.

Aspect 26. The method of Aspect 25, wherein the disease is selected from cancer, preferred breast cancer, lung cancer, pancreatic cancer, prostate cancer, bone cancer, liver cancer and endometrial cancer.

### DETAILED DESCRIPTION OF THE INVENTION

The following terms have the indicated meanings throughout the specification; Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”. “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”). 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane.

Alkylene includes but not limited to methylene, ethylene, propylene, and so on.

The term “halogen” includes fluoro (F), chloro (Cl), bromo (Br) and iodo (1).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C═C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkenylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈ cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2,3-dihydro-1H-indenyl, 1H-indenyl, 1,2,3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic. e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O. and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group, the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. C is formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel. E. and Wilen, S. Stereochemistry of Organic Compounds. New York: John Wiley & Sons, Inc., 1994: Lochmuller. C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods. (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed Drug Stereochemistry; Analytical Method and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”. “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆ and the like.

Unless specifically defined elsewhere in this document, all other technical and scientific terms used herein have the meaning commonly understood by one of ordinary skill in the art to which this invention belongs.

### EXAMPLES

### General Synthesis

Compounds disclosed herein, including salts thereof, can be prepared using known organic synthesis techniques and can be synthesized according to any of numerous possible synthetic routes. The reaction for preparing compounds disclosed herein can be carried out in suitable solvents which can be readily selected by one of skill in the art of organic synthesis. Suitable solvents can be substantially non-reactive with the starting materials, the intermediates, or products at the temperatures at which the reactions are carried out, e.g., temperatures which can range from the boiling temperature of solvent. A given reaction can be carried out in one solvent or mixture of solvents.

The selection of appropriate protecting group, can be readily determined by one skilled in the art.

Reactions can be monitored according to any suitable method known in the art, such as NMR, UV, HPLC, LC-MS and TLC. Compounds can be purified by a variety of methods, including HPLC and normal phase silica chromatography.

Chiral analytic HPLC was used for the retention time analysis of different chiral examples, the conditions were divided into the methods as below according to the column, mobile phase, solvent ration used.

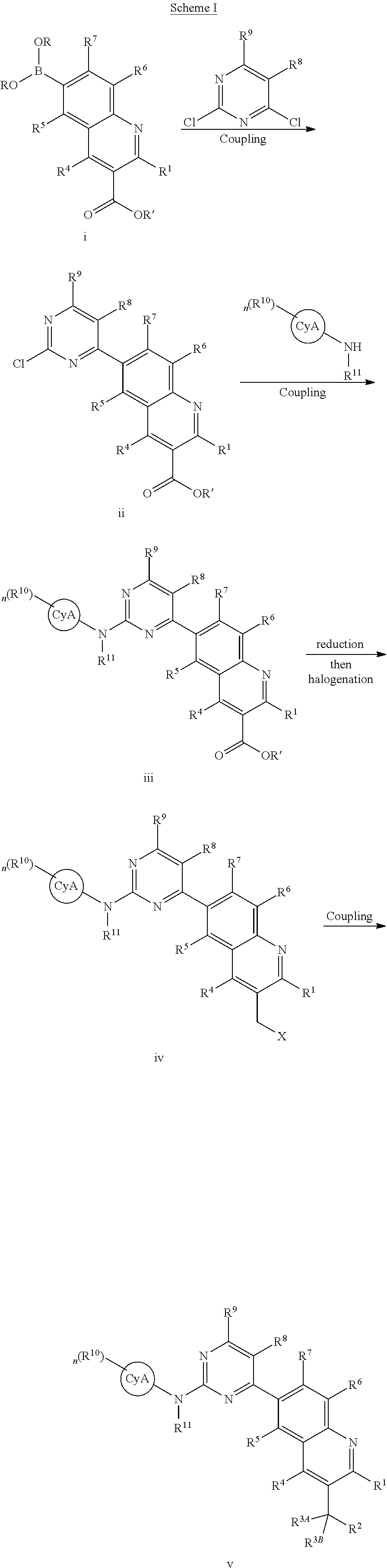

For example, compounds of Formulas (I), (II), (III), or (IV) can be formed as shown in Scheme I. The compound (i) can react with halogenated pyrimidine tinder palladium catalyzed reaction condition or base mediated coupling condition to give compound (ii) that can couple with amine to give compound (iii), reduction and halogenation of compound (iii) give compound (iv) which can be used for coupling to give compound (v).

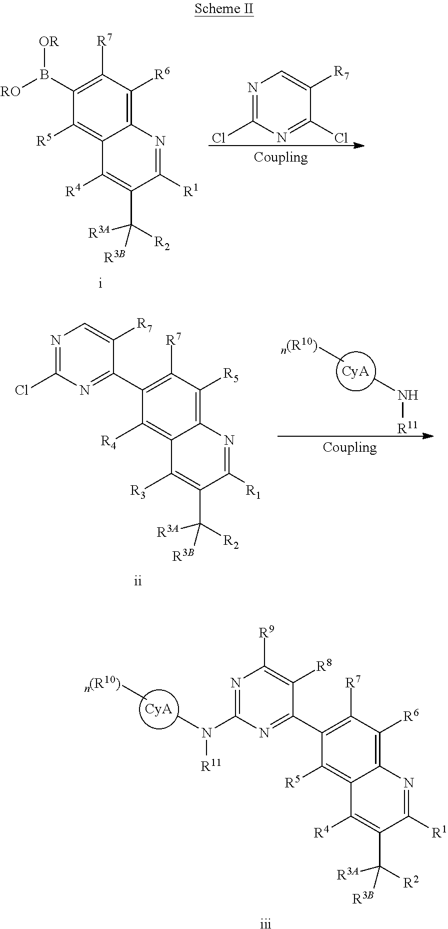

For example, compounds of Formulas (I), (II), (AIII), (III), (AIV) or (IV) can be formed as shown in Scheme II. The compound (i) can react with halogenated pyrimidine under palladium catalyzed reaction condition to give compound (ii) that can couple with amine to give compound (iii).

### Example 1: 6-(5-fluoro-2-(((3S,4R)-3-hydroxytetrahydro-2H-pyran-4-yl)amino)pyrimidin-4-yl)-4-isopropyl-N-methyl quinoline-3-carboxamide

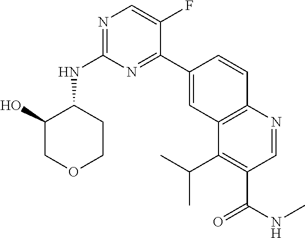 [SMILES: CNC(=O)c1cnc2ccc(-c3nc(N[C@@H]4CCOC[C@H]4O)ncc3F)cc2c1C(C)C]

### Step 1: ethyl 6-chloro-4-isopropylquinoline-3-carboxylate

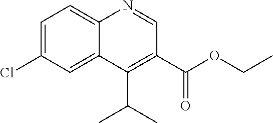 [SMILES: CCOC(=O)c1cnc2ccc(Cl)cc2c1C(C)C]

To the solution of isopropylmagnesium bromide (15 mL, 1M, 15 mmol) under nitrogen was added zinc chloride solution in tetrahydrofuran (21.4 mL, 0.7 M, 15 mmol) and the mixture was stirred at 50° C. for 2 h. The solution of ethyl 4,6-dichloroquinoline-3-carboxylate (2 g, 7.5 mmol) in dimethylformamide (10 mL), copper(I) iodide (143 mg, 0.75 mmol) and (1,1′-bis(diphenylphosphino)ferrocene)palladium(II) dichloride (275 mg, 0.375 mmol) were added and the resulting mixture was stirred at 50° C. for 2 h before cooled to room temperature. The reaction was quenched with methanol (10 mL) and the solvent was evaporated. The residue was suspended in ethyl acetate and water before filtration. The filtrate was washed with water and brine, dried over sodium sulfate, filtered and evaporated. The residue was purified over silica gel by combi-flash, eluting with ethyl acetate in petroleum ether (16%, v/v) to give the title compound (1.5 g, 71%). LC-MS (M+H)⁺=278.2.

### Step 2: ethyl 4-isopropyl-6-(4,4,5,5-tetramethyl-1,3,2-dioxaborolan-2-ylquinoline-3-carboxylate

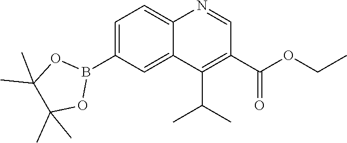 [SMILES: CCOC(=O)c1cnc2ccc(B3OC(C)(C)C(C)(C)O3)cc2c1C(C)C]

A mixture of ethyl 6-chloro-4-isopropylquinoline-3-carboxylate (1.3 g, 4, mmol), bis(pinacolato)diboron (1.4 g, 5.6 mmol), (1,1′-bis(diphenylphosphino)ferrocene)palladium(II) dichloride (172 mg, 0.24 mmol) and potassium acetate (921 mg, 9.4 mmol) in 1,4-dioxane (40 mL) was stirred at 80° C. under nitrogen for overnight before cooled to room temperature. The mixture was filtered and the filtrate was evaporated to give the crude product which was used for next step without further purification. LC-MS (M+H)⁺=370.3.

### Step 3: ethyl 6-(2-chloro-5-fluoropyrimidin-4-yl)-4-isopropylquinoline-3-carboxylate

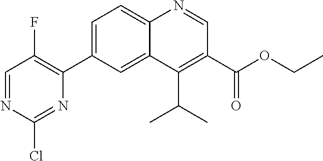 [SMILES: CCOC(=O)c1cnc2ccc(-c3nc(Cl)ncc3F)cc2c1C(C)C]

A mixture of ethyl 4-isopropyl-6-(4,4,5,5-tetramethyl-1,3,2-dioxaborolan-2-yl)quinoline-3-carboxylate, 2,4-dichloro-5-fluoropyrimidine (785 mg, 4.7 mmol). (1,1′-bis(diphenylphosphino)ferrocene)palladium(II) dichloride (176 mg, 0.24 mmol) and K₂CO₃ (1.3 g, 9.4 mmol) in 1,4-dioxane (40 mL) and water (5 mL) under nitrogen was stirred at 80° C. for overnight before cooled to room temperature. The mixture was diluted with ethyl acetate, washed with brine, dried over sodium sulfate, filtered and evaporated. The residue was purified over silica gel by combi-flash, eluting with methanol in dichloromethane (3%, v/v) to give the title compound (790 mg, 45% for two steps). LC-MS (M+H)⁺=374.1.

### Step 4: ethyl 6-(5-fluoro-2-(3S,4R)-3-hydroxytetrahydro-2H-pyran-4-ylamino)pyrimidin-4-yl)-4-isopropylquinoline-3-carboxylate

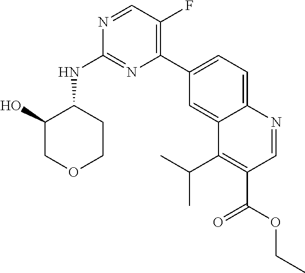 [SMILES: CCOC(=O)c1cnc2ccc(-c3nc(N[C@@H]4CCOC[C@H]4O)ncc3F)cc2c1C(C)C]

The reaction mixture of ethyl 6-(2-chloro-5-fluoropyrimidin-4-yl)-4-isopropylquinoline-3-carboxylate (790 mg, 2.11 mmol), (3S,4R)-4-aminotetrahydro-2H-pyran-3-ol (390 mg, 2.53 mmol), (SP-4-1)-[1,3-bis[2,6-bis(1-ethylpropyl)phenyl]-4,5-dichloro-1,3-dihydro-2H-imidazol-2-ylidene]dichloro(2-methylpyridine)palladium (89 mg, 0.11 mmol) and cesium carbonate (2.1 g, 6.33 mmol) in 1,4-dioxane (30 mL) under nitrogen was stirred at 100° C. for overnight. The mixture was cooled to room temperature and the solvent was evaporated. The residue was purified over silica gel by combi-flash, eluting with methanol in dichloromethane (6%, v/v) to give the title compound (780 mg, 81%). LC-MS (M+H)⁺=455.8.

### Step 5: 6-(5-fluoro-2-(((3S,4R)-3-hydroxytetrahydro-2H-pyran-4-ylamino)pyrimidin-4-yl)-4-isopropylquinoline-3-carboxylic acid

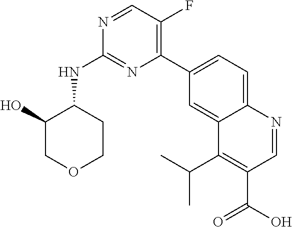 [SMILES: CC(C)c1c(C(=O)O)cnc2ccc(-c3nc(N[C@@H]4CCOC[C@H]4O)ncc3F)cc12]

To the solution of ethyl 6-(5-fluoro-2-(((3S,4R)-3-hydroxytetrahydro-2H-pyran-4-yl)amino)pyrimidin-4-yl)-4-isopropylquinoline-3-carboxylate (150 mg, 0.33 mmol) in tetrahydrofuran (15 mL) was added the aqueous solution of lithium hydroxide monohydrate (30 mg, 0.66 mmol) and the mixture was stirred at 55° C. for overnight before cooled to room temperature. The mixture was acidified with 1 N HCl to pH=6. The precipitate was filtered and dried to give the product (130 mg, 92%). LC-MS (M+H)⁺=427.3

### Step 6: 6-(5-fluoro-2-(3S,4R)-3-hydroxytetrahydro-2H-pyran-4-yl)amino)pyrimidin-4-yl)-4-isopropyl-N-methylquinoline-3-carboxamide

The reaction mixture of 6-(5-fluoro-2-(((3S,4R)-3-hydroxytetrahydro-2H-pyran-4-yl)amino)pyrimidin-4-yl)-4-isopropylquinoline-3-carboxylic acid (100 mg, 0.24 mmol), methylamine hydrochloride (30 mg, 0.47 mmol), 2-(1H-7-azabenzotriazol-1-yl)-1,1,3,3-tetramethyl uronium hexafluorophosphate (91 mg, 0.24 mmol), N,N-diisopropylethylamine (61 mg, 0.47 mmol) and dimethylformamide (5 mL) was stirred at 50° C. for 3 h before cooled to room temperature. Water was added and the aqueous phase was extracted with ethyl acetate (20 mL×3). The combined organic layers were washed with brine, dried over sodium sulfate, filtered and evaporated. The residue was purified by prep-HPLC (eluent: water (0.1% formic acid)/acetonitrile (0.1% formic acid)) to give the product (45 mg, 43%). ¹H-NMR (400 MHz, DMSO-d6) δ 9.08 (s, 1H), 8.74 (s, 1H), 8.60 (d, J=4.3 Hz, 1H), 8.51 (d, J=3.5 Hz, 1H), 8.35 (d, J=8.8 Hz, 1H), 8.19 (d, J=8.8 Hz, 1H), 7.28 (d, J=7.7 Hz, 1H), 4.97 (d, J=4.9 Hz, 1H), 4.00-3.79 (m, 3H), 3.72 (dd, J=14.2, 7.0 Hz, 1H), 3.55 (s, 1H), 3.38-3.36 (m, 1H), 3.06 (t, J=10.4 Hz, 1H), 2.84 (d, J=4.3 Hz, 3H), 2.03 (s, 1H), 1.55-1.49 (m, 7H). LC-MS (M+H)⁺=440.3.

### Example 2: (3S,4R)-4-(5-fluoro-4-(3-(2-hydroxypropan-2-yl)-4-isopropylquinolin-6-yl)pyrimidin-2-yl)amino)tetrahydro-2H-pyran-3-ol

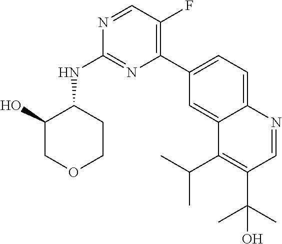 [SMILES: CC(C)c1c(C(C)(C)O)cnc2ccc(-c3nc(N[C@@H]4CCOC[C@H]4O)ncc3F)cc12]

To the solution of ethyl 6-(5-fluoro-2-(((3S,4R)-3-hydroxytetrahydro-2H-pyran-4-yl)amino)pyrimidin-4-yl)-4-isopropylquinoline-3-carboxylate (50 mg, 0.11 mmol) in tetrahydrofuran (10 mL) under nitrogen at −78° C. was added 3 M methylmagnesium bromide solution in tetrahydrofuran (0.1 mL, 0.33 mmol) and the mixture was stirred at room temperature for 4 h. The reaction was quenched with water and the mixture was evaporated. The residue was purified by prep-HPLC (eluent: water (0.1% formic acid)/acetonitrile (0.1% formic acid)) to give the product (6 mg, 12%). ¹H-NMR (400 MHz, DMSO-d6) δ 9.19 (s, 1H), 9.08 (s, 1H), 8.49 (d, J=3.8 Hz, 1H), 8.25 (t, J=9.2 Hz, 1H), 8.11 (d, J=8.8 Hz, 1H), 7.24 (d, J=7.9 Hz, 1H), 5.43 (s, 1H), 4.97 (d, J=4.7 Hz, 1H), 4.74-4.54 (m, 1H), 3.99-3.77 (m, 3H), 3.54 (s, 1H), 3.39-3.36 (m, 1H), 3.05 (t, J=10.4 Hz, 1H), 2.1-1.93 (m, 1H), 1.70 (s, 6H), 1.65-1.40 (m, 7H). LC-MS (M+H)⁺=441.3.

### Example 3: (3S,4R)-4-((5-fluoro-4-(3-(hydroxymethyl)-4-isopropylquinolin-6-yl)pyrimidin-2-yl)amino)tetrahydro-2H-pyran-3-ol

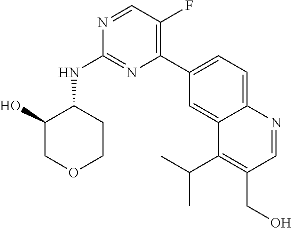 [SMILES: CC(C)c1c(CO)cnc2ccc(-c3nc(N[C@@H]4CCOC[C@H]4O)ncc3F)cc12]

To the solution of ethyl 6-(5-fluoro-2-(((3S,4R)-3-hydroxytetrahydro-2H-pyran-4-yl)amino)pyrimidin-4-yl)4-isopropylquinoline-3-carboxylate (50 mg, 0.11 mmol) in tetrahydrofuran (10 mL) under nitrogen at 0° C. was added lithium aluminium hydride (13 mg, 0.33 mmol) and stirred at room temperature for 3 h. The reaction was quenched with methanol and the solvent was evaporated. The residue was purified by prep-HPLC (eluent: water (0.1% formic acid)/acetonitrile (0.1% formic acid)) to give the product (6 mg, 12%). ¹H-NMR (400 MHz, DMSO-d6) δ 9.10 (s, 1H), 8.90 (s, 1H), 8.50 (d, J=3.7 Hz, 1H), 8.28 (d, J=8.8 Hz, 1H), 8.14 (d, J=8.8 Hz, 1H), 7.26 (d, J=7.8 Hz, 1H), 5.39 (s, 1H), 4.97 (s, 1H), 4.78 (s, 2H), 4.01-3.77 (m, 4H), 3.55 (s, 1H), 3.39-3.36 (m, 1H), 3.06 (t, J=10.4 Hz, 1H), 2.06-1.99 (m, 11H), 1.59-1.47 (m, 7H). LC-MS (M+H)⁺=413.3.

### Example 4: (3S,4R)-4-((5-chloro-4-(4-isopropylquinolin-6-yl)pyrimidin-2-yl)amino)tetrahydro-2H-pyran-3-ol

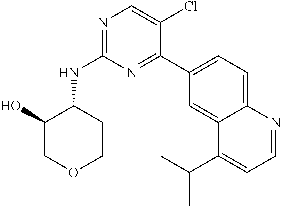 [SMILES: CC(C)c1ccnc2ccc(-c3nc(N[C@@H]4CCOC[C@H]4O)ncc3Cl)cc12]

### Step 1: 6-chloroquinolin-4-yl trifluoromethanesulfonate

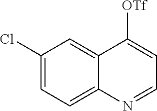 [SMILES: O=S(=O)(Oc1ccnc2ccc(Cl)cc12)C(F)(F)F]

6-Chloroquinolin-4-ol (3 g, 16.7 mmol) and N,N-diisopropylethylamine (2.59 g, 20.1 mmol) were dissolved in dichloromethane (30 mL). Trifluoromethanesulfonic anhydride (5.66 g, 20.1 mmol) was added dropwise at 0° C. and the reaction solution was stirred at 0° C. for 2 h before addition of water (20 mL). The aqueous layer was extracted with dichloromethane (50 mL×3). The combined organic layers were washed with brine, dried over sodium sulfate, filtered and concentrated. The residue was purified by flash chromatography over silica gel to give the title compound (4.1 g, 79%). LC-MS (M+H)⁺=312.0.

### Step 2: 6-chloro-44prop-1-en-2-ylquinoline

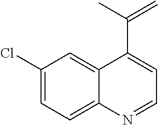

The title compound (700 mg, 26%) was prepared in a manner similar to Example 1 step 3 from 6-chloroquinolin-4-yl trifluoromethanesulfonate and 4,4,5,5-tetramethyl-2-(prop-1-en-2-yl)-1,3,2-dioxaborolane. LC-MS (M+H)⁺=204.0.

### Step 3: 6-chloro-4-isopropylquinoline

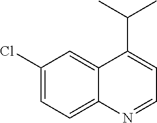

A mixture of 6-chloro-4-(prop-1-en-2-yl)quinoline (610 mg, 2.995 mmol) in methanol (10 mL) and PtO₂ (30.5 mg) was stirred under 1 atmosphere of hydrogen for 2.5 h. The mixture was filtered and the filtrate was concentrated under vacuum. The crude residue (616 mg) was used for next step without further purification. LC-MS (M+H)⁺=206.0.

### Step 4: 4-isopropyl-6-(4,4,5,5-tetramethyl-1,3,2-dioxaborolan-2-yl)quinoline

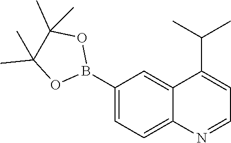 [SMILES: CC(C)c1ccnc2ccc(B3OC(C)(C)C(C)(C)O3)cc12]

The title compound (1.01 g, 100%) was prepared in a manner similar to Example 1 step 2 from 6-chloro-4-isopropylquinoline and 4,4,4′,4′,5,5,5′,5′-octamethyl-2,2′-bi(1,3,2-dioxaborolane). LC-MS (M+H)⁺=298.0.

### Step 5: 6-(2,5-dichloropyrimidin-4-yl)-4-isopropylquinoline

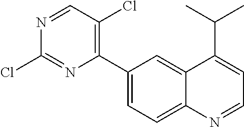 [SMILES: CC(C)c1ccnc2ccc(-c3nc(Cl)ncc3Cl)cc12]

The title compound (500 mg, 46%) was prepared in a manner similar to Example 1 step 3 from 2,4,5-trichloropyrimidine and 4-isopropyl-6-(4,4,5,5-tetramethyl-1,3,2-dioxaborolan-2-yl)quinoline. LC-MS (M+H)⁺=318.0.

### Step 6: (3S,4R)-4-((5-chloro-4-(4-isopropoylquinolin-6-yl)pyrimidin-2-yl)amino)tetrahydro-2H-pyran-3-ol

6-(2,5-dichloropyrimidin-4-yl)-4-isopropylquinoline (500 mg, 1.57 mmol) and N,N-diisopropylethylamine (609 mg, 4.71 mmol) were dissolved in acetonitrile (20 mL). (3S,4R)-4-aminotetrahydro-2H-pyran-3-ol hydrochloride (362 mg, 2.36 mmol) was added and the mixture was stirred at 80° C. for 36 h before cooled to room temperature. Solvent was removed under vacuum before addition of water (20 mL). Aqueous layer was extracted with ethyl acetate (40 mL×3). The combined organic layers were washed with brine, dried over sodium sulfate, filtered and concentrated. The crude was purified by flash chromatography over silica gel to give the title product (40 mg, 6.4%). ¹H-NMR (400 MHz, DMSO-d6) δ 8.92 (d, J=4.5 Hz, 1H), 8.60 (s, 1H), 8.47 (s, 1H), 8.14 (d, J=8.2 Hz, 1H), 8.09 (s, 1H), 7.56 (s, 1H), 7.51 (d, J=4.4 Hz, 1H), 4.95 (d, J=5.3 Hz, 1H), 3.91-3.72 (m, 4H), 3.51 (s, 1H), 3.33-3.26 (m, 1H), 3.03 (t, J=10.1 Hz, 1H), 1.97 (s, 1H), 1.61-1.41 (m, 1H), 1.38 (d, J=6.7 Hz, 6H).

LC-MS (M+H)⁺=399.0.

### Example 5: (3S,4R)-4-((5-chloro-4-(4-isopropyl-2-methylquinolin-6-yl)pyrimidin-2-yl)amino)tetrahydro-2H-pyran-3-ol

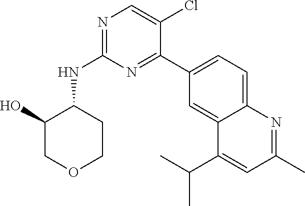 [SMILES: Cc1cc(C(C)C)c2cc(-c3nc(N[C@@H]4CCOC[C@H]4O)ncc3Cl)ccc2n1]

### Step 1: 6-chloro-2-methylquinolin-4-yl trifluoromethanesulfonate

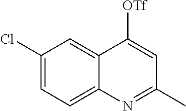 [SMILES: Cc1cc(OS(=O)(=O)C(F)(F)F)c2cc(Cl)ccc2n1]

To a solution of 6-chloro-2-methylquinolin-4-ol (800 mg, 4.13 mmol) and pyridine (653 mg, 8.26 mmol) in dichloromethane (20 mL) was added trifluoromethanesulfonic anhydride (1.75 g, 6.20 mmol) at 0° C. under nitrogen atmosphere. The resulting mixture was stirred for 12 h at room temperature before quenched by the addition of water (30 mL). The resulting mixture was extracted with dichloromethane (20 mL×3). The combined organic phases were washed with brine, dried over sodium sulfate, filtered and concentrated. The residue was purified over silica gel by combi-flash, eluting with ethyl acetate in petroleum ether (0-20% gradient, v/v) to give the title compound (700 mg, 52%). LC-MS (M+H)⁺=325.9.

### Step 2: 6-chloro-2-methyl-4-(prop-1-en-2-ylquinoline

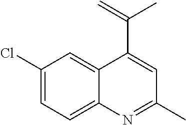

To a solution of 6-chloro-2-methylquinolin-4-yl trifluoromethanesulfonate (650 mg, 1.99 mmol) and 4,4,5,5-tetramethyl-2-(prop-1-en-2-yl)-1,3,2-dioxaborolane (302 mg, 1.79 mmol) in tetrahydrofuran (10 mL) and water (2 mL) were added potassium carbonate (552 mg, 3.99 mmol) and tetrakis(triphenylphosphine)palladium(0) (115 mg, 0.10 mmol). The resulting mixture was stirred at 70° C. for 3 h under nitrogen atmosphere before cooled to room temperature and concentrated under reduced pressure. The residue was purified over silica gel by combi-flash, eluting with ethyl acetate in petroleum ether (0-25% gradient, v/v) to give the title compound (368 mg, 84%/6). LC-MS (M+H)⁺=218.0.

### Step 3: 6-chloro-4-isopropyl-2-methylquinoline

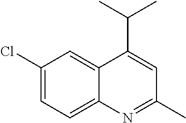

To a solution of 6-chloro-2-methyl-4-(prop-1-en-2-yl)quinoline (342 mg, 1.57 mmol) in methanol (10 mL) was added 5% Rh/C (324 mg, 0.16 mmol). The resulting mixture was stirred at room temperature for 12 h under 1 atmosphere of hydrogen. The mixture was filtered through Celite pad and the filtrate was concentrated under reduced pressure. The residue was purified over C18 column by combi-flash, eluting with acetonitrile in water (0-25% in 10 min. v/v, both eluents containing 0.1% trifluoroacetic acid) to give the title compound (285 mg, 82%). LC-MS (M+H)⁺=220.1.

### Step 4: 4-isopropyl-2-methyl-6-(4,4,5,5-tetramethyl-1,3,2-dioxaborolan-2-yl)quinoline

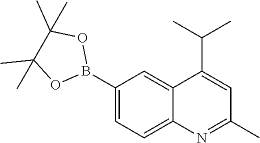

To a solution of 6-chloro-4-isopropyl-2-methylquinoline (238 mg, 1.08 mmol) and bis(pinacolato)diboron (411 mg, 1.62 mmol) in 1,4-dioxane (10 mL) were added potassium acetate (160 mg, 1.62 mmol) and dichlorobis(tricyclohexylphosphine)palladium(II) (80 mg, 0.108 mmol). The resulting mixture was stirred at 100° C. for 2 h under nitrogen atmosphere. The resulting mixture was concentrated under reduced pressure. The residue was purified over silica gel by combi-flash, eluting with methanol in dichloromethane (0-25% gradient, v/v) to give the title compound (144 mg, 42%). LC-MS (M+H)⁺=312.1.

### Step 5: 6-2,5-dichloropyrimidin-yl-4-isopropyl-2-methylquinoline

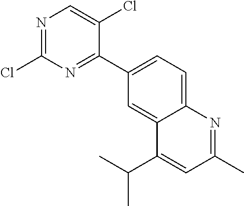 [SMILES: Cc1cc(C(C)C)c2cc(-c3nc(Cl)ncc3Cl)ccc2n1]

To a solution of 2,4,5-trichloropyrimidine (80 mg, 0.44 mmol) and 4-isopropyl-2-methyl-6-(4,4,5,5-tetramethyl-1,3,2-dioxaborolan-2-yl)quinoline (163 mg, 0.52 mmol) in dioxane (5 mL) and water (1 mL) were added potassium carbonate (90 mg, 0.65 mmol) and tetrakis(triphenylphosphine)palladium(0) (50 mg, 0.044 mmol). The resulting mixture was stirred at 100° C. for 2 h under nitrogen atmosphere. The resulting mixture was concentrated under reduced pressure. The residue was purified over C18 column by combi-flash, eluting with acetonitrile in water containing 0.1% trifluoroacetic acid (0-35% in 15 min. v/v) to give the title compound (80 mg, 55%). LC-MS (M+H)⁺=332.0.

### Step 6: (3S,4R)-44(5-chloro-4-(4-isopropyl-2-methylquinolin-6-yl)pyrimidin-2-yl)amino)tetrahydro-2H-pyran-3-ol

A solution of 6-(2,5-dichloropyrimidin-4-yl)-4-isopropyl-2-methylquinoline (70 mg, 0.21 mmol), (3S,4R)-4-aminooxan-3-ol hydrochloride (65 mg, 0.42 mmol) and N,N-diisopropylethylamine (0.11 mL, 0.63 mmol) in DMSO (4 mL) was stirred at 90° C. for 2 d under nitrogen atmosphere. The resulting mixture was concentrated under reduced pressure. The residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₄OH), 31% to 61% gradient in 9 min; detector. UV 254 nm. The title compound (10 mg, 12%) was obtained. ¹H-NMR (300 MHz, DMSO-d6) δ 8.55 (brs, 1H), 8.46 (s, 1H), 8.07-8.01 (m, 2H), 7.56-7.48 (m, 11H), 7.41 (s, 1H), 4.95 (d, J=5.3 Hz, 1H), 3.94-3.65 (m, 4H), 3.57-3.47 (m, 1H), 3.39-3.29 (m, 1H), 3.11-2.98 (m, 1H), 2.68 (s, 3H), 2.04-1.93 (m, 1H), 1.61-1.43 (m, 1H), 1.38 (d, J=6.7 Hz, 6H). LC-MS (M+H)⁺=413.1.

### Example 6: (3S,4R)-4-((5-fluoro-4-(4-isopropyl-3-methylquinolin-6-yl)pyrimidin-2-yl)amino)tetrahydro-2H-pyran-3-ol

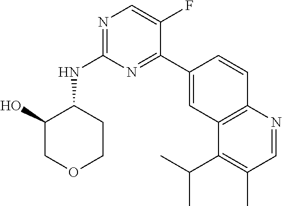 [SMILES: Cc1cnc2ccc(-c3nc(N[C@@H]4CCOC[C@H]4O)ncc3F)cc2c1C(C)C]

Step 1: (3S,4R)-4-(4-(3-(chloromethyl-4-isopropylquinolin-6-yl)-5-fluoropyrimidin-2-yl)amino)tetrahydro-2H-pyran-3-ol

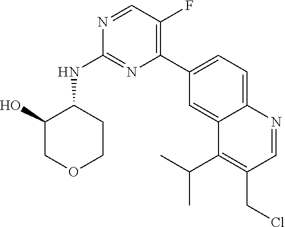 [SMILES: CC(C)c1c(CCl)cnc2ccc(-c3nc(N[C@@H]4CCOC[C@H]4O)ncc3F)cc12]

To the solution of (3S,4R)-4-((5-fluoro-4-(3-(hydroxymethyl)-4-isopropylquinolin-6-yl)pyrimidin-2-yl)amino)tetrahydro-2H-pyran-3-ol (80 mg, 0.19 mmol) in dichloromethane (5 mL) was added thionyl chloride (46 mg, 0.39 mmol) at 0° C. and the mixture was stirred for 1 h before quenched with saturated aqueous solution of sodium bicarbonate. The aqueous layer was extracted with dichloromethane (30 mL). The combined organic layers were washed with brine, dried over sodium sulfate, filtered and evaporated to give 85 mg crude product which was used for next step without further purification. LC-MS (M+H)⁺=431.2.

### Step 2: (3S,4R)-4-((5-fluoro-4-(4-isopropyl-3-methylquinolin-6-yl)pyrimidin-2-yl)amino)tetrahydro-2H-pyran-3-ol

A reaction mixture of (3S,4R)-4-((4-(3-(chloromethyl)4-isopropylquinolin-6-yl)-5-fluoropyrimidin-2-yl)amino)tetrahydro-2H-pyran-3-ol (85 mg, 0.19 mmol) and 5% Pd/C (15 mg) in methanol (5 mL) under 1 atmosphere of hydrogen was stirred at room temperature for 14 h. The mixture was filtered and evaporated. The residue was purified by prep-HPLC (eluent: water (0.1% formic acid)/acetonitrile (0.1% formic acid)) to give the product (4 mg, 0.5%). ¹H-NMR (400 MHz, DMSO, d6) δ 8.99 (s, 1H), 8.71 (s, 1H), 8.44 (d, J=2.9 Hz, 1H), 8.19 (d, J=9.0 Hz, 1H), 8.06 (d, J=8.7 Hz, 1H), 7.21 (d, J=7.5 Hz, 1H), 4.92 (s, 1H), 3.92-3.68 (m, 4H), 3.50 (s, 1H), 3.34-3.31 (m, 1H), 3.01 (t, J=10.4 Hz, 1H), 2.48 (s, 3H), 1.98 (s, 1H), 1.50-1.48 (m, 7H). LC-MS (M+H)⁺=397.3.

### Example 7: (3S,4R)-4-((5-fluoro-4-(4-isopropyl-2,3-dimethylquinolin-6-yl)pyrimidin-2-yl)amino)tetrahydro-2H-pyran-3-ol

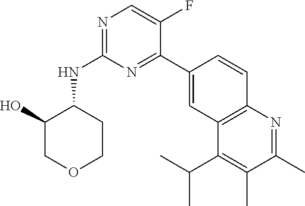 [SMILES: Cc1nc2ccc(-c3nc(N[C@@H]4CCOC[C@H]4O)ncc3F)cc2c(C(C)C)c1C]

### Step 1: 6-chloro-2,3-dimethylquinolin-4-ol

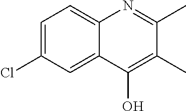

The reaction mixture of 4-chloroaniline (6 g, 46.9 mmol), ethyl 2-methyl-3-oxobutanoate (13.5 g, 93.8 mmol) in polyphosphoric acid (20 mL) was stirred at 175° C. for 4 h before cooled to room temperature. Water was added and formed precipitate was filtered and dried to give the title compound (8.1 g, 82%). LC-MS (M+H)⁺=208.0.

### Step 2: 4-bromo-6-chloro-2,3-dimethylquinoline

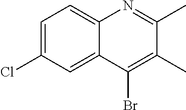

To the solution of 6-chloro-2,3-dimethylquinolin-4-ol (2.1 g, 10 mmol) in dimethylformamide (20 mL) was added phosphorus tribromide (3.3 g, 12.1 mmol) and the reaction solution was stirred at room temperature for overnight. The reaction was quenched with water and the precipitate was filtered. The crude was purified over silica gel by combi-flash, eluting with ethyl acetate in petroleum ether (33%, v/v) to give the title compound (2 g, 73%). LC-MS (M+H)⁺=270.0, 272.0.

### Step 3: 6-chloro-4-isopropyl-2,3-dimethylquinoline

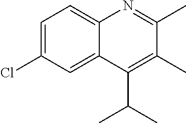 [SMILES: Cc1nc2ccc(Cl)cc2c(C(C)C)c1C]

To the solution of 1 M isopropylmagnesium bromide in tetrahydrofuran (3.7 mL, 3.7 mmol) under nitrogen was added 0.7 M zinc chloride solution in tetrahydrofuran (5.3 mL, 3.7 mmol) and the mixture was stirred at 50° C. for 2 h. A solution of 4-bromo-6-chloro-2,3-dimethylquinoline (500 mg, 1.85 mmol) in dimethylformamide (10 mL), copper(i) iodide (36 mg, 0.37 mmol) and (1,1′-bis(diphenylphosphino)ferrocene)palladium(II) dichloride (139 mg, 0.19 mmol) were added and the mixture was stirred at 50° C. for 2 h before cooled to room temperature. The reaction was quenched with methanol (10 mL) and the solvents were evaporated before addition of ethyl acetate and water. The formed precipitate was filtered. The filtrate was washed with water and brine, dried over sodium sulfate, filtered and evaporated. The residue was purified over silica gel by combi-flash, eluting with ethyl acetate in petroleum ether (0-16% gradient, v/v) to give the title compound (200 mg, 43%). ¹H-NMR (400 MHz, DMSO-d6) δ 8.16 (d, J=8.6 Hz, 1H), 7.86 (s, 1H), 7.39 (d, J=8.6 Hz, 1H), 6.08 (s, 1H), 5.05 (s, 1H), 2.52 (s, 3H), 1.62 (d, J=6.9 Hz, 7H). LC-MS (M+H)⁺=234.0.

### Step 4: 4-isopropyl-2,3-dimethyl-6-(4,4,5,5-tetramethyl-1,3,2-dioxaborolan-2-yl)quinoline

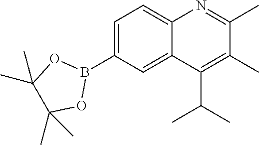

The reaction mixture of 6-chloro-4-isopropyl-2,3-dimethylquinoline (200 mg, 0.85 mmol), bis(pinacolato)diboron (326 mg, 1.28 mmol). (1,1′-bis(diphenylphosphino)ferrocene)palladium(II) dichloride (63 mg, 0.085 mmol) and potassium acetate (167 mg, 1.7 mmol) in 1,4-dioxane (10 mL) under nitrogen was stirred at 80° C. for overnight. The mixture was filtered and the filtrate was evaporated to give the crude product which was used for next step without further purification. LC-MS (M+H)⁺=244.1 for corresponding boronic acid.

### Step 5: 6-(2-chloro-5-fluoropyrimidin-4-yl)-4-isopropyl-2,3-dimethylquinoline

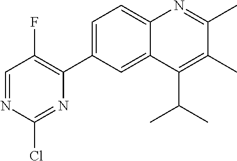 [SMILES: Cc1nc2ccc(-c3nc(Cl)ncc3F)cc2c(C(C)C)c1C]

A mixture of 4-isopropyl-2,3-dimethyl-6-(4,4,5,5-tetramethyl-1,3,2-dioxaborolan-2-yl)quinoline (crude), 2,4-dichloro-5-fluoropyrimidine (284 mg, 1.7 mmol), dichlorobis(tricyclohexylphosphine)palladium(II) (62 mg, 0.085 mmol) and potassium carbonate (235 mg, 1.7 mmol) in 1,4-dioxane (20 mL) and water (3 mL) under nitrogen was stirred at 70° C. for 2 h before cooled to room temperature and diluted with ethyl acetate. The organic layer was washed with brine, dried over sodium sulfate, filtered and evaporated. The residue was purified over silica gel by combi-flash, eluting with methanol in dichloromethane (0-3% gradient, v/v) to give the title compound (80 mg, 28% for two steps). LC-MS (M+H)⁺=330.1.

### Step 6: (3S,4R)-4-((5-fluoro-4-(4-isopropyl-2,3-dimethylquinolin-6-yl)pyrimidin-2-yl)amino)tetrahydro-2H-pyran-3-ol

The title compound (60 mg, 61%) was prepared in a manner similar to Example 1 step 4 from 6-(2-chloro-5-fluoropyrimidin-4-yl)-4-isopropyl-2,3-dimethylquinoline and (3S,4R)-4-aminotetrahydro-2H-pyran-3-ol. ¹H-NMR (400 MHz, DMSO-d6) δ 9.01 (s, 1H), 8.48 (s, 1H), 8.19 (d, J=8.1 Hz, 1H), 8.00 (d, J=8.7 Hz, 1H), 7.23 (d, J=7.5 Hz, 1H), 4.97 (d, J=5.2 Hz, 1H), 3.86-3.85 (m, 4H), 3.55 (s, 1H), 3.39-3.33 (m, 1H), 3.06 (t, J=10.4 Hz, 1H), 2.67 (s, 3H), 2.46 (s, 3H), 2.14-1.94 (m, 1H), 1.64-1.44 (m, 7H). LC-MS (M+H)⁺=411.1.

### Example 8: (3S,4R)-4-((5-fluoro-4-(4-isopropylquinolin-6-yl)pyrimidin-2-yl)amino)tetrahydro-2H-pyran-3-ol

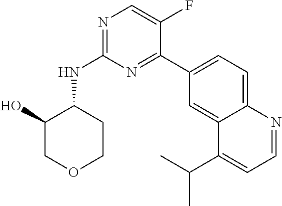 [SMILES: CC(C)c1ccnc2ccc(-c3nc(N[C@@H]4CCOC[C@H]4O)ncc3F)cc12]

### Step 1: 6-chloroquinolin-4-yl trifluoromethanesulfonate

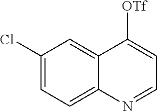 [SMILES: O=S(=O)(Oc1ccnc2ccc(Cl)cc12)C(F)(F)F]

6-Chloroquinolin-4-ol (3 g, 16.71 mmol) and N,N-diisopropylethylamine (2.59 g, 20.05 mmol) were dissolved in dichloromethane (30 mL). Trifluoromethanesulfonic anhydride (5.66 g, 20.05 mmol) was added dropwise at 0° C. and the reaction solution was stirred at 0° C. for 2 h before addition of water (20 mL). The aqueous layer was extracted with dichloromethane. The combined organic layers were washed with brine, dried over sodium sulfate, filtered and concentrated. The residue was purified over silica gel by combi-flash to give the title compound (4.1 g, 79%). LC-MS (M+H)⁺=312.0.

### Step 2: 6-chloro-4-(prop-1-en-2-yl)quinoline

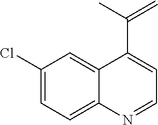

The title compound (700 mg, 26%) was prepared in a manner similar to Example 1 step 3 from 6-chloroquinolin-4-yl trifluoromethanesulfonate and 4,4,5,5-tetramethyl-2-(prop-1-en-2-yl)-1,3,2-dioxaborolane. LC-MS (M+H)⁺=204.0.

### Step 3: 6-chloro-4-isopropylquinoline

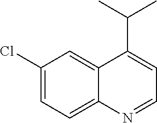

The title compound (616 mg, 100%) was prepared in a manner similar to Example 4 step 3 from 6-chloro-4-(prop-1-en-2-yl)quinoline. LC-MS (M+H)⁺=206.0.

### Step 4: 4-isopropyl-6-(4,4,5,5-tetramethyl-1,3,2-dioxaborolan-2-yl)quinoline

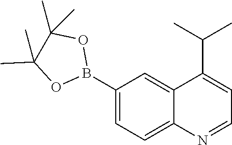 [SMILES: CC(C)c1ccnc2ccc(B3OC(C)(C)C(C)(C)O3)cc12]

The title compound (1.0 g, 100%) was prepared in a manner similar to Example 1 step 2 from 6-chloro-4-isopropylquinoline. LC-MS (M+H)⁺=298.0.

### Step 5: 6-(2-chloro-5-fluoropyrimidin-4-yl)-isopropylquinoline

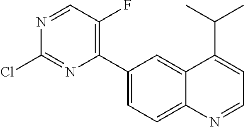 [SMILES: CC(C)c1ccnc2ccc(-c3nc(Cl)ncc3F)cc12]

The title compound (526 mg, 97%) was prepared in a manner similar to Example 1 step 3 from 2,4-dichloro-5-fluoropyrimidine and 4-isopropyl-6-(4,4,5,5-tetramethyl-1,3,2-dioxaborolan-2-yl)quinoline. LC-MS (M+H)⁺=302.0.

### Step 6: (3S,4R)-4-((5-fluoro-4-(4-isopropylquinolin-6-yl)pyrimidin-2-yl)amino)tetrahydro-2H-pyran-3-ol

The title compound (150 mg, 23%) was prepared in a manner similar to Example 1 step 4 from 6-(2-chloro-5-fluoropyrimidin-4-yl)-4-isopropylquinoline and (3S,4R)-4-aminotetrahydro-2H-pyran-3-ol hydrochloride. ¹H-NMR (400 MHz, DMSO-d6) δ 8.92 (d, J=4.5 Hz, 1H), 8.86 (s, 1H), 8.50 (d, J=3.6 Hz, 1H), 8.31 (d, J=8.7 Hz, 1H), 8.17 (d, J=9.3 Hz, 1H), 7.52 (d, J=4.4 Hz, 1H), 7.29 (d, J=7.3 Hz, 1H), 4.96 (s, 1H), 3.97-3.71 (m, 4H), 3.65-3.45 (m, 1H), 3.38 (m, 1H), 3.07 (t, J=10.4 Hz, 1H), 2.13-1.93 (m, 1H), 1.56-1.39 (m, 1H), 1.40 (d, J=6.6 Hz, 6H). LC-MS (M+H)⁺=383.0.

### Example 9: (3S,4R)-4-((5-fluoro-4-(4-isopropyl-5-methylquinolin-6-yl)pyrimidin-2-yl)amino)tetrahydro-2H-pyran-3-ol

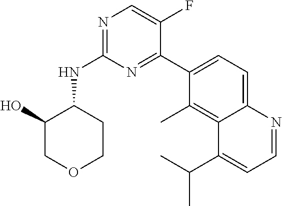 [SMILES: Cc1c(-c2nc(N[C@@H]3CCOC[C@H]3O)ncc2F)ccc2nccc(C(C)C)c12]

### Step 1: methyl 6-chloro-4-hydroxy-5-methylquinoline-2-carboxylate

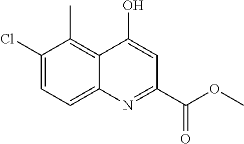 [SMILES: COC(=O)c1cc(O)c2c(C)c(Cl)ccc2n1]

To a solution of 4-chloro-3-methylaniline (8 g, 56.5 mmol) in methanol (110 mL), was added dimethyl but-2-ynedioate (8.11 g, 57.1 mmol) dropwise at 0° C. and the resulting solution was stirred at room temperature for 1 h. Solvent was removed in vacuum. The residue was purified over silica gel by combi-flash to give the title compound (4.24 g, 30%). LC-MS (M+H)⁺=252.0.

### Step 2: 6-chloro-4-hydroxy-5-methylquinoline-2-carboxylic acid

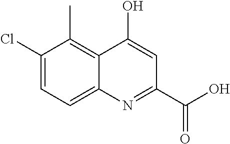 [SMILES: Cc1c(Cl)ccc2nc(C(=O)O)cc(O)c12]

The title compound (3.3 g, 82.5%) was prepared in a manner similar to Example 1 step 5 from methyl 6-chloro-4-hydroxy-5-methylquinoline-2-carboxylate. LC-MS (M+H)⁺=238.0.

### Step 3: 6-chloro-5-methylquinolin-4-ol

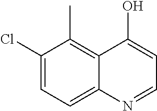

A solution of 6-chloro-4-hydroxy-5-methylquinoline-2-carboxylic acid (3.3 g, 13.9 mmol) in diphenyl ether (30 mL) was stirred at 260° C. for 1 h before cooled to room temperature and purified over silica gel by combi-flash to give the title compound (2.35 g, 87%). LC-MS (M+H)⁺=194.0.

### Step 4: 4,6-dichloro-5-methylquinoline

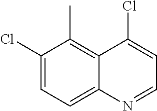

6-Chloro-5-methylquinolin-4-ol (553 mg, 2.86 mmol) was dissolved in phosphorus oxychloride (15 mL) and the solution was refluxed for 3 h before cooled to room temperature and concentrated in vacuum. Ethyl acetate (20 mL) was added and saturated sodium bicarbonate solution was added until pH=8. The aqueous layer was separated and extracted with ethyl acetate. The combined organic layers were washed with brine, dried over sodium sulfate, filtered and concentrated. The residue was purified over silica gel by combi-flash to give the title compound (491 mg, 81%). LC-MS (M+H)⁺=212.0.

### Step 5: 6-chloro-5-methyl-4-(prop-1-en-2-yl)quinoline

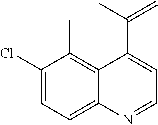

The title compound (191 mg, 51%) was prepared in a manner similar to Example 1 step 3 from 4,6-dichloro-5-methylquinoline and 4,4,5,5-tetramethyl-2-(prop-1-en-2-yl)-1,3,2-dioxaborolane. LC-MS (M+H)⁺=218.0.

### Step 6: 6-chloro-4-isopropyl-5-methylquinoline

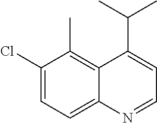

The title compound (193 mg, 100%) was prepared in a manner similar to Example 4 step 3 from 6-chloro-5-methyl-4-(prop-1-en-2-yl)quinoline. LC-MS (M+H)⁺=220.0.

### Step 7: 4-isopropyl-5-methyl-6-(4,4,5,5-tetramethyl-1,3,2-dioxaborolan-2-yl)quinoline

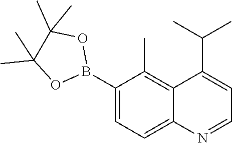 [SMILES: Cc1c(B2OC(C)(C)C(C)(C)O2)ccc2nccc(C(C)C)c12]

The title compound (302 mg, 100%) was prepared in a manner similar to Example 1 step 2 from 6-chloro-4-isopropyl-5-methylquinoline. LC-MS (M+H)⁺=312.0.

### Step 8: 6-(2-chloro-5-fluoropyrimidin-4-yl)-4-isopropyl-5-methylquinoline

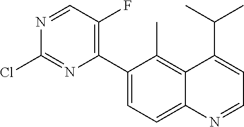 [SMILES: Cc1c(-c2nc(Cl)ncc2F)ccc2nccc(C(C)C)c12]

The title compound (72 mg, 24%) was prepared in a manner similar to Example 1 step 3 from 4-isopropyl-5-methyl-6-(4,4,5,5-tetramethyl-1,3,2-dioxaborolan-2-yl)quinoline. LC-MS (M+H)⁺=316.0.

### Step 9: (3S,4R)-4-((5-fluoro-4-(4-isopropyl-5-methylquinolin-6-yl)pyrimidin-2-yl)amino)tetrahydro-2H-pyran-3-ol

The title compound (25 mg, 28%) was prepared in a manner similar to Example 1 step 4 from 6-(2-chloro-5-fluoropyrimidin-4-yl)-4-isopropyl-5-methylquinoline. ¹H-NMR (400 MHz, DMSO-d6) δ 8.84 (d, J=4.5 Hz, 1H), 8.47 (s, 1H), 7.96 (d, J=8.6 Hz, 1H), 7.64 (d, J=8.6 Hz, 1H), 7.62 (d, J=4.5 Hz, 1H), 7.30 (d, J=7.6 Hz, 1H), 4.94 (d, J=5.1 Hz, 1H), 4.22-4.02 (m, 1H), 3.85-3.70 (m, 3H), 3.58-3.42 (m, 1H), 3.32-3.22 (m, 1H), 3.03 (t, J=10.3 Hz, 1H), 2.71 (s, 3H), 1.98 (d, J=11.5 Hz, 1H), 1.55-1.35 (m, 1H), 1.34 (d, J=6.4 Hz, 6H). LC-MS (M+H)⁺=397.0.

### Example 10 (3S,4R)-4-((5-fluoro-4-(4-isopropyl-2-methylquinolin-6-yl)pyrimidin-2-yl)amino)tetrahydro-2H-pyran-3-ol

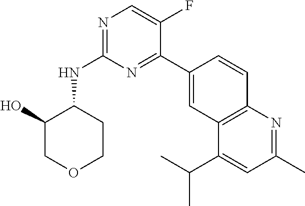 [SMILES: Cc1cc(C(C)C)c2cc(-c3nc(N[C@@H]4CCOC[C@H]4O)ncc3F)ccc2n1]

### Step 1: 6-(2-choro-5-fluoropyrimidin-4-yl-4-isopropyl-2-methylquinoline

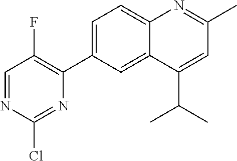 [SMILES: Cc1cc(C(C)C)c2cc(-c3nc(Cl)ncc3F)ccc2n1]

The title compound (180 mg, 69%) was prepared in a manner similar to that in Example 1 step 3 from 4-isopropyl-2-methyl-6-(4,4,5,5-tetramethyl-1,3,2-dioxaborolan-2-yl)quinoline and 2,4-dichloro-5-fluoropyrimidine. LC-MS (M+H)⁺=316.1.

### Step 2: (3S,4R)-44(5-fluoro-4(4-isopropyl-2-methylquinolin-6-yl)pyrimidin-2-yl)amino)tetrahydro-2H-pyran-3-ol

The title compound (25 mg, 11%) was prepared in a manner similar to that in Example 1 step 4 from 6-(2-chloro-5-fluoropyrimidin-4-yl)-4-isopropyl-2-methylquinoline and (3S,4R)-4-aminotetrahydro-2H-pyran-3-ol. ¹H-NMR (400 MHz, DMSO-d6) δ 8.80 (s, 1H), 8.48 (d, J=3.6 Hz, 1H), 8.26 (d, J=8.2 Hz, 1H), 8.05 (d, J=8.8 Hz, 1H), 7.42 (s, 1H), 7.25 (d, J=7.5 Hz, 1H), 4.96 (s, 1H), 3.90-3.79 (m, 3H), 3.74 (dt, J=14.0, 7.0 Hz, 1H), 3.54 (s, 1H), 3.39-3.33 (m, 1H), 3.07 (t, J=10.3 Hz, 1H), 2.68 (s, 3H), 2.12-1.92 (m, 1H), 1.55-1.47 (m, 1H), 1.42-1.35 (m, 6H). LC-MS (M+H)⁺=397.3.

### Example 11: (3S,4R)-4-((4-(3-((dimethylamino)methyl)-4-isopropylquinolin-6-yl)-5-fluoropyrimidin-2-yl)amino)tetrahydro-2H-pyran-3-ol

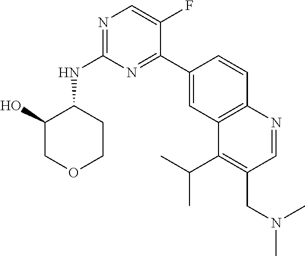 [SMILES: CC(C)c1c(CN(C)C)cnc2ccc(-c3nc(N[C@@H]4CCOC[C@H]4O)ncc3F)cc12]

To a solution of (3S,4R)-4-((4-(3-(chloromethyl)-4-isopropylquinolin-6-yl)-5-fluoropyrimidin-2-yl)amino)tetrahydro-2H-pyran-3-ol (25 mg, 0.058 mmol) in dimethylformamide (2 mL) were added dimethylamine hydrochloride (9.5 mg, 0.116 mmol) and N,N-diisopropylethylamine (22.4 mg, 0.174 mmol) at 25° C. The mixture was stirred at 25° C. for 5 h before diluted with ethyl acetate (20 mL). The organic solution was washed with brine (20 mL), dried over sodium sulfate, filtered and concentrated under reduce pressure. The residue was purified over silica gel by combi-flash, eluting with methanol in dichloromethane (5%, v/v) to give the title compound (7.4 mg, 29%). ¹H-NMR (400 MHz, DMSO-d6) δ; 9.12 (s, 1H), 8.78 (s, 1H), 8.50 (s, 1H), 8.34-8.25 (m, 1H), 8.18-8.10 (m, 1H), 7.26 (d, J=7.9 Hz, 1H), 4.52-4.94 (m, 1H), 4.04-3.90 (m, 1H), 3.92-3.80 (m, 3H), 3.66-3.61 (m, 2H), 3.57-3.50 (m, 11H), 3.42-3.35 (m, 1H), 3.05 (t, J=10.3 Hz, 1H), 2.20 (s, 6H), 2.09-2.01 (m, 1H), 1.62-1.49 (m, 7H). LC-MS (M+H)⁺=440.1.

### Example 12: 1-((6-(5-fluoro-2-(((3S,4R)-3-hydroxytetrahydro-2H-pyran-4-yl)amino)pyrimidin-4-yl)-4-isopropylquinolin-3-yl)methyl)pyrrolidin-2-one

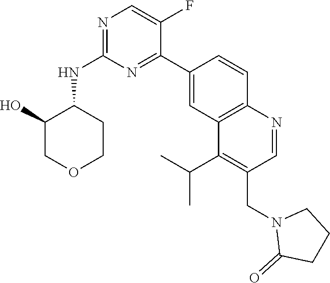 [SMILES: CC(C)c1c(CN2CCCC2=O)cnc2ccc(-c3nc(N[C@@H]4CCOC[C@H]4O)ncc3F)cc12]

### Step 1: (6-chloro-4-isopropylquinolin-3-yl)methanol

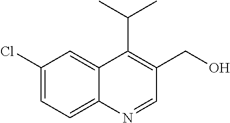 [SMILES: CC(C)c1c(CO)cnc2ccc(Cl)cc12]

Ethyl 6-chloro-4-isopropylquinoline-3-carboxylate (2.45 g, 8.82 mmol) was dissolved in dry tetrahydrofuran (20 mL). Diisobutylaluminium hydride solution in tetrahydrofuran (11.8 mL, 1.5 M, 17.64 mmol) was added dropwise at 0° C. and the reaction solution was stirred at room temperature for overnight. Saturated solution of NH₄Cl (30 mL) was added and the aqueous layer was extracted with ethyl acetate (100 mL×3). The combine organic layers were washed with brine, dried over sodium sulfate, filtered and concentrated. The residue was purified over silica gel by combi-flash to give the title compound (1.73 g, 56%). LC-MS (M+H)⁺=236.0.

### Step 2: (6-chloro-4-isopropylquinolin-3-yl)methyl methanesulfonate

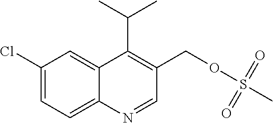 [SMILES: CC(C)c1c(COS(C)(=O)=O)cnc2ccc(Cl)cc12]

(6-Chloro-4-isopropylquinolin-3-yl)methanol (868 mg, 3.68 mmol) and methanesulfonic anhydride (770 mg, 4.42 mmol) were dissolved in dry dichloromethane (10 mL) at 0° C. N,N-diisopropylethylamine (571.2 mg, 4.42 mmol) was added at 0° C. and the reaction solution was stirred at 0° C. for 30 min and then room temperature for 2 h. Aqueous saturated solution of sodium bicarbonate (10 mL) was added and the aqueous layer was extracted with ethyl acetate (30 mL×3). The combined organic layers were washed with brine, dried over sodium sulfate, filtered and concentrated. The residue was purified over silica gel by combi-flash to give the title compound (736 mg, 63.4%). LC-MS (M+H)⁺=314.0.

### Step 3: 1-((6-chloro-4-isopropylquinolin-3-yl)methyl)pyrrolidin-2-one

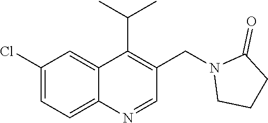 [SMILES: CC(C)c1c(CN2CCCC2=O)cnc2ccc(Cl)cc12]

To a solution of pyrrolidin-2-one (81.4 mg, 0.96 mmol) in dry dimethylformamide (3 mL) was added sodium hydride (5 mg, 60%, 1.28 mmol) at 0° C. under nitrogen atmosphere and the reaction mixture was stirred at 0° C. for 30 min. (6-Chloro-4-isopropylquinolin-3-yl)methyl methanesulfonate (200 mg, 0.64 mmol) was added and the mixture was stirred at room temperature for overnight before addition of water (20 mL). The aqueous layer was extracted with ethyl acetate (30 mL×3). The combined organic layers were washed with brine, dried over sodium sulfate, filtered and concentrated. The residue was purified over silica gel by combi-flash to give the title compound (96 mg, 50%). LC-MS (M+H)⁺=303.0.

### Step 4: 1-((4-isopropyl-6-(4,4,5,5-tetramethyl-1,3,2-dioxaborolan-2-yl)quinolin-3-yl)methyl)pyrrolidin-2-one

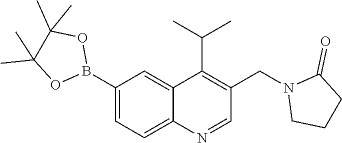 [SMILES: CC(C)c1c(CN2CCCC2=O)cnc2ccc(B3OC(C)(C)C(C)(C)O3)cc12]

The title compound (152 mg, 100%) was prepared in a manner similar to that in Example 1 step 2 from 1-((6-chloro-4-isopropylquinolin-3-yl)methyl)pyrrolidin-2-one. LC-MS (M+H)⁺=395.0.

### Step 5: 1-((6-(2-chloro-5-fluoropyrimidin-4-yl)-4-isopropylquinolin-3-yl)methyl)pyrrolidin-2-one

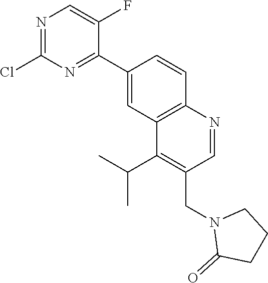 [SMILES: CC(C)c1c(CN2CCCC2=O)cnc2ccc(-c3nc(Cl)ncc3F)cc12]

The title compound (126 mg, 100%) was prepared in a manner similar to that in Example 1 step 3 from 1-((4-isopropyl-6-(4,4,5,5-tetramethyl-1,3,2-dioxaborolan-2-yl)quinolin-3-yl)methyl)pyrrolidin-2-one. LC-MS (M+H)⁺=399.0.

### Step 6: 1-((6-(5-fluoro-2-(((3S,4R)-3-hydroxytetrahydro-2H-pyran-4-yl)amino)pyrimidin-4-yl)-4-isopropylquinolin-3-yl)methyl)pyrrolidin-2-one

The title compound (46 mg, 30%) was prepared in a manner similar to that in Example 1 step 4 from 1-((6-(2-chloro-5-fluoropyrimidin-4-yl)-4-isopropylquinolin-3-yl)methyl)pyrrolidin-2-one. ¹H-NMR (400 MHz, DMSO-d6) δ 9.12 (s, 1H), 8.82 (s, 1H), 8.50 (d, J=3.7 Hz, 1H), 8.31 (d, J=8.8 Hz, 1H), 8.16 (d, J=8.8 Hz, 1H), 7.28 (d, J=7.8 Hz, 1H), 4.97 (d, J=5.2 Hz, 1H), 4.68 (s, 2H), 4.01-3.68 (m, 4H), 3.67-3.47 (m, 1H), 3.37 (d, J=11.9 Hz, 1H), 3.15 (t, J=6.9 Hz, 2H), 3.05 (t, J=10.4 Hz, 1H), 2.30 (t, J=8.0 Hz, 2H), 2.13-1.93 (m, 1H), 1.96-1.82 (m, 2H), 1.53 (t, J=6.3 Hz, 7H). LC-MS (M+H)⁺=480.0.

### Example 13: 4-((6-(5-fluoro-2-(((3S,4R)-3-hydroxytetrahydro-2H-pyran-4-yl)amino)pyrimidin-4-yl)-4-isopropylquinolin-3-yl)methyl)morpholin-3-one

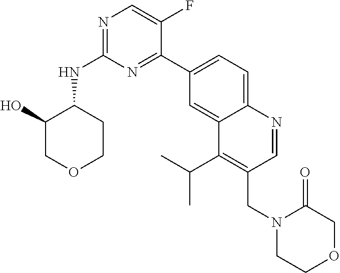 [SMILES: CC(C)c1c(CN2CCOCC2=O)cnc2ccc(-c3nc(N[C@@H]4CCOC[C@H]4O)ncc3F)cc12]

### Step 1: 4-((6-chloro-4-isopropylquinolin-3-yl)methyl)morpholin-3-one

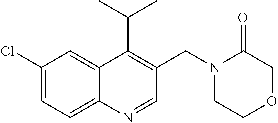 [SMILES: CC(C)c1c(CN2CCOCC2=O)cnc2ccc(Cl)cc12]

The title compound (152 mg, 100%) was prepared in a manner similar to that in Example 12 step 3 from (6-chloro-4-isopropylquinolin-3-yl)methyl methanesulfonate. LC-MS (M+H)⁺=319.0.

### Step 2: 4-((4-isopropyl-6-(4,4,5,5-tetramethyl-1,3,2-dioxaborolan-2-yl)quinolin-3-yl)methyl)morpholin-3-one

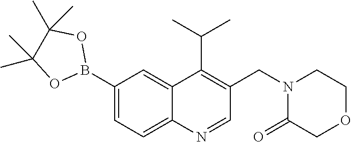 [SMILES: CC(C)c1c(CN2CCOCC2=O)cnc2ccc(B3OC(C)(C)C(C)(C)O3)cc12]

The title compound (205 mg, 100%) was prepared in a manner similar to that in Example 1 step 2 from 4-((6-chloro-4-isopropylquinolin-3-yl)methyl)morpholin-3-one. LC-MS (M+H)⁺=411.0.

### Step 3: 4-((6-(2-chloro-5-fluoropyrimidin-4-yl)-4-isopropylquinolin-3-yl)methyl)morpholin-3-one

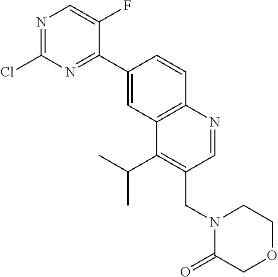 [SMILES: CC(C)c1c(CN2CCOCC2=O)cnc2ccc(-c3nc(Cl)ncc3F)cc12]

The title compound (120 mg, 100%) was prepared in a manner similar to that in Example 1 step 3 from 4-((4-isopropyl-6-(4,4,5,5-tetramethyl-1,3,2-dioxaborolan-2-yl)quinolin-3-yl)methyl)morpholin-3-one. LC-MS (M+H)⁺=415.15.

### Step 4: 4-((6-(5-fluoro-2-(((3S,4R)-3-hydroxytetrahydro-2H-pyran-4-yl)amino)pyrimidin-4-yl)-4-isopropylquinolin-3-yl)methyl)morpholin-3-one

The title compound (25 mg, 10%) was prepared in a manner similar to that in Example 1 step 4 from 4-((6-(2-chloro-5-fluoropyrimidin-4-yl)-4-isopropylquinolin-3-yl)methyl)morpholin-3-one. ¹H-NMR (400 MHz, DMSO-d6) a 9.13 (s, 1H), 8.82 (s, 1H), 8.50 (d, J=3.7 Hz, 1H), 8.31 (d, J=8.5 Hz, 1H), 8.16 (d, J=8.7 Hz, 1H), 7.28 (d, J=7.6 Hz, 1H), 4.97 (d, J=5.2 Hz, 1H), 4.92 (s, 2H), 4.14 (s, 2H), 3.93-3.81 (m, 3H), 3.89-3.69 (m, 3H), 3.60-3.48 (m, 1H), 3.37 (d, J=11.3 Hz, 1H), 3.17 (s, 2H), 3.05 (t.J=10.3 Hz, 1H), 2.12-1.92 (s, 1H), 1.64-1.44 (m, 7H). LC-MS (M+H)⁺=496.0.

### Example 14: (3S,4R)-4-((4-(3-((6-oxa-3-azabicyclo[3.1.1]heptan-3-yl)methyl)-4-isopropylquinolin-6-yl)-5-fluoropyrimidin-2-yl)amino)tetrahydro-2H-pyran-3-ol

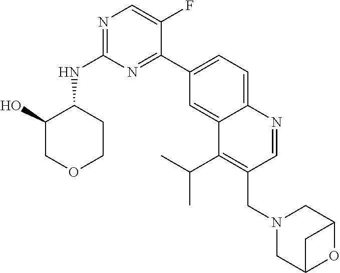 [SMILES: CC(C)c1c(CN2CC3CC(C2)O3)cnc2ccc(-c3nc(N[C@@H]4CCOC[C@H]4O)ncc3F)cc12]

The title compound (20 mg, 35%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and 6-oxa-3-azabicyclo[3.1.1]heptane. ¹H-NMR (400 MHz, DMSO-d6) δ 9.13 (s, 1H), 8.86 (s, 1H), 8.50 (d, J=3.8 Hz, 1H), 8.29 (d, J=8.9 Hz, 11H), 8.14 (d, J=8.8 Hz, 11H), 7.26 (d, J=7.8 Hz, 1H), 4.97 (d, J=5.3 Hz, 1H), 4.42 (d, J=6.0 Hz, 2H), 4.04 (dd, J=14.4, 7.0 Hz, 1H), 3.97 (s, 2H), 3.85 (dd, J=10.6, 5.1 Hz, 3H), 3.61-3.48 (m, 1H), 3.37 (d, J=11.4 Hz, 1H), 3.05 (t, J=10.4 Hz, 1H), 2.92 (d, J=11.1 Hz, 2H), 2.84 (q, J=6.6 Hz, 1H), 2.75 (d, J=11.2 Hz, 2H), 2.20 (d, J=7.6 Hz, 1H), 2.13-1.93 (s, 1H), 1.65-1.45 (m, 1H), 1.55 (d, J=9.0 Hz, 3H), 1.53 (d, J=9.0 Hz, 3H). LC-MS (M+H)⁺=494.0.

### Example 15: (3S,4R)-4-((5-fluoro-4-(3-(hydroxymethyl)-4-isopropyl-2-methylquinolin-6-yl)pyrimidin-2-yl)amino)tetrahydro-2H-pyran-3-ol

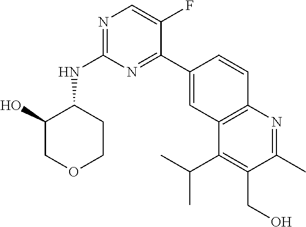 [SMILES: Cc1nc2ccc(-c3nc(N[C@@H]4CCOC[C@H]4O)ncc3F)cc2c(C(C)C)c1CO]

### Step 1: ethyl-6-chloro-2-methyl-4-oxo-1,4-dihydroquinoline-3-carboxylate

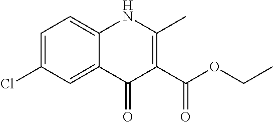 [SMILES: CCOC(=O)c1c(C)[nH]c2ccc(Cl)cc2c1=O]

To a solution of ethyl 3-oxobutanoate (7.9 g, 60.6 mmol) in dimethylacetamide (50 mL) under nitrogen was added sodium hydride (2.83 g, 60%, 70.7 mmol) followed by 6-chloro-2H-benzo[d][1.3]oxazine-2,4(1H)-dione (10 g, 50.5 mmol). The mixture was stirred at 120° C. for 1 h before cooled to room temperature. The mixture was concentrated in vacuum before water was added. The formed precipitate was filtered and dried to give the product (9 g, 67%). LC-MS (M+H)⁺=265.9.

### Step 2: ethyl 4-bromo-6-chloro-2-methylquinoline-3-carboxylate

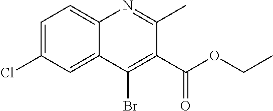 [SMILES: CCOC(=O)c1c(C)nc2ccc(Cl)cc2c1Br]

To a solution of ethyl 6-chloro-2-methyl-4-oxo-1,4-dihydroquinoline-3-carboxylate (1 g, 3.76 mmol) in dimethylformamide (20 mL) at 0° C. was added phosphorus tribromide (1.32 g, 4.89 mmol). The solution was stirred at room temperature for overnight before addition of saturated aqueous solution of sodium bicarbonate. The formed precipitate was filtered and dried to give the crude product (900 mg, 73%). LC-MS (M+H)⁺=328.0, 330.0.

### Step 3: ethyl 6-chloro-4-isopropyl-2-methylquinoline-3-carboxylate

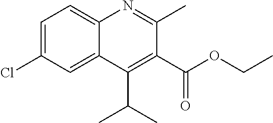 [SMILES: CCOC(=O)c1c(C)nc2ccc(Cl)cc2c1C(C)C]

The title compound (520 mg, 65%) was prepared in a manner similar to Example 1 step 1 from ethyl 4-bromo-6-chloro-2-methylquinoline-3-carboxylate. LC-MS (M+H)⁺=292.1.

### Step 4: ethyl 4-isopropyl-2-methyl-6-(4,4,5,5-tetramethyl-1,3,2-dioxaborolan-2-v)quinoline-3-carboxylate

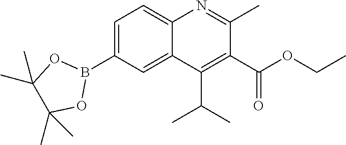 [SMILES: CCOC(=O)c1c(C)nc2ccc(B3OC(C)(C)C(C)(C)O3)cc2c1C(C)C]

The title compound was prepared in a manner similar to Example 1 step 2 from ethyl 6-chloro-4-isopropyl-2-methylquinoline-3-carboxylate. LC-MS (M+H)⁺=384.1.

### Step 5: ethyl 6-(2-chloro-5-fluoropyrimidin-4-yl)-4-isopropyl-2-methylquinoline-3-carboxylate

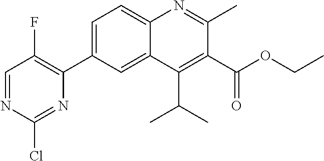 [SMILES: CCOC(=O)c1c(C)nc2ccc(-c3nc(Cl)ncc3F)cc2c1C(C)C]

The title compound (170 mg, 24% for two steps) was prepared in a manner similar to Example 1 step 3 from ethyl 4-isopropyl-2-methyl-6-(4,4,5,5-tetramethyl-1,3,2-dioxaborolan-2-yl)quinoline-3-carboxylate. LC-MS (M+H)⁺=388.2.

### Step 6: ethyl 6-(5-fluoro-2-(((3S,4R)-3-hydroxytetrahydro-2H-pyran-4-ylamino)pyrimidin-4-yl)-4-isopropyl-2-methylquinoline-3-carboxylate

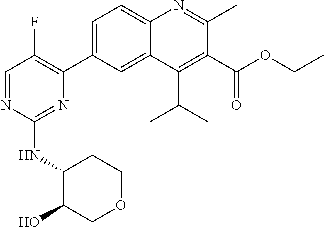 [SMILES: CCOC(=O)c1c(C)nc2ccc(-c3nc(N[C@@H]4CCOC[C@H]4O)ncc3F)cc2c1C(C)C]

The title compound (120 mg, 58%) was prepared in a manner similar to Example 1 step 4 from ethyl 6-(2-chloro-5-fluoropyrimidin-4-yl)-4-isopropyl-2-methylquinoline-3<carboxylate. LC-MS (M+H)⁺⁼469.3.

### Step 7: (3S,4R)-4-((5-fluoro-4-(3-(hydroxymethyl)-4-isopropyl-2-methylquinolin-6-yl)pyrimidin-2-yl)amino)tetrahydro-2H-pyran-3-ol

The title compound (2 mg, 5%) was prepared in a manner similar to Example 3 from ethyl 6-(5-fluoro-2-(((3S,4R)-3-hydroxy tetrahydro-2H-pyran-4-yl)amino)pyrimidin-4-yl)-4-isopropyl-2-methylquinoline-3-carboxylate. ¹H-NMR (400 MHz, DMSO-d6) δ 9.07 (s, 1H), 8.48 (d, J=3.8 Hz, 1H), 8.24 (d, J=8.9 Hz, 1H), 8.02 (d, J=8.8 Hz, 1H), 7.24 (d, J=7.8 Hz, 1H), 5.18 (s. H), 4.97 (d, J=5.4 Hz, 1H), 4.74 (d, J=4.0 Hz, 2H), 4.11-3.94 (m, 1H), 3.94-3.75 (m, 3H), 3.59-3.49 (m, 1H), 3.39-3.25 (m, 1H), 3.05 (t, J=10.4 Hz, 1H), 2.79 (s, 3H), 2.03-1.99 (m, 1H), 1.70-1.40 (m, 7H). LC-MS (M+H)⁺=427.3.

### Example 16: 1-((6-(5-fluoro-2-(((3S,4R)-3-hydroxytetrahydro-2H-pyran-4-yl)amino)pyrimidin-4-yl)-4-isopropylquinolin-3-yl)methyl)azetidin-3-ol

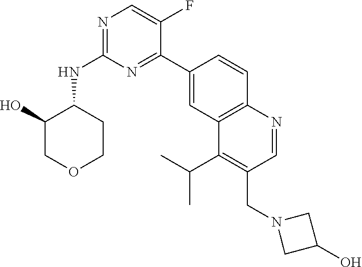 [SMILES: CC(C)c1c(CN2CC(O)C2)cnc2ccc(-c3nc(N[C@@H]4CCOC[C@H]4O)ncc3F)cc12]

The title compound (6.9 mg, 25%) was prepared in a manner similar to that in Example 11 step 1 from (3S,4R)-4-(4-(3-(chloromethyl)-4-isopropylquinolin-6-yl)-5-fluoropyrimidin-2-yl)amino)tetrahydro-2H-pyran-3-ol and azetidin-3-ol. ¹H-NMR (400 MHz, DMSO-d6) δ: 9.09 (s, 1H), 8.81 (s, 1H), 8.49 (d, J=5.2 Hz, 1H), 8.33-8.24 (m, 1H), 8.18-8.10 (m, 1H), 7.26 (d, J=7.9 Hz, 1H), 5.34 (d, J=8.4 Hz, 1H), 4.97 (d, J=6.8 Hz, 1H), 4.24-4.16 (m, 1H), 4.00-3.79 (m, 7H), 3.60-3.45 (m, 3H), 3.42-3.35 (m, 1H), 3.03 (t, J=10.3 Hz, 1H), 2.86 (t, J=6.5 Hz, 1H), 2.09-1.95 (m, 1H) 1.61-1.49 (m, 7H). LC-MS (M+H)⁺=468.1.

### Example 17: (3S,4R)-4-((5-fluoro-4-(4-isopropyl-3-(morpholinomethyl)quinolin-6-yl)pyrimidin-2-yl)amino)tetrahydro-2H-pyran-3-ol

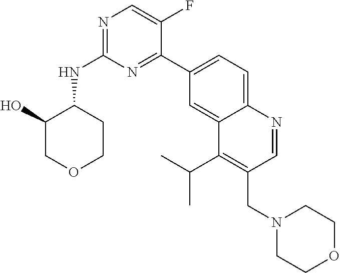 [SMILES: CC(C)c1c(CN2CCOCC2)cnc2ccc(-c3nc(N[C@@H]4CCOC[C@H]4O)ncc3F)cc12]

The title compound (9.1 mg, 15%) was prepared in a manner similar to that in Example 11 step 1 from (3S,4R)-4-(4-(3-(chloromethyl)-4-isopropylquinolin-6-yl)-5-fluoropyrimidin-2-yl)amino)tetrahydro-2H-pyran-3-ol and morpholine. ¹H-NMR (400 MHz, DMSO-d6) 3: 9.13 (s, 1H), 8.78 (s, 1H), 8.52-8.48 (m, 1H), 8.33-8.24 (m, 1H), 8.18-8.10 (m, 11H), 7.26 (d, J=7.9 Hz, 1H), 5.00-4.95 (m, 1H), 4.04-4.00 (m, 1H), 3.92-3.81 (m, 3H), 3.76-3.71 (m, 2H), 3.60-3.50 (m, 5H), 3.42-3.35 (m, 1H), 3.05 (t, J=10.3 Hz, 1H), 2.40 (s, 3H), 2.45-2.30 (m, 1H), 2.09-2.00 (m, 1H) 1.62-1.50 (m, 7H). LC-MS (M+H)⁺=482.1.

Example 17 can also be obtained by the following steps:

### Step 1: ethyl 6-chloro-4-isopropylquinoline-3-carboxylate

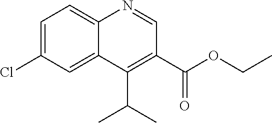 [SMILES: CCOC(=O)c1cnc2ccc(Cl)cc2c1C(C)C]

To the solution of isopropylmagnesium bromide (15 mL, 1M, 15 mmol) under nitrogen was added zinc chloride solution in tetrahydrofuran (21.4 mL, 0.7 M, 15 mmol) and the mixture was stirred at 50° C. for 2 h. The solution of ethyl 4,6-dichloroquinoline-3-carboxylate (2 g, 7.5 mmol) in dimethylformamide (10 mL), copper(I) iodide (143 mg, 0.75 mmol) and (1,1′-bis(diphenylphosphino)ferrocene)palladium(II) dichloride (275 mg, 0.375 mmol) were added and the resulting mixture was stirred at 50° C. for 2 h before cooled to room temperature. The reaction was quenched with methanol (10 mL) and the solvent was evaporated. The residue was suspended in ethyl acetate and water before filtration. The filtrate was washed with water and brine, dried over sodium sulfate, filtered and evaporated. The residue was purified over silica gel by combi-flash, eluting with ethyl acetate in petroleum ether (16%, v/v) to give the title compound (1.5 g, 71%). LC-MS (M+H)⁺=278.2.

### Step 2: ethyl 4-isopropyl-6-(4,4,5,5-tetramethyl-1,3,2-dioxaborolan-2-yl)quinoline-3-carboxylate

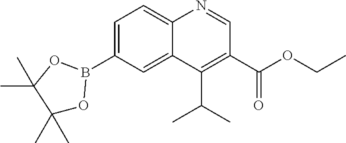 [SMILES: CCOC(=O)c1cnc2ccc(B3OC(C)(C)C(C)(C)O3)cc2c1C(C)C]

A mixture of ethyl 6-chloro-4-isopropylquinoline-3-carboxylate (1.3 g, 4, mmol), bis(pinacolato)diboron (1.4 g, 5.6 mmol), (1,1′-bis(diphenylphosphino)ferrocene)palladium(II) dichloride (172 mg, 0.24 mmol) and potassium acetate (921 mg, 9.4 mmol) in 1,4-dioxane (40 mL) was stirred at 80° C. under nitrogen for overnight before cooled to room temperature. The mixture was filtered and the filtrate was evaporated to give the crude product which was used for next step without further purification. LC-MS (M+H)⁺=370.3.

### Step 3: ethyl 6-(2-chloro-5-fluoropyrimidin-4-yl)-4-isopropylquinoline-3-carboxylate

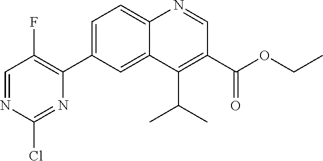 [SMILES: CCOC(=O)c1cnc2ccc(-c3nc(Cl)ncc3F)cc2c1C(C)C]

A mixture of ethyl 4-isopropyl-6-(4,4,5,5-tetramethyl-1,3,2-dioxaborolan-2-yl)quinoline-3-carboxylate, 2,4-dichloro-5-fluoropyrimidine (785 mg, 4.7 mmol). (1,1′-bis(diphenylphosphino)ferrocene)palladium(II) dichloride (176 mg, 0.24 mmol) and K₂CO₃ (1.3 g, 9.4 mmol) in 1,4-dioxane (40 mL) and water (5 mL) under nitrogen was stirred at 80° C. for overnight before cooled to room temperature. The mixture was diluted with ethyl acetate, washed with brine, dried over sodium sulfate, filtered and evaporated. The residue was purified over silica gel by combi-flash, eluting with methanol in dichloromethane (3%, v/v) to give the title compound (790 mg, 45% for two steps). LC-MS (M+H)⁺=374.1.

### Step 4: ethyl 6-(5-fluoro-2-(((3S,4R)-3-hydroxytetrahydro-2H-pyran-4-ylamino)pyrimidin-4-yl)-4-isopropylquinoline-3-carboxylate

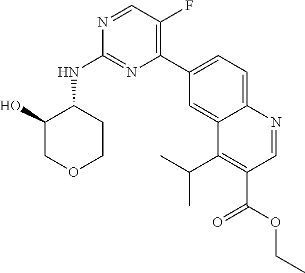 [SMILES: CCOC(=O)c1cnc2ccc(-c3nc(N[C@@H]4CCOC[C@H]4O)ncc3F)cc2c1C(C)C]

The reaction mixture of ethyl 6-(2-chloro-5-fluoropyrimidin-4-yl)-4-isopropylquinoline-3-carboxylate (790 mg, 2.11 mmol), (3S,4R)-4-aminotetrahydro-2H-pyran-3-ol (390 mg, 2.53 mmol), (SP-4-1)-[1,3-bis[2,6-bis(1-ethylpropyl)phenyl]-4,5-dichloro-1,3-dihydro-2H-imidazol-2-ylidene]dichloro(2-methylpyridine)palladium (89 mg, 0.11 mmol) and cesium carbonate (2.1 g, 6.33 mmol) in 1,4-dioxane (30 mL) under nitrogen was stirred at 10) ° C. for overnight. The mixture was cooled to room temperature and the solvent was evaporated. The residue was purified over silica gel by combi-flash, eluting with methanol in dichloromethane (6%, v/v) to give the title compound (780 mg, 81%). LC-MS (M+H)⁺⁼455.8.

### Step 5: (3S,4R)-44(5-fluoro-4-(3-(hydroxymethyl)-4-isopropylquinolin-6-yl)pyrimidin-2-yl)amino)tetrahydro-2H-pyran-3-ol

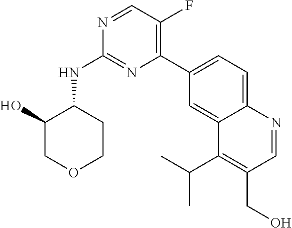 [SMILES: CC(C)c1c(CO)cnc2ccc(-c3nc(N[C@@H]4CCOC[C@H]4O)ncc3F)cc12]

To the solution of ethyl 6-(5-fluoro-2-(((3S,4R)-3-hydroxytetrahydro-2H-pyran-4-yl)amino)pyrimidin-4-yl)-4-isopropylquinoline-3-carboxylate (50 mg, 0.11 mmol) in tetrahydrofuran (10 mL) under nitrogen at 0° C. was added lithium aluminium hydride (13 mg, 0.33 mmol) and stirred at room temperature for 3 h. The reaction was quenched with methanol and the solvent was evaporated. The residue was purified by prep-HPLC (eluent: water (0.1% formic acid)/acetonitrile (0.1% formic acid)) to give the product (6 mg, 12%). LC-MS (M+H)⁺=413.3.

### Step 6: (3S,4R)-44(4-(3-(chloromethyl)-4-isopropylquinolin-6-yl)-5-fluoropyrimidin-2-ylamino)tetrahydro-2H-pyran-3-ol

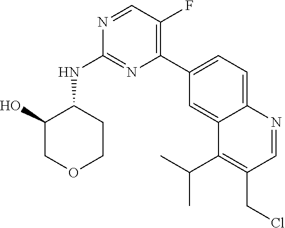 [SMILES: CC(C)c1c(CCl)cnc2ccc(-c3nc(N[C@@H]4CCOC[C@H]4O)ncc3F)cc12]

To the solution of (3S,4R)-4-((5-fluoro-4-(3-(hydroxymethyl)-4-isopropylquinolin-6-yl)pyrimidin-2-yl)amino)tetrahydro-2H-pyran-3-ol (80 mg, 0.19 mmol) in dichloromethane (5 mL) was added thionyl chloride (46 mg, 0.39 mmol) at 0° C. and the mixture was stirred for 1 h before quenched with saturated aqueous solution of sodium bicarbonate. The aqueous layer was extracted with dichloromethane (30 mL). The combined organic layers were washed with brine, dried over sodium sulfate, filtered and evaporated to give 85 mg crude product which was used for next step without further purification. LC-MS (M+H)⁺=431.2.

### Step 7: (3S,4R)-44(5-fluoro-4-(4-isopropyl-3-(morpholinomethyl quinolin-6-yl)pyrimidin-2-ylamino)tetrahydro-2H-pyran-3-ol

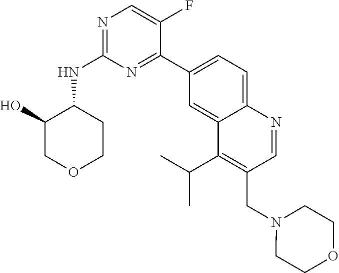 [SMILES: CC(C)c1c(CN2CCOCC2)cnc2ccc(-c3nc(N[C@@H]4CCOC[C@H]4O)ncc3F)cc12]

To a solution of (3S,4R)-4-((4-(3-(chloromethyl)-4-isopropylquinolin-6-yl)-5-fluoropyrimidin-2-yl)amino)tetrahydro-2H-pyran-3-ol (59 mg, 0.14 mmol) in dimethylformamide (2 mL) were added morpholine (24 mg, 0.27 mmol) and N,N-diisopropylethylamine (35 mg, 0.27 mmol) at 25° C. The mixture was stirred at 25° C. for 5 h before diluted with ethyl acetate (20 mL). The organic solution was washed with brine (20 mL), dried over sodium sulfate, filtered and concentrated under reduce pressure. The residue was purified over silica gel by combi-flash, eluting with methanol in dichloromethane (5%, v/v) to give the title compound (9.1 mg, 15%). ¹H-NMR (400 MHz, DMSO-d6) δ: 9.13 (s, 1H), 8.78 (s, 1H), 8.52-8.48 (m, 1H), 8.33-8.24 (m, 1H), 8.18-8.10 (m, 11H), 7.26 (d, J=7.9 Hz, 1H), 5.00-4.95 (m, 1H), 4.04-4.00 (m, 1H), 3.92-3.81 (m, 3H), 3.76-3.71 (m, 2H), 3.60-3.50 (m, 5H), 3.42-3.35 (m, 1H), 3.05 (t, J=10.3 Hz, 1H), 2.40 (s, 3H), 2.45-2.30 (m, 1H), 2.09-2.00 (m, 1H) 1.62-1.50 (m, 7H). LC-MS (M+H)⁺=482.1.

### Example 18: (3S,4R)-4-((5-fluoro-4-(3-(((S)-3-fluoropyrrolidin-1-yl)methyl)-4-isopropylquinolin-6-yl)pyrimidin-2-yl)amino)tetrahydro-2H-pyran-3-ol

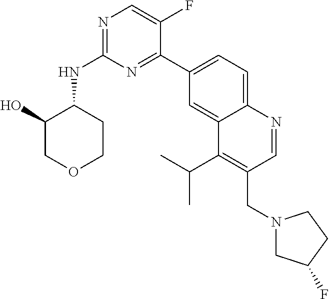 [SMILES: CC(C)c1c(CN2CC[C@H](F)C2)cnc2ccc(-c3nc(N[C@@H]4CCOC[C@H]4O)ncc3F)cc12]

The title compound (8.1 mg, 18%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (S)-3-fluoropyrrolidine. ¹H-NMR (400 MHz, DMSO-d6) δ: 9.12 (s, 1H), 8.83 (s, 1H), 8.52-8.48 (m, 1H), 8.33-8.24 (m, 1H), 8.18-8.10 (m, 1H), 7.26 (d, J=7.9 Hz, 1H), 5.30-5.10 (m, 1H), 5.00-4.94 (m, 1H), 4.04-3.98 (m, 1H), 3.92-3.80 (m, 5H), 3.58-3.50 (m, 1H), 3.42-3.36 (m, 1H), 3.03 (t, J=10.3 Hz, 1H), 2.85-2.73 (m, 2H), 2.71-2.60 (m, 1H), 2.40-2.31 (m, 1H), 2.23-2.00 (m, 2H), 1.93-1.78 (m, 1H), 1.60-1.49 (m, 7H). LC-MS (M+H)⁺=484.1.

### Example 19: (3S,4R)-4-((4-(3-((cyclobutylamino)methyl)-4-isopropylquinolin-6-yl)-5-fluoropyrimidin-2-yl)amino)tetrahydro-2H-pyran-3-ol

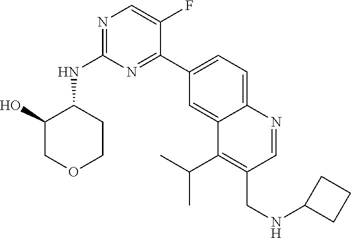 [SMILES: CC(C)c1c(CNC2CCC2)cnc2ccc(-c3nc(N[C@@H]4CCOC[C@H]4O)ncc3F)cc12]

The title compound (20 mg, 37%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and cyclobutanamine. ¹H-NMR (400 MHz, DMSO-d6) δ 9.10 (brs, 1H), 8.83 (s, 1H), 8.50 (d, J=3.9 Hz, 1H), 8.32-8.23 (m, 1H), 8.13 (d, J=8.8 Hz, 1H), 7.25 (d, J=7.8 Hz, 1H), 4.97 (d, J=5.3 Hz, 1H), 4.06-3.77 (m, 6H), 3.63-3.49 (m, 1H), 3.43-3.35 (m, 1H), 3.26-3.13 (m, 1H), 3.13-3.00 (m, 1H), 2.19-2.00 (m, 3H), 1.82-1.69 (m, 3H), 1.67-1.48 (m, 9H). LC-MS (M+H)⁺=466.1.

### Example 20: (3S,4R)-4-((4-(3-(((3,3-difluorocyclobutyl)amino)methyl)-4-isopropylquinolin-6-yl)-5-fluoropyrimidin-2-yl)amino)tetrahydro-2H-pyran-3-ol

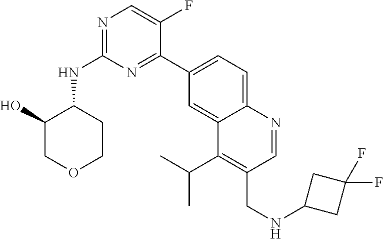 [SMILES: CC(C)c1c(CNC2CC(F)(F)C2)cnc2ccc(-c3nc(N[C@@H]4CCOC[C@H]4O)ncc3F)cc12]

The title compound (15 mg, 34%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and 3,3-difluorocyclobutan-1-amine. ¹H-NMR (300 MHz, DMSO-d6) δ 9.11 (brs, 1H), 8.84 (s, 1H), 8.50 (d, J=3.9 Hz, 1H), 8.28 (d, J=8.8 Hz, 1H), 8.13 (d, J=8.8 Hz, 1H), 7.26 (d, J=7.7 Hz, 1H), 4.97 (d, J=5.3 Hz, 11H), 4.06-3.78 (m, 6H), 3.59-3.53 (m, 11H), 3.43-3.35 (m, 11H), 3.23-3.17 (m, 1H), 3.13-3.00 (m, 1H), 2.79-2.73 (m, 3H), 2.44-2.30 (m, 2H), 2.11-2.01 (m, 1H), 1.68-1.47 (m, 7H). LC-MS (M+H)⁺=502.2.

### Example 21: (3S,4R)-4-((5-fluoro-4-(4-isopropyl-3-(((tetrahydrofuran-3-yl)amino)methyl)quinolin-6-yl)pyrimidin-2-yl)amino)tetrahydro-2H-pyran-3-ol

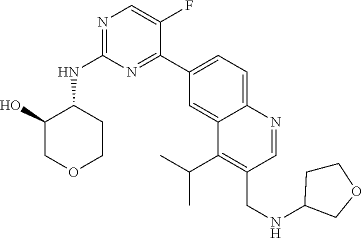 [SMILES: CC(C)c1c(CNC2CCOC2)cnc2ccc(-c3nc(N[C@@H]4CCOC[C@H]4O)ncc3F)cc12]

The title compound (18 mg, 44%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and tetrahydrofuran-3-amine. ¹H-NMR (300 MHz, DMSO-d6) δ 9.11 (brs, 1H), 8.86 (s, 1H), 8.50 (d, J=3.9 Hz, 1H), 8.32-8.22 (m, 1H), 8.13 (d, J=8.9 Hz, 1H), 7.26 (d, J=7.8 Hz, 1H), 4.98 (d, J=5.2 Hz, 1H), 4.03-3.81 (m, 6H), 3.80-3.63 (m, 3H), 3.62-3.44 (m, 2H), 3.42-3.35 (m, 2H), 3.13-3.00 (m, 1H), 2.36-2.30 (m, 1H), 2.10-1.89 (m, 2H), 1.83-1.67 (m, 1H), 1.64-1.47 (m, 7H).

LC-MS (M+H)⁺=482.1.

### Example 22: (3S,4R)-4-((5-fluoro-4-(4-isopropyl-3-((isopropylamino)methyl)quinolin-6-yl)pyrimidin-2-yl)amino)tetrahydro-2H-pyran-3-ol

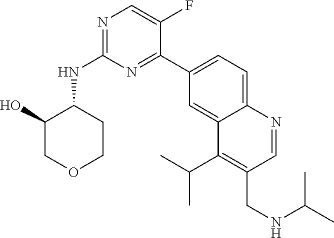 [SMILES: CC(C)NCc1cnc2ccc(-c3nc(N[C@@H]4CCOC[C@H]4O)ncc3F)cc2c1C(C)C]

The title compound (10 mg, 12%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and isopropylamine. ¹H-NMR (300 MHz, DMSO-d6) δ 9.10 (brs, 11H), 8.85 (s, 11H), 8.49 (d, J=3.9 Hz, 11H), 8.32-8.22 (m, 11H), 8.12 (d, J=8.8 Hz, 1H), 7.26 (d, J=7.7 Hz, 1H), 4.98 (d, J=5.2 Hz, 11H), 4.05-3.80 (m, 6H), 3.63-3.48 (m, 11H), 3.43-3.34 (m, 1H), 3.13-3.00 (m, 1H), 2.86-2.72 (m, 11H), 2.11-2.01 (m, 11H), 1.64-1.48 (m, 7H), 1.10-1.02 (m, 6H). LC-MS (M+H)⁺=454.3.

### Example 23: (3S,4R)-4-((5-fluoro-4-(3-(((R)-3-fluoropyrrolidin-1-yl)methyl)-4-isopropylquinolin-6-yl)pyrimidin-2-yl)amino)tetrahydro-2H-pyran-3-ol

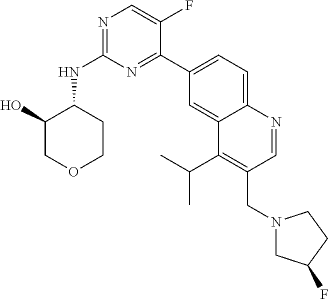 [SMILES: CC(C)c1c(CN2CC[C@@H](F)C2)cnc2ccc(-c3nc(N[C@@H]4CCOC[C@H]4O)ncc3F)cc12]

The title compound (9.2 mg, 19%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (R)-3-fluoropyrrolidine. ¹H-NMR (400 MHz, DMSO-d6) a: 9.12 (s, 1H), 8.83 (s, 1H), 8.52-8.48 (m, 1H), 8.33-8.24 (m, 1H), 8.18-8.10 (m, 1H), 7.26 (d, J=7.9 Hz, 1H), 5.30-5.10 (m, 1H), 5.00-4.94 (m, 1H), 4.04-3.98 (m, 1H), 3.92-3.80 (m, 5H), 3.58-3.50 (m, 1H), 3.42-3.36 (m, 1H), 3.03 (t, J=10.3 Hz, 1H), 2.85-2.73 (m, 2H), 2.71-2.60 (m, 1H), 2.40-2.31 (m, 1H), 2.23-2.00 (m, 2H), 1.93-1.78 (m, 1H), 1.60-1.49 (m, 7H). LC-MS (M+H)⁺=484.1.

### Example 24: (3S,4R)-4-((5-fluoro-4-(4-isopropyl-3-(((((R)-tetrahydrofuran-2-yl)methyl)amino)methyl)quinolin-6-yl)pyrimidin-2-yl)amino)tetrahydro-2H-pyran-3-ol

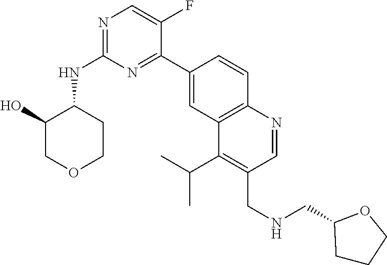 [SMILES: CC(C)c1c(CNC[C@H]2CCCO2)cnc2ccc(-c3nc(N[C@@H]4CCOC[C@H]4O)ncc3F)cc12]

The title compound (14 mg, 19%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (R)-(tetrahydrofuran-2-yl)methanamine. ¹H-NMR (400 MHz, DMSO-d6) δ 9.11 (s, 1H), 8.84 (s, 1H), 8.52-8.48 (m, 1H), 8.33-8.24 (m, 1H), 8.18-8.10 (m, 1H), 7.26 (d, J=7.9 Hz, 1H), 4.97 (s, 1H), 4.00-3.81 (m, 7H), 3.76-3.69 (m, 1H), 3.63-3.50 (m, 2H), 3.40-3.33 (m, 2H), 3.05 (t, J=10.3 Hz, 1H), 2.64-2.59 (m, 2H), 2.10-2.00 (m, 1H), 1.93-1.84 (m, 1H), 1.83-1.75 (m, 2H), 1.62-1.51 (m, 8H). LC-MS (M+H)⁺=496.1.

### Example 25: (3S,4R)-4-((4-(3-((cyclopropylamino)methyl)-4-isopropylquinolin-6-yl)-5-fluoropyrimidin-2-yl)amino)tetrahydro-2H-pyran-3-ol

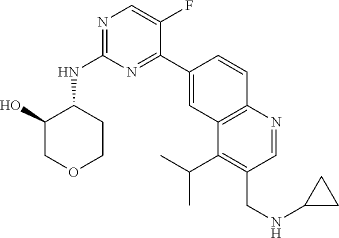 [SMILES: CC(C)c1c(CNC2CC2)cnc2ccc(-c3nc(N[C@@H]4CCOC[C@H]4O)ncc3F)cc12]

The title compound (20 mg, 48%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and cyclopropanamine. ¹H-NMR (400 MHz, DMSO-d6) δ 9.10 (s, 1H), 8.84 (s, 1H), 8.49 (d, J=3.1 Hz, 1H), 8.26 (d, J=8.6 Hz, 1H), 8.12 (d, J=8.8 Hz, 1H), 7.26 (d, J=7.6 Hz, 1H), 4.97 (s, 1H), 4.08-3.75 (m, 6H), 3.65-3.45 (m, 1H), 3.39-3.33 (m, 2H), 3.05 (t, J=10.4 Hz, 1H), 2.20-1.93 (m, 2H), 1.65-1.42 (m, 7H), 0.38 (d, J=5.5 Hz, 2H), 0.23 (s, 2H). LC-MS (M+H)⁺=452.1.

### Example 26: (3S,4R)-4-((4-(3-((cyclopentylamino)methyl)-4-isopropylquinolin-6-yl)-5-fluoropyrimidin-2-yl)amino)tetrahydro-2H-pyran-3-ol

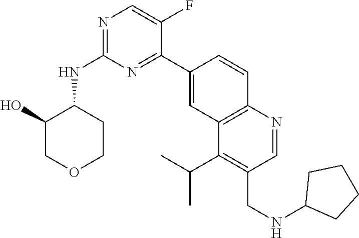 [SMILES: CC(C)c1c(CNC2CCCC2)cnc2ccc(-c3nc(N[C@@H]4CCOC[C@H]4O)ncc3F)cc12]

The title compound (20 mg, 45%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and cyclopentanamine. ¹H-NMR (400 MHz, DMSO-d6) δ 9.10 (s, 1H), 8.84 (s, 1H), 8.50 (s, 1H), 8.27 (d, J=8.5 Hz, 1H), 8.12 (d, J=8.8 Hz, 1H), 7.26 (d, J=7.4 Hz, 1H), 4.97 (s, 1H), 3.99-3.86 (m, 6H), 3.55 (s, 1H), 3.36 (t, J=11.3 Hz, 2H), 3.14-2.99 (m, 2H), 2.04 (s, 1H), 1.80-1.55 (m, 10H), 1.54-1.42 (m). LC-MS (M+H)⁺=480.1.

### Example 27: (3S,4R)-4-((5-fluoro-4-(4-isopropyl-3-(((((R)-tetrahydrofuran-3-yl)methyl)amino)methyl)quinolin-6-yl)pyrimidin-2-yl)amino)tetrahydro-2H-pyran-3-ol

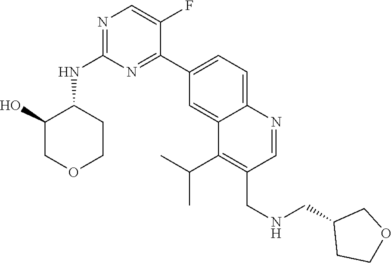 [SMILES: CC(C)c1c(CNC[C@H]2CCOC2)cnc2ccc(-c3nc(N[C@@H]4CCOC[C@H]4O)ncc3F)cc12]

The title compound (10 mg, 16%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (R)-(tetrahydrofuran-3-yl)methanamine. ¹H-NMR (400 MHz, DMSO-d6) δ 9.12 (s, 1H), 8.84 (s, 1H), 8.52-8.48 (m, 1H), 8.33-8.24 (m, 1H), 8.18-8.10 (m, 1H), 7.26 (d, J=7.9 Hz, 1H), 4.98 (br s, 1H), 4.00-3.80 (m, 6H), 3.80-3.65 (m, 2H), 3.59-3.46 (m, 2H), 3.42-3.36 (m, 3H), 3.03 (t, J=10.3 Hz, 1H), 2.65-2.57 (m, 2H), 2.40-2.30 (m, 1H), 2.10-2.00 (m, 1H) 2.00-1.88 (m, 1H), 1.62-1.50 (m, 8H). LC-MS (M+H)⁺=496.2.

### Example 28: (3S,4R)-4-((4-(3-(cyclohexylamino)methyl)-4-isopropylquinolin-6-yl)-5-fluoropyrimidin-2-yl)amino)tetrahydro-2H-pyran-3-ol

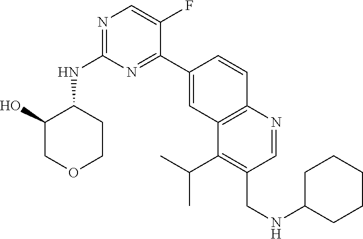 [SMILES: CC(C)c1c(CNC2CCCCC2)cnc2ccc(-c3nc(N[C@@H]4CCOC[C@H]4O)ncc3F)cc12]

The title compound (8 mg, 35%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and cyclohexylamine. ¹H-NMR (400 MHz, DMSO-d6) δ 9.11 (s, 1H), 8.85 (s, 1H), 8.49 (s, 1H), 8.27 (d, J=8.7 Hz, 1H), 8.12 (d, J=8.8 Hz, 1H), 7.26 (d, J=7.4 Hz, 1H), 4.97 (s, 1H), 3.96 (s, 3H), 3.86 (s, 3H), 3.65-3.45 (m, 1H), 3.45-3.25 (m, 1H), 3.05 (t, J=10.5 Hz, 1H), 2.49-2.41 (m, 2H), 2.14-1.94 (m, 1H), 1.89 (d, J=10.6 Hz, 2H), 1.77-1.57 (m, 2H), 1.66-1.46 (m, 8H), 1.30-1.06 (m, 5H). LC-MS (M+H)⁺=494.0.

### Example 29: (3S,4R)-4-((5-fluoro-4-(4-isopropyl-3-(((tetrahydro-2H-pyran-4-yl)amino)methyl)quinolin-6-yl)pyrimidin-2-yl)amino)tetrahydro-2H-pyran-3-ol

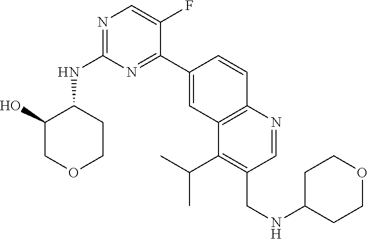 [SMILES: CC(C)c1c(CNC2CCOCC2)cnc2ccc(-c3nc(N[C@@H]4CCOC[C@H]4O)ncc3F)cc12]

The title compound (16 mg, 70%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and tetrahydro-2H-pyran-4-amine. ¹H-NMR (400 MHz, DMSO-d6) δ 9.11 (s, 1H), 8.86 (s, 1H), 8.50 (s, 1H), 8.27 (d, J=8.3 Hz, 1H), 8.12 (d, J=8.7 Hz, 1H), 7.26 (d, J=7.4 Hz, 1H), 4.97 (s, 1H), 3.98 (s, 3H), 3.84 (d, J=10.0 Hz, 5H), 3.64-3.44 (m, 1H), 3.46-3.19 (m, 4H), 3.05 (t, J=10.6 Hz, 1H), 2.75-2.55 (m, 1H), 2.13-1.93 (m, 1H), 1.84 (d, J=12.3 Hz, 2H), 1.68-1.44 (m, 7H), 1.32 (d, J=10.1 Hz, 2H). LC-MS (M+H)⁺=496.0.

### Example 30: (3S,4R)-4-((5-fluoro-4-(4-isopropyl-3-((((R)-tetrahydro-2H-pyran-3-yl)amino)methyl)quinolin-6-yl)pyrimidin-2-yl)amino)tetrahydro-2H-pyran-3-ol

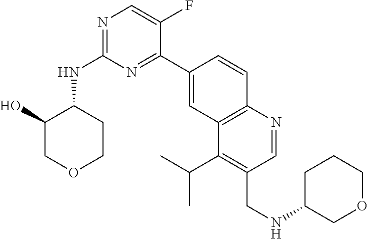 [SMILES: CC(C)c1c(CN[C@@H]2CCCOC2)cnc2ccc(-c3nc(N[C@@H]4CCOC[C@H]4O)ncc3F)cc12]

The title compound (16 mg, 70)%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (R)-tetrahydrofuran-3-amine. ¹H-NMR (400 MHz, DMSO-d6) δ 9.10 (s, 1H), 8.85 (s, 1H), 8.50 (s, 11H), 8.27 (d, J=8.9 Hz, 11H), 8.12 (d, J=8.8 Hz, 11H), 7.26 (d, J=7.6 Hz, 1H), 4.97 (d, J=4.9 Hz, 1H), 4.06-3.92 (m, 3H), 3.91-3.71 (m, 4H), 3.69 (d, J=11.4 Hz, 1H), 3.64-3.44 (m, 1H), 3.48-3.17 (m, 2H), 3.06 (t, J=10.0 Hz, 2H), 2.66-2.46 (m, 2H), 2.09-1.89 (m, 2H), 1.71-1.37 (m, 9H), 1.40-1.20 (m, 1H). LC-MS (M+H)⁺=496.0.

### Example 31: (3S,4R)-4-((5-fluoro-4-(4-isopropyl-3-(methoxymethyl)quinolin-6-yl)pyrimidin-2-yl)amino)tetrahydro-2H-pyran-3-ol

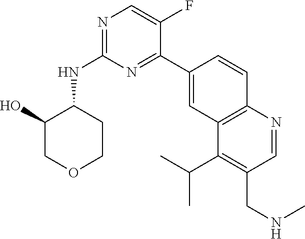 [SMILES: CNCc1cnc2ccc(-c3nc(N[C@@H]4CCOC[C@H]4O)ncc3F)cc2c1C(C)C]

To a solution of (3S,4R)-4-((5-fluoro-4-(4-isopropyl-3-(methoxymethyl)quinolin-6-yl)pyrimidin-2-yl)amino)tetrahydro-2H-pyran-3-ol (20 mg, 0.046 mmol) in dimethylfornamide (1 mL) was added sodium methoxide (5 mg, 0.092 mmol, 2 eq.) at room temperature. The mixture was stirred at 80° C. for 16 h before cooled to room temperature and addition of water (10 mL). The aqueous layer was extracted with ethyl acetate (30 mL). The organic phase was washed with brine (20 mL), dried with over sodium sulfate, filtered and concentrated under reduced pressure. The residue was purified over silica gel by combi-flash, eluting with methanol in dichloromethane (5%, v/v) to give the title compound (17 mg, 80%). ¹H-NMR (400 MHz, DMSO-d6) δ 9.12 (s, 1H), 8.87 (s, 1H), 8.50 (d, J=3.0 Hz, 1H), 8.30 (d, J=8.8 Hz, 1H), 8.15 (d, J=8.7 Hz, 1H), 7.27 (d, J=7.8 Hz, 1H), 4.97 (s, 11H), 4.70 (s, 2H), 4.00-3.75 (m, J=4.6 Hz, 4H), 3.54 (s, 1H), 3.36 (s, 3H), 3.46-3.26 (m, 1H), 3.05 (t, J=10.6 Hz, 1H), 2.12-1.93 (m, 1H), 1.65-1.45 (m, 7H). LC-MS (M+H)⁺=427.1.

### Example 32: (3S,4R)-4-((5-fluoro-4-(4-isopropyl-3-((((S)-tetrahydrofuran-3-yl)amino)methyl)quinolin-6-yl)pyrimidin-2-yl)amino)tetrahydro-2H-pyran-3-ol

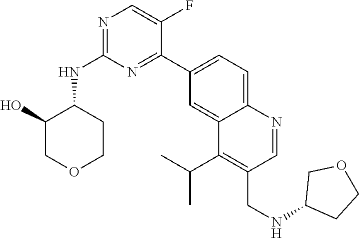 [SMILES: CC(C)c1c(CN[C@H]2CCOC2)cnc2ccc(-c3nc(N[C@@H]4CCOC[C@H]4O)ncc3F)cc12]

The title compound (8.5 mg, 15%) was prepared in a manner similar to that in Example 11 step 1 from (3S,4R)-4-(4-(3-(chloromethyl)-4-isopropylquinolin-6-yl)-5-fluoropyrimidin-2-yl)amino)tetrahydro-2H-pyran-3-ol and (S)-tetrahydrofuran-3-amine. ¹H-NMR (400 MHz, DMSO-d6) δ 9.11 (s, 11H), 8.85 (s, 11H), 8.52-8.48 (m, 1H), 8.33-8.24 (m, 11H), 8.18-8.10 (m, 1H), 7.26 (d, J=7.9 Hz, 1H), 4.98 (s, 1H), 4.00-3.80 (m, 6H), 3.80-3.65 (m, 3H), 3.59-3.46 (m, 2H), 3.42-3.36 (m, 3H), 3.03 (t, J=10.3 Hz, 1H), 2.00-1.93 (m, 2H) 1.80-1.70 (m, 1H), 1.61-1.50 (m, 7H). LC-MS (M+H)⁺=482.1.

### Example 33: (3S,4R)-4-((5-fluoro-4-(4-isopropyl-3-(((((S)-tetrahydrofuran-3-yl)methyl)amino)methyl)quinolin-6-yl)pyrimidin-2-yl)amino)tetrahydro-2H-pyran-3-ol

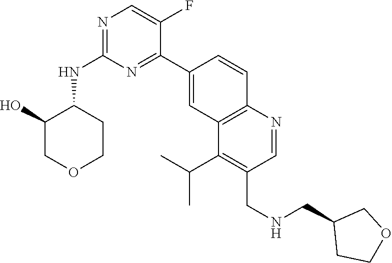 [SMILES: CC(C)c1c(CNC[C@@H]2CCOC2)cnc2ccc(-c3nc(N[C@@H]4CCOC[C@H]4O)ncc3F)cc12]

The title compound (9.5 mg, 16%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (S)-(tetrahydrofuran-3-yl)methanamine. ¹H-NMR (400 MHz, DMSO-d6) δ 9.11 (s, 1H), 8.84 (s, 1H), 8.52-8.48 (m, 1H), 8.33-8.24 (m, 1H), 8.18-8.10 (m, 1H), 7.26 (d, J=7.9 Hz, 1H), 4.97 (s, 1H), 4.00-3.80 (m, 6H), 3.80-3.65 (m, 2H), 3.59-3.46 (m, 2H), 3.42-3.36 (m, 3H), 3.03 (t, J=10.3 Hz, 1H), 2.65-2.57 (m, 2H), 2.40-2.30 (m, 1H), 2.10-2.00 (m, 1H), 2.00-1.88 (m, 1H), 1.62-1.50 (m, 8H). LC-MS (M+H)⁺=496.1.

### Example 34: (3S,4R)-4-((5-fluoro-4-(4-isopropyl-3-(((((S)-tetrahydrofuran-2-yl)methyl)amino)methyl)quinolin-6-yl)pyrimidin-2-yl)amino)tetrahydro-2H-pyran-3-ol

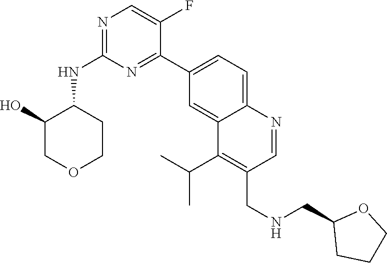 [SMILES: CC(C)c1c(CNC[C@@H]2CCCO2)cnc2ccc(-c3nc(N[C@@H]4CCOC[C@H]4O)ncc3F)cc12]

The title compound (6 mg, 12%) was prepared in a manner similar to in example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (S)-tetrahydrofuran-2-yl)methanamine. ¹H-NMR (400 MHz, DMSO-d6) δ 9.11 (s, 1H), 8.84 (s, 1H), 8.52-8.48 (m, 1H), 8.33-8.24 (m, 1H), 8.18-8.10 (m, 1H), 7.26 (d, J=7.9 Hz, 1H), 4.98 (s, 1H), 4.00-3.81 (m, 7H), 3.76-3.69 (m, 1H), 3.63-3.50 (m, 2H), 3.40-3.33 (m, 2H), 3.05 (t, J=10.3 Hz, 1H), 2.64-2.59 (m, 2H), 2.10-2.00 (m, 1H), 1.93-1.84 (m, 1H), 1.83-1.75 (m, 2H), 1.62-1.51 (m, 8H). LC-MS (M+H)⁺=496.1.

### Example 35: (3S,4R)-4-((5-fluoro-4-(4-isopropyl-3-((((R)-tetrahydrofuran-3-yl)amino)methyl)quinolin-6-yl)pyrimidin-2-yl)amino)tetrahydro-2H-pyran-3-ol

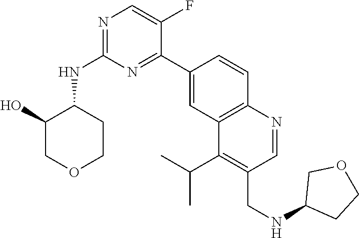 [SMILES: CC(C)c1c(CN[C@@H]2CCOC2)cnc2ccc(-c3nc(N[C@@H]4CCOC[C@H]4O)ncc3F)cc12]

The title compound (11.5 mg, 18%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (R)-tetrahydrofuran-3-amine. ¹H-NMR (400 MHz, DMSO-d6) δ 9.11 (s, 1H), 8.85 (s, 1H), 8.52-8.48 (m, 1H), 8.33-8.24 (m, 1H), 8.18-8.10 (m, 1H), 7.26 (d, J=7.9 Hz, 1H), 4.98 (s, 1H), 4.00-3.80 (m, 6H), 3.80-3.65 (m, 3H), 3.59-3.46 (m, 2H), 3.42-3.36 (m, 3H), 3.03 (t, J=10.3 Hz, 1H), 2.00-1.93 (m, 2H), 1.80-1.70 (m, 1H), 1.61-1.50 (m, 7H). LC-MS (M+H)⁺=482.1.

### Example 36: (3S,4R)-4-((4-(3-((7-oxa-4-azaspiro[2.5]octan-4-yl)methyl)-4-isopropylquinolin-6-yl)-5-fluoropyrimidin-2-yl)amino)tetrahydro-2H-pyran-3-ol

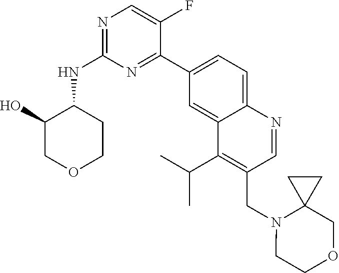 [SMILES: CC(C)c1c(CN2CCOCC23CC3)cnc2ccc(-c3nc(N[C@@H]4CCOC[C@H]4O)ncc3F)cc12]

The title compound (4 mg, 17%) was prepared in a manner similar to that in Example 11 step 1 from (3S,4R)-4-((4-(3-chloromethyl)-4-isopropylquinolin-6-yl)-5-fluoropyrimidin-2-yl)amino)tetrahydro-2H-pyran-3-ol and 7-oxa-4-azaspiro[2.5]octane hydrochloride. ¹H-NMR (400 MHz, DMSO-d6) δ 9.17-9.04 (m, 1H), 8.79 (s, 1H), 8.50 (s, 1H), 8.27 (d, J=8.6 Hz, 1H), 8.12 (d, J=8.8 Hz, 1H), 7.26 (d, J=8.5 Hz, 1H), 4.97 (s, 1H), 4.13-4.01 (m, 2H), 3.97-3.79 (m, 4H), 3.68 (s, 2H), 3.52 (s, 3H), 3.31-3.29 (m, 1H), 3.10-3.00 (m, 11H), 2.75-2.59 (m, 2H), 2.10-1.99 (m, 1H), 1.60-1.45 (m, 7H), 0.69 (s, 2H), 0.50 (s, 2H). LC-MS (M+H)⁺=508.1.

### Example 37: (3S,4R)-4-((5-fluoro-4-(4-isopropyl-3-((((S)-tetrahydro-2H-pyran-3-yl)amino)methyl)quinolin-6-yl)pyrimidin-2-yl)amino)tetrahydro-2H-pyran-3-ol

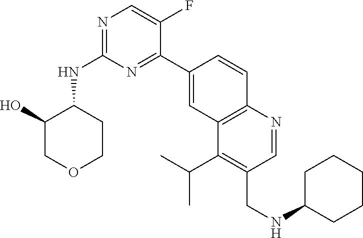

The title compound (12 mg, 41%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (S)-tetrahydro-2H-pyran-3-amine. ¹H-NMR (400 MHz, DMSO-d6) δ 9.10 (s, 1H), 8.85 (s, 1H), 8.50 (s, 1H), 8.27 (d, J=8.4 Hz, 11H), 8.12 (d, J=9.3 Hz, 1H), 7.26 (d, J=7.4 Hz, 1H), 4.98 (s, 1H), 4.05-3.91 (m, 3H), 3.86 (d, J=8.1 Hz, 4H), 3.69 (d, J=11.4 Hz, 1H), 3.55 (s, 1H), 3.36 (t, J=11.2 Hz, 2H), 3.27 (t, J=10.9 Hz, 2H), 3.06 (t, J=9.8 Hz, 2H), 2.00 (s, 2H), 1.71-1.39 (m, 9H), 1.30 (d, J=10.4 Hz, 1H). LC-MS (M+H)⁺=496.0.

### Example 38: (3S,4R)-4-((4-(3-(aminomethyl)-4-isopropylquinolin-6-yl)-5-fluoropyrimidin-2-yl)amino)tetrahydro-2H-pyran-3-ol

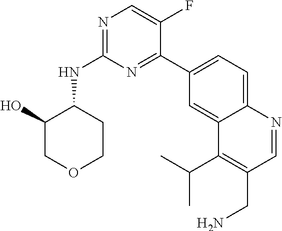 [SMILES: CC(C)c1c(CN)cnc2ccc(-c3nc(N[C@@H]4CCOC[C@H]4O)ncc3F)cc12]

### Step 1: (3S,4R)-44(4-(3-(azidomethyl)-4-isopropylquinolin-6-yl)-5-fluoropyrimidin-2-yl)amino)tetrahydro-2H-pyran-3-ol

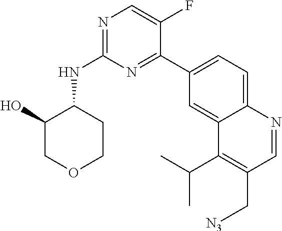 [SMILES: CC(C)c1c(CN=[N+]=[N-])cnc2ccc(-c3nc(N[C@@H]4CCOC[C@H]4O)ncc3F)cc12]

A solution of (3S,4R)-4-({5-fluoro-4-[3-(hydroxymethyl)-4-isopropylquinolin-6-yl]pyrimidin-2-yl}amino)oxan-3-ol (190 mg, 0.46 mmol) and diphenylphosphoryl azide (255 mg, 0.92 mmol) in dimethylformamide (2 mL) was stirred at 50° C. for 5 h under nitrogen atmosphere. The solution was concentrated under reduced pressure and the residue was purified by flash chromatography eluting with MeOH in dichloromethane (0% to 20% gradient, v/v) to yield the title compound (66 mg, 32%). LC-MS (M+H)⁺=438.2.

### Step 2: (3S,4R)-4(4-(3-(aminomethyl)-4-isopropylquinolin-6-yl)-5-fluoropyrimidin-2-yl)amino)tetrahydro-2H-pyran-3-ol

A solution of (3S,4R)-4-((4-(3-(azidomethyl)-4-isopropylquinolin-6-yl)-5-fluoropyrimidin-2-yl)amino)tetrahydro-2H-pyran-3-ol (66 mg, 0.15 mmol) and triphenylphosphine (79 mg, 0.30 mmol) in tetrahydrofuran/water (11 mL, 10/1, v/v) was stirred at 50° C. for 3 h under nitrogen atmosphere. The reaction solution was concentrated under reduced pressure. The residue was purified by flash chromatography eluting with MeOH in dichloromethane (0% to 20% gradient) and then prep-HPLC under the following conditions: column. XBridge Shield RP18 OBD Column, 30×150 mm, 5 um; mobile phase, acetonitrile in water (with 10 mmol/L NH₄HCO₃ and 0.1% NH₄OH), 17% to 47% gradient in 9 min; detector, UV 254 nm. The title compound (14 mg, 24%) obtained. ¹H-NMR (400 MHz, DMSO-d6) δ 9.10 (s, 1H), 8.90 (s, 1H), 8.49 (d, J=3.9 Hz, 1H), 8.26 (d, J=8.8 Hz, 1H), 8.12 (d, J=8.8 Hz, 1H), 7.26 (d, J=7.8 Hz, 1H), 5.01-4.95 (m, 1H), 4.04-3.80 (m, 6H), 3.60-3.50 (m, 1H), 3.42-3.34 (m, 1H), 3.11-3.01 (m, 1H), 2.10-1.85 (m, 3H), 1.65-1.46 (m, 7H). LC-MS (M+H)⁺=412.1.

### Example 39 and Example 40: (3S,4R)-4-((5-fluoro-4-(4-isopropyl-3-((((S)-tetrahydrofuran-3-yl)amino)methyl)quinolin-6-yl)pyrimidin-2-yl)amino)tetrahydro-2H-pyran-3-ol & (3S,4R)-4-((5-fluoro-4-(4-isopropyl-3-((((R)-tetrahydrofuran-3-yl)amino)methyl)quinolin-6-yl)pyrimidin-2-yl)amino)tetrahydro-2H-pyran-3-ol

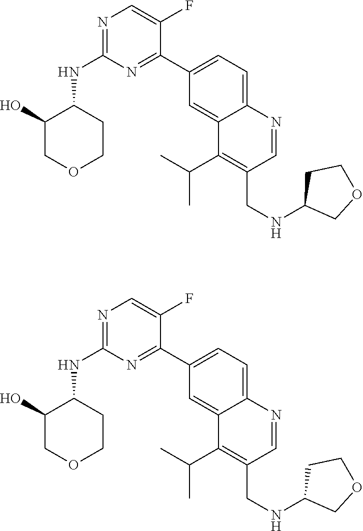 [SMILES: CC(C)c1c(CN[C@H]2CCOC2)cnc2ccc(-c3nc(N[C@@H]4CCOC[C@H]4O)ncc3F)cc12 ; CC(C)c1c(CN[C@@H]2CCOC2)cnc2ccc(-c3nc(N[C@@H]4CCOC[C@H]4O)ncc3F)cc12]

The title compounds were prepared in a manner similar to that in Example 1 step 1 from (3S,4R)-4-((4-(3-(chloromethyl)-4-isopropylquinolin-6-yl)-5-fluoropyrimidin-2-yl)amino)tetrahydro-2H-pyran-3-ol and racemic tetrahydrofuran-3-amine. Example 39 and Example 40 were separated by chiral HPLC to give (3S,4R)-4-((5-fluoro-4-(4-isopropyl-3-((((S)-tetrahydrofuran-3-yl)amino)methyl)quinolin-6-yl)pyrimidin-2-yl)amino)tetrahydro-2H-pyran-3-ol and (3S,4R)-4-((5-fluoro-4-(4-isopropyl-3-((((R)-tetrahydrofuran-3-yl)amino)methyl)quinolin-6-yl)pyrimidin-2-yl)amino)tetrahydro-2H-pyran-3-ol. Analytical chiral HPLC condition; CHIRALPAK ID-3, 0.46×5 cm, 3.0 um. Mobile phase: (hexane:dichloromethane=3:1, v/v, containing 0.5% 2M solution of NH₃ in methanol):EtOH, 20 mL/min in 13 min.

Example 39 (5 mg, 10%) ¹H NMR (400 MHz, DMSO-d6) δ 9.11 (brs, 1H), 8.86 (s, 1H), 8.50 (d, J=3.9 Hz, 1H), 8.31-8.24 (m, 1H), 8.13 (d, J=8.8 Hz, 1H), 7.27 (d, J=7.8 Hz, 1H), 4.98 (d, J=5.3 Hz, 1H), 4.04-3.80 (m, 6H), 3.78-3.65 (m, 3H), 3.61-3.44 (m, 2H), 3.41-3.36 (m, 1H), 3.34-3.26 (m, 1H), 3.13-3.00 (m, 1H), 2.40-2.22 (m, 1H), 2.09-1.91 (m, 2H), 1.81-1.69 (m, 1H), 1.62-1.42 (m, 7H). LC-MS (M+H)⁺=482.3. Chiral HPLC: RT=2.083 min.

Example 40: (2 mg, 3%) 1H NMR (400 MHz, DMSO-d6) δ 9.12 (brs, 1H), 8.86 (s, 1H), 8.50 (d, J=3.8 Hz, 1H), 8.31-8.24 (m, 1H), 8.13 (d, J=8.8 Hz, 1H), 7.28 (d, J=7.8 Hz, 1H), 4.99 (d, J=5.3 Hz, 1H), 4.02-3.81 (m, 6H), 3.80-3.63 (m, 3H), 3.60-3.44 (m, 2H), 3.42-3.37 (m, 1H), 3.35-3.32 (m, 1H), 3.11-3.01 (m, 1H), 2.37-2.32 (m, 1H), 2.09-1.88 (m, 2H), 1.81-1.69 (m, 1H), 1.62-1.45 (m, 7H). LC-MS (M+H)⁺=482.3. Chiral HPLC: RT=2.674 min.

### Example 41 and Example 42: (3S,4R)-4-((5-fluoro-4-(3-((S)-1-hydroxyethyl)-4-isopropylquinolin-6-yl)pyrimidin-2-yl)amino)tetrahydro-2H-pyran-3-ol and (3S,4R)-4-((5-fluoro-4-(3-((R)-1-hydroxyethyl)-4-isopropylquinolin-6-yl)pyrimidin-2-yl)amino)tetrahydro-2H-pyran-3-ol

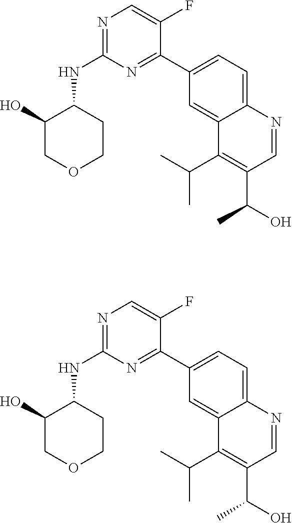 [SMILES: CC(C)c1c([C@H](C)O)cnc2ccc(-c3nc(N[C@@H]4CCOC[C@H]4O)ncc3F)cc12 ; CC(C)c1c([C@@H](C)O)cnc2ccc(-c3nc(N[C@@H]4CCOC[C@H]4O)ncc3F)cc12]

Step 1: 645-fluoro-2-((3S,4R)-3-hydroxytetrahydro-2H-pyran-4-yl)amino)pyrimidin-4-yl)-4-isopropylquinoline-3-carbaldehyde

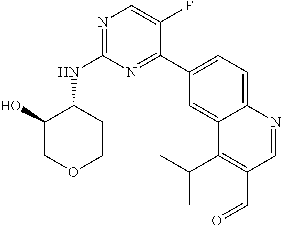 [SMILES: CC(C)c1c(C=O)cnc2ccc(-c3nc(N[C@@H]4CCOC[C@H]4O)ncc3F)cc12]

A mixture of (3S,4R)-4-({5-fluoro-4-[3-(hydroxymethyl)-4-isopropylquinolin-6-yl]pyrimidin-2-yl}amino)oxan-3-ol (790 mg, 1.92 mmol) and manganese dioxide (8.33 g, 95.85 mmol) in dichloromethane (20 mL) was stirred for 4 h at 40° C. before cooled to room temperature. The resulting mixture was filtered and the filter cake was washed with dichloromethane (6 mL×4). The filtrate was concentrated under reduced pressure and the residue was purified by flash chromatography eluting with methanol in dichloromethane (0% to 10% gradient, v/v) to yield the title compound (748 mg, 95%). LC-MS (M+H)⁺=411.2.

### Step 2: (3S,4R)-4-((5-fluoro-4-(3-((S)-1-hydroxyethyl)-4-isopropylquinolin-6-yl)pyrimidin-2-yl amino)tetrahydro-2H-pyran-3-ol and (3S,4R)-4-((5-fluoro-4-(3-(R-1-hydroxyethyl)-4-isopropylquinolin-6-yl)pyrimidin-2-yl)amino)tetrahydro-2H-pyran-3-ol

To a solution of methylmagnesium bromide (1.50 mL, 3 M solution in diethyl ether, 4.50 mmol) in tetrahydrofuran (15 mL) was added 6-(5-fluoro-2-(((3S,4R)-3-hydroxytetrahydro-2H-pyran-4-yl)amino)pyrimidin-4-yl)-4-isopropylquinoline-3-carbaldehyde (360 mg, 0.877 mmol) in tetrahydrofuran (2 mL) dropwise at −10° C. under nitrogen atmosphere. The resulting mixture was stirred for 3 h at room temperature under nitrogen atmosphere before quenched by the addition of aqueous saturated ammonium chloride (20 mL) at room temperature. The aqueous layer was extracted with ethyl acetate (20 mL×2). The organic phases were combined, washed with brine, dried over sodium sulfate, filtered and concentrated under reduced pressure. The residue was purified by flash chromatography eluting with methanol in dichloromethane (0% to 20% gradient, v/v). The residue was purified by prep-HPLC under the following conditions: column, XBridge Shield RP18 OBD Column, 30×150 mm, 5 um; mobile phase, acetonitrile in water (with 10 mmol/L NH₄HCO₃ and 0.1% NH₃·H₂O). 20% to 50% gradient in 9 min; detector. UV 254 nm. The obtained mixture of Example 59 and Example 60 was separated by chiral separation on Chiral-HPLC. Column: CHIRALPAK OD-H 4.6*150 mm 5 μm. Mobile phase: Hexane:EtOH (containing 0.1% diethylamine)=9:1, 1 mL/min in 18 min.

Example 41 (2.8 mg, 31%): RT=6.355 min. 1H-NMR (400 MHz, CD₃OD) δ 9.22 (s, 1H), 9.08 (s, 1H), 8.42-8.37 (m, 1H), 8.37-8.33 (m, 1H), 8.13-8.08 (m, 1H), 5.49 (s, 1H), 4.10-3.90 (m, 4H), 3.70-3.60 (m, 1H), 3.57-3.47 (m, 1H), 3.28-3.20 (m, 1H), 2.25-2.16 (m, 1H), 1.71-1.52 (in, 11H), 1.37-1.26 (m, 2H). LC-MS (M+H)⁺=427.1.

Example 42 (3.2 mg, 36%): RT=9.244 min. 1H-NMR (400 MHz, CD₃OD) δ: 9.22 (s, 1H), 9.08 (s, 1H), 8.42-8.37 (m, 1H), 8.37-8.33 (m, 1H), 8.13-8.08 (m, 1H), 5.48 (s, 1H), 4.08-3.90 (m, 4H), 3.70-3.60 (m, 1H), 3.57-3.47 (m, 1H), 3.28-3.20 (m, 1H), 2.25-2.16 (m, 1H), 1.72-1.26 (m, 13H). LC-MS (M+H)⁺=427.2.

### Example 43: (3S,4R)-4-((5-fluoro-4-(4-isopropyl-3-(((1-methyl-1H-pyrazol-4-yl)amino)methyl)quinolin-6-yl)pyrimidin-2-yl)amino)tetrahydro-2H-pyran-3-ol

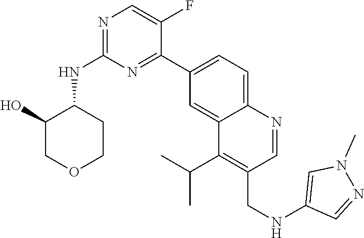 [SMILES: CC(C)c1c(CNc2cnn(C)c2)cnc2ccc(-c3nc(N[C@@H]4CCOC[C@H]4O)ncc3F)cc12]

The title compound (7.9 mg, 26.5%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and 1-methyl-1H-pyrazol-4-amine. ¹H-NMR (400 MHz, DMSO-d6) δ 9.11 (s, 1H), 8.88 (s, 1H), 8.50 (s, 1H), 8.31-8.25 (m, 1H), 8.16-8.11 (m, 1H), 7.26 (d, J=7.9 Hz, 1H), 7.13 (s, 1H), 7.00 (s, 1H), 4.99-4.90 (m, 2H), 4.27 (s, 2H), 3.94-3.80 (m, 4H), 3.69 (s, 3H), 3.60-3.50 (m, 1H), 3.40-3.34 (m, 1H), 3.05 (t, J=10.3 Hz, 1H), 2.10-2.00 (m, 1H), 1.62-1.50 (m, 7H). LC-MS (M+H)⁺=492.1.

### Example 44: (3S,4R)-4-((4-(3-((1,4-oxazepan-4-yl)methyl)-4-isopropylquinolin-6-yl)-5-fluoropyrimidin-2-yl)amino)tetrahydro-2H-pyran-3-ol

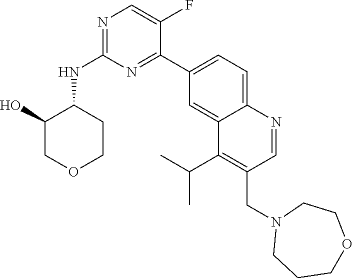 [SMILES: CC(C)c1c(CN2CCCOCC2)cnc2ccc(-c3nc(N[C@@H]4CCOC[C@H]4O)ncc3F)cc12]

The title compound (6.2 mg, 27%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and 1,4-oxazepane hydrochloride. ¹H-NMR (40) MHz, DMSO-d6) δ 9.12 (s, 1H), 8.79 (s, 1H), 8.50 (s, 1H), 8.28 (d, J=8.8 Hz, 1H), 8.13 (d, J=8.5 Hz, 1H), 7.27 (d, J=7.5 Hz, 1H), 5.10-4.87 (m, 1H), 4.13-3.97 (m, 1H), 3.94-3.79 (m, 5H), 3.74-3.65 (m, 2H), 3.65-3.49 (m, 3H), 3.43-3.35 (m. J=12.2 Hz, 1H), 3.06 (t, J=10.3 Hz, 1H), 2.74-2.59 (m, 4H), 2.13-1.98 (m, 1H), 1.89-1.76 (m, 2H), 1.66-1.57 (m, J=6.3 Hz, 6H), 1.57-1.44 (m, J=11.9 Hz, 1H). LC-MS (M+H)⁺=496.1.

### Example 45: (3S,4R)-4-((5-fluoro-4-(4-isopropyl-3-((pyrimidin-2-ylamino)methyl)quinolin-6-yl)pyrimidin-2-yl)amino)tetrahydro-2H-pyran-3-ol

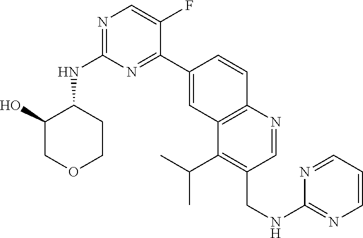 [SMILES: CC(C)c1c(CNc2ncccn2)cnc2ccc(-c3nc(N[C@@H]4CCOC[C@H]4O)ncc3F)cc12]

The title compound (1.08 mg, 3.67%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and pyrimidin-2-amine. ¹H-NMR (400 MHz, DMSO-d6) δ 9.10 (s, 1H), 8.91 (s, 1H), 8.49 (s, 1H), 8.37-8.17 (m, 3H), 8.12 (d, J=8.8 Hz, 1H), 7.81 (s, 1H), 7.26 (d, J=7.2 Hz, 1H), 6.60 (s, 1H), 4.97 (s, 1H), 4.73 (s, 2H), 3.90 (d, J=39.7 Hz, 4H), 3.54 (s, 1H), 3.38 (s, 1H), 3.05 (t, J=10.6 Hz, 1H), 2.03 (s, 1H), 1.53 (d, J=24.2 Hz, 7H). LC-MS (M+H)⁺=490.0.

### Example 46: (3S,4R)-4-((5-fluoro-4-(4-isopropyl-3-(((4-methoxyphenyl)amino)methyl)quinolin-6-yl)pyrimidin-2-yl)amino)tetrahydro-2H-pyran-3-ol

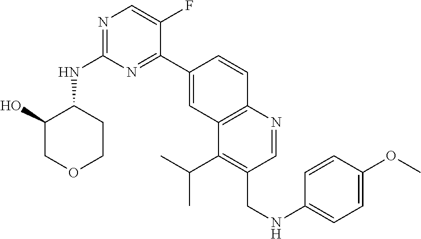 [SMILES: COc1ccc(NCc2cnc3ccc(-c4nc(N[C@@H]5CCOC[C@H]5O)ncc4F)cc3c2C(C)C)cc1]

The title compound (18 mg, 50%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and 4-methoxyaniline. ¹H-NMR (400 MHz, DMSO-d6) δ 9.12 (s, 1H), 8.89 (s, 1H), 8.50 (s, 1H), 8.28 (d, J=8.2 Hz, 1H), 8.13 (d, J=8.8 Hz, 1H), 7.27 (d, J=7.6 Hz, 1H), 6.73 (d, J=8.0 Hz, 2H), 6.62 (d, J=7.9 Hz, 2H), 5.72 (s, 1H), 4.98 (s, 1H), 4.43 (s, 2H), 3.96-3.76 (m, 4H), 3.63 (s, 3H), 3.54 (s, 1H), 3.47-3.27 (m, 1H), 3.05 (t, J=10.3 Hz, 1H), 2.03 (s, 1H), 1.68-1.43 (m, 4H). LC-MS (M+H)⁺=518.0.

### Example 47 and Example 48: (3S,4R)-4-((5-fluoro-4-(3-((S)-1-((R)-3-fluoropyrrolidin-1-yl)ethyl)-4-isopropylquinolin-6-yl)pyrimidin-2-yl)amino)tetrahydro-2H-pyran-3-ol and (3S,4R)-4-((5-fluoro-4-(3-((R)-1-((R)-3-fluoropyrrolidin-1-yl)ethyl)-4-isopropylquinolin-6-yl)pyrimidin-2-yl)amino)tetrahydro-2H-pyran-3-ol

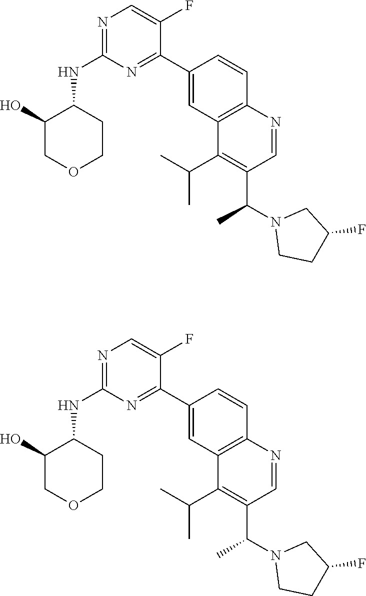 [SMILES: CC(C)c1c([C@H](C)N2CC[C@@H](F)C2)cnc2ccc(-c3nc(N[C@@H]4CCOC[C@H]4O)ncc3F)cc12 ; CC(C)c1c([C@@H](C)N2CC[C@@H](F)C2)cnc2ccc(-c3nc(N[C@@H]4CCOC[C@H]4O)ncc3F)cc12]

### Step 1: 6-chloro-4-isopropylquinoline-3-carboxylic acid

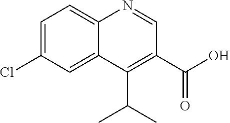 [SMILES: CC(C)c1c(C(=O)O)cnc2ccc(Cl)cc12]

The title compound (39) mg, 82%) was prepared in a manner similar to Example 1 step 5 from ethyl 6-chloro-4-isopropylquinoline-3-carboxylate. ¹H-NMR (400 MHz, DMSO-d6) δ 13.75 (s, 1H), 8.89 (s, 1H), 8.39 (s, 1H), 8.09 (d, J=8.8 Hz, 1H), 7.86 (d, J=8.8 Hz, 1H), 4.06-3.84 (m, 1H), 1.48 (d, J=6.9 Hz, 6H). LC-MS (M+H)⁺=250.1.

### Step 2: 6-chloro-4-isopropyl-N-methoxy-N-methylquinoline-3-carboxamide

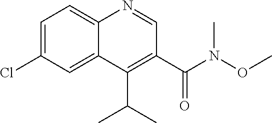 [SMILES: CON(C)C(=O)c1cnc2ccc(Cl)cc2c1C(C)C]

A mixture of 6-chloro-4-isopropylquinoline-3-carboxylic acid (390 mg, 1.56 mmol), N,O-dimethylhydroxylamine hydrochloride (183 mg, 1.87 mmol), (benzotriazol-1-yloxy)tripyrrolidinophosphonium hexafluorophosphate (811 mg, 1.56 mmol) and N,N-diisopropylethylamine (402 mg, 3.12 mmol) in dimethylformamide (20 mL) was stirred at room temperature for overnight before addition of water. The mixture was extracted with ethyl acetate (40 mL×3). The combined organic layers were washed with water and brine, dried over sodium sulfate, filtered and concentrated. The residue was purified over silica gel by combi-flash, eluting with ethyl acetate in petroleum ether (50%, v/v) to give the title compound (340 mg, 74%). ¹H-NMR (400 MHz, DMSO-d6) δ 8.76 (s, 1H), 8.34 (s, 1H), 8.09 (d, J=8.9 Hz, 1H), 7.83 (d, J=8.5 Hz, 1H), 3.66 (s, 1H), 3.49 (s, 3H), 3.35 (s, 3H), 1.41 (d, J=6.6 Hz, 6H). LC-MS (M+H)⁺=293.1.

### Step 3: 1-(6-choro-4-isopropylquinolin-3-yl)ethan-1-one

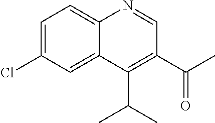 [SMILES: CC(=O)c1cnc2ccc(Cl)cc2c1C(C)C]

To the solution of 6-chloro-4-isopropyl-N-methoxy-N-methlyquinoline-3-carboxamide (340 mg, 1.16 mmol) of tetrahydrofuran (10 m L) under nitrogen at 0° C. was added 1.6 M methyllithium in diethyl ether (0.9 mL, 1.4 mmol). The reaction solution was stirred for 2 h before quenched with saturated aqueous solution of ammonium chloride. The mixture was extracted with ethyl acetate (20 mL×3). The organic layers were combined, washed with water and brine, dried over sodium sulfate, filtered and evaporated. The residue was purified over silica gel by combi-flash, eluting with ethyl acetate in petroleum ether (50%, v/v) to give the title compound (150 mg, 51%). LC-MS (M+H)⁺=248.1.

### Step 4: 1-(6-chloro-4-isopropylquinolin-3-yl)ethan-1-ol

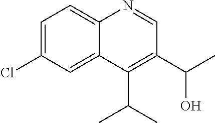 [SMILES: CC(C)c1c(C(C)O)cnc2ccc(Cl)cc12]

To a solution of 1-(6-chloro-4-isopropylquinolin-3-yl)ethan-1-one (150 mg, 0.6 mmol) in ethanol (5 mL) was added sodium borohydride (46 mg, 1.2 mmol) at 0° C. and the resulting mixture was stirred for 1 h before quenched with 1 N HCl. The formed precipitate (130 mg, crude) was filtered and used for next step without further purification. LC-MS (M+H)⁺=250.1.

### Step 5: 6-chloro-3-(1-chloroethyl-4-isopropylquinoline

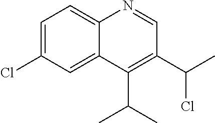 [SMILES: CC(C)c1c(C(C)Cl)cnc2ccc(Cl)cc12]

To a solution of 1-(6-chloro-4-isopropylquinolin-3-yl)ethan-1-ol (130 mg, crude) in dichloromethane (10 mL) was added thionyl chloride (0.2 mL) at 0° C. and the reaction solution was stirred for 1 h before diluted with dichloromethane. The solution was washed with saturated aqueous solution of sodium bicarbonate. The mixture was washed with water and brine, dried over sodium sulfate, filtered and evaporated to give the crude product (130 mg, crude). LC-MS (M+H)⁺=268.0.

### Step 6: 6-chloro-3-(1-((R)-3-fluoropyrrolidin-1-yl)ethyl)-4-isopropylquinoline

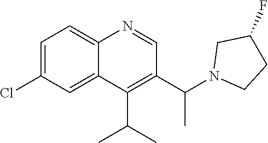 [SMILES: CC(C)c1c(C(C)N2CC[C@@H](F)C2)cnc2ccc(Cl)cc12]

A reaction mixture of 6-chloro-3-(1-chloroethyl)-4-isopropylquinoline (130 mg, 0.49 mmol), (R)-3-fluoropyrrolidine hydrochloride (123 mg, 0.97 mmol) and N,N-diisopropylethylamine (125 mg, 0.97 mmol) in dimethylformamide (5 mL) was stirred at 100° C. for overnight before cooled to room temperature. The solvent was evaporated and the residue was purified over silica gel by combi-flash, eluting with ethyl acetate in petroleum ether (67%, v/v) to give the title compound (90 mg, 57%). LC-MS (M+H)⁺=321.2.

### Step 7: 3-(1((R)-3-fluoropyrrolidin-1-yl)ethyl)-4-isopropyl-6-(4,4,5,5-tetramethyl-1,3,2-dioxaborolan-2-yl)quinoline

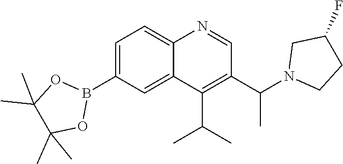 [SMILES: CC(C)c1c(C(C)N2CC[C@@H](F)C2)cnc2ccc(B3OC(C)(C)C(C)(C)O3)cc12]

The title compound was prepared in a manner similar to Example 1 step 2 from 6-chloro-3-(1-((R)-3-fluoropyrrolidin-1-yl)ethyl)-4-isopropylquinoline. LC-MS (M+H)⁺=413.4.

### Step 8: 642-chloro-5-fluoropyrimidin-4-yl)-3-(1-((R)-3-fluoropyrrolidin-1-yl)ethyl)-4-isopropylquinoline

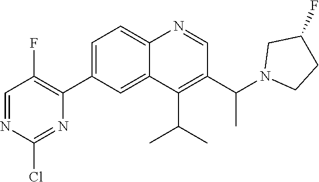 [SMILES: CC(C)c1c(C(C)N2CC[C@@H](F)C2)cnc2ccc(-c3nc(Cl)ncc3F)cc12]

The title compound (80 mg, 68% for two steps) was prepared in a manner similar to Example 1 step 3 from 3-(1-((R)-3-fluoropyrrolidin-1-yl)ethyl)-4-isopropyl-6-(4,4,5,5-tetramethyl-1,3,2-dioxaborolan-2-yl)quinoline. LC-MS (M+H)⁺=417.3

### Step 9: (3S,4R)-4-((5-fluoro-4-(3-((S)-1-((R)-3-fluoropyrrolidin-1-yl)ethyl)-4-isopropylquinolin-6-yl)pyrimidin-2-yl)amino)tetrahydro-2H-pyran-3-ol and (3S,4R)-4-((5-fluoro-44(3-((R)-4(R)-3-fluoropyrrolidin-1-yl)ethyl)-4-isopropylquinolin-6-yl)pyrimidin-2-yl)amino)tetrahydro-2H-pyran-3-ol

Mixture of Example 47 and Example 48 was prepared in a manner similar to Example 1 step 4 from 6-(2-chloro-5-fluoropyrimidin-4-yl)-3-(1-((R)-3-fluoropyrrolidin-1-yl)ethyl)-4-isopropylquinoline. Example 47 and Example 48 were separated on chiral-HPLC to give (3S,4R)-4-((5-fluoro-4-(3-((S)-1-((R)-3-fluoropyrrolidin-1-yl)ethyl)-4-isopropylquinolin-6-yl)pyrimidin-2-yl)amino)tetrahydro-2H-pyran-3-ol & (3S,4R)-4-((5-fluoro-4-(3-((R)-1-((R)-3-fluoropyrrolidin-1-yl)ethyl)-4-isopropylquinolin-6-yl)pyrimidin-2-yl)amino)tetrahydro-2H-pyran-3-ol. Analytical chiral HPLC condition: CHIRALPAK ID-3, 0.46×5 cm, 3.0 um. Mobile phase: (hexane:dichloromethane=1:1)((0.5% 2M NH₃-MeOH)): EtOH, 20 mL/min in 11.5 min.

Example 47 (10 mg, 27%) ¹H-NMR (400 MHz, DMSO-d6) δ 9.13-8.87 (m, 2H), 8.52-8.48 (m, 1H), 8.30-8.25 (m, 1H), 8.16-8.09 (m, 1H), 7.30-7.24 (m, 1H), 5.32-5.09 (m, 1H), 5.00-4.96 (m, 1H), 4.16-4.12 (m, 1H), 3.88-3.84 (m, 4H), 3.76-3.45 (m, 2H), 3.07-3.03 (m, 1H), 2.97-2.92 (m, 1H), 2.62-2.57 (m, 2H), 2.46-2.37 (m, 2H), 2.16-2.04 (m, 2H), 1.89-1.84 (m, 1H), 1.60-1.55 (m, 6H), 1.45-1.41 (m, 3H). LC-MS (M+H)⁺=498.2. Chiral HPLC: tR=2.127 min.

Example 47 (11 mg, 29%) ¹H-NMR (400 MHz, DMSO-d6) δ 9.17-9.12 (m, 2H), 8.67-8.10 (m, 3H), 7.45-7.18 (m, 1H), 5.42-4.90 (m, 2H), 4.40-3.49 (m, 7H), 3.07-3.03 (m, 2H), 2.76-2.47 (m, 3H), 2.38-2.14 (m, 2H), 2.08-2.03 (m, 2H), 1.73-1.52 (m, 6H), 1.48-1.43 (m, 3H). LC-MS (M+H)⁺=498.2. Chiral HPLC: tR=2.760 min.

¹H-NMR (400 MHz, DMSO-d6) δ 9.11 (s, 1H), 9.01 (s, 1H), 8.50 (s, 1H), 8.27 (d, J=8.2 Hz, 1H), 8.12 (d, J=9.3 Hz, 1H), 7.27 (d, J=6.9 Hz, 1H), 5.21 (dd, J=55.7, 16.8 Hz, 1H), 4.98 (s, 1H), 4.16 (s, 1H), 3.86 (s, 4H), 3.54 (s, 1H), 3.38-3.33 (m, 1H), 3.03-2.92 (m, 2H), 2.62 (d, J=27.6 Hz, 2H), 2.44-1.78 (m, 4H), 1.58-1.51 (m, 7H), 1.44 (s, 3H). LC-MS (M+H)⁺=498.1.

### Example 49: (3S,4R)-4-((4-(3-(((1R,4R)-2-oxa-5-azabicyclo[2.2.1]heptan-5-yl)methyl)-4-isopropylquinolin-6-yl)-5-fluoropyrimidin-2-yl)amino)tetrahydro-2H-pyran-3-ol

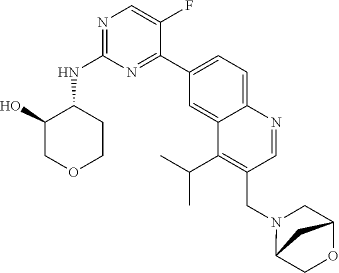 [SMILES: CC(C)c1c(CN2C[C@H]3C[C@@H]2CO3)cnc2ccc(-c3nc(N[C@@H]4CCOC[C@H]4O)ncc3F)cc12]

The title compound (11 mg, 41%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (1R,4R)-2-oxa-5-azabicyclo[2.2.1]heptane hydrochloride. ¹H-NMR (300 MHz, DMSO-d6) δ 9.10 (brs, 1H), 8.84 (s, 1H), 8.48 (d, J=3.9 Hz, 1H), 8.26 (d, J=9.1 Hz, 1H), 8.11 (d, J=8.8 Hz, 1H), 7.24 (d, J=7.8 Hz, 11H), 4.95 (d, J=5.2 Hz, 11H), 4.36 (s, 1H), 4.12-3.77 (m, 7H), 3.61-3.46 (m, 2H), 3.44-3.31 (m, 3H), 3.11-2.98 (m, 1H), 2.77-2.68 (m, 1H), 2.09-1.99 (m, 1H), 1.86-1.77 (m, 1H), 1.63-1.41 (m, 8H). LC-MS (M+H)⁺=494.3.

### Example 50 (3S,4R)-4-((5-fluoro-4-(3-(1-hydroxycyclopentyl)-4-isopropylquinolin-6-yl)pyrimidin-2-yl)amino)tetrahydro-2H-pyran-3-ol

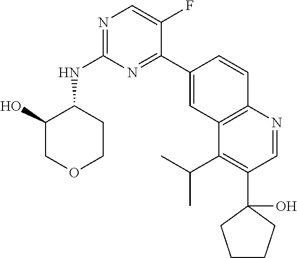 [SMILES: CC(C)c1c(C2(O)CCCC2)cnc2ccc(-c3nc(N[C@@H]4CCOC[C@H]4O)ncc3F)cc12]

### Step 1: 146-chloro-4-isopropylquinolin-3-yl)cyclopentan-1-ol

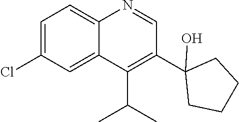 [SMILES: CC(C)c1c(C2(O)CCCC2)cnc2ccc(Cl)cc12]

The reaction mixture of 1,4-dibromobutane (500 mg, 2.31 mmol), magnesium (556 mg, 23.1 mmol) and iodine (10 mg) in tetrahydrofuran (3 mL) was heated to 50° C. for 5 min and then stirred at room temperature for 1 h before addition of ethyl 6-chloro-4-isopropylquinoline-3-carboxylate (200 mg, 0.72 mmol). The resulting mixture was stirred at 50° C. for 6 h before cooled to room temperature. The reaction was quenched with an aqueous saturated solution of ammonium chloride and the aqueous layer was extracted with ethyl acetate. The combined organic layers were washed with brine, dried over sodium sulfate, filtered and evaporated. The residue was purified by column chromatography using methanol in dichloromethane (3%, v/v) to give the title compound (70 mg, 33%). ¹H-NMR (400 MHz, DMSO-d6) δ 8.97 (s, 1H), 8.31 (s, 1H), 8.02 (d, J=8.9 Hz, 1H), 7.71 (d, J=8.8 Hz, 1H), 5.20 (s, 1H), 4.55-4.41 (m, 1H), 2.18 (s, 2H), 2.09 (s, 2H), 1.89 (s, 2H), 1.72 (s, 2H), 1.52 (d, J=7.1 Hz, 6H). LC-MS (M+H)⁺=290.2.

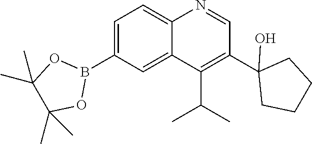 [SMILES: CC(C)c1c(C2(O)CCCC2)cnc2ccc(B3OC(C)(C)C(C)(C)O3)cc12]

The title compound (1.8 g, 91%) was prepared in a manner similar to Example 1 step 2 from 1-(6-chloro-4-isopropylquinolin-3-yl)cyclopentan-1-ol and bis(pinacolato)diboron. LC-MS (M+H)⁺=300.3 for corresponding boric acid.

This compound can also be obtained by the following step:

A mixture of 1-(6-chloro-4-isopropylquinolin-3-yl)cyclopentan-1-ol (1.5 g, 5.19 mmol), bis(pinacolato)diboron (1.84 g, 7.3 mmol). (1,1′-bis(diphenylphosphino)ferrocene)palladium(II) dichloride (223 mg, 0.31 mmol) and potassium acetate (1.32 g, 13.5 mmol) in 1,4-dioxane (50 mL) was stirred at 80° C. under nitrogen for overnight before cooled to room temperature. The mixture was filtered and the filtrate was evaporated to give the crude product (1.8 g, 91%) which was used for next step without further purification. LC-MS (M+H)⁺=300.3 for corresponding boric acid.

### Step 3: 1-(6-(2-chloro-5-fluoropyrimidin-4-yl)-4-isopropylquinolin-3-yl)cyclopentan-ol

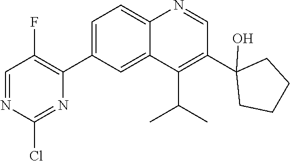 [SMILES: CC(C)c1c(C2(O)CCCC2)cnc2ccc(-c3nc(Cl)ncc3F)cc12]

The title compound (80 mg, 86%) was prepared in a manner similar to Example 1 step 3 from 1-(6-chloro-4-isopropylquinolin-3-yl)cyclopentan-1-ol and 2,4-dichloro-5-fluoropyrimidine. LC-MS (M+H)⁺=386.2.

This compound can also be obtained by the following step:

A mixture of 1-(6-chloro-4-isopropylquinolin-3-yl)cyclopentan-1-ol (92 mg, 0.24 mmol), 2,4-dichloro-5-fluoropyrimidine (40 mg, 0.24 mmol), (1,1′-bis(diphenylphosphino)ferrocene)palladium(II) dichloride (8.3 mg, 0.012 mmol) and potassium carbonate (65 mg, 0.48 mmol) in 1,4-dioxane (2 mL) and water (0.25 mL) under nitrogen was stirred at 80° C. for overnight before cooled to room temperature. The mixture was diluted with ethyl acetate, washed with brine, dried over sodium sulfate, filtered and evaporated. The residue was purified over silica gel by combi-flash, eluting with methanol in dichloromethane (3%, v/v) to give the title compound (80 mg, 86%). LC-MS (M+H)⁺=386.2.

### Step 4: (3S,4R)-44(5-fluoro-4-(3-(1-hydroxycyclopentyl)-4-isopropylquinolin-6-yl)pyrimidin-2-yl)amino)tetrahydro-2H-pyran-3-ol

The title compound (5 mg, 61%) was prepared in a manner similar to Example 1 step 4 from 1-(6-(2-chloro-5-fluoropyrimidin-4-yl)-4-isopropylquinolin-3-yl)cyclopentan-1-ol and (3S,4R)-4-aminotetrahydro-2H-pyran-3-ol hydrochloride. ¹H-NMR (400 MHz, DMSO-d6) δ 9.20 (s, 1H), 9.03 (s, 1H), 8.50 (s, 1H), 8.27 (d, J=8.7 Hz, 1H), 8.11 (d, J=9.1 Hz, 1H), 7.25 (d, J=7.8 Hz, 1H), 5.21 (s, 1H), 4.98 (s, 1H), 4.63-4.44 (m, 1H), 3.90-3.83 (m, 3H), 3.54 (s, 1H), 3.38-3.36 (m, 1H), 3.05 (t. J=10.1 Hz, 1H), 2.30-1.99 (m, 5H), 1.91 (s, 2H), 1.74 (s, 2H), 1.65-1.43 (m, 7H). LC-MS (M+H)⁺⁼467.1.

This compound can also be obtained by the following step:

The reaction mixture of 1-(6-(2-chloro-5-fluoropyrimidin-4-yl)-4-isopropylquinolin-3-yl)cyclopentan-1-ol (6.6 mg, 0.018 mmol), (3S,4R)-4-aminotetrahydro-2H-pyran-3-ol hydrochloride (5.5 mg, 0.035 mmol). (SP-4-1)-[1,3-bis[2,6-bis(1-ethylpropyl)phenyl]-4,5-dichloro-1,3-dihydro-2H-imidazol-2-ylidene]dichloro(2-methylpyridine)palladium (1.6 mg, 0.002 mmol) and cesium carbonate (18 mg, 0.054 mmol) in 1,4-dioxane (0.4 mL) under nitrogen was stirred at 100° C. for overnight. The mixture was cooled to room temperature and the solvent was evaporated. The residue was purified over silica gel by combi-flash, eluting with methanol in dichloromethane (6%, v/v) to give the title compound (5 mg, 61%). ¹H-NMR (400 MHz, DMSO-d6) δ 9.20 (s, 1H), 9.03 (s, 1H), 8.50 (s, 1H), 8.27 (d, J=8.7 Hz, 1H), 8.11 (d, J=9.1 Hz, 1H), 7.25 (d, J=7.8 Hz, 1H), 5.21 (s, 1H), 4.98 (s, 11H), 4.63-4.44 (m, 1H), 3.90-3.83 (m, 3H), 3.54 (s, 1H), 3.38-3.36 (m, 1H), 3.05 (t, J=10.1 Hz, 1H), 2.30-1.99 (m, 5H), 1.91 (s, 2H), 1.74 (s, 2H), 1.65-1.43 (m, 7H). LC-MS (M+H)⁺=467.1.

### Example 51: (3S,4R)-4-((5-fluoro-4-(8-fluoro-3-(hydroxymethyl)-4-isopropylquinolin-6-yl)pyrimidin-2-yl)amino)tetrahydro-2H-pyran-3-ol

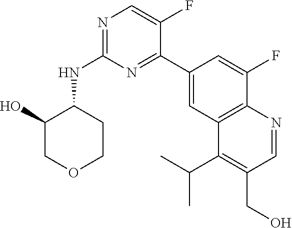 [SMILES: CC(C)c1c(CO)cnc2c(F)cc(-c3nc(N[C@@H]4CCOC[C@H]4O)ncc3F)cc12]

### Step 1: diethyl 2-(((4-chloro-2-fluorophenyl)amino)methylene)malonate

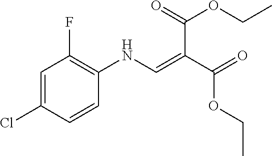 [SMILES: CCOC(=O)C(=CNc1ccc(Cl)cc1F)C(=O)OCC]

A solution of 4-chloro-2-fluoroaniline (25 g, 171.2 mmol) and diethyl 2-(ethoxymethylene)malonate (55 g) was stirred at 110° C. for 4 h. After cooled to room temperature, the reaction mixture was treated with petroleum ether (600 mL). The precipitate was filtered and dried to give the title compound (43.9 g, 81%). ¹H-NMR (400 MHz, DMSO-d6) δ 10.87 (d, J=13.3 Hz, 1H), 8.44 (d, J=13.4 Hz, 1H), 7.72-7.67 (m, 1H), 7.60 (d, J=10.9 Hz, 1H), 7.33 (d, J=8.7 Hz, 1H), 4.22 (q, J=6.9 Hz, 2H), 4.14 (q, J=6.9 Hz, 2H), 1.33-1.19 (m, 6H). LC-MS (M+H)⁺=316.2.

### Step 2: ethyl 6-chloro-8-fluoro-4-hydroxyquinoline-3-carboxylate

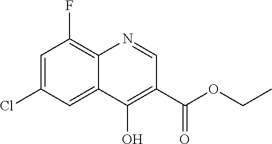 [SMILES: CCOC(=O)c1cnc2c(F)cc(Cl)cc2c1O]

The reaction mixture of diethyl 2-(((4-chloro-2-fluorophenyl)amino)methylene)malonate (34.6 g, 109.5 mmol) and oxydibenzene (50 mL) was stirred at 260° C. for 9 h. After cooled to room temperature, the mixture was treated with petroleum ether (600 mL). The precipitate was filtered and dried to give the title product (43.9 g, 81%). ¹H-NMR (400 MHz, DMSO-d6) δ 12.65 (s, 1H), 8.39 (s, 1H), 7.91 (d, J=9.8 Hz, 2H), 4.23 (q, J=6.8 Hz, 2H), 1.28 (t, J=7.0 Hz, 3H). LC-MS (M+H)⁺=270.1.

### Step 3: ethyl 4-bromo-6-chloro-8-fluoroquinoline-3-carboxylate

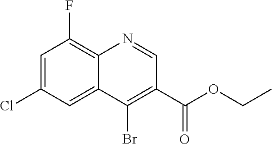 [SMILES: CCOC(=O)c1cnc2c(F)cc(Cl)cc2c1Br]

To the solution of ethyl 6-chloro-8-fluoro-4-hydroxyquinoline-3-carboxylate (5.5 g, 20.4 mmol) in dimethylformamide (80 mL) was added phosphorus tribromide (11 g, 40.8 mmol) and the mixture was stirred at 80° C. for 4 h. The mixture was cooled to room temperature and poured to ice water. The precipitate was filtered and dissolved with ethyl acetate. The mixture was washed with water and brine, dried over sodium sulfate, filtered and concentrated. The residue was purified by column chromatography eluting with ethyl acetate in petroleum ether (16%, v/v) to give the title compound (5.2 g, 76%). ¹H-NMR (400 MHz, DMSO-d₆) δ 9.09 (s, 1H), 8.15 (s, 1H), 8.10 (d, J=10.0 Hz, 1H), 4.46 (q, J=7.0 Hz, 2H), 1.39 (t, J=7.0 Hz, 3H). LC-MS (M+H)⁺=332.0, 334.0.

### Step 4: ethyl 6-chloro-8-fluoro-4-isopropylquinoline-3-carboxylate

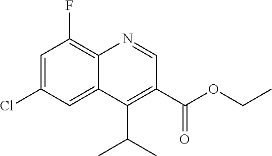 [SMILES: CCOC(=O)c1cnc2c(F)cc(Cl)cc2c1C(C)C]

The title compound (950 mg, 86%) was prepared in a manner similar to Example 1 step 1 from ethyl 4-bromo-6-chloro-8-fluoroquinoline-3-carboxylate. ¹H-NMR (400 MHz, DMSO-d6) δ 8.92 (s, 1H), 8.25 (s, 1H), 7.93 (d, J=10.0 Hz, 1H), 4.42 (q, J=7.0 Hz, 2H), 4.04-3.82 (m, 1H), 1.44 (d, J=6.9 Hz, 6H), 1.37 (t, J=7.0 Hz, 3H). LC-MS (M+H)⁺=296.1.

### Step 5: ethyl 8-fluoro-4-isopropyl-6-(4,4,5,5-tetramethyl-1,3,2-dioxaborolan-2-yl)quinoline-3-carboxylate

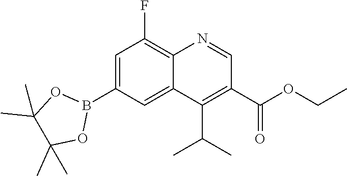 [SMILES: CCOC(=O)c1cnc2c(F)cc(B3OC(C)(C)C(C)(C)O3)cc2c1C(C)C]

The title compound (0.7 g, 1(0)%) was prepared in a manner similar to Example 1 step 2 from ethyl 6-chloro-8-fluoro-4-isopropylquinoline-3-carboxylate and bis(pinacolato)diboron. LC-MS (M+H)⁺=306.1 for corresponding boric acid.

### Step 6: ethyl 6-(2-chloro-5-fluoropyrimidin-4-yl)-8-fluoro-4-isopropylquinoline-3-carboxylate

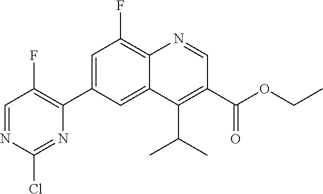 [SMILES: CCOC(=O)c1cnc2c(F)cc(-c3nc(Cl)ncc3F)cc2c1C(C)C]

The title compound (420 mg, 45%) was prepared in a manner similar to Example 1 step 3 from ethyl 8-fluoro-4-isopropyl-6-(4,4,5,5-tetramethyl-1,3,2-dioxaborolan-2-yl)quinoline-3-carboxylate and 2,4-dichloro-5-fluoropyrimidine. ¹H-NMR (400 MHz, DMSO-d6) δ9.08 (d, J=14.4 Hz, 2H), 8.90 (s, 1H), 8.18 (d, J=11.1 Hz, 1H), 4.44 (q, J=6.3 Hz, 2H), 4.04-3.86 (m, 1H), 1.54 (d, J=6.9 Hz, 6H), 1.38 (t, J=6.9 Hz, 3H). LC-MS (M+H)*=392.2.

### Step 7: ethyl 8-fluoro-6-(5-fluoro-2-((3S,4R)-3-hydroxytetrahydro-2H-pyran-4-yl)amino)pyrimidin-4-yl)-4-isopropylquinoline-3-carboxylate

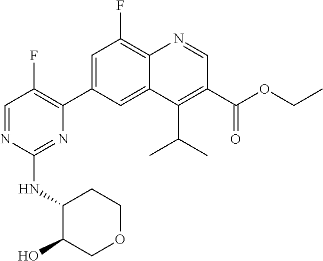 [SMILES: CCOC(=O)c1cnc2c(F)cc(-c3nc(N[C@@H]4CCOC[C@H]4O)ncc3F)cc2c1C(C)C]

The title compound (30) mg, 59%) was prepared in a manner similar to Example 1 step 4 from ethyl 6-(2-chloro-5-fluoropyrimidin-4-yl)-8-fluoro-4-isopropylquinoline-3-carboxylate and (3S,4R)-4-aminotetrahydro-2H-pyran-3-ol hydrochloride. LC-MS (M+H)⁺=473.3.

### Step 8: (3S,4R)-44(5-fluoro-4-(8-fluoro-3-hydroxymethyl)-4-isopropylquinolin-6-yl)pyrimidin-2-yl)amino)tetrahydro-2H-pyran-3-ol

The title compound (56 mg, 26%) was prepared in a manner similar to Example 3 from ethyl 8-fluoro-6(5-fluoro-2-(((3S,4R)-3-hydroxytetrahydro-2H-pyran-4-yl)amino)pyrimidin-4-yl)-4-isopropylquinoline-3-carboxylate. ¹H-NMR (400 MHz, DMSO-d6) δ 8.96 (s, 1H), 8.93 (s, 1H), 8.52 (d, J=2.7 Hz, 1H), 8.07 (d, J=11.6 Hz, 1H), 7.32 (d, J=7.7 Hz, 1H), 5.48 (s, 1H), 4.97 (s, 1H), 4.81 (s, 2H), 4.02-3.73 (m, 4H), 3.54 (s, 1H), 3.39-3.36 (m, 1H), 3.07 (t, J=10.4 Hz, 11H), 2.02 (s, 1H), 1.63-1.42 (m, 7H). LC-MS (M+H)⁺=431.1.

### Example 52: (3S,4R)-4-((4-(3-(((3,3-difluorocyclobutyl)amino)methyl)-8-fluoro-4-isopropylquinolin-6-yl)-5-fluoropyrimidin-2-yl)amino)tetrahydro-2H-pyran-3-ol

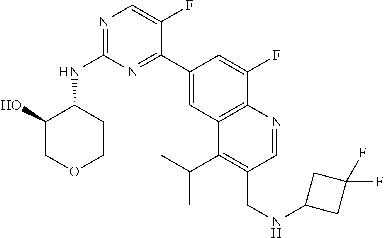 [SMILES: CC(C)c1c(CNC2CC(F)(F)C2)cnc2c(F)cc(-c3nc(N[C@@H]4CCOC[C@H]4O)ncc3F)cc12]

### Step 1: (3S,4R)-4-((4-(3-(chloromethyl)-8-fluoro-4-isopropylquinolin-6-yl)-5-fluoropyrimidin-2-yl)amino)tetrahydro-2H-pyran-3-ol

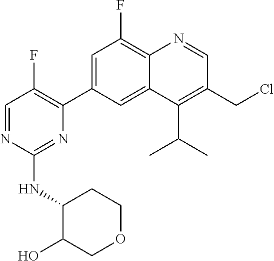 [SMILES: CC(C)c1c(CCl)cnc2c(F)cc(-c3nc(N[C@@H]4CCOCC4O)ncc3F)cc12]

To a solution of (3S,4R)-4-((5-fluoro-4-(8-fluoro-3-(hydroxymethyl)-4-isopropylquinolin-6-yl)pyrimidin-2-yl)amino)tetrahydro-2H-pyran-3-ol (40 mg, 0.093 mmol) in dichloromethane (10 mL) was added thionyl chloride (0.1 mL) at 0° C. and the reaction solution was stirred for 1 h before diluted with dichloromethane. The solution was washed with saturated aqueous solution of sodium bicarbonate, water and brine, dried over sodium sulfate, filtered and evaporated to give the crude title compound (40 mg). LC-MS (M+H)⁺=449.3.

### Step 2: (3S,4R)-4(4-(3-(((3,3-difluorocyclobutyl)amino)methyl)-8-fluoro-4-isopropylquinolin-6-yl)-5-fluoropyrimidin-2-yl)amino)tetrahydro-2H-pyran-3-ol

The title compound (17 mg, 72%) was prepared in a manner similar to that in Example 11 step 1 from (3S,4R)-4-(4-(3-(chloromethyl)-8-fluoro-4-isopropylquinolin-6-yl)-5-fluoropyrimidin-2-yl)amino)tetrahydro-2H-pyran-3-ol and 3,3-difluorocyclobutan-1-amine hydrochloride. ¹H-NMR (400 MHz, DMSO-d6) δ 8.95 (s, 1H), 8.90 (s, 11H), 8.52 (s, 1H), 8.07 (d, J=11.5 Hz, 1H), 7.32 (d, J=7.7 Hz, 1H), 4.97 (s, 1H), 4.06-3.76 (m, 6H), 3.54 (s, 1H), 3.39-3.36 (m, 11H), 3.19 (s, 1H), 3.06 (t, J=10.1 Hz, 1H), 2.77 (s, 2H), 2.41-2.37 (m, 2H), 2.02 (s, 1H), 1.57-1.51 (m, 7H). LC-MS (M+H)⁺=520.1.

### Example 53: (3S,4R)-4-((5-fluoro-4-(8-fluoro-4-isopropyl-3-((((R)-tetrahydro-2H-pyran-3-yl)amino)methyl)quinolin-6-yl)pyrimidin-2-yl)amino)tetrahydro-2H-pyran-3-ol

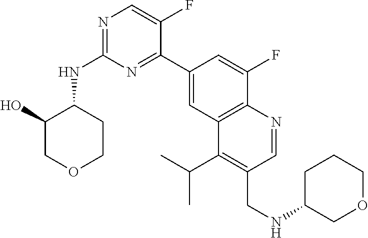 [SMILES: CC(C)c1c(CN[C@@H]2CCCOC2)cnc2c(F)cc(-c3nc(N[C@@H]4CCOC[C@H]4O)ncc3F)cc12]

The title compound (14 mg, 61%) was prepared in a manner similar to that in Example 11 step 1 from (3S,4R)-4-((4-(3-(chloromethyl)-8-fluoro-4-isopropylquinolin-6-yl)-5-fluoropyrimidin-2-yl)amino)tetrahydro-2H-pyran-3-ol and (R)-tetrahydro-2H-pyran-3-amine hydrochloride. ¹H-NMR (400 MHz, DMSO-d6) δ 8.95 (s, 1H), 8.91 (s, 1H), 8.52 (s, 1H), 8.06 (d, J=11.6 Hz, 1H), 7.32 (d, J=7.5 Hz, 1H), 4.97 (s, 1H), 4.10-3.77 (m, 7H), 3.69 (d, J=11.0 Hz, 1H), 3.54 (s, 1H), 3.39-3.24 (m, 3H), 3.06 (t, J=10.1 Hz, 2H), 2.56 (s, 1H), 2.01-1.97 (m, 2H), 1.75-1.38 (m, 9H), 1.38-1.17 (m, 1H). LC-MS (M+H)⁺=514.1.

Example 53 can also be obtained by the following steps:

### Step 1: diethyl 2-(((4-chloro-2-fluorophenyl)amino)methylene)malonate

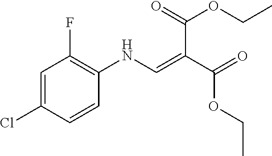 [SMILES: CCOC(=O)C(=CNc1ccc(Cl)cc1F)C(=O)OCC]

A solution of 4-chloro-2-fluoroaniline (25 g, 171.2 mmol) and diethyl 2-(ethoxymethylene)malonate (55 g) was stirred at 110° C. for 4 h. After cooled to room temperature, the reaction mixture was treated with petroleum ether (600 mL). The precipitate was filtered and dried to give the title compound (43.9 g, 81%). ¹H-NMR (400 MHz, DMSO-d6) δ 10.87 (d, J=13.3 Hz, 1H), 8.44 (d, J=13.4 Hz, 1H), 7.72-7.67 (m, 1H), 7.60 (d, J=10.9 Hz, 1H), 7.33 (d, J=8.7 Hz, 1H), 4.22 (q, J=6.9 Hz, 2H), 4.14 (q, J=6.9 Hz, 2H), 1.33-1.19 (m, 6H). LC-MS (M+H)⁺=316.2.

### Step 2: ethyl 6-chloro-8-fluoro-4-hydroxyquinoline-3-carboxylate

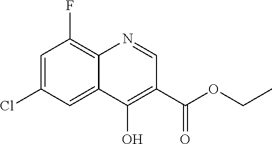 [SMILES: CCOC(=O)c1cnc2c(F)cc(Cl)cc2c1O]

The reaction mixture of diethyl 2-(((4-choro-2-fluorophenyl)amino)methylene)malonate (34.6 g, 109.5 mmol) and oxydibenzene (50 mL) was stirred at 260° C. for 9 h. After cooled to room temperature, the mixture was treated with petroleum ether (600 mL). The precipitate was filtered and dried to give the title product (43.9 g, 81%). ¹H-NMR (400 MHz, DMSO-d6) δ 12.65 (s, 1H), 8.39 (s, 1H), 7.91 (d, J=9.8 Hz, 2H), 4.23 (q, J=6.8 Hz, 2H), 1.28 (t, J=7.0 Hz, 3H). LC-MS (M+H)⁺=270.1.

### Step 3: ethyl 4-bromo-6-chloro-8-fluoroquinoline-3-carboxylate

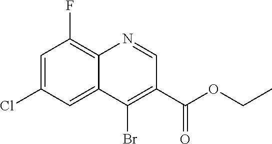 [SMILES: CCOC(=O)c1cnc2c(F)cc(Cl)cc2c1Br]

To the solution of ethyl 6-chloro-8-fluoro-4-hydroxyquinoline-3-carboxylate (5.5 g, 20.4 mmol) in dimethylformamide (80 mL) was added phosphorus tribromide (11 g, 40.8 mmol) and the mixture was stirred at 80° C. for 4 h. The mixture was cooled to room temperature and poured to ice water. The precipitate was filtered and dissolved with ethyl acetate. The mixture was washed with water and brine, dried over sodium sulfate, filtered and concentrated. The residue was purified by column chromatography eluting with ethyl acetate in petroleum ether (16%, v/v) to give the title compound (5.2 g, 76%). ¹H-NMR (400 MHz, DMSO-d₆) δ 9.09 (s, 1H), 8.15 (s, 1H), 8.10 (d, J=10.0 Hz, 1H), 4.46 (q, J=7.0 Hz, 2H), 1.39 (t, J=7.0 Hz, 3H). LC-MS (M+H)⁺=332.0, 334.0.

### Step 4: ethyl 6-chloro-8-fluoro-4-isopropylquinoline-3-carboxylate

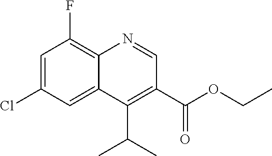 [SMILES: CCOC(=O)c1cnc2c(F)cc(Cl)cc2c1C(C)C]

To the solution of isopropylmagnesium bromide (7.5 mL, 1M, 7.5 mmol) under nitrogen was added zinc chloride solution in tetrahydrofuran (10.7 mL, 0.7 M, 7.5 mmol) and the mixture was stirred at 50° C. for 2 h. The solution of ethyl 4-bromo-6-chloro-8-fluoroquinoline-3-carboxylate (1.24 g, 3.74 mmol) in dimethylformamide (5 mL), copper(I) iodide (71 mg, 0.37 mmol) and (1,1′-bis(diphenylphosphino)ferrocene)palladium(II) dichloride (139 mg, 0.19 mmol) were added and the resulting mixture was stirred at 50° C. for 2 h before cooled to room temperature. The reaction was quenched with methanol (5 mL) and the solvent was evaporated. The residue was suspended in ethyl acetate and water before filtration. The filtrate was washed with water and brine, dried over sodium sulfate, filtered and evaporated. The residue was purified over silica gel by combi-flash, eluting with ethyl acetate in petroleum ether (16%, v/v) to give the title compound (0.95 mg, 86%). ¹H-NMR (400 MHz, DMSO-d6) δ 8.92 (s, 1H), 8.25 (s, 1H), 7.93 (d, J=10.0 Hz, 1H), 4.42 (q, J=7.0 Hz, 2H), 4.04-3.82 (m, 1H), 1.44 (d, J=6.9 Hz, 6H), 1.37 (t, J=7.0 Hz, 3H). LC-MS (M+H)⁺=296.1.

### Step 5: ethyl 8-fluoro-4-isopropyl-6-(4,4,5,5-tetramethyl-1,3,2-dioxaborolan-2-yl)quinoline-3-carboxylate

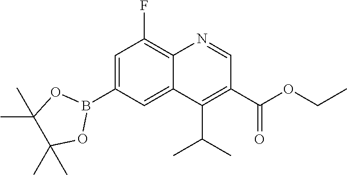 [SMILES: CCOC(=O)c1cnc2c(F)cc(B3OC(C)(C)C(C)(C)O3)cc2c1C(C)C]

A mixture of ethyl 6-chloro-8-fluoro-4-isopropylquinoline-3-carboxylate (532 mg, 1.8 mmol), bis(pinacolato)diboron (635 mg, 2.5 mmol), (1,1′-bis(diphenylphosphino)ferrocene)palladium(II) dichloride (72 mg, 0.1 mmol) and potassium acetate (459 mg, 4.7 mmol) in 1,4-dioxane (18 mL) was stirred at 80° C. under nitrogen for overnight before cooled to room temperature. The mixture was filtered and the filtrate was evaporated to give the product (0.7 g, 100%) which was used for next step without further purification. LC-MS (M+H)⁺=306.1 for corresponding boric acid.

### Step 6: ethyl 6-(2-chloro-5-fluoropyrimidin-4-yl)-8-fluoro-4-isopropylquinoline-3-carboxylate

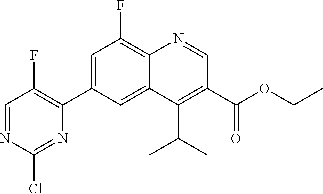 [SMILES: CCOC(=O)c1cnc2c(F)cc(-c3nc(Cl)ncc3F)cc2c1C(C)C]

A mixture of ethyl 8-fluoro-4-isopropyl-6-(4,4,5,5-tetramethyl-1,3,2-dioxaborolan-2-yl)quinoline-3-carboxylate (921 mg, 2.38 mmol), 2,4-dichloro-5-fluoropyrimidine (478 mg, 2.86 mmol), (1,1′-bis(diphenylphosphino)ferrocene)palladium(II) dichloride (103 mg, 0.14 mmol) and potassium carbonate (0.86 g, 6.2 mmol) in 1,4-dioxane (20 mL) and water (2.5 mL) under nitrogen was stirred at 80° C. for overnight before cooled to room temperature. The mixture was diluted with ethyl acetate, washed with brine, dried over sodium sulfate, filtered and evaporated. The residue was purified over silica gel by combi-flash, eluting with methanol in dichloromethane (3%, v/v) to give the title compound (420 mg, 45%). LC-MS (M+H)⁺=392.2.

### Step 7: ethyl 8-fluoro-6-(5-fluoro-2-(((3S,4R)-3-hydroxytetrahydro-2H-pyran-4-yl)amino)pyrimidin-4-yl)-4-isopropylquinoline-3-carboxylate

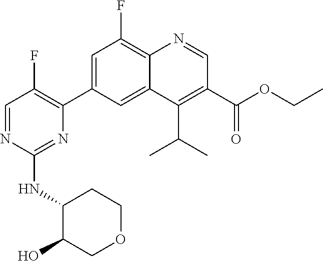 [SMILES: CCOC(=O)c1cnc2c(F)cc(-c3nc(N[C@@H]4CCOC[C@H]4O)ncc3F)cc2c1C(C)C]

The reaction mixture of ethyl 6-(2-chloro-5-fluoropyrimidin-4-yl)-8-fluoro-4-isopropylquinoline-3-carboxylate (423 mg, 1.08 mmol). (3S,4R)-4-aminotetrahydro-2H-pyran-3-ol hydrochloride (198 mg, 1.29 mmol). (SP-4-1)-[1,3-bis[2,6-bis(1-ethylpropyl)phenyl]-4,5-dichloro-1,3-dihydro-2H-imidazol-2-ylidene]dichloro(2-methylpyridine)palladium (44 mg, 0.054 mmol) and cesium carbonate (1.1 g, 3.24 mmol) in 1,4-dioxane (15 mL) under nitrogen was stirred at 100° C. for overnight. The mixture was cooled to room temperature and the solvent was evaporated. The residue was purified over silica gel by combi-flash, eluting with methanol in dichloromethane (6%, v/v) to give the title compound (300 mg, 59%). LC-MS (M+H)⁺=473.3.

### Step 8: (3S,4R)-4(4(5-fluoro-4-(8-fluoro-3-(hydroxymethyl)-4-isopropylquinolin-6-yl)pyrimidin-2-yl)amino)tetrahydro-2H-pyran-3-ol

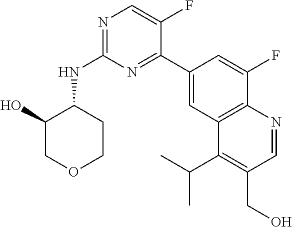 [SMILES: CC(C)c1c(CO)cnc2c(F)cc(-c3nc(N[C@@H]4CCOC[C@H]4O)ncc3F)cc12]

To the solution of ethyl 8-fluoro-6-(5-fluoro-2-(((3S,4R)-3-hydroxytetrahydro-2H-pyran-4-yl)amino)pyrimidin-4-yl)-4-isopropylquinoline-3-carboxylate (236 mg, 0.5 mmol) in tetrahydrofuran (50 mL) under nitrogen at 0° C. was added lithium aluminium hydride (57 mg, 1.5 mmol) and stirred at room temperature for 3 h. The reaction was quenched with methanol and the solvent was evaporated. The residue was purified by prep-HPLC (eluent: water (0.1% formic acid)/acetonitrile (0.1% formic acid)) to give the product (56 mg, 26%). ¹H-NMR (400 MHz, DMSO-d6) δ8.96 (s, 1H), 8.93 (s, 1H), 8.52 (d, J=2.7 Hz, 1H), 8.07 (d, J=11.6 Hz, 1H), 7.32 (d, J=7.7 Hz, 1H), 5.48 (s, 1H), 4.97 (s, 1H), 4.81 (s, 2H), 4.02-3.73 (m, 4H), 3.54 (s, 1H), 3.39-3.36 (m, 1H), 3.07 (t, J=10.4 Hz, 1H), 2.02 (s, 1H), 1.63-1.42 (m, 7H). LC-MS (M+H)⁺=431.1.

### Step 9: (3S,4R)-4-((4-(3-(chloromethyl-8-fluoro-4-isopropylquinolin-6-yl)-5-fluoropyrimidin-2-yl)amino)tetrahydro-2H-pyran-3-ol

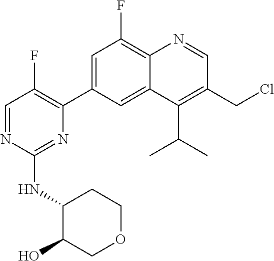 [SMILES: CC(C)c1c(CCl)cnc2c(F)cc(-c3nc(N[C@@H]4CCOC[C@H]4O)ncc3F)cc12]

To a solution of (3S,4R)-4-((5-fluoro-4-(8-fluoro-3-(hydroxymethyl)-4-isopropylquinolin-6-yl)pyrimidin-2-yl)amino)tetrahydro-2H-pyran-3-ol (40 mg, 0.093 mmol) in dichloromethane (10 mL) was added thionyl chloride (0.1 mL) at 0° C. and the reaction solution was stirred for 1 h before diluted with dichloromethane. The solution was washed with saturated aqueous solution of sodium bicarbonate, water and brine, dried over sodium sulfate, filtered and evaporated to give the crude title compound (40 mg). LC-MS (M+H)⁺=449.3.

### Step 10: (3S,4R)-4-((5-fluoro-4-(8-fluoro-4-isopropyl-3-((((R)-tetrahydro-2H-pyran-3-yl)amino)methyl)quinolin-6-yl)pyrimidin-2-yl)amino)tetrahydro-2H-pyran-3-ol

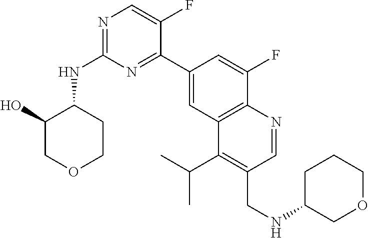 [SMILES: CC(C)c1c(CN[C@@H]2CCCOC2)cnc2c(F)cc(-c3nc(N[C@@H]4CCOC[C@H]4O)ncc3F)cc12]

To a solution of (3S,4R)-4-((4-(3-(chloromethyl)-8-fluoro-4-isopropylquinolin-6-yl)-5-fluoropyrimidin-2-yl)amino)tetrahydro-2H-pyran-3-ol (20 mg, 0.045 mmol) in dimethylformamide (1.5 mL) were added (R)-tetrahydro-2H-pyran-3-amine hydrochloride

(12 mg, 0.089 mmol) and N,N-diisopropylethylamine (17 mg, 0.13 mmol) at 25° C. The mixture was stirred at 25° C. for 5 h before diluted with ethyl acetate (15 mL). The organic solution was washed with brine (20 mL), dried over sodium sulfate, filtered and concentrated under reduce pressure. The residue was purified over silica gel by combi-flash, eluting with methanol in dichloromethane (5%, v/v) to give the title compound (14 mg, 61%). ¹H-NMR (400 MHz, DMSO-d6) δ 8.95 (s, 1H), 8.91 (s, 1H), 8.52 (s, 1H), 8.06 (d, J=11.6 Hz, 1H), 7.32 (d, J=7.5 Hz, 1H), 4.97 (s, 1H), 4.10-3.77 (m, 7H), 3.69 (d, J=11.0 Hz, 1H), 3.54 (s, 1H), 3.39-3.24 (m, 3H), 3.06 (t, J=10.1 Hz, 2H), 2.56 (s, 1H), 2.01-1.97 (m, 2H), 1.75-1.38 (m, 9H), 1.38-1.17 (m, 1H). LC-MS (M+H)⁺=514.1.

### Example 54: (3R,4R)-4-fluoro-1-((6-(5-fluoro-2-(((3S,4R)-3-hydroxytetrahydro-2H-pyran-4-yl)amino)pyrimidin-4-yl)-4-isopropylquinolin-3-yl)methyl)pyrrolidin-3-ol

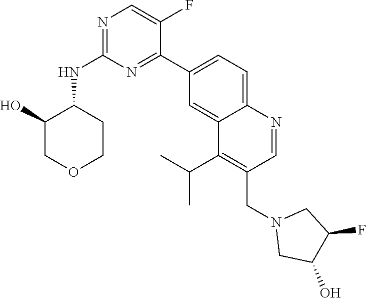 [SMILES: CC(C)c1c(CN2C[C@@H](O)[C@H](F)C2)cnc2ccc(-c3nc(N[C@@H]4CCOC[C@H]4O)ncc3F)cc12]

The title compound (11.5 mg, 16.5%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (3R,4R)-4-fluoropyrrolidin-3-ol. ¹H-NMR (400 MHz, DMSO-d6) δ 9.11 (s, 1H), 8.82 (s, 1H), 8.52-8.48 (m, 1H), 8.31-8.25 (m, 1H), 8.17-8.12 (m, 1H), 7.26 (d, J=7.9 Hz, 1H), 5.37-5.32 (m, 1H), 4.99-4.95 (m, 1H), 4.90-4.72 (m, 1H), 4.22-4.09 (m, 1H), 4.02-3.95 (m, 1H), 3.94-3.78 (m, 5H), 3.60-3.50 (m, 1H), 3.40-3.35 (m, 1H), 3.12-3.00 (m, 2H), 2.84-2.68 (m, 2H), 2.24-2.18 (m, 1H), 2.10-2.00 (m, 1H) 1.61-1.48 (m, 7H). LC-MS (M+H)⁺=500.1.

### Example 55 and Example 56: (3S,4R)-4-((4-(3-((S)-1-aminoethyl)-4-isopropylquinolin-6-yl)-5-fluoropyrimidin-2-yl)amino)tetrahydro-2H-pyran-3-ol & (3S,4R)-4-((4-(3-((R)-1-aminoethyl)-4-isopropylquinolin-6-yl)-5-fluoropyrimidin-2-yl)amino)tetrahydro-2H-pyran-3-ol

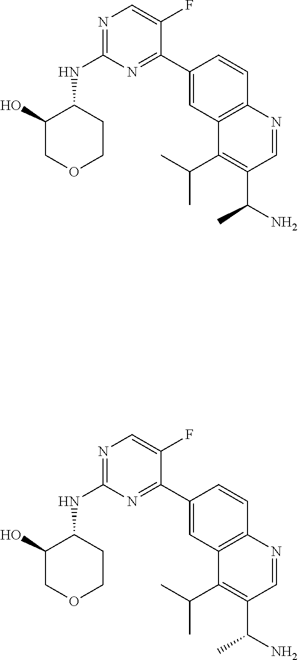 [SMILES: CC(C)c1c([C@H](C)N)cnc2ccc(-c3nc(N[C@@H]4CCOC[C@H]4O)ncc3F)cc12 ; CC(C)c1c([C@@H](C)N)cnc2ccc(-c3nc(N[C@@H]4CCOC[C@H]4O)ncc3F)cc12]

### Step 1: (3S,4R)-44(4-(3-(1-azidoethyl)-4-isopropylquinolin-6-yl)-5-fluoropyrimidin-2-yl)amino)tetrahydro-2H-pyran-3-ol

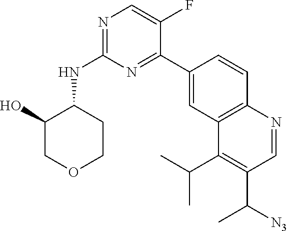 [SMILES: CC(C)c1c(C(C)N=[N+]=[N-])cnc2ccc(-c3nc(N[C@@H]4CCOC[C@H]4O)ncc3F)cc12]

The title compound (71 mg, 37%) was prepared in a manner similar to that in Example 38 step 1 from (3S,4R)-4-((5-fluoro-4-(3-(1-hydroxyethyl)-4-isopropylquinolin-6-yl)pyrimidin-2-yl)amino)tetrahydro-2H-pyran-3-ol. LC-MS (M+H)⁺=452.2.

### Step 2: (3S,4R)-4-((4-(3-((S)-1-aminoethyl)4-isopropylquinolin-6-yl)-5-fluoropyrimidin-2-yl)amino)tetrahydro-2H-pyran-3-ol & (3S,4R)-4-((4-(3-((R)-1-aminoethyl)-4-isopropylquinolin-6-yl)-5-fluoropyrimidin-2-yl)amino)tetrahydro-2H-pyran-3-ol

The mixture of Example 55 and Example 56 was prepared in a manner similar to that in Example 38 step 2 from (3S,4R)-4-((4-(3-(I-azidoethyl)-4-isopropylquinolin-6-yl)-5-fluoropyrimidin-2-yl)amino)tetrahydro-2H-pyran-3-ol. Example 55 and Example 56 were separated by chiral-HPLC to give (3S,4R)-4-((4-(3-((S)-1-aminoethyl)-4-isopropylquinolin-6-yl)-5-fluoropyrimidin-2-yl)amino)tetrahydro-2H-pyran-3-ol & (3S,4R)-4-((4-(3-((R)-1-aminoethyl)-4-isopropylquinolin-6-yl)-5-fluoropyrimidin-2-yl)amino)tetrahydro-2H-pyran-3-ol. Analytical chiral HPLC condition: CHIRALPAK ID-3, 0.46×5 cm, 3.0 um. Mobile phase: (hexanes:dichloromethane=1:1, containing 0.5% 2M NH₃-MeOH): EtOH, 20 mL/min in 22.5 min.

Example 55 (5 mg, 174): ¹H-NMR (400 MHz, DMSO-d6) δ9.17 (s, 1H), 9.08 (brs, 1H), 8.49 (d, J=3.9 Hz, 1H), 8.25 (d, J=8.8 Hz, 1H), 8.11 (d, J=8.8 Hz, 1H), 7.24 (d, J=7.8 Hz, 1H), 5.00-4.94 (m, 1H), 4.71-4.61 (m, 1H), 4.03-3.80 (m, 4H), 3.60-3.49 (m, 1H), 3.41-3.32 (m, 1H), 3.11-3.01 (m, 1H), 2.10-2.01 (m, 1H), 1.61-1.45 (m, 7H), 1.38 (d, J=6.5 Hz, 3H). LC-MS (M+H)⁺=426.1. Chiral HPLC: RT=3.148 min.

Example 56 (5 mg, 17%) ¹H-NMR (400 MHz, DMSO-d6) δ 9.18 (s, 1H), 9.08 (brs, 1H), 8.49 (d, J=3.9 Hz, 1H), 8.25 (d, J=8.8 Hz, 1H), 8.11 (d, J=8.8 Hz, 1H), 7.24 (d, J=7.8 Hz, 1H), 4.99-4.95 (m, 1H), 4.71-4.61 (m, 1H), 4.06-3.75 (m, 4H), 3.57-3.49 (m, 1H), 3.42-3.31 (m, 1H), 3.11-3.01 (m, 1H), 2.09-2.01 (m, 1H), 1.60-1.44 (m, 7H), 1.38 (d, J=6.6 Hz, 3H). LC-MS (M+H)⁺=426.1. Chiral HPLC: RT=5.669 min.

### Example 57: (S)-1-((6-(5-fluoro-2-(((3S,4R)-3-hydroxytetrahydro-2H-pyran-4-yl)amino)pyrimidin-4-yl)-4-isopropylquinolin-3-yl)methyl)piperidin-3-ol

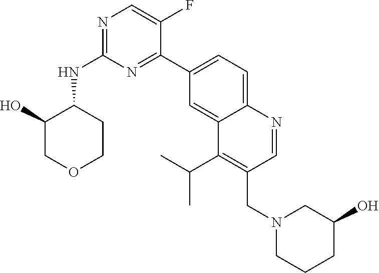 [SMILES: CC(C)c1c(CN2CCC[C@H](O)C2)cnc2ccc(-c3nc(N[C@@H]4CCOC[C@H]4O)ncc3F)cc12]

The title compound (16 mg, 44%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (3S)-piperidin-3-ol hydrochloride. ¹H-NMR (300 MHz, DMSO-d6) δ 9.12 (brs, 1H), 8.76 (s, 1H), 8.50 (d, J=3.9 Hz, 1H), 8.33-8.25 (m, 1H), 8.14 (d, J=8.9 Hz, 1H), 7.27 (d, J=7.8 Hz, 1H), 4.99 (d, J=5.3 Hz, 1H), 4.59 (d, J=4.6 Hz, 1H), 4.04-3.92 (m, 1H), 3.90-3.81 (m, 3H), 3.80-3.72 (m, 1H), 3.68-3.61 (m, 1H), 3.61-3.50 (m, 1H), 3.43-3.35 (m, 2H), 3.11-3.01 (m, 1H), 2.79-2.71 (m, 1H), 2.69-2.61 (m, 1H), 2.10-2.02 (m, 1H), 2.00-1.90 (m, 1H), 1.84-1.74 (m, 2H), 1.65-1.45 (m, 8H), 1.41-1.33 (m, 1H), 1.16-1.02 (m, 1H). LC-MS (M+H)⁺=496.3.

### Example 58: (3S,4R)-4-((5-fluoro-4-(4-isopropyl-3-(((R)-3-methylmorpholino)methyl)quinolin-6-yl)pyrimidin-2-yl)amino)tetrahydro-2H-pyran-3-ol

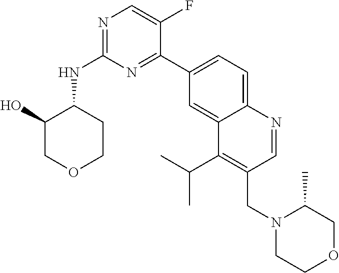 [SMILES: CC(C)c1c(CN2CCOC[C@H]2C)cnc2ccc(-c3nc(N[C@@H]4CCOC[C@H]4O)ncc3F)cc12]

The title compound (8 mg, 24%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (R)-3-methylmorpholine. ¹H-NMR (30) MHz, DMSO-d6) δ 9.12 (brs, 1H), 8.80 (s, 1H), 8.50 (d, J=3.9 Hz, 1H), 8.34-8.24 (m, 1H), 8.14 (d, J=8.8 Hz, 1H), 7.75-7.45 (m, 1H), 7.27 (d, J=7.8 Hz, 1H), 5.03-4.94 (m, 1H), 4.35-4.24 (m, 1H), 4.14-3.98 (m, 1H), 3.93-3.81 (m, 3H), 3.70-3.48 (m, 3H), 3.44-3.28 (m, 2H), 3.23-3.00 (m, 2H), 2.51-2.45 (m, 1H), 2.42-2.32 (m, 1H), 2.22-2.00 (m, 2H), 1.65-1.49 (m, 7H), 1.11 (d, J=6.2 Hz, 3H). LC-MS (M+H)⁺=496.4.

### Example 59: (3S,4R)-4-((5-fluoro-4-(4-isopropyl-3-((pyridin-2-ylamino)methyl)quinolin-6-yl)pyrimidin-2-yl)amino)tetrahydro-2H-pyran-3-ol

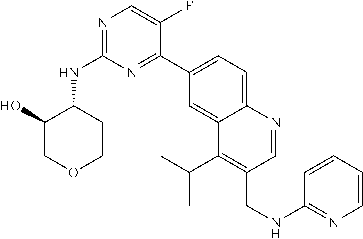 [SMILES: CC(C)c1c(CNc2ccccn2)cnc2ccc(-c3nc(N[C@@H]4CCOC[C@H]4O)ncc3F)cc12]

The title compound (6.8 mg, 24%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and pyridin-2-amine. ¹H-NMR (400 MHz, DMSO-d6) δ 9.14 (s, 1H), 8.60 (s, 1H), 8.52 (s, 1H), 8.48 (s, 1H), 8.39-8.34 (m, 1H), 8.23-8.18 (m, 1H), 7.77-7.70 (m, 1H), 7.69-7.64 (m, 1H), 7.38-7.32 (m, 1H), 7.17-7.11 (m, 1H), 6.70-6.64 (m, 1H), 5.62 (s, 2H), 3.92-3.80 (m, 2H), 3.60-3.54 (m, 2H), 3.39-3.31 (m, 2H), 3.05 (t, J=10.3 Hz, 1H), 2.11-2.00 (m, 1H), 1.60-1.48 (m, 7H). LC-MS (M+H)⁺=489.2.

### Example 60: (1R,3R,5S)-9-((6-(5-fluoro-2-(((3S,4R)-3-hydroxytetrahydro-2H-pyran-4-yl)amino)pyrimidin-4-yl)-4-isopropylquinolin-3-yl)methyl)-9-azabicyclo[3.3.1]nonan-3-ol

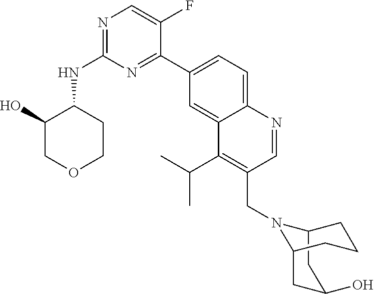 [SMILES: CC(C)c1c(CN2C3CCCC2CC(O)C3)cnc2ccc(-c3nc(N[C@@H]4CCOC[C@H]4O)ncc3F)cc12]

The title compound (9 mg, 32%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (1R,3r,5S)-9-azabicyclo[3.3.1]nonan-3-ol hydrochloride. H-NMR (300 MHz, DMSO-d6) δ 9.10 (brs, 1H), 8.77 (s, 1H), 8.48 (d, J=3.9 Hz, 1H), 8.30-8.21 (m, 11), 8.11 (d, J=8.8 Hz, 1H), 7.24 (d, J=7.8 Hz, 1H), 4.95 (d, J=5.2 Hz, 1H), 4.42 (d, J=4.5 Hz, 1H), 4.07-3.91 (m, 4H), 3.90-3.78 (m, 3H), 3.61-3.46 (m, 1H), 3.42-3.32 (m, 1H), 3.11-2.98 (m, 1H), 2.93-2.84 (m, 2H), 2.40-2.09 (m, 3H), 2.09-1.83 (m, 3H), 1.62-1.52 (m, 6H), 1.51-1.35 (m, 2H), 1.31-1.17 (m, 2H), 1.14-1.04 (m, 2H). LC-MS (M+H)⁺=536.4.

### Example 61: (S)-1-((6-(5-fluoro-2-(((3S,4R)-3-hydroxytetrahydro-2H-pyran-4-yl)amino)pyrimidin-4-yl)-4-isopropylquinolin-3-yl)methyl)piperidine-3-carbonitrile

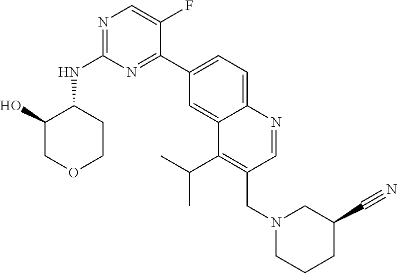 [SMILES: CC(C)c1c(CN2CCC[C@H](C#N)C2)cnc2ccc(-c3nc(N[C@@H]4CCOC[C@H]4O)ncc3F)cc12]

The title compound (12 mg, 38%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (S)-piperidine-3-carbonitrile. ¹H-NMR (300 MHz, DMSO-d6) δ 9.13 (brs, 1H), 8.80-8.74 (m, 1H), 8.55-8.45 (m, 1H), 8.34-8.23 (m, 1H), 8.20-8.09 (m, 1H), 7.31-7.23 (m, 1H), 4.99-4.93 (m, 1H), 4.10-3.66 (m, 6H), 3.58-3.52 (m, 1H), 3.43-3.33 (m, 2H), 3.07-3.01 (m, 2H), 2.76-2.70 (m, 1H), 2.29-2.19 (m, 1H), 2.07-2.01 (m, 1H), 1.81-1.41 (m, 12H). LC-MS (M+H)⁺=505.4.

### Example 62: (R)-1-((6-(5-fluoro-2-(((3S,4R)-3-hydroxytetrahydro-2H-pyran-4-yl)amino)pyrimidin-4-yl)-4-isopropylquinolin-3-yl)methyl)piperidine-3-carbonitrile

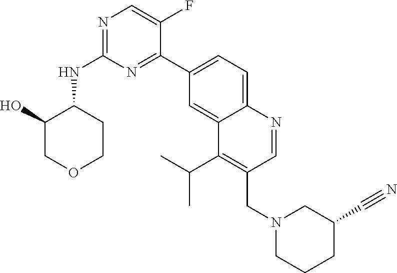 [SMILES: CC(C)c1c(CN2CCC[C@@H](C#N)C2)cnc2ccc(-c3nc(N[C@@H]4CCOC[C@H]4O)ncc3F)cc12]

The title compound (13 mg, 37%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (R)-piperidine-3-carbonitrile. ¹H-NMR (300 MHz, DMSO-d6) δ 9.14 (brs, 1H), 8.80-8.74 (m, 1H), 8.53-8.46 (m, 1H), 8.29 (d, J=8.8 Hz, 1H), 8.14 (d, J=8.6 Hz, 1H), 7.26 (d, J=7.6 Hz, 1H), 5.02-4.94 (m, 1H), 4.04-3.80 (m, 4H), 3.77-3.65 (m, 2H), 3.60-3.54 (m, 1H), 3.45-3.36 (m, 2H), 3.13-2.99 (m, 2H), 2.78-2.68 (m, 1H), 2.28-2.22 (m, 1H), 2.11-2.00 (m, 1H), 1.82-1.42 (m, 12H). LC-MS (M+H)⁺=505.4.

### Example 67: (3S,4R)-4-((5-fluoro-4-(8-fluoro-3-(2-hydroxypropan-2-yl)-4-isopropylquinolin-6-yl)pyrimidin-2-yl)amino)tetrahydro-2H-pyran-3-ol

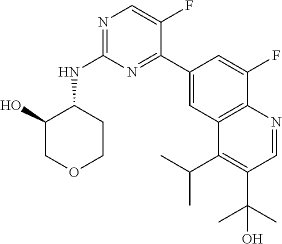 [SMILES: CC(C)c1c(C(C)(C)O)cnc2c(F)cc(-c3nc(N[C@@H]4CCOC[C@H]4O)ncc3F)cc12]

### Step 1: 2-(4-bromo-6-chloro-8-fluoroquinolin-3-yl)propan-2-ol

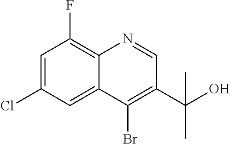 [SMILES: CC(C)(O)c1cnc2c(F)cc(Cl)cc2c1Br]

To a solution of ethyl 4-bromo-6-chloro-8-fluoroquinoline-3-carboxylate (333 mg, 1 mmol) in tetrahydrofuran (8 mL) under nitrogen at −78° C. was added methylmagnesium bromide (3 mL, 3 mmol, 1M in tetrahydrofuran) and the mixture was slowly warmed to 0° C., in 2 h before quenched by saturated aqueous solution of ammonium chloride. The aqueous layer was extracted with ethyl acetate 3 times. The combined organic layers were washed with brine, dried over sodium sulfate, filtered and concentrated. The residue was purified by column chromatography, eluting with ethyl acetate in petroleum ether (33%, v/v) to give the title compound (240 mg, 75%). LC-MS (M+H)⁺=318.0, 320.0.

### Step 2: 2-(6-chloro-8-fluoro-4-isopropylquinolin-3-yl)propan-2-ol

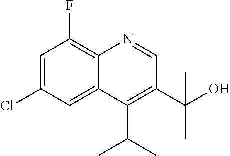 [SMILES: CC(C)c1c(C(C)(C)O)cnc2c(F)cc(Cl)cc12]

To a solution of isopropylmagnesium bromide (3 mL, 1M in tetrahydrofuran, 3 mmol) under nitrogen was added zinc chloride (3 mL, 1 M in tetrahydrofuran, 3 mmol) and the mixture was stirred at 50° C. for 2 h before addition of 2-(4-bromo-6-chloro-8-fluoroquinolin-3-yl)propan-2-ol (240 mg, 0.75 mmol) in N,N-dimethylformamide (10 mL), CuI (14 mg, 0.075 mmol) and (1,1′-bis(diphenylphosphino)ferrocene)palladium(II) dichloride (28 mg, 0.038 mmol). The reaction mixture was stirred at 50° C. for 2 h before cooled to room temperature. The reaction was quenched with methanol (10 mL) and the solvents were concentrated. The residue was diluted with ethyl acetate, and the organic solution was washed with water and saturated aqueous solution of ammonium chloride. The aqueous phase was extracted with ethyl acetate 3 times. The organic layers were combined, washed with water and brine, dried over sodium sulfate, filtered and evaporated. The residue was purified by column chromatography, eluting with ethyl acetate in petroleum ether (50%, v/v) to give the title compound (75 mg, 35%). LC-MS (M+H)⁺=282.2.

### Step 3: 2-(8-fluoro-4-isopropyl-6-(4,4,5,5-tetramethyl-1,3,2-dioxaborolan-2-yl)quinolin-3-yl)propan-2-ol

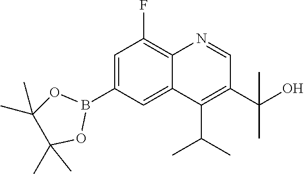 [SMILES: CC(C)c1c(C(C)(C)O)cnc2c(F)cc(B3OC(C)(C)C(C)(C)O3)cc12]

The title compound (99 mg, 100%) was prepared in a manner similar to Example 1 step 2 from 2-(6-chloro-8-fluoro-4-isopropylquinolin-3-yl)propan-2-ol and bis(pinacolato)diboron. LC-MS (M+H)⁺=292.2 for corresponding boronic acid.

### Step 4: 2-chloro-5-fluoro-4-(4-methoxybenzyl(oxy)pyrimidine

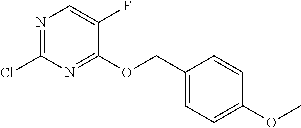 [SMILES: COc1ccc(COc2nc(Cl)ncc2F)cc1]

To a solution of sodium hydride (925 mg, 23.13 mmol, 60%) in tetrahydrofuran (30 mL) was added (4-methoxyphenyl)methanol (2350 mg, 17.018 mmol) in tetrahydrofuran (12.0 mL) dropwise at 0° C. under nitrogen atmosphere. The resulting mixture was stirred for 30 min at 0° C. under nitrogen atmosphere. To the above mixture was added 2,4-dichloro-5-fluoropyrimidine (2375 mg, 14.23 mmol) in tetrahydrofuran (12 mL) dropwise over 20 min at 0° C. The resulting mixture was stirred for additional 2 h at 0° C. under nitrogen atmosphere before quenched by the addition of saturated aqueous ammonium chloride solution (30 mL). The resulting solution was extracted with ethyl acetate (40 mL×2). The organic phases were combined, washed with brine, dried over sodium sulfate, filtered and concentrated. The residue was purified by flash chromatography eluting with ethyl acetate in petroleum ether (0% to 10% gradient, v/v) to yield the title compound (3300 mg, 86%). LC-MS (M+H)⁺=269.2.

### Step 5: (3S,4R)-4-((5-fluoro-4-((4-methoxybenzyl)oxy)pyrimidin-2-yl)amino)tetrahydro-2H-pyran-3-01

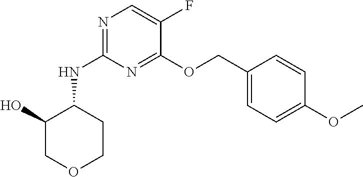 [SMILES: COc1ccc(COc2nc(N[C@@H]3CCOC[C@H]3O)ncc2F)cc1]

The title compound (1900 mg, 73%) was prepared in a manner similar to that in Example 1 step 4 from 2-chloro-5-fluoro-4-((4-methoxybenzyl)oxy)pyrimidine and (3S,4R)-4-aminooxan-3-ol hydrochloride. LC-MS (M+H)⁺=350.1.

### Step 6: (3S,4R)-44(5-fluoro-44(4-methoxybenzyl)oxy)pyrimidin-2-yl)amino)tetrahydro-2H-pyran-3-yl acetate

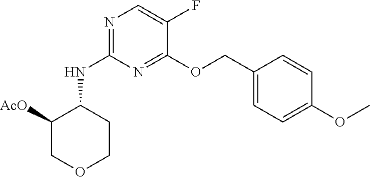 [SMILES: COc1ccc(COc2nc(N[C@@H]3CCOC[C@H]3OC(C)=O)ncc2F)cc1]

To a solution of (3S,4R)-4-((5-fluoro-4-((4-methoxybenzyl)oxy)pyrimidin-2-yl)amino)tetrahydro-2H-pyran-3-ol (1900 mg, 5.43 mmol) and triethylamine (4400 mg, 43.47 mmol) in dichloromethane (40 mL) was added acetic anhydride (3325 mg, 32.57 mmol) dropwise at 0° C. under nitrogen atmosphere. The resulting mixture was stirred for 16 h at room temperature under nitrogen atmosphere before quenched by the addition of aqueous saturated sodium bicarbonate solution (30 mL). The resulting mixture was extracted with dichloromethane (40 mL×2). The organic phases were combined, washed with brine, dried over sodium sulfate, filtered and concentrated. The residue was purified by flash chromatography eluting with ethyl acetate in dichloromethane (0% to 25% gradient, v/v) to yield the title compound (1950 mg, 91%). LC-MS (M+H)⁺=392.0.

### Step 7: (3S,4R)-4-(5-fluoro-4-hydroxypyrimidin-2-yl)amino)tetrahydro-2H-pyran-3-yl acetate

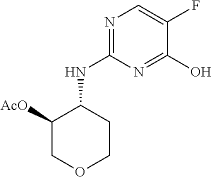 [SMILES: CC(=O)O[C@@H]1COCC[C@H]1Nc1ncc(F)c(O)n1]

To a solution of (3S,4R)-4-((5-fluoro-4-((4-methoxybenzyl)oxy)pyrimidin-2-yl)amino)tetrahydro-2H-pyran-3-yl acetate (1950 mg, 4.98 mmol) in methanol (30 mL) was added 10% Pd/C (945 mg, 0.888 mmol) under nitrogen atmosphere. The mixture was hydrogenated at room temperature for 6 h under 1 atmosphere of hydrogen. The reaction was filtered through a Celite pad and concentrated under reduced pressure to yield the title compound (1200 mg, 88%). LC-MS (M+H)⁺=272.0.

### Step 8: (3S,4R)-44(4-chloro-5-fluoropyrimidin-2-yl)amino)tetrahydro-2H-pyran-3-yl acetate

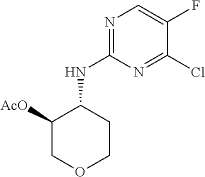 [SMILES: CC(=O)O[C@@H]1COCC[C@H]1Nc1ncc(F)c(Cl)n1]

To a solution of (3S,4R)-4-((5-fluoro-4-hydroxypyrimidin-2-yl)amino)tetrahydro-2H-pyran-3-yl acetate (1200 mg, 4.42 mmol) and N,N-diethylaniline (1985 mg, 13.31 mmol) in 1,2-dichloroethane (25 mL) was added phosphoryl chloride (2055 mg, 13.42 mmol) dropwise at room temperature under nitrogen atmosphere. The resulting mixture was stirred for 3 h at 100° C. under nitrogen atmosphere before cooled to room temperature and concentrated. The residue was purified by flash chromatography eluting with ethyl acetate in petroleum ether (0% to 30% gradient) to yield the title compound (820 mg, 63%). LC-MS (M+H)⁺=290.0.

### Step 9: (3S,4R)-44(5-fluoro-4-(8-fluoro-3-(2-hydroxypropan-2-yl)-4-isopropylquinolin-6-yl)pyrimidin-2-yl)amino)tetrahydro-2H-pyran-3-ol

The title compound (11 mg, 20%) was prepared in a manner similar to Example 1 step 3 from 2-(8-fluoro-4-isopropyl-6-(4,4,5,5-tetramethyl-1,3,2-dioxaborolan-2-yl)quinolin-3-yl)propan-2-ol and (3S,4R)-4-((4-chloro-5-fluoropyrimidin-2-yl)amino)tetrahydro-2H-pyran-3-yl acetate, 1H NMR (400 MHz, d-DMSO) δ 9.14 (s, 1H), 9.02 (s, 1H), 8.52 (s, 1H), 8.05 (d, J=11.7 Hz, 1H), 7.31 (d, J=7.7 Hz, 1H), 5.51 (s, 1H), 4.97 (s, 1H), 4.81-4.55 (m, 1H), 3.95-3.75 (m, 3H), 3.54 (s, 1H), 3.40-3.30 (m, 1H), 3.06 (t, J=10.1 Hz, 1H), 2.02 (s, 1H), 1.70 (s, 6H), 1.64-1.43 (m, 7H). LC-MS (M+H)⁺=459.1.

### Example 68: (3S,4R)-4-((4-(3-((3R,5S)-3,5-dimethylmorpholino)methyl)-4-isopropylquinolin-6-yl)-5-fluoropyrimidin-2-yl)amino)tetrahydro-2H-pyran-3-ol

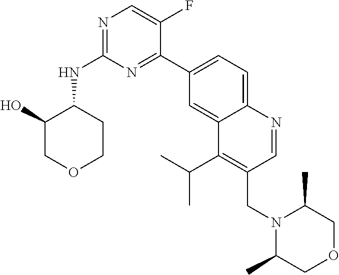 [SMILES: CC(C)c1c(CN2[C@H](C)COC[C@@H]2C)cnc2ccc(-c3nc(N[C@@H]4CCOC[C@H]4O)ncc3F)cc12]

The title compound (5.3 mg, 18%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (3R,5S)-3,5-dimethylmorpholine hydrochloride. ¹H-NMR (400 MHz, DMSO-d6) δ: 9.31 (s, 1H), 9.07 (s, 1H), 8.49 (s, 1H), 8.29-8.21 (m, 1H), 8.15-8.08 (m, 1H), 7.30-7.20 (m, 1H), 5.03-4.91 (m, 1H), 4.05-3.97 (m, 2H), 3.80-3.97 (m, 4H), 3.75-3.65 (m, 2H), 3.60-3.50 (m, 1H), 3.40-3.35 (m, 1H), 3.27-3.18 (m, 2H), 3.05 (t, J=10.3 Hz, 1H), 2.71-2.60 (m, 2H), 2.10-1.95 (m, 1H), 1.62-1.50 (m, 7H), 0.86-0.78 (m, 6H). LC-MS (M+H)⁺=510.1.

### Example 69: (3S,4R)-4-((4-(3-(((3R,5R)-3,5-dimethylmorpholino)methyl)-4-isopropylquinolin-6-yl)-5-fluoropyrimidin-2-yl)amino)tetrahydro-2H-pyran-3-ol

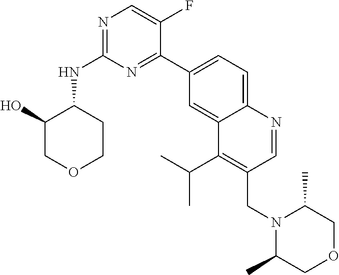 [SMILES: CC(C)c1c(CN2[C@H](C)COC[C@H]2C)cnc2ccc(-c3nc(N[C@@H]4CCOC[C@H]4O)ncc3F)cc12]

The title compound (5.6 mg, 19%) was prepared in a manner similar to that in Example 11 step 1 from (3S,4R)-4-((4-(3-(chloromethyl)-4-isopropylquinolin-6-yl)-5-fluoropyrimidin-2-yl)amino)tetrahydro-2H-pyran-3-ol and (3R,5R)-3,5-dimethylmorpholine hydrochloride. ¹H-NMR (400 MHz, DMSO-d6) δ: 9.12 (s, 1H), 8.89 (s, 1H), 8.50 (s, 1H), 8.31-8.24 (m, 1H), 8.15-8.10 (m, 1H), 7.30-7.22 (m, 1H), 5.00-4.94 (m, 1H), 4.30-4.21 (m, 1H), 4.20-4.10 (m, 1H), 3.95-3.80 (m, 3H), 3.63-3.50 (m, 4H), 3.40-3.35 (m, 1H), 3.28-3.19 (m, 2H), 3.05 (t, J=10.3 Hz, 1H), 2.71-2.60 (m, 2H), 2.10-1.95 (m, 1H), 1.62-1.50 (m, 7H), 1.07-1.00 (m, 6H). LC-MS (M+H)⁺⁼510.1.

### Example 70: (3S,4R)-4-((4-(7-chloro-3-(2-hydroxypropan-2-yl)4-isopropylquinolin-6-yl)-5-fluoropyrimidin-2-yl)amino)tetrahydro-2H-pyran-3-ol

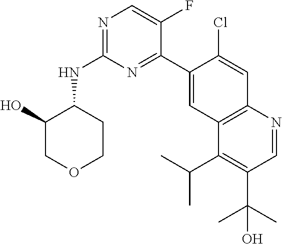 [SMILES: CC(C)c1c(C(C)(C)O)cnc2cc(Cl)c(-c3nc(N[C@@H]4CCOC[C@H]4O)ncc3F)cc12]

### Step 1: diethyl 2-(((3-chloro-4-methoxyphenyl)amino)methylene)malonate

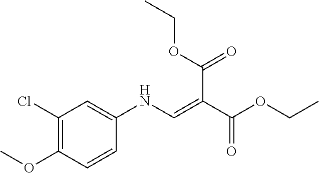 [SMILES: CCOC(=O)C(=CNc1ccc(OC)c(Cl)c1)C(=O)OCC]

To 3-chloro-4-methoxyaniline (8.0 g, 50.76 mmol) were added diethyl 2-(ethoxymethylene)malonate (16.4 g, 76.14 mmol). The reaction mixture was stirred at 110° C. for 3 h before cooled to 60° C. The mixture was poured into petroleum ether (500 mL) and the precipitate was filtered to give the title compound (14.1 g, 85%). LC-MS (M+H)⁺=328.1

### Step 2: ethyl 7-chloro-4-hydroxy-6-methoxyquinoline-3-carboxylate

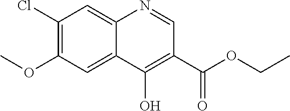 [SMILES: CCOC(=O)c1cnc2cc(Cl)c(OC)cc2c1O]

A mixture of diethyl 2-(((3-chloro-4-methoxyphenyl)amino)methylene)malonate (10.0 g, 30.57 mmol) and diphenyl ether (50 mL) was stirred at 260° C. for 2 h before cooled to 60° C. The mixture was poured into petroleum ether (500 mL) and the precipitate was filtered to give the title compound (7.0 g, 82%). LC-MS (M+H)⁺=282.1.

### Step 3: ethyl 4,7-dichloro-6-methoxyquinoline-3-carboxylate

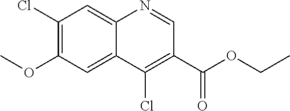 [SMILES: CCOC(=O)c1cnc2cc(Cl)c(OC)cc2c1Cl]

To a solution of ethyl 7-chloro-4-hydroxy-6-methoxyquinoline-3-carboxylate (5.0 g, 17.73 mmol) in dichloromethane (50 mL) and N,N-dimethylformamide (0.4 mL) was added oxalyl dichloride (2.7 g, 17.73 mmol). The reaction mixture was stirred at 40° C. for 3 h before cooled to room temperature and diluted with ethyl acetate (50 mL). The organic solution was washed with brine (30 mL), dried over sodium sulfate, filtered and concentrated under reduce pressure. The residue was purified by silica gel column chromatography, eluting with ethyl acetate in petroleum ether (50%, v/v) to give the title compound (3.9 g, 76%). LC-MS (M+H)⁺=300.1

### Step 4: ethyl 7-chloro-4-isopropyl-6-methoxyquinoline-3-carboxylate

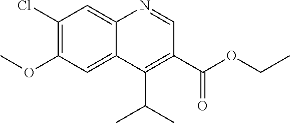 [SMILES: CCOC(=O)c1cnc2cc(Cl)c(OC)cc2c1C(C)C]

To isopropylmagnesium bromide (4.0 mL, 10.0 mmol, 2.5 M in tetrahydrofuran) was added zinc chloride (14.3 mL, 10.0 mmol, 0.7M in tetrahydrofuran). The reaction mixture was stirred at 50° C. for 1 h before cooled to room temperature. To the mixture were added ethyl 4,7-dichloro-6-methoxyquinoline-3-carboxylate (2.0 g, 6.67 mmol), CuI (128 mg, 0.67 mmol), (1,1′-bis(diphenylphosphino)ferrocene)palladium(II) dichloride (248 mg, 0.34 mmol). Then reaction mixture was stirred at 50° C. for 3 h before cooled to room temperature and diluted with ethyl acetate (50 mL). The mixture was washed with brine, dried over sodium sulfate, filtered and concentrated under reduce pressure. The residue was purified by silica gel column chromatography, eluting with ethyl acetate in petroleum ether (50%, v/v) to give the title compound (1.4 g, 69%). LC-MS (M+H)⁺=308.3.

### Step 5: ethyl 7-chloro-6-hydroxy-4-isopropylquinoline-3-carboxylate

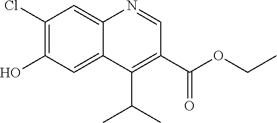 [SMILES: CCOC(=O)c1cnc2cc(Cl)c(O)cc2c1C(C)C]

To a solution of ethyl 7-chloro-4-isopropyl-6-methoxyquinoline-3-carboxylate (1.1 g, 3.57 mmol) in dichloromethane (10 mL) was added BBr₃ (17.8 mL, 17.85 mmol, 1 M in dichloromethane) at 0° C. Then reaction mixture was stirred at 0° C. for 2 h before diluted with ethyl acetate (30 mL). The mixture was washed with brine (20 mL), dried over sodium sulfate, filtered and concentrated under reduce pressure. The residue was purified by silica gel column chromatography, eluting with ethyl acetate in petroleum ether (50%, v/v) to give the title compound (750 mg, 72%). LC-MS (M+H)⁺=294.3.

### Step 6: ethyl 7-chloro-4-isopropyl-6-(((trifluoromethyl)sulfonyl)oxy)quinoline-3-carboxylate

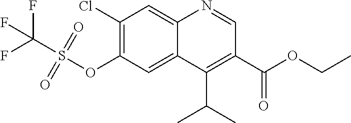 [SMILES: CCOC(=O)c1cnc2cc(Cl)c(OS(=O)(=O)C(F)(F)F)cc2c1C(C)C]

To a solution of ethyl 7-chloro-6-hydroxy-4-isopropylquinoline-3-carboxylate (720 mg, 2.46 mmol) in dichloromethane (10 mL) were added triethylamine (596 mg, 5.90 mmol) and trifluoromethanesulfonic anhydride (1.39 g, 4.92 mmol) at 0° C. and then reaction mixture was stirred at 0° C. for 2 h before diluted with ethyl acetate (30 mL). The mixture was washed with brine (20 mL), dried over sodium sulfate, filtered and concentrated under reduce pressure. The residue was purified by silica gel column chromatography, eluting with ethyl acetate in petroleum ether (50%, v/v) to give the title compound to give the title compound (889 mg, 85%). LC-MS (M+H)⁺=426.4.

### Step 7: ethyl 7-chloro-4-isopropyl-4-(4,4,5,5-tetramethyl-1,3,2-dioxaborolan-2-yl)quinoline-3-carboxylate

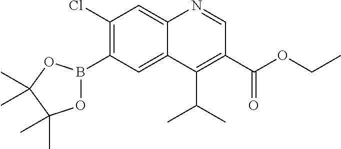 [SMILES: CCOC(=O)c1cnc2cc(Cl)c(B3OC(C)(C)C(C)(C)O3)cc2c1C(C)C]

The title compound (508 mg, 92%) was prepared in a manner similar to Example 1 step 2 from ethyl 7-chloro-4-isopropyl-6-(((trifluoromethyl)sulfonyl)oxy)quinoline-3-carboxylate and 4,4,4′,4′,5,5,5′,5′-octamethyl-2,2′-bi(1,3,2-dioxaborolane). LC-MS (M+H)⁺=404.4.

### Step 8: ethyl 7-chloro-6-(2-chloro-5-fluoropyrimidin-4-yl)-4-isopropylquinoline-3-carboxylate

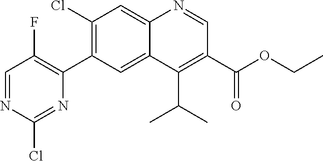 [SMILES: CCOC(=O)c1cnc2cc(Cl)c(-c3nc(Cl)ncc3F)cc2c1C(C)C]

The title compound (396 mg, 76%) was prepared in a manner similar to Example 1 step 3 from ethyl 7-chloro-4-isopropyl-6-(4,4,5,5-tetramethyl-1,3,2-dioxaborolan-2-yl)quinoline-3-carboxylate and 2,4-dichloro-5-fluoropyrimidine. LC-MS (M+H)⁺=408.1.

### Step 9: ethyl 7-chloro-6-(5-fluoro-2-(((3S,4R)-3-hydroxytetrahydro-2H-pyran-4-yl)amino)pyrimidin-4-yl)-4-isopropylquinoline-3-carboxylate

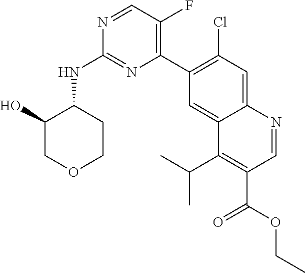 [SMILES: CCOC(=O)c1cnc2cc(Cl)c(-c3nc(N[C@@H]4CCOC[C@H]4O)ncc3F)cc2c1C(C)C]

The title compound (221 mg, 72%) was prepared in a manner similar to Example 4 step 6 from ethyl 7-chloro-6-(2-chloro-5-fluoropyrimidin-4-yl)-4-isopropylquinoline-3-carboxylate and (3S,4R)-4-aminotetrahydro-2H-pyran-3-ol hydrochloride. LC-MS (M+H)⁺=489.3.

### Step 10: (3S,4R)-4-((4-(7-chloro-3-(2-hydroxypropan-2-yl)-4-isopropylquinolin-6-yl-5-fluoropyrimidin-2-yl)amino)tetrahydro-2H-pyran-3-ol

To a solution of ethyl 7-chloro-6-(5-fluoro-2-(((3S,4R)-3-hydroxytetrahydro-2H-pyran-4-yl)amino)pyrimidin-4-yl)-4-isopropylquinoline-3-carboxylate (100 mg, 0.20 mmol) in tetrahydrofuran (4 mL) were added methyllithium (0.6 mL, 0.96 mmol, 1.6 M in tetrahydrofuran) at −40° C. Then reaction mixture was stirred at 0° C. for 2 h before diluted with ethyl acetate (30 mL). The mixture was washed with brine (20 mL), dried over sodium sulfate, filtered and concentrated under reduce pressure. The residue was purified by silica gel column chromatography, eluting with ethyl acetate in petroleum ether (50%, v/v) to give the title compound to give the title compound (12 mg, 13%). ¹H-NMR (400 MHz, DMSO-d6) δ: 9.10 (s, 1H), 8.53 (s, 1H), 8.50 (s, 1H), 8.20 (s, 1H), 7.40-7.34 (m, 1H), 5.47 (s, 1H), 4.99-4.95 (m, 1H), 4.65-4.55 (m, 1H), 3.85-3.74 (m, 3H), 3.56-3.45 (m, 1H), 3.38-3.28 (m, 1H), 3.05-2.95 (m, 1H), 2.09-1.96 (m, 1H), 1.68 (s, 6H), 1.58-1.45 (m, 7H). LC-MS (M+H)⁺=475.3.

### Example 116: (3S,4R)-4-((5-fluoro-4-(7-fluoro-3-(2-hydroxypropan-2-yl)-4-isopropylquinolin-6-yl)pyrimidin-2-yl)amino)tetrahydro-2H-pyran-3-ol

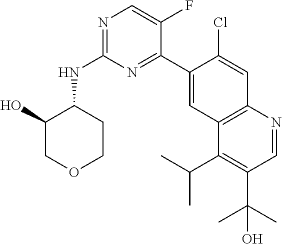 [SMILES: CC(C)c1c(C(C)(C)O)cnc2cc(Cl)c(-c3nc(N[C@@H]4CCOC[C@H]4O)ncc3F)cc12]

### Step 1: diethyl 2-(((4-chloro-3-fluorophenyl)amino)methylene)malonate

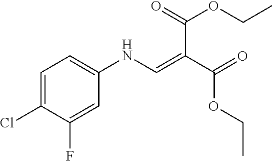 [SMILES: CCOC(=O)C(=CNc1ccc(Cl)c(F)c1)C(=O)OCC]

The title compound (26.4 g, 81%) was prepared in a manner similar to Example 51 step 1 from 4-chloro-3-fluoroaniline and diethyl 2-(ethoxymethylene)malonate. LC-MS (M+H)⁺=316.2.

### Step 2: ethyl 6-chlor-7-fluoro-4-hydroxyquinoline-3-carboxylat

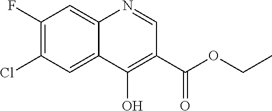 [SMILES: CCOC(=O)c1cnc2cc(F)c(Cl)cc2c1O]

The mixture of the title compound and ethyl 6-chloro-5-fluoro-4-hydroxyquinoline-3-carboxylate (9.5 g, 43%) was prepared in a manner similar to Example 51 step 2 from diethyl 2-(((4-chloro-3-fluorophenyl)amino)methylene)malonate. LC-MS (M+H)⁺=270.1.

### Step 3: ethyl 4,6-dichloro-7-fluoroquinoline-3-carboxylate and ethyl 4,6-dichloro-5-fluoroquinoline-3-carboxylate

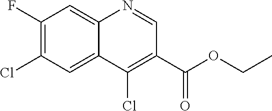 [SMILES: CCOC(=O)c1cnc2cc(F)c(Cl)cc2c1Cl]

The title compound (5.5 g, 84%) was prepared in a manner similar to Example 51 step 3 from the mixture of ethyl 6-chloro-7-fluoro-4-hydroxyquinoline-3-carboxylate and ethyl 6-chloro-5-fluoro-4-hydroxyquinoline-3-carboxylate. LC-MS (M+H)⁺=288.1.

### Step 4: ethyl 6-chloro-7-fluoro-4-isopropylquinoline-3-carboxylate

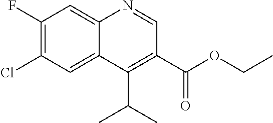 [SMILES: CCOC(=O)c1cnc2cc(F)c(Cl)cc2c1C(C)C]

The title compound (2.2 g, 66%) was prepared in a manner similar to Example 1 step 1 from ethyl 4,6-dichloro-7-fluoroquinoline-3-carboxylate. LC-MS (M+H)⁺=296.3.

### Step 5: ethyl 7-fluoro-4-isopropyl-6-44.4,5,5-tetramethyl-1,3,2-dioxaborolan-2-yl)quinoline-3-carboxylate

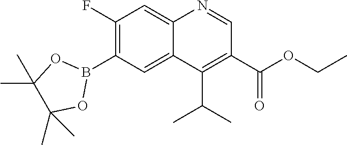 [SMILES: CCOC(=O)c1cnc2cc(F)c(B3OC(C)(C)C(C)(C)O3)cc2c1C(C)C]

The title compound (1.9 g, crude used for next step) was prepared in a manner similar to Example 1 step 2 from ethyl 6-chloro-7-fluoro-4-isopropylquinoline-3-carboxylate. LC-MS (M+H)⁺=306.3 for corresponding boronic acid.

### Step 6: ethyl 6-(2-chloro-5-fluoropyrimidin-4-yl)-7-fluoro-4-isopropylquinoline-3-carboxylate

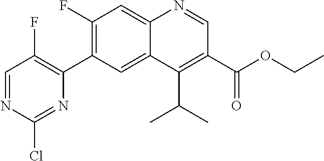 [SMILES: CCOC(=O)c1cnc2cc(F)c(-c3nc(Cl)ncc3F)cc2c1C(C)C]

The title compound (1.5 g, 52% for 2 steps) was prepared in a manner similar to Example 1 step 3 from ethyl 7-fluoro-4-isopropyl-6-(4,4,5,5-tetramethyl-1,3,2-dioxaborolan-2-yl)quinoline-3-carboxylate and 2,4-dichloro-5-fluoropyrimidine. LC-MS (M+H)⁺=392.1.

### Step 7: ethyl 7-fluoro-6-(5-fluoro-2-4-((3S,4R)-3-hydroxytetrahydro-2H-pyran-4-yl)amino)pyrimidin-4-yl-4-isopropylquinolin-3-carboxylate

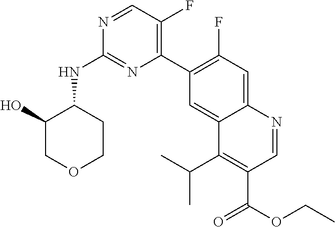 [SMILES: CCOC(=O)c1cnc2cc(F)c(-c3nc(N[C@@H]4CCOC[C@H]4O)ncc3F)cc2c1C(C)C]

The title compound (1.1 g, 61%) was prepared in a manner similar to Example 4 step 6 from ethyl 6-(2-chloro-5-fluoropyrimidin-4-yl)-7-fluoro-4-isopropylquinoline-3-carboxylate and (3S,4R)-4-aminotetrahydro-2H-pyran-3-ol. LC-MS (M+H)⁺=473.4.

### Step 8: (3S,4R)-4-((5-fluoro-4-(7-fluoro-3-(2-hydroxypropoan-2-yl)-4-isopropylquinolin-6-yl)pyrimidin-2-yl)amino)tetrahydro-2H-pyran-3-ol

The title compound (9 mg, 5%) was prepared in a manner similar to Example 70 step 10 from ethyl 7-fluoro-6-(5-fluoro-2-(((3S,4R)-3-hydroxytetrahydro-2H-pyran-4-yl)amino)pyrimidin-4-yl)-4-isopropylquinoline-3-carboxylate and methyllithium, 1H-NMR (40) MHz, DMSO-d6) δ 9.09 (s, 1H), 8.74 (s, 1H), 8.51 (s, 1H), 7.87 (d, J=11.1 Hz, 1H), 7.36 (d, J=7.3 Hz, 1H), 5.45 (s, 1H), 4.96 (s, 1H), 4.76-4.47 (m, 1H), 3.95-3.75 (m, 3H), 3.54 (s, 1H), 3.27-3.18 (m, 1H), 3.01-3.00 (m, 1H), 2.02 (s, 1H), 1.69 (s, 6H), 1.56-1.49 (m, 7H). LC-MS (M+H)⁺=459.5.

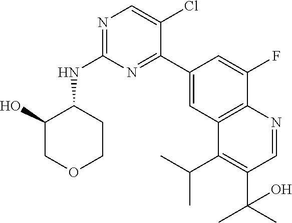 [SMILES: CC(C)c1c(C(C)(C)O)cnc2c(F)cc(-c3nc(N[C@@H]4CCOC[C@H]4O)ncc3Cl)cc12]

### Example 117: (3S,4R)-4-((5-chloro-4-(8-fluoro-3-(2-hydroxypropan-2-yl)-4-isopropylquinolin-6-yl)pyrimidin-2-yl)amino)tetrahydro-2H-pyran-3-ol

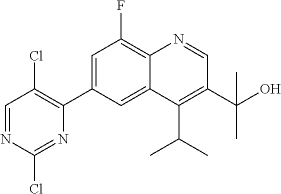 [SMILES: CC(C)c1c(C(C)(C)O)cnc2c(F)cc(-c3nc(Cl)ncc3Cl)cc12]

The title compound (50 mg, 48%) was prepared in a manner similar to Example 1 step 3 from 2-(8-fluoro-4-isopropyl-6-(4,4,5,5-tetramethyl-1,3,2-dioxaborolan-2-yl)quinolin-3-yl)propan-2-ol and 2,4,5-trichloropyrimidine. LC-MS (M+H)⁺=394.1.

### Step 2: (3S,4R)-44(5-chloro-4-(8-fluoro-3-(2-hydroxypropan-2-yl-4-isopropylquinolin-6-yl)pyrimidin-2-yl)amino)tetrahydro-2H-pyran-3-ol

The title compound (17 mg, 40%) was prepared in a manner similar to Example 4 step 6 from 2-(6-(2,5-dichloropyrimidin-4-yl)-8-fluoro-4-isopropylquinolin-3-yl)propan-2-ol and (3S,4R)-4-aminotetrahydro-2H-pyran-3-ol, 1H-NMR (400 MHz, DMSO-d₆) δ: 9.13 (s, 1H), 8.71 (br, 1H), 8.48 (s, 1H), 7.93 (br, 1H), 7.57 (d, J=8.0 Hz, 1H), 5.50 (s, 1H), 4.96 (s, 1H), 4.75-4.55 (m, 1H), 3.95-3.80 (m, 3H), 3.55-3.45 (m, 1H), 3.25-3.35 (m, 1H), 3.10-2.95 (m, 1H), 2.05-1.90 (m, 1H), 1.70 (s, 6H), 1.56 (d, J=8.0 Hz, 6H), 1.60-1.45 (m, 1H). LC-MS (M+H)⁺=475.1.

Example 117 can be obtained by the following procedures:

### Step 1: diethyl 2-(((4-chloro-2-fluorophenyl)amino)methylene)malonate

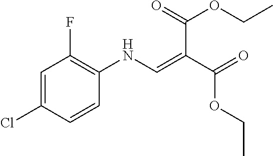 [SMILES: CCOC(=O)C(=CNc1ccc(Cl)cc1F)C(=O)OCC]

A solution of 4-chloro-2-fluoroaniline (25 g, 171.2 mmol) and diethyl 2-(ethoxymethylene)malonate (55 g) was stirred at 110° C. for 4 h. After cooled to room temperature, the reaction mixture was treated with petroleum ether (600 mL). The precipitate was filtered and dried to give the title compound (43.9 g, 81%). ¹H-NMR (400 MHz, DMSO-d6) δ 10.87 (d, J=13.3 Hz, 1H), 8.44 (d, J=13.4 Hz, 1H), 7.72-7.67 (m, 1H), 7.60 (d, J=10.9 Hz, 1H), 7.33 (d, J=8.7 Hz, 1H), 4.22 (q, J=6.9 Hz, 2H), 4.14 (q, J=6.9 Hz, 2H), 1.33-1.19 (m, 6H). LC-MS (M+H)⁺=316.2.

### Step 2: ethyl 6-chloro-8-fluoro-4-hydroxyquinoline-3-carboxylate

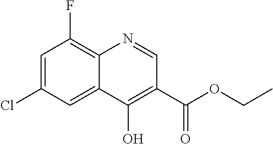 [SMILES: CCOC(=O)c1cnc2c(F)cc(Cl)cc2c1O]

The reaction mixture of diethyl 2-(((4-chloro-2-fluorophenyl)amino)methylene)malonate (34.6 g, 109.5 mmol) and oxydibenzene (50 mL) was stirred at 260° C. for 9 h. After cooled to room temperature, the mixture was treated with petroleum ether (60(0 mL). The precipitate was filtered and dried to give the title product (43.9 g, 81%). ¹H-NMR (400 MHz, DMSO-d6) δ 12.65 (s, 1H), 8.39 (s, 1H), 7.91 (d, J=9.8 Hz, 2H), 4.23 (q, J=6.8 Hz, 2H), 1.28 (t, J=7.0 Hz, 3H). LC-MS (M+H)⁺=270.1.

### Step 3: ethyl 4-bromo-6-chloro-8-fluoroquinoline-3-carboxylate

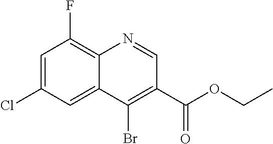 [SMILES: CCOC(=O)c1cnc2c(F)cc(Cl)cc2c1Br]

To the solution of ethyl 6-chloro-8-fluoro-4-hydroxyquinoline-3-carboxylate (5.5 g, 20.4 mmol) in dimethylformamide (80 mL) was added phosphorus tribromide (11 g, 40.8 mmol) and the mixture was stirred at 80° C. for 4 h. The mixture was cooled to room temperature and poured to ice water. The precipitate was filtered and dissolved with ethyl acetate. The mixture was washed with water and brine, dried over sodium sulfate, filtered and concentrated. The residue was purified by column chromatography eluting with ethyl acetate in petroleum ether (16%, v/v*) to give the title compound (5.2 g, 76%). ¹H-NMR (400 MHz, DMSO-d₆) δ 9.09 (s, 1H), 8.15 (s, 1H), 8.10 (d, J=10.0 Hz, 1H), 4.46 (q, J=7.0 Hz, 2H), 1.39 (t, J=7.0 Hz, 3H). LC-MS (M+H)⁺=332.0, 334.0.

### Step 4: 2-(4-bromo-6-chloro-8-fluoroquinolin-3-yl)propan-2-ol

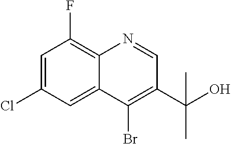 [SMILES: CC(C)(O)c1cnc2c(F)cc(Cl)cc2c1Br]

To a solution of ethyl 4-bromo-6-chloro-8-fluoroquinoline-3-carboxylate (333 mg, 1 mmol) in tetrahydrofuran (8 mL) under nitrogen at −78° C. was added methylmagnesium bromide (3 mL, 3 mmol, 1M in tetrahydrofuran) and the mixture was slowly warmed to 0° C. in 2 h before quenched by saturated aqueous solution of ammonium chloride. The aqueous layer was extracted with ethyl acetate 3 times. The combined organic layers were washed with brine, dried over sodium sulfate, filtered and concentrated. The residue was purified by column chromatography, eluting with ethyl acetate in petroleum ether (33%, v/v) to give the title compound (240 mg, 75%). LC-MS (M+H)⁺=318.0, 320.0.

### Step 5: 2-(6-chloro-8-fluoro-4-isopropyl)quinolin-3-yl)propan-2-ol

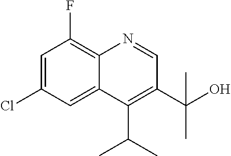 [SMILES: CC(C)c1c(C(C)(C)O)cnc2c(F)cc(Cl)cc12]

To a solution of isopropylmagnesium bromide (3 mL, 1M in tetrahydrofuran, 3 mmol) under nitrogen was added zinc chloride (3 mL, 1 M in tetrahydrofuran, 3 mmol) and the mixture was stirred at 50° C. for 2 h before addition of 2-(4-bromo-6-chloro-8-fluoroquinolin-3-yl)propan-2-ol (240 mg, 0.75 mmol) in N,N-dimethylformamide (10 mL). CuI (14 mg, 0.075 mmol) and (1,1′-bis(diphenylphosphino)ferrocene)palladium(II) dichloride (28 mg, 0.038 mmol). The reaction mixture was stirred at 50° C. for 2 h before cooled to room temperature. The reaction was quenched with methanol (10 mL) and the solvents were concentrated. The residue was diluted with ethyl acetate, and the organic solution was washed with water and saturated aqueous solution of ammonium chloride. The aqueous phase was extracted with ethyl acetate 3 times. The organic layers were combined, washed with water and brine, dried over sodium sulfate, filtered and evaporated. The residue was purified by column chromatography, eluting with ethyl acetate in petroleum ether (50%, v/v) to give the title compound (75 mg, 35%). LC-MS (M+H)⁺=282.2.

### Step 6: 2-(8-fluoro-4-isopropyl-6-(4,4,5,5-tetramethyl-1,3,2-dioxaborolan-2-yl)quinolin-3-yl)propan-2-ol

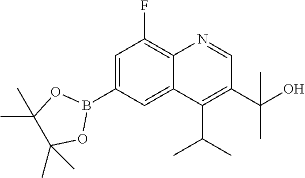 [SMILES: CC(C)c1c(C(C)(C)O)cnc2c(F)cc(B3OC(C)(C)C(C)(C)O3)cc12]

A mixture of 2-(6-chloro-8-fluoro-4-isopropylquinolin-3-yl)propan-2-ol (75 mg, 0.265 mmol) and bis(pinacolato)diboron (94 mg, 0.37 mmol), (1,1′-bis(diphenylphosphino)ferrocene)palladium(II) dichloride (9 mg, 0.013 mmol) and potassium acetate (39 mg, 0.4 mmol) in 1,4-dioxane (2.5 mL) was stirred at 80° C. under nitrogen for overnight before cooled to room temperature. The mixture was filtered and the filtrate was evaporated to give the title compound (99 mg, 100%). LC-MS (M+H)⁺=292.2 for corresponding boronic acid.

### Step 7: 2-(6-(2,5-dichloropyrimidin-4-yl-8-fluoro-4-isopropylquinolin-3-yl)propan-2-ol

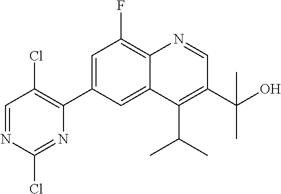 [SMILES: CC(C)c1c(C(C)(C)O)cnc2c(F)cc(-c3nc(Cl)ncc3Cl)cc12]

A mixture of 2-(8-fluoro-4-isopropyl-6-(4,4,5,5-tetramethyl-1,3,2-dioxaborolan-2-yl)quinolin-3-yl)propan-2-ol (99 mg, 0.264 mmol), 2,4,5-trichloropyrimidine (97 mg, 0.53 mmol), (1,1′-bis(diphenylphosphino)ferrocene)palladium(II) dichloride (19 mg, 0.026 mmol) and potassium carbonate (0.14 g, 1 mmol) in 1,4-dioxane (4 mL) and water (0.5 mL) under nitrogen was stirred at 80° C. for overnight before cooled to room temperature. The mixture was diluted with ethyl acetate, washed with brine, dried over sodium sulfate, filtered and evaporated. The residue was purified over silica gel by combi-flash, eluting with methanol in dichloromethane (3%, v/v) to give the title compound (50 mg, 48%). LC-MS (M+H)⁺=394.1.

### Step 8: (3S,4R)-44(5-chloro-4-(8-fluoro-3-(2-hydroxypropan-2-yl)-4-isopropylquinolin-6-yl)pyrimidin-2-yl)amino)tetrahydro-2H-pyran-3-ol

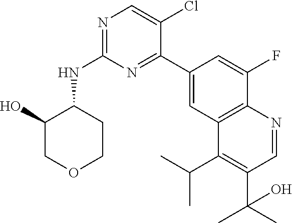 [SMILES: CC(C)c1c(C(C)(C)O)cnc2c(F)cc(-c3nc(N[C@@H]4CCOC[C@H]4O)ncc3Cl)cc12]

2-(6-(2,5-dichloropyrimidin-4-yl)-8-fluoro-4-isopropylquinolin-3-yl)propan-2-ol (35 mg, 0.089 mmol) and N,N-diisopropylethylamine (35 mg, 0.27 mmol) were dissolved in acetonitrile (1 mL). (3S,4R)-4-aminotetrahydro-2H-pyran-3-ol (20 mg, 0.17 mmol) was added and the mixture was stirred at 80° C. for 36 h before cooled to room temperature. Solvent was removed under vacuum before addition of water (2 mL). Aqueous layer was extracted with ethyl acetate (3 mL×3). The combined organic layers were washed with brine, dried over sodium sulfate, filtered and concentrated. The crude was purified by flash chromatography over silica gel to give the title product (17 mg, 40%), 1H-NMR (400 MHz, DMSO-do) S: 9.13 (s, 1H), 8.71 (br, 1H), 8.48 (s, 1H), 7.93 (br, 1H), 7.57 (d, J=8.0 Hz, 1H), 5.50 (s, 1H), 4.96 (s, 1H), 4.75-4.55 (m, 1H), 3.95-3.80 (m, 3H), 3.55-3.45 (m, 1H), 3.25-3.35 (m, 1H), 3.10-2.95 (m, 1H), 2.05-1.90 (m, 1H), 1.70 (s, 6H), 1.56 (d, J=8.0 Hz, 6H), 1.60-1.45 (m, 1H). LC-MS (M+H)⁺=475.1.

### Example 120: (3S,4R)-4-((5-fluoro-4-(3-(hydroxymethyl)-4-isopropyl-5-methylquinolin-6-yl)pyrimidin-2-yl)amino)tetrahydro-2H-pyran-3-ol

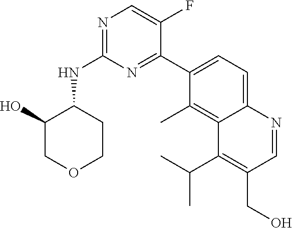 [SMILES: Cc1c(-c2nc(N[C@@H]3CCOC[C@H]3O)ncc2F)ccc2ncc(CO)c(C(C)C)c12]

### Step 1: 4-chloro-2-iodo-5-methylaniline

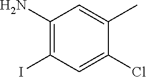

4-chloro-3-methylaniline (12.7 g, 89.69 mmol) was dissolved in acetic acid (150 mL). N-Iodosuccinimide (20.2 g, 89.69 mmol) was added at 0° C. and the reaction solution was stirred at room temperature for 14 h. Acetic acid was removed by reduced pressure before addition of water (50 mL) and ethyl acetate (200 mL). Aqueous saturated sodium bicarbonate solution was added until pH=8. The aqueous layer was separated from organic layer and then extracted with ethyl acetate 3 times. The combined organic layers were washed with brine, dried over sodium sulfate, filtered and concentrated. The residue was purified over silica gel by combi-flash to give the title compound (23.35 g, 97%). LC-MS (M+H)⁺=268.0.

### Step 2: ethyl 6-chloro-4-hydroxy-8-iodo-5-methylquinoline-3-carboxylate

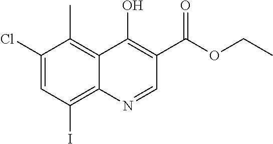 [SMILES: CCOC(=O)c1cnc2c(I)cc(Cl)c(C)c2c1O]

A mixture of 4-chloro-2-iodo-5-methylaniline (14.35 g, 53.65 mmol) and diethyl 2-(ethoxymethylene)malonate (12.2 g, 56.33 mmol) was stirred to 120° C. for 4 h. Diphenyl ether (60 mL) was added and resulting mixture was heated to 260° C. for 3 h before cooled to 60° C., Ethyl acetate (100 mL) was added and the resulting slurry was stirred at room temperature for 2 h. The title compound (10.5 g, 50%) was obtained by filtration. LC-MS (M+H)⁺=392.0.

### Step 3: ethyl 6-chloro-4-hydroxy-5-methylquinoline-3-carboxylate

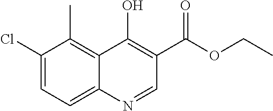 [SMILES: CCOC(=O)c1cnc2ccc(Cl)c(C)c2c1O]

A solution of ethyl 6-chloro-4-hydroxy-8-iodo-5-methylquinoline-3-carboxylate (1.76 g, 4.49 mmol), sodium formate (1.53 g, 22.47 mmol) and tetrakis(triphenylphosphine)palladium(0) (259.7 mg, 0.2247 mmol) in N,N-dimethylformamide (20 mL) was stirred at 120° C. under nitrogen atmosphere for 1 h before cooled to room temperature followed by addition of water (50 mL). The precipitate was filtered, washed with water and ethyl acetate to give the title compound (1.224 g, 100%). LC-MS (M+H)⁺=266.0.

### Step 4: ethyl 4,6-dichloro-5-methylquinoline-3-carboxylate

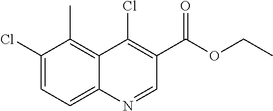 [SMILES: CCOC(=O)c1cnc2ccc(Cl)c(C)c2c1Cl]

Ethyl 6-chloro-4-hydroxy-5-methylquinoline-3-carboxylate (1.224 g, 4.61 mmol) was dissolved in dry dichloromethane (20 mL) and N,N-dimethylformamide (0.1 mL). Oxalyl dichloride (643.2 mg, 5.07 mmol) was added dropwise at 35° C. and the reaction solution was stirred at 40° C. for overnight before cooled to 0° C. Saturated sodium bicarbonate solution was added until pH=8 and aqueous layer was separated from the organic layer. The aqueous layer was extracted with dichloromethane 3 times. The combine organic layers were washed with brine, dried over sodium sulfate, filtered and concentrated. The residue was purified over silica gel by combi-flash to give the title compound (1.31 g, 100%). LC-MS (M+H)⁺=284.0.

### Step 5: ethyl 6-chlor-4-isopropyl-5-methylquinoline-carboxylate

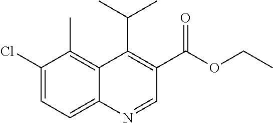 [SMILES: CCOC(=O)c1cnc2ccc(Cl)c(C)c2c1C(C)C]

Zinc chloride solution in tetrahydrofuran (1N, 6.92 mL, 6.92 mmol) was added to isopropylmagnesium chloride solution in tetrahydrofuran (6.92 mL, 1N, 6.92 mmol) at 0° C. and the reaction mixture was heated to 50° C. under nitrogen for 1 h before cooled to 0° C. Ethyl 4,6-dichloro-5-methylquinoline-3-carboxylate (1.31 g, 4.61 mmol), CuI (87.8 mg, 0.461 mmol) and (1,1′-bis(diphenylphosphino)ferrocene)palladium(II) dichloride (168.7 mg, 0.231 mmol) were added and the resulting mixture was heated to 50° C. under nitrogen for 2 h before cooled to 0° C. Saturated ammonium chloride solution (20 mL) was added and the aqueous layer was extracted with ethyl acetate 3 times. The combined organic layers were washed with brine, dried over sodium sulfate, filtered and concentrated. The residue was purified over silica gel by combi-flash to give the title compound (1.35 g, 100%). LC-MS (M+H)⁺=292.0.

### Step 6: 6-chloro-4-isopropyl-5-methylquinoline-3-carboxylic acid

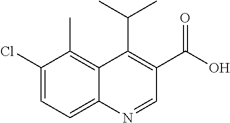 [SMILES: Cc1c(Cl)ccc2ncc(C(=O)O)c(C(C)C)c12]

To a solution of ethyl 6-chloro-4-isopropyl-5-methylquinoline-3-carboxylate (20) mg, 0.686 mmol) in methanol (3 mL) was added potassium hydroxide solution (2N, 1.02 mL, 2.04 mmol). The resulting mixture was stirred at 60° C. for overnight before cooled to room temperature and concentrated. Water (2 mL) was added and 2 N HCl solution was added until pH=6. The aqueous layer was extracted with ethyl acetate 3 times. The combined organic layers were washed with brine, dried over sodium sulfate, filtered and concentrate. The residue was purified over silica gel by combi-flash to give the title compound (180 mg, 99.6%). LC-MS (M+H)⁺=264.0.

### Step 7: (6-chloro-4-isopropyl-5-methylquinolin-3-yl(1H-imidazol-1H-1-yl)methanone

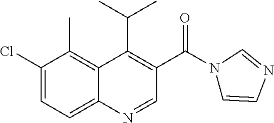 [SMILES: Cc1c(Cl)ccc2ncc(C(=O)n3ccnc3)c(C(C)C)c12]

To a solution of 6-chloro-4-isopropyl-5-methylquinoline-3-carboxylic acid (60 mg, 0.228 mmol) in tetrahydrofuran was added carbonyl diimidazole (73.81 mg, 0.455 mmol) and the reaction mixture was stirred at room temperature for overnight before addition of water at 0° C. The aqueous layer was extracted with ethyl acetate 3 times. The combined layers were washed with brine, dried over sodium sulfate, filtered and concentrated. The residue was purified over silica gel by combi-flash to give the title compound (71.4 mg, 100%). LC-MS (M+H)⁺=314.0.

### Step 8: (6-chloro-4-isopropyl-5-methylquinolin-3-yl)methanol

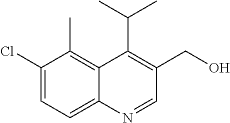 [SMILES: Cc1c(Cl)ccc2ncc(CO)c(C(C)C)c12]

To a solution of (6-chloro-4-isopropyl-5-methylquinolin-3-yl)(1H-imidazol-1-yl)methanone (71.4 mg, 0.228 mmol) in tetrahydrofuran was added lithium aluminium hydride (9.5 mg, 0.250 mmol) at 0° C. and the reaction mixture was stirred at 0° C. for 1 h, then room temperature for overnight. Sodium sulfate decahydrate was added to reaction mixture at 0° C. and the mixture was stirred for 30 min before addition of ethyl acetate. The mixture was stirred at room temperature for another 1 h before filtration. The filtrate was concentrated and the residue was purified over silica gel by combi-flash to give the title compound (25.6 mg, 45%). LC-MS (M+H)⁺=250.0.

### Step 9: 3-(((tert-butyldimethylsilyloxy)methyl)-6-chloro-4-isopropyl-5-methylquinoline

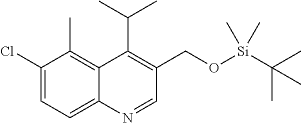 [SMILES: Cc1c(Cl)ccc2ncc(CO[Si](C)(C)C(C)(C)C)c(C(C)C)c12]

To a solution of (6-chloro-4-isopropyl-5-methylquinolin-3-yl)methanol (184 mg, 0.737 mmol) and 1H-imidazole (88.53 mg, 1.474 mmol) in dichloromethane was added tert-butyldimethylsilyl chloride (166.6 mg, 1.105 mmol) at 0° C. and the reaction solution was stirred at room temperature for overnight before addition of water. The aqueous layer was extracted with dichloromethane 3 times. The combined layers were washed with brine, dried over sodium sulfate, filtered and concentrated. The residue was purified over silica gel by combi-flash to give the title compound (212 mg, 79%). LC-MS (M+H)⁺=364.0.

### Step 10: 3-(((tert-butyldimethylsilyl)oxy)methyl)-6-(2-chloro-5-fluoropyrimidin-4-yl)-4-isopropyl-5-methylquinoline

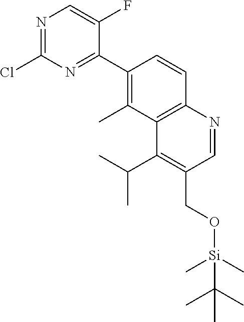 [SMILES: Cc1c(-c2nc(Cl)ncc2F)ccc2ncc(CO[Si](C)(C)C(C)(C)C)c(C(C)C)c12]

A mixture of 3-(((tert-butyldimethylsilyl)oxy)methyl)-6-chloro-4-isopropyl-5-methylquinoline (212 mg, 0.583 mmol), 4,4,4′,4′,5,5,5′,5′-octamethyl-2,2′-bi(1,3,2-dioxaborolane) (295.8 mg, 1.165 mmol), potassium acetate (171.5 mg, 1.75 mmol) and dichlorobis(tricyclohexylphosphine)palladium (43 mg, 0.0583 mmol) in dry 1,4-dioxane (10 mL) was stirred at 110° C. under nitrogen for overnight before cooled to room temperature. Potassium carbonate (241 mg, 1.75 mmol) in water (3 mL), (1,1′-bis(diphenylphosphino)ferrocene)palladium(II) dichloride (42.6 mg, 0.0582 mmol) and 2,4-dichloro-5-fluoropyrimidine (291.8 mg, 1.75 mmol) were added and the mixture was heated to 70° C. for 2 h before cooled to room temperature and concentrated. Water (2 mL) was added and the aqueous layer was extracted with ethyl acetate 3 times. The combined organic layers were washed with brine, dried over sodium sulfate, filtered and concentrated. The residue was purified over silica gel by combi-flash to give the title compound (88 mg, 32.8%). LC-MS (M+H)⁺=460.0.

### Step 11: (3S,4R)-4-((4-(3-(((tert-butyldimethylsilyl)oxymethyl)-4-isopropyl-5-methylquinolin-6-yl)-5-fluoropyrimidin-2-yl)amino)tetrahydro-2H-pyran-3-ol

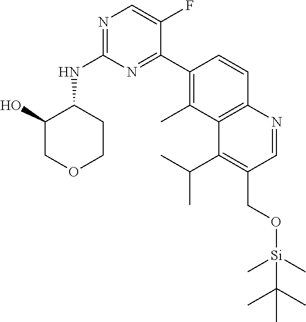 [SMILES: Cc1c(-c2nc(N[C@@H]3CCOC[C@H]3O)ncc2F)ccc2ncc(CO[Si](C)(C)C(C)(C)C)c(C(C)C)c12]

The title compound (103 mg, 100%) was prepared in a manner similar to Example 1 step 4 from 3-(((tert-butyldimethylsilyl)oxy)methyl)-6-(2-chloro-5-fluoropyrimidin-4-yl)-4-isopropyl-5-methylquinoline and (3S,4R)-4-aminotetrahydro-2H-pyran-3-olhydrogen chloride. LC-MS (M+H)⁺=541.2.

### Step 12: (3S,4R)-4-((5-fluoro-4-(3-(hydroxymethyl)-4-isopropyl-5-methylquinolin-6-yl)pyrimidin-2-yl)amino)tetrahydro-2H-pyran-3-ol

To a solution of (3S,4R)-4-((4-(3-(((tert-butyldimethylsilyl)oxy)methyl)-4-isopropyl-5-methylquinolin-6-yl)-5-fluoropyrimidin-2-yl)amino)tetrahydro-2H-pyran-3-ol (103.43 mg, 0.191 mmol) in tetrahydrofuran (1 mL) was added tetrabutylammonium fluoride in tetrahydrofuran (1N, 0.57 mL, 0.57 mmol) and the reaction solution was stirred at room temperature for 2 h before addition of water (5 mL). The aqueous layer was extracted with ethyl acetate 3 times. The combined organic layers were washed with brine, dried over sodium sulfate, filtered and concentrated. The residue was purified by prep-HPLC to give the title compound (46 mg, 56%). ¹H-NMR (400 MHz, DMSO-d6) δ 8.90 (s, 1H), 8.48 (s, 1H), 7.87 (d, J=8.5 Hz, 1H), 7.60 (d, J=8.6 Hz, 1H), 7.29 (d, J=7.5 Hz, 1H), 5.45 (t, J=5.4 Hz, 1H), 4.94 (s, 1H), 4.90 (s, 2H), 4.10-3.95 (m, 1H), 3.85-3.70 (m, 3H), 3.55-3.40 (m, 1H), 3.30 (s, 1H), 3.04 (t, J=10.3 Hz, 1H), 2.64 (s, 3H), 1.98 (d, J=10.8 Hz, 1H), 1.55-1.35 (m, 1H), 1.43 (d, J=6.4 Hz, 6H). LC-MS (M+H)⁺=427.0.

### Example 145: (3S,4R)-4-((4-(3aminomethyl)-4-isopropyl-5-methylquinolin-6-yl)-5-fluoropyrimidin-2-yl)amino)tetrahydro-2H-pyran-3-ol

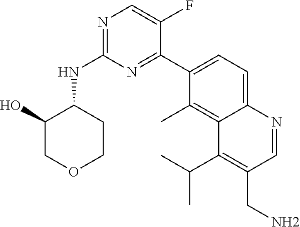 [SMILES: Cc1c(-c2nc(N[C@@H]3CCOC[C@H]3O)ncc2F)ccc2ncc(CN)c(C(C)C)c12]

### Step 1: (3S,4R)-4-((4-(3-chloromethyl)-4-isopropyl-5-methylquinolin-6-yl)-5-fluoropyrimidin-2-yl)amino)tetrahydro-2H-pyran-3-ol

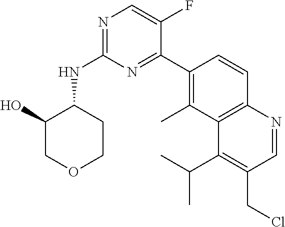 [SMILES: Cc1c(-c2nc(N[C@@H]3CCOC[C@H]3O)ncc2F)ccc2ncc(CCl)c(C(C)C)c12]

To (3S,4R)-4-((5-fluoro-4-(3-(hydroxymethyl)-4-isopropyl-5-methylquinolin-6-yl)pyrimidin-2-yl)amino)tetrahydro-2H-pyran-3-ol (349 mg, 0.818 mmol) in dichloromethane (20 mL) was added thionyl chloride (116.8 mg, 0.982 mmol) at 0° C. and the reaction mixture was stirred at room temperature for 3 h. Saturated sodium bicarbonate solution was added until pH=8 and the aqueous layer was extracted with dichloromethane 3 times. The combined organic layers were washed with brine, dried over sodium sulfate, filtered and concentrated. The title compound (364 mg, 100%) was obtained. LC-MS (M+H)⁺=445.0.

### Step 2: (3S,4R)-4-((4-(3-(aminomethyl)-4-isopropyl-5-methylquinolin-6-yl)-5-fluoropyrimidin-2-yl)amino)tetrahydro-2H-pyran-3-ol

A solution of (3S,4R)-4-((4-(3-(chloromethyl)-4-isopropyl-5-methylquinolin-6-yl)-5-fluoropyrimidin-2-yl)amino)tetrahydro-2H-pyran-3-ol (24 mg, 0.054 mmol) and 7 N ammonia in methanol (5 mL) in sealed tube was stirred at 50° C. for overnight before cooled to room temperature and concentrated. The residue was purified by prep-HPLC to give the title compound (6 mg, 25%). ¹H-NMR (400 MHz, DMSO-d6) δ 8.92 (s, 1H), 8.47 (d, J=1.7 Hz, 1H), 8.26 (s, 1H), 7.87 (d, J=8.5 Hz, 1H), 7.60 (d, J=8.5 Hz, 1H), 7.30 (d, J=7.7 Hz, 1H), 5.75-4.25 (br s, 2H), 4.19 (s, 2H), 4.05-3.90 (m, 1H), 3.85-3.70 (m, 3H), 3.55-3.45 (m, 1H), 3.32 (t, J=11.2 Hz, 1H), 3.04 (t, J=10.4 Hz, 1H), 2.62 (s, 3H), 1.98 (d, J=10.6 Hz, 1H), 1.57-1.31 (m, 1H), 1.44 (d, J=8.0 Hz, 3H), 1.44 (d, J=8.0 Hz, 3H). LC-MS (M+H)⁺=426.0.

### Example 206: (3S,4R)-4-((5-fluoro-4-(2-(2-hydroxypropan-2-yl)-4-isopropylquinolin-6-yl)pyrimidin-2-yl)amino)tetrahydro-2H-pyran-3-ol

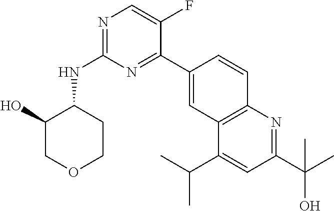 [SMILES: CC(C)c1cc(C(C)(C)O)nc2ccc(-c3nc(N[C@@H]4CCOC[C@H]4O)ncc3F)cc12]

### Step 1: dimethyl 2-((4-chlorophenyl)amino)fumarate

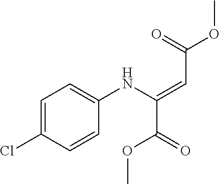 [SMILES: COC(=O)/C=C(\Nc1ccc(Cl)cc1)C(=O)OC]

To a solution of 4-chloroaniline (3.0 g, 23.5 mmol) in ethanol (40 mL) was added dimethyl but-2-ynedioate (3.4 g, 23.5 mmol). The mixture was stirred at room temperature for 15 h before concentrated under vacuum. The title compound (6.5 g crude) was obtained. LC-MS (M+H)⁺=270.1.

### Step 2: methyl 6-chloro-4-oxo-1,4-dihydroquinoline-2-carboxylate

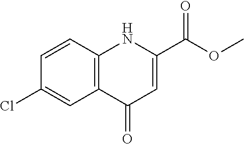 [SMILES: COC(=O)c1cc(=O)c2cc(Cl)ccc2[nH]1]

A mixture of dimethyl 2-((4-chlorophenyl)amino)fumarate (5.5 g, 20 mmol) and polyphosphoric acid (30 mL) was stirred at 100° C. for 1 h before cooled to room temperature. The mixture was diluted with 0° C. water (300 mL) and stirred for 1 h. The precipitate was collected by filtration and dried under vacuum to give the title compound (3.0 g, 63%). LC-MS (M+H)⁺=238.1.

### Step 3: methyl 4,6-dichloroquinoline-2-carboxylate

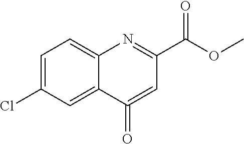

A mixture of methyl 6-chloro-4-oxo-1,4-dihydroquinoline-2-carboxylate (3.0 g, 12.6 mmol) and phosphorus oxychloride (30 mL) was stirred at 100° C. for 8 h. The mixture was cooled to room temperature and poured into ice water (200 mL). Then the mixture was stirred for 1 h before filtration. The precipitate was dried under vacuum to give the title compound (2.4 g, 74%). LC-MS (M+H)⁺=256.1.

### Step 4: methyl 6-chloro-4-(prop-1-en-2-yl)quinoline-2-carboxylate

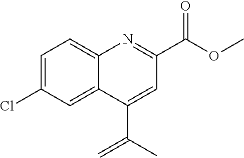 [SMILES: C=C(C)c1cc(C(=O)OC)nc2ccc(Cl)cc12]

The title compound (1.5 g, 73%) was prepared in a manner similar to Example 1 step 3 from methyl 4,6-dichloroquinoline-2-carboxylate (2.0 g, 7.8 mmol) and potassium isopropenyltrifluoroborate. LC-MS (M+H)⁺=262.2.

### Step 5: methyl 4-(prop-1-en-2-yl)-6-(4,4,5,5-tetramethyl-1,3,2-dioxaborolan-2-yl)quinoline-2-carboxylate

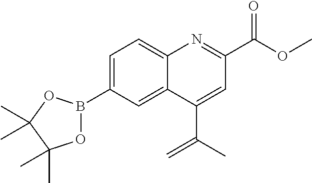

The title compound (1.2 g, 59%) was prepared in a manner similar to Example 1 step 2 from methyl 6-chloro-4-(prop-1-en-2-yl)quinoline-2-carboxylate and 4,4,4′,4′,5,5,5′,5′-octamethyl-2,2′-bi(1,3,2-dioxaborolane). LC-MS (M+H)⁺=354.2.

### Step 6: methyl 4-isopropyl-6-(4,4,5,5-tetramethyl-1,3,2-dioxaborolan-2-yl)quinoline-2-carboxylate

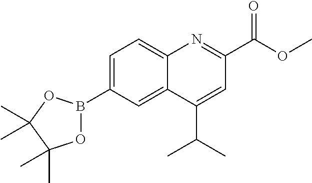

To a solution of methyl 4-(prop-1-en-2-yl)-6-(4,4,5,5-tetramethyl-1,3,2-dioxaborolan-2-yl)quinoline-2-carboxylate (1.0 g, 3.8 mmol) in methanol (30 mL) was added palladium 10% on carbon powder (100 mg). The mixture was stirred under hydrogen atmosphere (1 atm) overnight. The mixture was filtered and concentrated to give the title compound (900 mg, 90%). LC-MS (M+H)⁺=356.2.

### Step 7: methyl 6-(2-chloro-5-fluoropyrimidin-4-yl)-4-isopropylquinoline-2-carboxylate

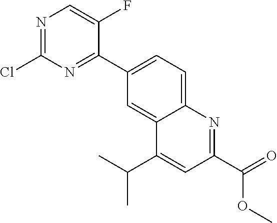 [SMILES: COC(=O)c1cc(C(C)C)c2cc(-c3nc(Cl)ncc3F)ccc2n1]

The title compound (500 mg, 52%) was prepared in a manner similar to Example 1 step 3 from methyl 4-isopropyl-6-(4,4,5,5-tetramethyl-1,3,2-dioxaborolan-2-yl)quinoline-2-carboxylate and 2,4-dichloro-5-fluoropyrimidine. LC-MS (M+H)⁺=360.1.

### Step 8: methyl 6-(5-fluoro-2-(((3S,4R)-3-hydroxytetrahydro-2H-pyran-4-yl)amino)pyrimidin-4-yl)-4-isopropylquinoline-2-carboxylate

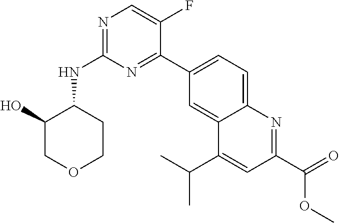 [SMILES: COC(=O)c1cc(C(C)C)c2cc(-c3nc(N[C@@H]4CCOC[C@H]4O)ncc3F)ccc2n1]

The title compound (200 mg, 32%) was prepared in a manner similar to Example 1 step 4 from methyl 6-(2-chloro-5-fluoropyrimidin-4-yl)-4-isopropylquinoline-2-carboxylate and (3S,4R)-4-aminotetrahydro-2H-pyran-3-ol. LC-MS (M+H)⁺=441.2.

### Step 9: (3S,4R)-4-((5-fluoro-4-(2-(2-hydroxypropan-2-yl)-4-isopropylquinolin-6-yl)pyrimidin-2-yl)amino)tetrahydro-2H-pyran-3-ol

To a solution of methyl 6-(5-fluoro-2-(((3S,4R)-3-hydroxytetrahydro-2H-pyran-4-yl)amino)pyrimidin-4-yl)-4-isopropylquinoline-2-carboxylate (100 mg, 0.23 mmol) in tetrahydrofuran (5 mL) was added a solution of methylmagnesium iodide (3M in tetrahydrofuran, 0.4 mL, 1.2 mmol) dropwise at −78° C. The mixture was stirred at −78° C. for 1 h. The mixture was diluted with water (10 ml), extracted with ethyl acetate (3×10 mL). The combined organic layers were washed with brine (20 mL), dried over sodium sulfate, filtered and concentrated under vacuum. The residue was purified by TLC eluting with methanol in dichloromethane (5%, v/v) to give the title compound (10 mg, 10%). ¹H-NMR (400 MHz, DMSO-d6) δ 8.84 (s, 1H), 8.49 (d, J=3.7 Hz, 1H), 8.28 (d, J=8.8 Hz, 1H), 8.10 (d, J=8.8 Hz, 1H), 7.87 (s, 1H), 7.28 (d, J=7.4 Hz, 1H), 5.49 (s, 1H), 4.97 (d, J=5.2 Hz, 1H), 3.92-3.73 (m, 4H), 3.59-3.49 (m, 1H), 3.41-3.30 (m, 1H), 3.11-3.02 (m, 1H), 2.10-1.98 (m, 1H), 1.55 (s, 6H), 1.62-1.46 (m, 1H), 1.45-1.38 (m, 6H). LC-MS (M+H)⁺=441.6.

### Example 207: (3S,4R)-4-((5-fluoro-4-(2-(2-hydroxypropan-2-yl)-4-isopropylquinazolin-6-yl)pyrimidin-2-yl)amino)tetrahydro-2H-pyran-3-ol

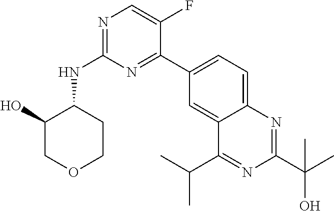 [SMILES: CC(C)c1nc(C(C)(C)O)nc2ccc(-c3nc(N[C@@H]4CCOC[C@H]4O)ncc3F)cc12]

### Step 1: ethyl 2-((2-carbamoyl-4-chlorophenyl)amino)-2-oxoacetate

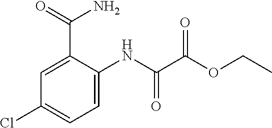 [SMILES: CCOC(=O)C(=O)Nc1ccc(Cl)cc1C(N)=O]

To a solution of 2-amino-5-chlorobenzamide (10 g, 58.6 mmol) and triethylamine (18 g, 178 mmol) in tetrahydrofuran (170 mL) was added a solution of ethyl 2-chloro-2-oxoacetate (10 g, 73.3 mmol) in tetrahydrofuran (30 mL) dropwise at 0° C. The mixture was stirred for 1 h at 0° C. The mixture was diluted with water (200 mL) and extracted with ethyl acetate (3×300 mL). The combined organic layers were washed with brine, dried over sodium sulfate, filtered and concentrated under vacuum to give the title compound (15 g, 93%). LC-MS (M+H)⁺=271.2.

### Step 2: methyl 6-chloro-4-oxo-1,4-dihydroquinazoline-2-carboxylate

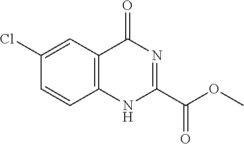 [SMILES: COC(=O)c1nc(=O)c2cc(Cl)ccc2[nH]1]

To a solution of ethyl 2-((2-carbamoyl-4-chlorophenyl)amino)-2-oxoacetate (15 g, 55 mmol) in methanol (500 mL) was added sodium ethoxide (15 g, 221 mmol). The mixture was stirred at room temperature for 15 h before the solvent was removed under vacuum. The residue was treated with hydrochloride solution (1 M, 500 mL) and the mixture was stirred at room temperature for 30 min. The precipitate was collected by filtration, washed with water (200 mL) and dried under vacuum at 50° C. for 2 h to give the title compound (10 g, 72%). LC-MS (M+H)⁺=239.2.

### Step 3: methyl 4,6-dichloroquinazoline-2-carboxylate

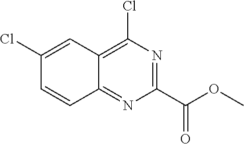 [SMILES: COC(=O)c1nc(Cl)c2cc(Cl)ccc2n1]

The title compound (50) mg, 46%) was prepared in a manner similar to Example 1 step 3 from methyl 6-chloro-4-oxo-1,4-dihydroquinazoline-2-carboxylate. LC-MS (M+H)⁺=257.2.

### Step 4: methyl 6-chloro-4-(prop-1-en-2-yl)quinazoline-2-carboxylate

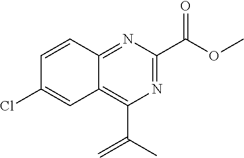 [SMILES: C=C(C)c1nc(C(=O)OC)nc2ccc(Cl)cc12]

The title compound (2.0 g, 46%) was prepared in a manner similar to Example 206 step 3 from methyl 4,6-dichloroquinazoline-2-carboxylate and potassium isopropenyltrifluoroborate, potassium salt. LC-MS (M+H)⁺=263.2.

### Step 5: methyl 4-(prop-1-en-2-yl)-6-(4,4,5,5-tetramethyl-1,3,2-dioxaborolan-2-yl)quinazoline-2-carboxylate

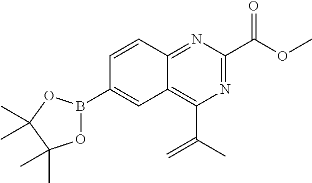

The title compound (1.2 g, 59%) was prepared in a manner similar to Example 1 step 2 from methyl 6-chloro-4-(prop-1-en-2-yl)quinazoline-2-carboxylate and 4,4,4′,4′,5,5,5′,5′-octamethyl-2,2′-bi(1,3,2-dioxaborolane). LC-MS (M+H)⁺=355.2.

### Step 6: methyl 4-isopropyl-6-(4,4,5,5-tetramethyl-1,3,2-dioxaborolan-2-yl)quinazoline-2-carboxylate

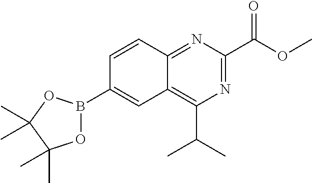

The title compound (1.0 g, 90%) was prepared in a manner similar to Example 206 step 6 from methyl 4-(prop-1-en-2-yl)-6-(4,4,5,5-tetramethyl-1,3,2-dioxaborolan-2-yl)quinazoline-2-carboxylate. LC-MS (M+H)⁺=357.2.

### Step 7: methyl 6-(2-chloro-5-fluoropyrimidin-4-yl)-4-isopropylquinazoline-2-carboxylate

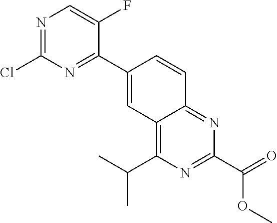 [SMILES: COC(=O)c1nc(C(C)C)c2cc(-c3nc(Cl)ncc3F)ccc2n1]

The title compound (600 mg, 59%) was prepared in a manner similar to Example 1 step 3 from methyl 4-isopropyl-6-(4,4,5,5-tetramethyl-1,3,2-dioxaborolan-2-yl)quinazoline-2-carboxylate and 2,4-dichloro-5-fluoropyrimidine. LC-MS (M+H)⁺=361.2.

### Step 8: methyl 6-(5-fluoro-2-(((3S,4R)-3-hydroxytetrahydro-2H-pyran-4-yl)amino)pyrimidin-4-yl)-4-isopropylquinazoline-2-carboxylate

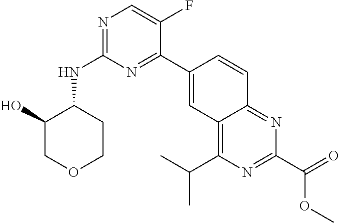 [SMILES: COC(=O)c1nc(C(C)C)c2cc(-c3nc(N[C@@H]4CCOC[C@H]4O)ncc3F)ccc2n1]

The title compound (350 mg, 46%) was prepared in a manner similar to Example 1 step 4 from methyl 6-(2-chloro-5-fluoropyrimidin-4-yl)-4-isopropylquinazoline-2-carboxylate and (3S,4R)-4-aminotetrahydro-2H-pyran-3-ol. LC-MS (M+H)⁺=442.2.

### Step 9: (3S,4R)-4-((5-fluoro-4-(2-(2-hydroxypropan-2-yl)-4-isopropylquinazolin-6-yl)pyrimidin-2-yl)amino)tetrahydro-2H-pyran-3-ol

The title compound (10 mg, 10%) was prepared in a manner similar to Example 206 step 9 from methyl 6-(5-fluoro-2-(((3S,4R)-3-hydroxytetrahydro-2H-pyran-4-yl)amino)pyrimidin-4-yl)-4-isopropylquinazoline-2-carboxylate and methylmagnesium iodide. ¹H-NMR (400 MHz, DMSO-d6) δ 8.93 (s, 1H), 8.57-8.46 (m, 2H), 8.15 (d, J=8.8 Hz, 1H), 7.34 (d, J=7.6 Hz, 1H), 5.16 (s, 1H), 4.% (d, J=5.2 Hz, 1H), 4.10-4.00 (m, 1H), 3.89-3.79 (m, 3H), 3.58-3.48 (m, 1H), 3.36 (t, J=10.3 Hz, 1H), 3.07 (t, J=10.3 Hz, 1H), 2.02 (s, 1H), 1.60 (s, 6H), 1.64-1.45 (m, 1H), 1.41 (d, J=6.7 Hz, 6H). LC-MS (M+H)⁺=442.5.

### Example 208: (3S,4R)-4-((5-chloro-4-(8-fluoro-2-(2-hydroxypropan-2-yl)-4-isopropylquinolin-6-yl)pyrimidin-2-yl)amino)tetrahydro-2H-pyran-3-ol

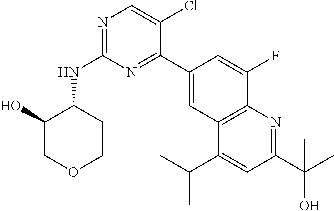 [SMILES: CC(C)c1cc(C(C)(C)O)nc2c(F)cc(-c3nc(N[C@@H]4CCOC[C@H]4O)ncc3Cl)cc12]

### Step 1: dimethyl 2-((4-chloro-2-fluorophenyl)amino)fumarate

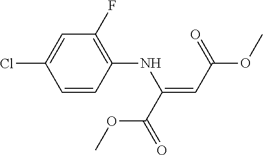 [SMILES: COC(=O)/C=C(\Nc1ccc(Cl)cc1F)C(=O)OC]

The title compound (10.5 g, 52%) was prepared in a manner similar to Example 206 step 1 from 4-chloro-2-fluoroaniline and dimethyl acetylenedicarboxylate. LC-MS (M+H)⁺=288.0.

### Step 2: methyl 6-chloro-8-fluoro-4-oxo-1,4-dihydroquinoline-2-carboxylate

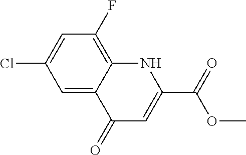 [SMILES: COC(=O)c1cc(=O)c2cc(Cl)cc(F)c2[nH]1]

The title compound (3.5 g, 87%) was prepared in a manner similar to Example 206 step 2 from dimethyl 2-((4-chloro-2-fluorophenyl)amino)fumarate. LC-MS (M+H)⁺=256.2.

### Step 3: methyl 4-bromo-6-chloro-8-fluoroquinoline-2-carboxylate

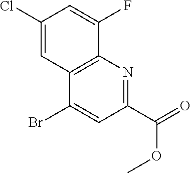 [SMILES: COC(=O)c1cc(Br)c2cc(Cl)cc(F)c2n1]

The title compound (2.38 g, 63%) was prepared m a manner similar to Example 206 step 3 from methyl 6-chloro-8-fluoro-4-oxo-1,4-dihydroquinoline-2-carboxylate and phosphorus tribromide. LC-MS (M+H)⁺=318.0.

### Step 4: methyl 6-chloro-8-fluoro-4-isopropylquinoline-2-carboxylate

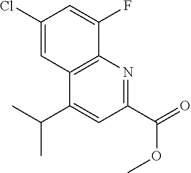 [SMILES: COC(=O)c1cc(C(C)C)c2cc(Cl)cc(F)c2n1]

The title compound (270 mg, 30%) was prepared in a manner similar to that in Example 1 step 1 from methyl 4-bromo-6-chloro-8-fluoroquinoline-2-carboxylate. LC-MS (M+H)⁺=282.2.

### Step 5: 2-(6-chloro-8-fluoro-4-isopropylquinolin-2-yl)propan-2-ol

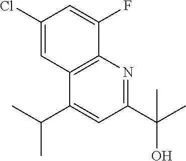 [SMILES: CC(C)c1cc(C(C)(C)O)nc2c(F)cc(Cl)cc12]

The title compound (45 mg, 26%) was prepared in a manner similar to that in Example 70 step from methyl 6-chloro-8-fluoro-4-isopropylquinoline-2-carboxylate. LC-MS (M+H)⁺=282.0.

### Step 6: 2-(8-fluoro-4-isopropyl-6-(4,4,5,5-tetramethyl-1,3,2-dioxaborolan-2-yl)quinolin-2-yl)propan-2-ol

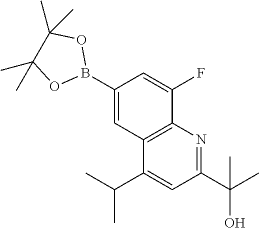 [SMILES: CC(C)c1cc(C(C)(C)O)nc2c(F)cc(B3OC(C)(C)C(C)(C)O3)cc12]

The title compound (60 mg, 90%) was prepared in a manner similar to that in Example 1 step 2 from 2-(6-chloro-8-fluoro-4-isopropylquinolin-2-yl)propan-2-ol. LC-MS (M+H)⁺=374.1.

### Step 7: 2-(6-(2,5-dichloropyrimidin-4-yl)-8-fluoro-4-isopropylquinolin-2-yl)propan-2-ol

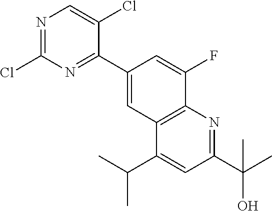 [SMILES: CC(C)c1cc(C(C)(C)O)nc2c(F)cc(-c3nc(Cl)ncc3Cl)cc12]

The title compound ((27 mg, 42%) was prepared in a manner similar to that in Example 1 step 3 from 2-(8-fluoro-4-isopropyl-6-(4,4,5,5-tetramethyl-1,3,2-dioxaborolan-2-yl)quinolin-2-yl)propan-2-ol and 2,4,5-trichloropyrimidine. LC-MS (M+H)⁺=394.0.

### Step 8: (3S,4R)-4-((5-chloro-4-(8-fluoro-2-(2-hydroxypropan-2-yl)-4-isopropylquinolin-6-yl)pyrimidin-2-yl)amino)tetrahydro-2H-pyran-3-ol

The title compound (16 mg, 44%) was prepared in a manner similar to Example 4 step 6 from 2-(6-(2,5-dichloropyrimidin-4-yl)-8-fluoro-4-isopropylquinolin-2-yl)propan-2-ol and (3S,4R)-4-aminooxan-3-ol hydrochloride. H-NMR (400 MHz, DMSO-d6) δ 8.48 (s, 1H), 8.50-8.37 (m, 1H), 7.96 (s, 1H), 7.98-7.84 (m, 1H), 7.60-7.56 (m, 1H), 5.52 (s, 1H), 4.94 (d, J=5.4 Hz, 1H), 3.89-3.68 (m, 4H), 3.53-3.49 (m, 1H), 3.38-3.32 (m, 1H), 3.09-2.99 (m, 1H), 1.99-1.95 (m, 1H), 1.56 (s, 6H), 1.58-1.48 (m, 1H), 1.39 (d, J=6.7 Hz, 6H). LC-MS (M+H)⁺=475.1.

### Example 209: (3S,4R)-4-((5-fluoro-4-(2-(2-hydroxypropan-2-yl)-4-isopropylquinazolin-6-yl)pyrimidin-2-yl)amino)tetrahydro-2H-pyran-3-ol

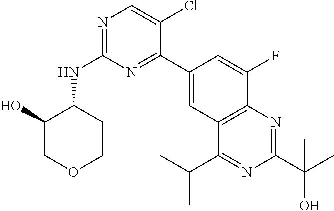 [SMILES: CC(C)c1nc(C(C)(C)O)nc2c(F)cc(-c3nc(N[C@@H]4CCOC[C@H]4O)ncc3Cl)cc12]

### Step 1: 2-amino-5-chloro-3-fluorobenzamide

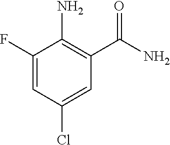 [SMILES: NC(=O)c1cc(Cl)cc(F)c1N]

The title compound (1.85 g, 81%) was prepared in a manner similar to Example 1 step 6 from 2-amino-5-chloro-3-fluorobenzoic acid and ammonium chloride. LC-MS (M+H)⁺=189.0.

### Step 2: ethyl 6-chloro-8-fluoro-4-hydroxyquinazoline-2-carbo late

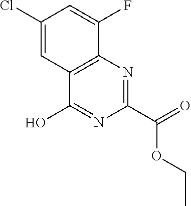 [SMILES: CCOC(=O)c1nc(O)c2cc(Cl)cc(F)c2n1]

To a solution of 2-amino-5-chloro-3-fluorobenzamide (1.57 g, 8.38 mmol) and diisopropylethylamine (1.72 g, 12.64 mmol) in tetrahydrofuran (30 mL) was added ethyl chloroglyoxylate (1.275 g, 9.32 mmol) dropwise at 0° C. under nitrogen atmosphere. The resulting mixture was stirred for 3 h at room temperature before quenched by the addition of saturated sodium bicarbonate (30 mL) at room temperature. The resulting mixture was stirred for 1 h and the organic layer was separated from aqueous layer. The aqueous layer was extracted with ethyl acetate (80 mL×2). The combined organic layers were washed with brine, dried over sodium sulfate, filtered and concentrated under reduced pressure. The residue was purified by flash chromatography eluting with ethyl acetate in petroleum ether (0% to 30% gradient, v/v) to yield the title compound (850 mg, 37%). LC-MS (M+H)⁺=270.9.

### Step 3: ethyl 4-bromo-6-chloro-8-fluoroquinazoline-2-carboxy late

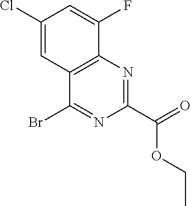 [SMILES: CCOC(=O)c1nc(Br)c2cc(Cl)cc(F)c2n1]

The title compound (0.6 g, 67%) was prepared in a manner similar to Example 206 step 3 from ethyl 6-chloro-8-fluoro-4-hydroxyquinazoline-2-carboxylate and phosphorus oxybromide. LC-MS (M+H)⁺=332.9.

### Step 4: ethyl 6-chloro-8-fluoro-4-isopropylquinazoline-2-carboxylate

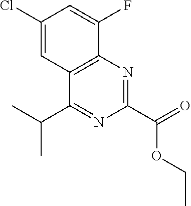 [SMILES: CCOC(=O)c1nc(C(C)C)c2cc(Cl)cc(F)c2n1]

The title compound (120 mg, 22%) was prepared in a manner similar to Example 1 step 1 from ethyl 4-bromo-6-chloro-8-fluoroquinazoline-2-carboxylate. LC-MS (M+H)⁺=297.1.

### Step 5: 2-(6-chloro-8-fluoro-4-isopropylquinazolin-2-yl)propan-2-ol

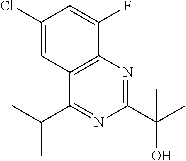 [SMILES: CC(C)c1nc(C(C)(C)O)nc2c(F)cc(Cl)cc12]

The title compound (70 mg, 61%) was prepared in a manner similar to that in Example 70 step from ethyl 6-chloro-8-fluoro-4-isopropylquinazoline-2-carboxylate. LC-MS (M+H)⁺=283.0.

### Step 6: 2-(8-fluoro-4-isopropyl-6-(4,4,5,5-tetramethyl-1,3,2-dioxaborolan-2-yl)quinazolin-2-yl)propan-2-ol

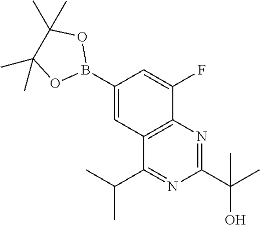 [SMILES: CC(C)c1nc(C(C)(C)O)nc2c(F)cc(B3OC(C)(C)C(C)(C)O3)cc12]

The title compound (100 mg, crude used for next step) was prepared in a manner similar to that in Example 1 step 2 from 2-(6-chloro-8-fluoro-4-isopropylquinazolin-2-yl)propan-2-ol. LC-MS (M+H)⁺=375.2

### Step 7: 2-(6-(2,5-dichloropyrimidin-4-yl)-8-fluoro-4-isopropylquinazolin-2-yl)propan-2-ol

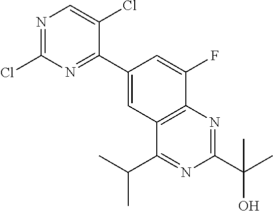 [SMILES: CC(C)c1nc(C(C)(C)O)nc2c(F)cc(-c3nc(Cl)ncc3Cl)cc12]

The title compound (40 mg, 41% for 2 steps) was prepared in a manner similar to that in Example 1 step 3 from 2-(8-fluoro-4-isopropyl-6-(4,4,5,5-tetramethyl-1,3,2-dioxaborolan-2-yl)quinazolin-2-yl)propan-2-ol and 2,4,5-trichloropyrimidine. LC-MS (M+H)⁺=395.0

### Step 8: (3S,4R)-4-((5-chloro-4-(8-fluoro-2-(2-hydroxypropan-2-yl)-4-isopropylquinazolin-6-yl)pyrimidin-2-yl)amino)tetrahydro-2H-pyran-3-ol

The title compound (28 mg, 41%) was prepared in a manner similar to that in Example 4 step 6 from 2-(6-(2,5-dichloropyrimidin-4-yl)-8-fluoro-4-isopropylquinazolin-2-yl)propan-2-ol and (3S,4R)-4-aminooxan-3-ol hydrochloride. ¹H-NMR (400 MHz, DMSO-d6) δ 8.41 (s, 1H), 7.95-7.90 (m, 1H), 7.88-7.81 (m, 1H), 7.53-7.47 (m, 1H), 5.19 (s, 1H), 4.91 (d, J=5.5 Hz, 1H), 3.89-3.75 (m, 3H), 3.53-3.41 (m, 1H), 3.39-3.28 (m, 1H), 3.10-3.00 (m, 1H), 2.97-2.85 (m, 1H), 1.96-1.89 (m, 1H), 1.55-1.39 (m, 7H), 1.19-1.13 (m, 6H). LC-MS (M+H)⁺=476.2.

### Biological Assays

Compounds disclosed herein were tested for inhibition of CDK4/Cyclin D1 or CDK6/Cyclin D3 kinase in an assay based on the time-resolved fluorescence-resonance energy transfer (TR-FRET) methodology. The assay was carried out in 384-well low volume black plates in a reaction mixture containing CDK4/Cyclin D1 or CDK6/Cyclin D3, 1 mM ATP, 0.15 μM Rb (Ser780)-biotin substrate and 0-10 μM compound in buffer containing 50 mM HEPES pH7.0, 0.02% NaN3, 0.01% BSA, 0.1 mM Orthovanadate, 50 mM MgCl2, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 120 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK4/Cyclin D1 or CDK6/Cyclin D3 kinase activity in presence of increasing concentrations of compounds was calculated based on the ratio of fluorescence at 665 nm to that at 620 nm. The IC50 for each compound was derived from fitting the data to the four-parameter logistic equation by Dotmatics.

TABLE 1
Enzymatic activity IC50 (nM) for the compounds disclosed herein

| Example No. | CDK4D1 IC50 (nM) | CDK6D3 IC50 (nM) |
|---|---|---|
| 1 | 67 | 670 |
| 2 | 4.6 | 69 |
| 3 | 4.9 | 73.5 |
| 4 | 38 | 418 |
| 5 | 108 | 810 |
| 6 | 9.3 | 158 |
| 7 | 44 | 528 |
| 8 | 14 | 252 |
| 9 | 21 | 92.4 |
| 10 | 77 | 924 |
| 11 | 21 | 567 |
| 12 | 19 | 323 |
| 13 | 16 | 464 |
| 14 | 12 | 228 |
| 15 | 43 | 559 |
| 16 | 15 | 600 |
| 17 | 11 | 187 |
| 18 | 26 | 598 |
| 19 | 20 | 1220 |
| 20 | 12 | 600 |
| 21 | 15 | 495 |
| 22 | 7.1 | 397.6 |
| 23 | 19 | 361 |
| 24 | 20 | 620 |
| 25 | 8.3 | 224.1 |
| 26 | 33 | 990 |
| 27 | 23 | 828 |
| 28 | 19 | 703 |
| 29 | 20 | 860 |
| 30 | 11 | 550 |
| 31 | 25 | 275 |
| 32 | 11 | 484 |
| 33 | 22 | 792 |
| 34 | 27 | 783 |
| 35 | 16 | 592 |
| 36 | 14 | 224 |
| 37 | 27 | 945 |
| 38 | 4.4 | 176 |
| 39 | 25 | 750 |
| 40 | 33 | 759 |
| 41 | 10 | 170 |
| 42 | 32 | 480 |
| 43 | 41 | 533 |
| 44 | 25 | 475 |
| 45 | 22 | 286 |
| 46 | 65 | 1040 |
| 47 | 25 | 250 |
| 48 | 21 | 756 |
| 49 | 14 | 392 |
| 50 | 4.4 | 184.8 |
| 51 | 4.2 | 67.2 |
| 52 | 10 | 240 |
| 53 | 12 | 348 |
| 54 | 19 | 361 |
| 55 | 34 | 1088 |
| 56 | 3.5 | 213.5 |
| 57 | 18 | 486 |
| 58 | 14 | 406 |
| 59 | 46 | 3588 |
| 60 | 15 | 585 |
| 61 | 8.7 | 426.3 |
| 62 | 11 | 572 |
| 67 | 2.5 | 42.7 |
| 68 | 8.7 | 395 |
| 69 | 8.3 | 355 |
| 70 | 31 | 508 |
| 116 | 10.9 | 198 |
| 117 | 2.1 | 54 |
| 120 | 23.5 | 149 |
| 145 | 1.7 | 66.1 |
| 206 | 185 | 1500 |
| 207 | 536 | 3019 |
| 208 | 146 | 497 |
| 209 | 1772 | 4986 |

1-18. (canceled)

19. A compound is selected from 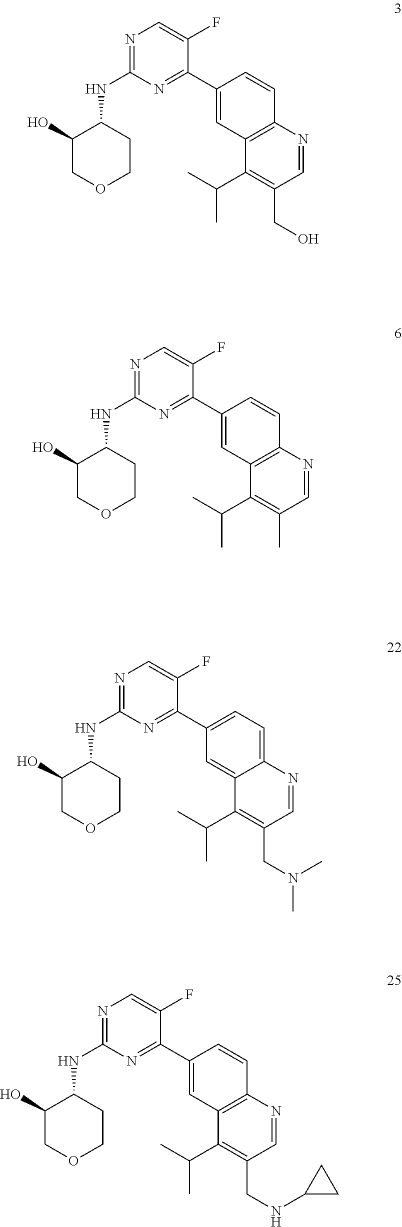 [SMILES: CC(C)c1c(CO)cnc2ccc(-c3nc(N[C@@H]4CCOC[C@H]4O)ncc3F)cc12 ; Cc1cnc2ccc(-c3nc(N[C@@H]4CCOC[C@H]4O)ncc3F)cc2c1C(C)C ; CC(C)c1c(CN(C)C)cnc2ccc(-c3nc(N[C@@H]4CCOC[C@H]4O)ncc3F)cc12 ; CC(C)c1c(CNC2CC2)cnc2ccc(-c3nc(N[C@@H]4CCOC[C@H]4O)ncc3F)cc12] 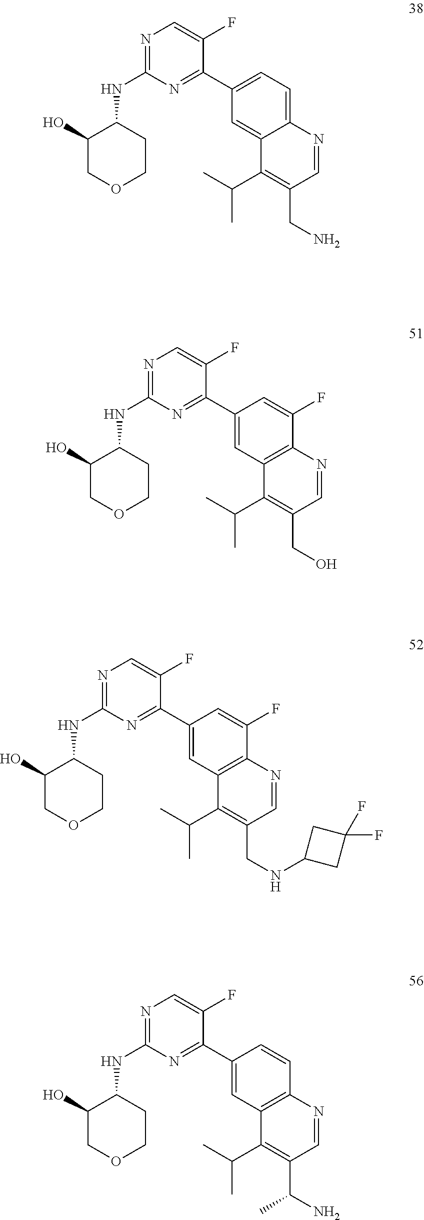 [SMILES: CC(C)c1c(CN)cnc2ccc(-c3nc(N[C@@H]4CCOC[C@H]4O)ncc3F)cc12 ; CC(C)c1c(CO)cnc2c(F)cc(-c3nc(N[C@@H]4CCOC[C@H]4O)ncc3F)cc12 ; CC(C)c1c(CNC2CC(F)(F)C2)cnc2c(F)cc(-c3nc(N[C@@H]4CCOC[C@H]4O)ncc3F)cc12 ; CC(C)c1c([C@@H](C)N)cnc2ccc(-c3nc(N[C@@H]4CCOC[C@H]4O)ncc3F)cc12] 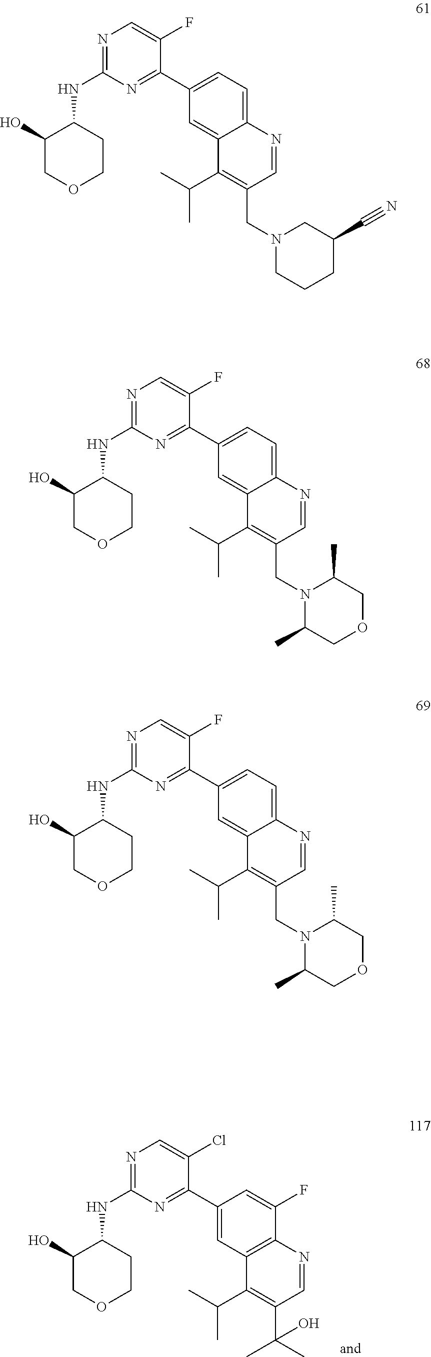 [SMILES: CC(C)c1c(CN2CCC[C@H](C#N)C2)cnc2ccc(-c3nc(N[C@@H]4CCOC[C@H]4O)ncc3F)cc12 ; CC(C)c1c(CN2[C@H](C)COC[C@@H]2C)cnc2ccc(-c3nc(N[C@@H]4CCOC[C@H]4O)ncc3F)cc12 ; CC(C)c1c(CN2[C@H](C)COC[C@H]2C)cnc2ccc(-c3nc(N[C@@H]4CCOC[C@H]4O)ncc3F)cc12 ; CC(C)c1c(C(C)(C)O)cnc2c(F)cc(-c3nc(N[C@@H]4CCOC[C@H]4O)ncc3Cl)cc12] 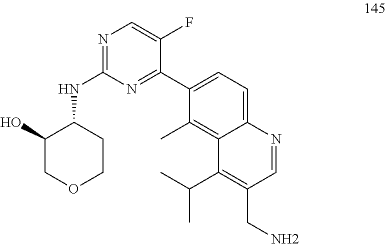 [SMILES: Cc1c(-c2nc(N[C@@H]3CCOC[C@H]3O)ncc2F)ccc2ncc(CN)c(C(C)C)c12]
or a pharmaceutically acceptable salt thereof.

20. A pharmaceutical composition comprising a compound of claim 19 or a pharmaceutically acceptable salt or tautomer thereof, together with a pharmaceutically acceptable excipient.

21. A method of decreasing CDK4 activity by inhibition, comprising administering to a subject in need thereof a therapeutically effective amount of a compound of claim 19, or a pharmaceutically acceptable salt or tautomer thereof.

22-24. (canceled)

25. A method of treating a disease or disorder in a patient comprising administering to the patient a therapeutically effective amount of a compound of claim 19, or a pharmaceutically acceptable salt or tautomer thereof, wherein the disease or disorder is associated with inhibition of CDK4.

26. (canceled)

27. The compound of claim 19, of the following formula: 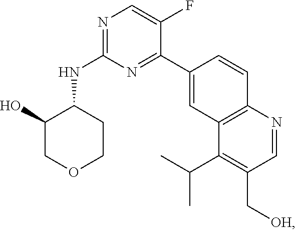 [SMILES: CC(C)c1c(CO)cnc2ccc(-c3nc(N[C@@H]4CCOC[C@H]4O)ncc3F)cc12]
or a pharmaceutically acceptable salt thereof.

28. The compound of claim 19, of the following formula: 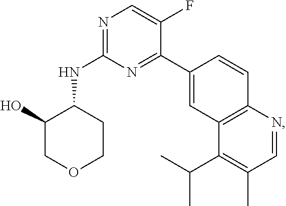 [SMILES: Cc1cnc2ccc(-c3nc(N[C@@H]4CCOC[C@H]4O)ncc3F)cc2c1C(C)C]
or a pharmaceutically acceptable salt thereof.

29. The compound of claim 19, of the following formula: 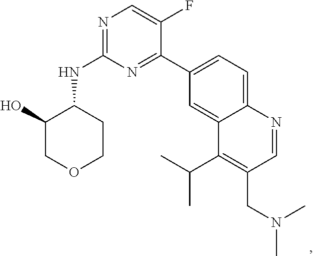 [SMILES: CC(C)c1c(CN(C)C)cnc2ccc(-c3nc(N[C@@H]4CCOC[C@H]4O)ncc3F)cc12]
or a pharmaceutically acceptable salt thereof.

30. The compound of claim 19, of the following formula: 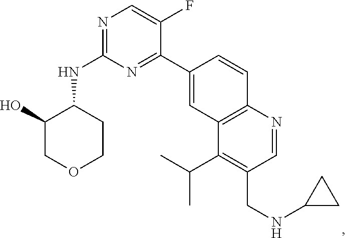 [SMILES: CC(C)c1c(CNC2CC2)cnc2ccc(-c3nc(N[C@@H]4CCOC[C@H]4O)ncc3F)cc12]
or a pharmaceutically acceptable salt thereof.

31. The compound of claim 19, of the following formula: 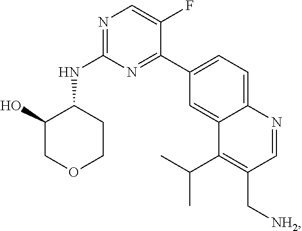 [SMILES: CC(C)c1c(CN)cnc2ccc(-c3nc(N[C@@H]4CCOC[C@H]4O)ncc3F)cc12]
or a pharmaceutically acceptable salt thereof.

32. The compound of claim 19, of the following formula: 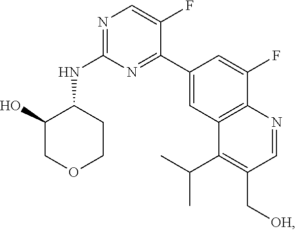 [SMILES: CC(C)c1c(CO)cnc2c(F)cc(-c3nc(N[C@@H]4CCOC[C@H]4O)ncc3F)cc12]
or a pharmaceutically acceptable salt thereof.

33. The compound of claim 19, of the following formula: 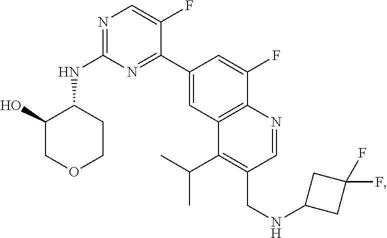 [SMILES: CC(C)c1c(CNC2CC(F)(F)C2)cnc2c(F)cc(-c3nc(N[C@@H]4CCOC[C@H]4O)ncc3F)cc12]
or a pharmaceutically acceptable salt thereof.

34. The compound of claim 19, of the following formula: 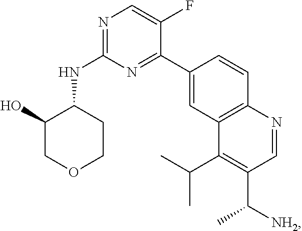 [SMILES: CC(C)c1c([C@@H](C)N)cnc2ccc(-c3nc(N[C@@H]4CCOC[C@H]4O)ncc3F)cc12]
or a pharmaceutically acceptable salt thereof.

35. The compound of claim 19, of the following formula: 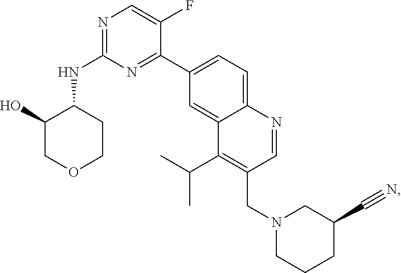 [SMILES: CC(C)c1c(CN2CCC[C@H](C#N)C2)cnc2ccc(-c3nc(N[C@@H]4CCOC[C@H]4O)ncc3F)cc12]
or a pharmaceutically acceptable salt thereof.

36. The compound of claim 19, of the following formula: 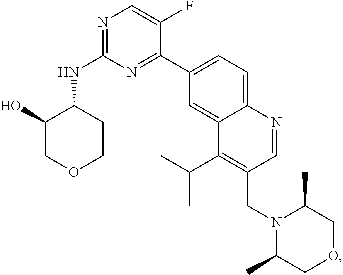 [SMILES: CC(C)c1c(CN2[C@H](C)COC[C@@H]2C)cnc2ccc(-c3nc(N[C@@H]4CCOC[C@H]4O)ncc3F)cc12]
or a pharmaceutically acceptable salt thereof.

37. The compound of claim 19, of the following formula: 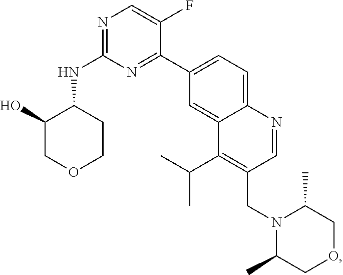 [SMILES: CC(C)c1c(CN2[C@H](C)COC[C@H]2C)cnc2ccc(-c3nc(N[C@@H]4CCOC[C@H]4O)ncc3F)cc12]
or a pharmaceutically acceptable salt thereof.

38. The compound of claim 19, of the following formula: 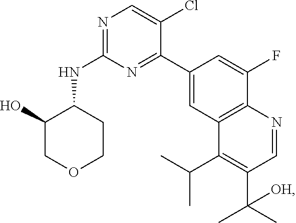 [SMILES: CC(C)c1c(C(C)(C)O)cnc2c(F)cc(-c3nc(N[C@@H]4CCOC[C@H]4O)ncc3Cl)cc12]
or a pharmaceutically acceptable salt thereof.

39. The compound of claim 19, of the following formula: 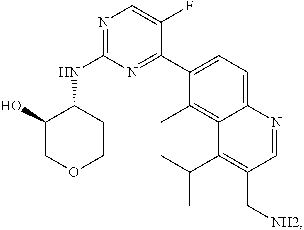 [SMILES: Cc1c(-c2nc(N[C@@H]3CCOC[C@H]3O)ncc2F)ccc2ncc(CN)c(C(C)C)c12]
or a pharmaceutically acceptable salt thereof.
